(19) United States
(12) Patent Application Publication
(10) Pub. No.: US 2006/0063785 A1
(43) Pub. Date: Mar. 23, 2006

(54) Substituted pyrazolo[1,5-a] pyrimidines and process for making same

(76) Inventors: Yanong Daniel Wang, Green Brook, NJ (US); Ariamala Gopalsamy, Mahwah, NJ (US); Dennis William Powell, Cortlandt Manor, NY (US); Hwei-Ru Tsou, New City, NY (US); Nan Zhang, Bayside, NY (US)

Correspondence Address: FITZPATRICK CELLA HARPER & SCINTO, 30 ROCKEFELLER PLAZA, NEW YORK, NY, 10112, US

(21) Appl. No.: 11/221,847

(22) Filed: Sep. 9, 2005

Related U.S. Application Data
(60) Provisional application No. 60/610,520, filed on Sep. 17, 2004.

(51) Int. Cl. A61K 31/519; C07D 487/04

(52) U.S. Cl. 514/259.3; 544/281

(57) ABSTRACT

This invention relates to novel pyrazolo[1,5-a]pyrimidine compounds and the therapeutically acceptable salts thereof. These compounds are useful as anti-proliferative agents in mammals, including humans.

This application claims the benefit of U.S. Provisional Application No. 60/610,520, filed Sep. 17, 2004, which is incorporated by reference herein.

### BACKGROUND OF THE INVENTION

This invention relates to novel pyrazolo[1,5-a]pyrimidine compounds as well as the pharmaceutically acceptable salts thereof. The compounds of this invention may be used as anti-proliferative agents in mammals, including humans.

Deregulation of cell proliferation, or a lack of appropriate cell death, has a wide range of clinical implications, including cancers, restenosis, angiogenesis, hyperplasia, endometriosis, lymphoproliferative disorders, graft rejection and the like. Such cells may lack the normal regulatory control of cell division, and therefore fail to undergo appropriate cell death.

Progression from one phase of the cell division cycle to the next phase is controlled by a series of sensors and arresting mechanisms called cell cycle checkpoints [Zhou, B. B, et al. Nature 408, 433 (2000) and Weinert, T. A., et al, Genes Dev., 8, 652 (1994)]. Through regulation of the cyclin-dependent kinases and their obligate activating partners, the cyclins, checkpoints ensure that each step in the cell cycle has been successfully completed before the onset of the next phase. At each checkpoint, the cell determines whether it is ready for progression to the next phase or halts the progression if conditions are unfavorable, for example, if the nutrients are insufficient or if DNA damage has not been repaired [Keith, C. T., et al,. Science, 270, 50 (1995)]. Deregulation of a cell cycle phase transition may occur as a consequence of the aberrant expression of positive regulators, such as the cyclins, loss of negative regulators (CDK inhibitors), e.g., p21, p27, p15, p16, p18, and p19, or the inactivation of tumor suppressor genes, such as p53 and pRb. Loss of cell cycle checkpoint control is a hallmark of tumor cells, as it increases the mutation rate and allows a more rapid progression to the tumorigenic state. Inactivation of these check points can result in abberant responses to cellular damage. For example, a cell with intact DNA damage control checkpoints will arrest at the G1/S and G2/M boundaries of the cell cycle in response to low levels of DNA damaging agents. Disruption of the checkpoint leads to the failure of the cell to arrest, multiple rounds of DNA synthesis in the presence of damaged DNA, and ultimately, apoptosis. This failure of cell cycle arrest responses in malignant cells can be exploited therapeutically in an innovative screening approach: identification of compounds that by selectively killing checkpoint-deficient cells compared with checkpoint-proficient cells can be expected to preferentially target tumor cells, while sparing normal cells. Novel anti-tumor agents identified by these screening methods are likely to be more effective and safer than current therapies for cancer. The publication WO 97/34640 describes this strategy for drug screening that was developed based on isogenic human cancer cell lines in which key checkpoint regulators have been deleted by targeted homologous recombination. These isogenic cell lines can then be used in parallel with the corresponding unmodified cells to screen for therapeutic compounds with selective toxicity toward any desired genotype [Torrance, C. J., et al, Nature Biotech., 19, 940 (2001)].

A major cell cycle checkpoint regulator, the protein p₂₁^{Waf1/Cip/Sid1} (hereafter referred to as p21) was originally isolated as a general inhibitor of CDKs [El-Deiry, W. S., et al, Cell, 75, 817 (1993) and Harper, J. W., et al, Cell, 75, 805 (1993)]. p21 inhibits progression of the cell cycle by inhibiting the activity of GI kinases (cyclin D/cdk4 and cyclin E-cdk2) and the G2 kinase (cyclin B/cdk1) in response to DNA damage or abnormal DNA content [Xiong, Y., et al, Nature, 366, 701 (1993) and Sherr, C. J., et al, Genes Dev., 9, 1149 (1995)]. Regulation of p21 levels occurs transcriptionally by p53-dependent and p53-independent mechanisms. Upon DNA damage, p21 is strongly up-regulated, reaching the levels that completely arrest proliferation. Cells derived from p21-null mice arrest proliferation inefficiently after p53 activation [Brugarolas, J., et al, Nature, 377, 552, (1995) and Brugarolas, J., et al, Proc. Natl. Acad. Sci. USA, 96, 1002 (1999)]. The expression of p21 is often low in human cancer cells due to frequent loss of the upstream activator, p53, and is associated with poor prognosis in some cancer patients [Karjalainen, J. M., Br. J. Cancer, 79, 895 (1999) and Komiya, T., et al, Clin. Cancer Res., 3, 1831 (1997)]. A colon cancer cell line and the isogenic p21-deficient cells generated by targeted gene deletion [Waldman, T. et al. Cancer Res., 55, 5187-5190 (1995); Waldman T. et al. Nature, 381, 713-716 (1996)] was used to identify compounds that preferentially inhibit the p21-deficient cells. It would be useful to have a method of selectively inhibiting the growth of tumor cells compared with normal cells. Small drug molecules should represent an important and useful approach to treating cancer and aberrant cell growth. Pyrazolo[1,5-a]pyrimidines compounds with antitumor activity were identified using the above described screening method.

Other pyrazolo[1,5-a]pyrimidines have been described as having anxiolytic activity (U.S. Pat. No. 4,654,347; U.S. Pat. No. 4,236,005; U.S. Pat. No. 4,521,422; U.S. Pat. No. 4,281,000; U.S. Pat. No. 4,626,538; U.S. Pat. No. 4,576,943; U.S. Pat. No. 5,059,691; U.S. Pat. No. 5,538,977; EP 0129847; and EP 0208846).

DE4333705 described substituted pyrazolo[1,5-a]pyrimidines as useful medicinal agents. These pyrazolopyrimidines however must contain a substituent consisting of an arylmethyl group at the C-3 position.

International patent publication WO 96/35690 described substituted pyrazolo[1,5-a]pyrimidines as pesticides and fungicides. These pyrazolopyrimidines however must contain phenyl substituents linked by O or S at the C-2 position.

EP0941994 described substituted pyrazolo[1,5-a]pyrimidines as having selective affinity to 5HT-6 receptors. However, these pyrazolopyrimidines must contain an arylsulphonyl or alkylsulphonyl group at the C-3 position.

International patent publication WO 02/12244 described alternative methods for making polymorphic, crystalline forms of substituted pyrazolopyrimidines in general, and more particularly, N-[3-(3cyanopyrazolo[1,5a]pyrimidin-7-yl)phenyl]-N-ethylacetamide (Zaleplon), which is used as an anxiolytic or antiepileptic agent.

### SUMMARY OF THE INVENTION

The present invention is directed to novel compounds used for treating cancer and aberrant cell growth, or neoplasms. It is further directed to compounds, e.g. certain substituted pyrazolo[1,5-a]pyrimidines, and the therapeutically acceptable salts thereof, that selectively inhibit the proliferation of p21 deficient cells. Thus, the compounds of the present invention, involving the pyrrazolo[1,5-a]pyrimidine system, will be numbered as indicated in the formula below:

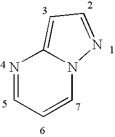

The compounds of this invention include compounds represented by the following structural formula: 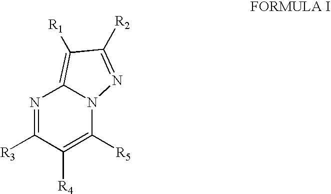
and the pharmaceutically acceptable salts and prodrugs thereof, wherein:

R, is selected from the groups consisting of hydrogen, cyano, halogen, carbamoyl, formyl, carboxy, C(O)O-alkyl, C(O)O-cycloalkyl, C(O)cycloalkyl, R₆, C(O)R₆, and C(S)R₆.

R₆ is unsubstituted, monosubstituted or disubstituted aryl or heteroaryl wherein the aryl or heteroaryl is phenyl, naphthalenyl, thiazolyl, biphenyl, thienyl, furanyl, and pyridinyl; and the substituents are selected from the groups consisting of hydrogen, halogen, nitro, cyano, CF₃, OCF₃, alkyl, alkoxy, alkanol, trifluoromethyl, alkylamino, alkylthio, dialkylamino, methylenedioxy, alkylsulfonyl and alkanoylamino.

R₂, R₃, and R₄ are independently hydrogen, CF₃, or alkyl. R₅ is unsubstituted aryl or heteroaryl, or aryl or heteroaryl substituted by R₇, R₈, R₉, or R₁₀ with the proviso that R₁₀ must be present if the aryl or heteroaryl is substituted. Preferred moieties for R₅ are phenyl, naphthalenyl, thiazolyl, biphenyl, thienyl, furanyl and pyridinyl.

R₇, R₈, and R₉ are independently selected from the groups consisting of hydrogen, nitro, cyano, carboxy, carbamoyl, halogen, N(CH₃)₂, CF₃, OCF₃, alkyl, and alkoxy.

R₁₀ is selected from nitro, cyano, carboxy, carbamoyl, halogen, CF₃, OCF₃, alkyl, alkoxy, alkanol, NR₁₁R₁₂, N(R₁₃)COR₁₁, N(R₁₃)CONR₁₁R₁₂, OCONR₁₁R₁₂, N(R₁₃)CO₂R₁₁, N(R₁₃)CSR₁₁, N(R₁₃)CS(NR₁₁R₁₂), N(R₁₃)CS(OR₁₁), N(R₁₃)SO₂R₁₁, N(CONR₁₃R₁₁)₂, N(CO₂R₁₁)₂, N(COR₁₁)₂, N(CONR₁₃R₁₁)₂, CONR₁₁R₁₂, CO₂R₁₁, NHC(═NH)NHR₁₁, NHC(═NH—CN)NR₁₁R₁₂, NHC(═NH—CN)OR₁₁, C(═NH)NHR₁₁, C(═NH)NR₁₁R₁₂, LCONH₂, LCONR₁₁R₁₂, or LCO₂R₁₁ wherein L is alkyl, alkenyl or alkynyl or R₁₀ is selected from unsubstituted, monosubstituted, or disubstituted aryl or heteroaryl which is a 5- or 6-membered aromatic ring moiety containing at least 1-4 heteroatoms selected from O, S, and N. Preferred aryl or heteroaryl moieties for R₁₀ are phenyl, naphthalenyl, thiazolyl, biphenyl, thienyl, furanyl and pyridinyl.

R₁₁ and R₁₂ are independently selected from the groups consisting of hydrogen, alkyl, alkenyl, alkynyl, cycloalkyl, Q₁, Q₂, -L-Q₁ and -L-Q₂. Q₁ is unsubstituted aryl or heteroaryl, monosubstituted or disubstituted aryl or heteroaryl. Preferred moieties for Q₁ are phenyl, naphthalenyl, biphenyl, thiazolyl, oxazolyl, pyrrolyl, pyrrazolyl, thienyl, furanyl, 1,2,3-triazolyl, 1,2,4-triazolyl, tetrazolyl, pyrrolidinyl, imidazolyl, and pyridinyl, and the substituents are selected from halogen, alkyl, CF₃, OCF₃, cyano, nitro, carboxy, hydroxy, alkoxy. Q₂ is alkyl or heterocycle containing at least one and up to 4 heteroatoms selected from O, S, and N, optionally including their common protecting groups. Q₂ further comprises alkyl or cycloalkyl containing or substituted by functional groups selected from halogen, carboxy, carbamoyl, hydroxy, carbonyl, and cycloalkyl with a site of unsaturation.

R₁₁ and R₁₂ together with the N to which they are attached may join to form a 3 to 8 membered ring.

R₁₃ is hydrogen, alkyl, alkenyl, alkynyl or cycloalkyl; R₁₁ and R₁₃ together with the N to which they are attached may join to form a 3 to 8 membered ring.

The present invention, as described above, is described with the following provisos. When R₁ is C(O)R₆ and when R₆ is unsubstituted phenyl; phenyl monosubstituted or di-substituted by halogen, alkoxy or alkyl; phenyl monosubstituted by alkylthio, alkylamino, dialkylamino, methylenedioxy, alkylsulfonyl, alkanoylamino; naphthalenyl; thiazolyl; biphenyl; thienyl; furanyl; pyridinyl; substituted thiazolyl; substituted biphenyl; thienyl or pyridinyl substituted by one or two of the group consisting of halogen, alkoxy and alkyl, then R₅ may not be unsubstituted phenyl, phenyl monosubstituted by halogen, CF₃, alkoxy, amino, alkyl, alkylamino, dialkylamino, alkanoylamino, N-alkylalkanoylamino, cyano or alkylthio; unsubstituted furanyl; unsubstituted thienyl; unsubstituted pyridinyl; or unsubstituted pyridine-1-oxide.

When R₁ is hydrogen, fluoro, chloro, bromo, formyl, carboxy, cyano, C(O)alkyl, or CO(O)alkyl, then R₅ may not be phenyl monosubstituted by halogen, CF₃, alkoxy; unsubstituted pyridinyl, pyridine N-oxide, thienyl, furanyl; or pyridinyl, pyridine N-oxide, thienyl and furanyl monosubstituted by alkyl.

When R₁ is hydrogen, halogen, cyano, formyl, C(O)alkyl, CO(O)alkyl and R₅ is phenyl substituted by R₁₀; and when R₁₀ is N(R₁₃)COR₁₁ where R₁₃ is hydrogen, alkyl, alkenyl, propynyl, or cycloalkylmethyl, then R₁₁ may not be alkyl, cycloalkyl, alkoxy, alkylamino, dialkylamino, —(CH₂)ₙ—O-alkyl, —(CH₂)ₙ—NH-alkyl, —(CH₂)ₙ—N-dialkyl wherein n is an integer 1 to 3 inclusive.

When R₁ is hydrogen, cyano, chloro, or C(O)R₆ wherein R₆ is phenyl, phenyl monosubstituted by halogen, alkoxy, alkyl, trifluoromethyl, alkylthio, alkylamino, or dialkylamino; thienyl, furanyl, pyridinyl; furanyl, thienyl, pyridinyl monosubstituted by halogen, alkoxy or alkyl, then R₅ may not be meta-substituted phenyl wherein the substituent is an imidazolyl or imidazolyl substituted by 1-3 alkyl groups with the imidazolyl nitrogen directly attached to the phenyl ring.

When R₁ is C(O)R and R₆ is unsubstituted phenyl; phenyl monosubstituted or di-substituted by halogen, alkoxy or alkyl; phenyl monosubstituted by alkylthio, alkylamino, dialkylamino, methylenedioxy, alkylsulfonyl, alkanoylamino; naphthalenyl; thiazolyl; biphenyl; thienyl; furanyl; pyridinyl; substituted thiazolyl; substituted biphenyl; thienyl or pyridinyl substituted by one or two of the group consisting of halogen, alkoxy and alkyl; and when R₅ is phenyl substituted by R₁₀; wherein R₁₀ is N(R₁₃)COR₁₁, R₁₃ is hydrogen, alkenyl, propynyl, or cycloalkylmethyl, then R₁₁ may not be alkyl, cycloalkyl, alkoxy, alkylamino, dialkylamino, —(CH₂)ₙ—O-alkyl, —(CH₂)ₙ—NH-alkyl, —(CH₂)ₙ—N-dialkyl wherein n is an integer 1 to 3 inclusive.

Compounds of this invention include pyrazolo[1,5-a]pyrimidines of Formula I wherein R₁ is selected from the groups consisting of cyano, halogen, C(O)Oalkyl, C(O)O-cycloalkyl, C(O)cycloalkyl, R₆, C(O)R₆, C(S)R₆; R₂, R₃, and R₄ are hydrogen, CF₃, or alkyl; R₅ is unsubstituted aryl or heteroaryl, or aryl or heteroaryl substituted by R₇, R₈, R₉, or R₁₀ with the proviso that R₁₀ must be present; R₆, R₇, R₈, R₉, and R₁₀ are as defined before.

In another embodiment of this invention, the compounds include pyrazolo[1, 5-a]pyrimidines of Formula I wherein R₁ is C(O)R₆, C(S)R₆; R₂, R₃, and R₄ are hydrogen, CF₃, or alkyl; R₅ is unsubstituted aryl or heteroaryl, or aryl or heteroaryl substituted by R₇, R₈, R₉, or R₁₀ with the proviso that R₁₀ must be present; R₆, R₇, R₈, R₉, and R₁₀ are as defined before.

Furthermore compounds of this invention include pyrazolo[1,5-a]pyrimidines of Formula I wherein R₁ is C(O)R₆, C(S)R₆; R₂, R₃, and R₄ are hydrogen; R₅ is unsubstituted aryl or heteroaryl, or aryl or heteroaryl substituted by R₇, R₈, R₉, or R₁₀ with the proviso that R₁₀ must be present; R₆, R₇, R₈, R₉, and R₁₀ are as defined before.

Another embodiment of the present invention includes pyrazolo[1,5-a]pyrimidines of Formula I wherein R₁ is C(O)R₆, C(S)R₆; R₂, R₃, and R₄ are hydrogen; R₅ is aryl or heteroaryl substituted by R₇, R₃, R₉, or R₁₀ with the proviso that R₁₀ must be present; R₆, R₇, R₈, R₉, and R₁₀ are as defined before.

The present also includes compounds or the pharmaceutically acceptable salt thereof, of the Formula II, 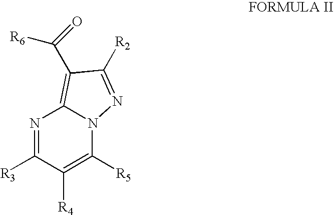
and pharmaceutically acceptable salts and prodrugs thereof,

R₂, R₃, and R₄ are hydrogen, CF₃, or alkyl;

R₆ is unsubstituted, monosubstituted or disubstituted aryl or heteroaryl wherein the aryl or heteroaryl group is phenyl, naphthalenyl, thiazolyl, biphenyl, thienyl, furanyl, or pyridinyl; and the substituents on the aryl or heteroaryl ring are selected from the groups consisting of halogen, nitro, cyano, CF₃, OCF₃, alkyl, alkoxy, trifluoromethyl, alkanol, alkylamino, alkylthio, dialkylamino, methylenedioxy, alkylsulfonyl and alkanoylamino;

R₅ is unsubstituted aryl or heteroaryl, or aryl or heteroaryl substituted by R₇, R₈, R₉, or R₁₀ with the proviso that R₁₀ must be present if the aryl or heteroaryl is substituted; wherein the aryl or heteroaryl is phenyl, naphthalenyl, thiazolyl, biphenyl, thienyl, furanyl, or pyridinyl;

R₇, R₈, and R₉ are independently selected from the groups consisting of hydrogen, nitro, cyano, carbamoyl, halogen, N(CH₃)₂, CF₃, OCF₃, alkyl, alkoxy, and carboxy;

R₁₀ is selected from nitro, cyano, carboxy, carbamoyl, halogen, CF₃, OCF₃, alkyl, alkoxy, alkanol, NR₁₁R₁₂, N(R₁₃)COR₁₁, N(R₁₃)CONR₁₁R₁₂, OCONR₁₁R₁₂, N(R₁₃)CO₂R₁₁, N(R₁₃)CSR₁₁, N(R₁₃)CS(NR₁₁R₁₂), N(R₁₃)CS(OR₁₁), N(R₁₃)SO₂R₁₁, N(CONR₁₃R₁₁)₂, N(CO₂R₁₁)₂, N(COR₁₁)₂, N(CONR₁₃R₁₁)₂, CONR₁₁R₁₂, CO₂R₁₁, NHC(═NH)NHR₁₁, NHC(═NH—CN)NR₁₁R₁₂, NHC(═NH—CN)OR₁₁, C(═NH)NHR₁₁, C(═NH)NR₁₁R₁₂, LCONH₂, LCONR₁₁R₁₂, or LCO₂R₁₁ wherein L is alkyl, alkenyl or alkynyl or R₁₀ is selected from unsubstituted, monosubstituted or disubstituted aryl or heteroaryl which is a 5- or 6-membered aromatic ring moiety containing at least 1-4 heteroatoms selcted from O, S, and N;

and R₁₁, R₁₂ and R₁₃ are defined as before.

The present invention also relates to compounds and their pharmaceutically acceptable salts thereof according to Formula III: 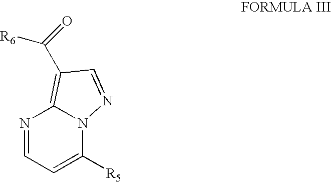
wherein R₆ and R₅ are as defined as above.

### DETAILED DESCRIPTION OF THE INVENTION

The definitions set forth below apply to the terms used herein unless otherwise defined.

### DEFINITIONS

Halogen is defined as fluoro, chloro, bromo, and iodo.

In this specification the term “alkyl” includes straight, branched alkyl groups, such as iso-propyl, n-butyl, tert-butyl, and cycloalkyl groups. The length of a straight alkyl moiety can be from 1 to 12 carbon atoms, but is preferably 1 to 8 carbon atoms. Also preferred are the alkyl chain lengths of 1 to 6 carbon atoms. Another preferred embodiment includes alkyl groups that contain 1 to 4 carbon atoms. Further preferred moieties are 1 to 3 carbon atoms in length. Furthermore, branched alkyl moieties can contain 3 to 12 carbon atoms. These alkyl moieties may be substituted or unsubstituted. The term “alkenyl” refers to a substituted or unsubstituted radical aliphatic hydrocarbon containing one double bond and includes alkenyl moieties of both straight, preferably of 2 to 7 carbon atoms and branched, preferably of 3 to 7 carbon atoms. Such alkenyl moieties may exist in the E or Z configurations; the compounds of this invention include both configurations. The term “alkynyl” includes substituted and unsubstituted alkynyl moieties of both straight chain containing 2 to 7 carbon atoms and branched containing 4 to 7 carbon atoms having at least one triple bond.

An alkoxy group is defined as an alkyl group attached to an oxygen atom such as methoxy, t-butoxy and the like. It includes polyethers such as —O—(CH₂)₂OCH₃ and the like. It also includes cycloalkyl ethers, such as an epoxide, in which the oxygen atom is a member of the cyclic ring. The alkyl group is as defined as above (it can thus be straight, branched, or cyclic).

A substituted phenyl or heteroaryl ring may have substituents in the ortho, meta, and/or para positions. The heteroaryl ring is defined as an aromatic heterocyclic ring system, preferably with a 5 or 6 membered aromatic moiety, containing at least 1-4 heteroatoms selected from O, S, and N. The heteroaryl moieties are preferably selected from the group consisting of thiophene, furan, pyrrole, pyrazole, imidazole, 1,2,3-triazole, 1,2,4-triazole, tetrazole, thiazole, oxazole, isothiazole, isoxazole, 1,3,4-oxadiazole, 1,2,4-oxadiazole, 1,3,4-thiadiazole, pyridine, pyrimidine, pyrazine, pyridazine and 1,3,5-triazine. The heteroaryl ring may be oxidized on a nitrogen atom to provide the corresponding N-oxide, such as pyridine N-oxide, or the heterocyclic ring may contain a carbonyl group on one of the carbon atoms, such as 1,3,4-oxadiazol-2-one. Heteroatoms in any ring system can be protected with their known protecting groups common in the art. (Greene, T.; Wuts, P. Protective Groups in Organic Synthesis, 2^{nd} Ed., 1991).

Bicyclic ring systems include both bicyclic aryl and bicyclic heteroaryl and are preferably selected from naphthalene, 1,2,3,4-tetrahydronaphthalene, indan, indene, isoindene, indole, 2,3-dihydroindole, 2-indazole, isoindazole, quinoline, isoquinoline, tetrahydroquinoline, benzofuran, benzothiophene, benzimidazole, benzotriazole, benzothiazole, benzoxazole, benzisoxazole, 1,2-benzopyran, cinnoline, phthalazine, quinazoline, 1,8-naphthyridine, pyrido[3,2-b]pyridine, pyrido[3,4-b]pyridine, pyrido[4,3-b]pyridine, pyrido[2,3-d]pyrimidine, purine, pteridine and others. Nitrogen atoms contained in either or both rings of the bicyclic group may be oxidized to provide the corresponding N-oxide, such as quinoline N-oxide. The bicyclic ring system may be oxidized at the carbon atoms to provide a carbonyl group, such as 2-indanone.

Within the present invention, it is to be understood that a pyrazolo[1,5-a]pyrimidine compound of this invention may exhibit the phenomenon of tautomerism and that the formula drawings within this specification can represent only one of the tautomeric forms. It is to be understood that this invention encompasses any tautomeric form and is not limited merely to any one tautomeric form utilized within the formula drawings.

The compounds of this invention may contain one or more stereogenic carbon atoms. In such cases, the compounds of this invention include the individual diasteromers, the racemates and the enantiomers thereof. The compounds of this invention may contain one or more double bonds. In such cases, the compounds of this invention include each of the possible configuration isomers as well as mixtures of these isomers.

The subject invention also includes pharmaceutically acceptable prodrugs of compounds of this invention. A “pharmaceutically acceptable prodrug” is intended to mean a compound that may be converted under physiological conditions or by solvolysis to a compound or derivative of Formula I.

The subject invention also includes isotopically-labelled compounds, which are identical to those recited in the formulae of the compound of this invention, but for the fact that one or more atoms are replaced by an atom having an atomic mass or mass number different from the atomic mass or mass number usually found in nature.

A “pharmaceutically acceptable salt” is intended to mean a salt that retains the biological effectiveness and properties of the free acids and bases of compounds and derivatives of the compounds of this invention, and that is not biologically or otherwise undesirable. Though not intending to be limiting in any way, the pharmaceutically acceptable salts of the compounds of Formula I with a basic moiety can be formed from organic and inorganic acids, such as acetic, citric, tartaric, succinic, maleic, malonic, gluconic, hydrochloric, hydrobromic, phosphoric, nitric, sulfuric, methanesulfonic, and similarly known acceptable acids. Similarly, when a compound of this invention contains a acidic moiety, salts can be formed from organic and inorganic bases. For example, alkali metal salts might include: sodium, lithium, potassium and N-tetraalkylammonium salts such as N-tetrabutylammonium. The literature of this art is replete with the possible salts and the methods for preparing them. One skilled in the art would be knowledgeable of the pharmaceutically acceptable salts and could easily prepare salts of the inventive compounds.

Solid or liquid pharmaceutically acceptable carriers, diluents, vehicles, or excipients may be employed in the pharmaceutical compositions. Illustrative solid carriers include starch, lactose, calcium sulphate dihydrate, terra alba, sucrose, talc, gelatin, pectin, acacia, magnesium stearate, and stearic acid. Illustrative liquid carriers may include syrup, peanut oil, olive oil, saline solution, and water.

A “therapeutically effective amount” is intended to mean that amount of a compound of this invention that, when administered to a human or mammal in need thereof, is sufficient to effect treatment for cancer. The amount of a given compound of this invention that will correspond to a “therapeutically effective amount” will vary depending upon factors such as the particular compound, the disease condition and the severity thereof, the identity of the human or mammal in need thereof, but it can nevertheless be readily determined by one of skill in the art.

A “neoplasm” is any new and abnormal growth; specifically a new growth of tissue in which the growth is uncontrolled and progressive. A neoplasm can be benign or malignant. A neoplasm furthermore can be the result or symptom of cancer.

“Treating” or “treatment” is intended to mean at least the slowing of the progression of a neoplastic cell in a mammal, such as a human; preferably stopping the progression of the neoplasm, and more preferably curing the condition. Treatment relates to the inhibition of proliferation of p21-deficient cells, and may include:

(a) prophylactic treatment in a mammal, particularly when the mammal is found to be predisposed to having the disease condition but not yet diagnosed as having it;

(b) inhibiting the disease condition; and/or

(c) alleviating, in whole or in part, the disease condition.

The compounds of this invention may be prepared by the procedures described herein, or otherwise known in the art as detailed in the following references: U.S. Pat. No. 4,654,347; U.S. Pat. No. 4,236,005; U.S. Pat. No. 4,521,422; U.S. Pat. No. 4,281,000; U.S. Pat. No. 4,626,538; U.S. Pat. No. 4,576,943; U.S. Pat. No. 5,059,691; EP 0129847; and EP 0208846, the disclosures of which are hereby incorporated by reference.

The preferred compounds of this invention include the following:
N-Phenyl-N′-{3-[3-(thien-2-ylcarbonyl)pyrazolo[1,5-a]pyrimidin-7-yl]phenyl}urea;
N-(2-Phenylethyl)-N′-{3-[3-(thien-2-ylcarbonyl)pyrazolo[1,5-a]pyrimidin-7-yl]phenyl}urea;
N-Cyclohexyl-N′-{3-[3-(thien-2-ylcarbonyl)pyrazolo[1,5-a]pyrimidin-7-yl]phenyl}urea;
Methyl N-[({3 -[3-(thien-2-ylcarbonyl)pyrazolo[1,5 -a]pyrimidin-7-yl]phenyl}amino)carbonyl]-L valinate;
N-Allyl-N′-{3-[3-(thien-2-ylcarbonyl)pyrazolo[1,5-a]pyrimidin-7-yl]phenyl}urea;
N-Butyl-N′-{3-[3-(thien-2-ylcarbonyl)pyrazolo[1,5-a]pyrimidin-7-yl]phenyl}urea;
N-{3-[3-(Thien-2-ylcarbonyl)pyrazolo[1,5-a]pyrimidin-7-yl]phenyl}-N′-(trichloroacetyl)urea;
Ethyl N-[({3-[3-(thien-2-ylcarbonyl)pyrazolo[1,5-a]pyrimidin-7-yl]phenyl}amino)carbonyl]glycinate;
N-[(1S)-1-Phenylethyl]-N′-{3-[3-(thien-2-ylcarbonyl)pyrazolo[1,5-a]pyrimidin-7-yl]phenyl}urea;
N-[(1S,2R)-2-Phenylcyclopropyl]-N′-{3-[3-(thien-2-ylcarbonyl)pyrazolo[1,5-a]pyrimidin-7-yl]phenyl }urea;
Allyl 3-[3-(thien-2-ylcarbonyl)pyrazolo[1,5-a]pyrimidin-7-yl]phenylcarbamate;
2,2,2-Trichloroethyl 3-[3-(thien-2-ylcarbonyl)pyrazolo[1,5-a]pyrimidin-7-yl]phenylcarbamate;
Prop-2-ynyl 3-[3-(thien-2-ylcarbonyl)pyrazolo[1,5-a]pyrimidin-7-yl]phenylcarbamate;
3-Butenyl 3-[3-(2-thienylcarbonyl)pyrazolo[1,5-a]pyrimidin-7-yl]phenylcarbamate;
Isopropenyl 3-[3-(thien-2-ylcarbonyl)pyrazolo[1,5-a]pyrimidin-7-yl]phenylcarbamate;
2-Chloroethyl 3-[3-(thien-2-ylcarbonyl)pyrazolo[1,5-a]pyrimidin-7-yl]phenylcarbamate;
4-Methylphenyl 3-[3-(thien-2-ylcarbonyl)pyrazolo[1,5-a]pyrimidin-7-yl]phenylcarbamate;
Benzyl 3-[3-(thien-2-ylcarbonyl)pyrazolo[1,5-a]pyrimidin-7-yl]phenylcarbamate;
4-Chlorobutyl 3-[3-(thien-2-ylcarbonyl)pyrazolo[1,5-a]pyrimidin-7-yl]phenylcarbamate;
N-{3-[3-(Thien-2-ylcarbonyl)pyrazolo[1,5-a]pyrimidin-7-yl]phenyl}methanesulfonamide;
N-{3-[3-(Thien-2-ylcarbonyl)pyrazolo[1,5-a]pyrimidin-7-yl]phenyl}ethanesulfonamide;
N-{3-[3-(Thien-2-ylcarbonyl)pyrazolo[1,5-a]pyrimidin-7-yl]phenyl}butane-1-sulfonamide;
N-{3-[3-(Thien-2-ylcarbonyl)pyrazolo[1,5-a]pyrimidin-7-yl]phenyl}propane-2-sulfonamide;
N-{3-[3-(Thien-2-ylcarbonyl)pyrazolo[1,5-a]pyrimidin-7-yl]phenyl}benzenesulfonamide;
4-Methyl-N-{3-[3-(thien-2-ylcarbonyl)pyrazolo[1,5-a]pyrimidin-7-yl]phenyl}benzenesulfonamide;
2-Methyl-N-{3-[3-(thien-2-ylcarbonyl)pyrazolo[1,5-a]pyrimidin-7-yl]phenyl}benzenesulfonamide;
4-Fluoro-N-{3-[3-(thien-2-ylcarbonyl)pyrazolo[1,5-a]pyrimidin-7-yl]phenyl}benzenesulfonamide;
4-Bromo-N-{3-[3-(thien-2-ylcarbonyl)pyrazolo[1,5-a]pyrimidin-7-yl]phenyl}benzenesulfonamide;
4-Methoxy-N-{3-[3-(thien-2-ylcarbonyl)pyrazolo[1,5 -a]pyrimidin-7-yl]phenyl}benzenesulfonamide;
(E)-2-Phenyl-N-{3-[3-(thien-2-ylcarbonyl)pyrazolo[1,5-a]pyrimidin-7-yl]phenyl}ethenesulfonamide;
N-{3-[3-(Thien-2-ylcarbonyl)pyrazolo[1,5-a]pyrimidin-7-yl]phenyl}thiophene-2-sulfonamide;
1-Methyl-N-{3-[3-(thien-2-ylcarbonyl)pyrazolo[1,5-a]pyrimidin-7-yl]phenyl}-1H-imidazole-4-sulfonamide;
N,N-Dimethyl-N′-{3-[3-(thien-2-ylcarbonyl)pyrazolo[1,5-a]pyrimidin-7-yl]phenyl}sulfamide;
Ethyl oxo({3-[3-(thien-2-ylcarbonyl)pyrazolo[1,5-a]pyrimidin-7-yl]phenyl}amino)acetate;
Methyl 4-oxo-4-({3-[3 -(thien-2-ylcarbonyl)pyrazolo[1,5-a]pyrimidin-7-yl]phenyl}amino)butanoate;
2-Methyl-N-{3-[3-(thien-2-ylcarbonyl)pyrazolo[1,5-a]pyrimidin-7-yl]phenyl}acrylamide;
3-Methyl-N-{3-[3 -(thien-2-ylcarbonyl)pyrazolo[1,5-a]pyrimidin-7-yl]phenyl}but-2-enamide;
2,2,2-Trichloro-N-{3-[3-(thien-2-ylcarbonyl)pyrazolo[1,5-a]pyrimidin-7-yl]phenyl}acetamide;
3-(Methylthio)-N-{3 -[3 -(thien-2-ylcarbonyl)pyrazolo[1,5 -a]pyrimidin-7-yl]phenyl}propanamide;
N-{3-[3-(Thien-2-ylcarbonyl)pyrazolo[1,5-a]pyrimidin-7-yl]phenyl}thiophene-2-carboxamide;
N-{3 -[3 -(Thien-2-ylcarbonyl)pyrazolo[1,5-a]pyrimidin-7-yl]phenyl}-1-(trifluoroacetyl)-L-prolinamide;
N-{3-[3-(Thien-2-ylcarbonyl)pyrazolo[1,5-a]pyrimidin-7-yl]phenyl}adamantane-1-carboxamide;
4,7,7-Trimethyl-3-oxo-N-{3-[3-(thien-2-ylcarbonyl)pyrazolo[1,5-a]pyrimidin-7-yl]phenyl}-2-oxabicyclo[2.2.1]heptane-1-carboxamide;
N-{3-[3-(Thien-2-ylcarbonyl)pyrazolo[1,5-a]pyrimidin-7-yl]phenyl}pent-4-enamide;
N-{3-[3-(Thien-2-ylcarbonyl)pyrazolo[1,5-a]pyrimidin-7-yl]phenyl}pent-4-ynamide;
4,4,4-Trifluoro-3-methyl-N-{3-[3-(thien-2-ylcarbonyl)pyrazolo[1,5-a]pyrimidin-7-yl]phenyl}butanamide;
2-Cyano-N-{3-[3-(thien-2-ylcarbonyl)pyrazolo[1,5-a]pyrimidin-7-yl]phenyl}acetamide;
2-Oxo-2-({3-[3-(thien-2-ylcarbonyl)pyrazolo[1,5-a]pyrimidin-7-yl]phenyl}amino)ethyl acetate;
2-(2-Methoxyethoxy)-N-{3-[3-(thien-2-ylcarbonyl)pyrazolo [1,5-a]pyrimidin-7-yl]phenyl}acetamide;
2-Thien-2-yl-N-{3-[3-(thien-2-ylcarbonyl)pyrazolo[1,5-a]pyrimidin-7-yl]phenyl}acetamide;
2-(2,3-Dihydro-1H-inden-2-yl)-N-{3-[3-(thien-2-ylcarbonyl)pyrazolo[1,5-a]pyrimidin-7-yl[phenyl}acetamide;
2-(2-Methylphenyl)-N-{3-[3-(thien-2-ylcarbonyl)pyrazolo[1,5-a]pyrimidin-7-yl]phenyl}acetamide;
2-(1,3-Benzodioxol-5-yl)-N-{3-[3-(thien-2-ylcarbonyl)pyrazolo[1,5-a]pyrimidin-7-yl]phenyl}acetamide;
N-{3-[3-(Thien-2-ylcarbonyl)pyrazolo[1,5-a]pyrimidin-7-yl]phenyl}quinoline-3-carboxamide;
(2E)-3-Phenyl-N-{3-[3-(thien-2-ylcarbonyl)pyrazolo[1,5-a]pyrimidin-7-yl]phenyl}prop-2-enamide;
2-Cyclohexyl-N-{3-[3-(thien-2-ylcarbonyl)pyrazolo[1,5-a]pyrimidin-7-yl]phenyl}acetamide;
2-Cycloheptyl-N-{3-[3-(thien-2-ylcarbonyl)pyrazolo[1,5-a]pyrimidin-7-yl]phenyl}acetamide;
2-Bicyclo[2.2.1]hept-2-yl-N-{3-[3-(thien-2-ylcarbonyl)pyrazolo[1,5-a]pyrimidin-7-yl]phenyl}acetamide;
N-1-{3-[3-(Thien-2-ylcarbonyl)pyrazolo[1,5-a]pyrimidin-7-yl]phenyl}glycinamide;
N-1-{3-[3-(Thien-2-ylcarbonyl)pyrazolo[1,5-a]pyrimidin-7-yl]phenyl}-L-alaninamide;
N-1-{3-[3-(Thien-2-ylcarbonyl)pyrazolo[1,5-a]pyrimidin-7-yl]phenyl}-L-leucinamide;
N-1-{3-[3-(Thien-2-ylcarbonyl)pyrazolo[1,5-a]pyrimidin-7-yl]phenyl}-L-isoleucinamide;
N-{3-[3-(Thien-2-ylcarbonyl)pyrazolo[1,5-a]pyrimidin-7-yl]phenyl}-L-phenylalaninamide;
3-Cyclohexyl-N-1-{3-[3-(thien-2-ylcarbonyl)pyrazolo[1,5-a]pyrimidin-7-yl]phenyl}-L-alaninamide;
N-1-{3-[3-(Thien-2-ylcarbonyl)pyrazolo[1,5-a]pyrimidin-7-yl]phenyl}-L-valinamide;
4-Amino-N-{3-[3-(thien-2-ylcarbonyl)pyrazolo[1,5-a]pyrimidin-7-yl]phenyl}butanamide;
N-{3-[3-(Thien-2-ylcarbonyl)pyrazolo[1,5-a]pyrimidin-7-yl]phenyl}-L-proliniamide;
(2S)-2-Amino-2-cyclohexyl-N-{3-[3-(thien-2-ylcarbonyl)pyrazolo[1,5-a]pyrimidin-7-yl]phenyl}ethanamide;
4-Methyl-N-{3-[3-(thien-2-ylcarbonyl)pyrazolo[1,5-a]pyrimidin-7-yl]phenyl}benzamide;
4-Methoxy-N-{3-[3-(thien-2-ylcarbonyl)pyrazolo[1,5-a]pyrimidin-7-yl]phenyl}benzamide;
4-Chloro-N-{3-[3-(thien-2-ylcarbonyl)pyrazolo[1,5-a]pyrimidin-7-yl]phenyl}benzamide;
4-Bromo-N-{3-[3-(thien-2-ylcarbonyl)pyrazolo[1,5-a]pyrimidin-7-yl]phenyl}benzamide;
4-(Heptyloxy)-N-{3-[3-(thien-2-ylcarbonyl)pyrazolo[1,5-a]pyrimidin-7-yl]phenyl}benzamide;
4-Pentyl-N-{3-[3-(thien-2-ylcarbonyl)pyrazolo[1,5-a]pyrimidin-7-yl]phenyl}benzamide,
2-Methoxy-N-{3-[3-(thien-2-ylcarbonyl)pyrazolo[1,5-a]pyrimidin-7-yl]phenyl}benzamide;
2-Methyl-N-{3-[3-(thien-2-ylcarbonyl)pyrazolo[1,5-a]pyrimidin-7-yl]phenyl}benzamide;
2-Chloro-N-{3-[3-(thien-2-ylcarbonyl)pyrazolo[1,5-a]pyrimidin-7-yl]phenyl}benzamide;
N-{3-[3-(Thien-2-ylcarbonyl)pyrazolo[1,5-a]pyrimidin-7-yl]phenyl}-2-(trifluoromethyl)benzamide;
3-Methoxy-N-{3-[3-(thien-2-ylcarbonyl)pyrazolo[1,5-a]pyrimidin-7-yl]phenyl}benzamide;
3-Fluoro-N-{3-[3-(thien-2-ylcarbonyl)pyrazolo[1,5-a]pyrimidin-7-yl]phenyl}benzamide;
3,4-Dimethoxy-N-{3-[3-(thien-2-ylcarbonyl)pyrazolo[1,5-a]pyrimidin-7-yl]phenyl}benzamide;
3,4-Dichloro-N-{3-[3-(thien-2-ylcarbonyl)pyrazolo[1,5-a]pyrimidin-7-yl]phenyl}benzamide;
2,6-Dimethoxy-N-{3-[3-(thien-2-ylcarbonyl)pyrazolo[1,5-a]pyrimidin-7-yl]phenyl}benzamide;
3,4-Difluoro-N-{3-[3-(thien-2-ylcarbonyl)pyrazolo[1,5-a]pyrimidin-7-yl]phenyl}benzamide;
N-{3-[3-(Thien-2-ylcarbonyl)pyrazolo[1,5-a]pyrimidin-7-yl]phenyl}-1-naphthamide;
N-{3-[3-(Thien-2-ylcarbonyl)pyrazolo[1,5-a]pyrimidin-7-yl]phenyl}-2-naphthamide;
2-Phenyl-N-{3-[3-(thien-2-ylcarbonyl)pyrazolo[1,5-a]pyrimidin-7-yl]phenyl}acetamide;
2-(4-Methoxyphenyl)-N-{3-[3-(thien-2-ylcarbonyl)pyrazolo[1,5-a]pyrimidin-7-yl]phenyl}acetamide;
2-(3-Methoxyphenyl)-N-{3-[3-(thien-2-ylcarbonyl)pyrazolo[1,5-a]pyrimidin-7-yl]phenyl}acetamide;
2-(4-Chlorophenyl)-N-{3-[3-(thien-2-ylcarbonyl)pyrazolo[1,5-a]pyrimidin-7-yl]phenyl}acetamide;
2-(4-Fluorophenyl)-N-{3-[3-(thien-2-ylcarbonyl)pyrazolo[1,5-a]pyrimidin-7-yl]phenyl}acetamide;
2-(2,5-Dimethoxyphenyl)-N-{3-[3-(thien-2-ylcarbonyl)pyrazolo[1,5-a]pyrimidin-7-yl]phenyl}acetamide;
2-Phenoxy-N-{3-[3-(thien-2-ylcarbonyl)pyrazolo[1,5-a]pyrimidin-7-yl]phenyl}acetamide;
4-Fluoro-N-{3-[3-(thien-2-ylcarbonyl)pyrazolo [1,5-a]pyrimidin-7-yl]phenyl}benzamide;
Ethyl 2-oxo-2-({3-[3-(thien-2-ylcarbonyl)pyrazolo[1,5-a]pyrimidin-7-yl]phenyl}amino)ethylcarbamate;
Propyl 2-oxo-2-({3-[3-(thien-2-ylcarbonyl)pyrazolo[1,5-a]pyrimidin-7-yl]phenyl}amino)ethylcarbamate;
Isopropyl 2-oxo-2-({3-[3-(thien-2-ylcarbonyl)pyrazolo[1,5-a]pyrimidin-7-yl]phenyl}amino)ethylcarbamate;
Butyl 2-oxo-2-({3-[3-(thien-2-ylcarbonyl)pyrazolo[1,5-a]pyrimidin-7-yl]phenyl}amino)ethylcarbamate;
Isobutyl 2-oxo-2-({3-[3-(thien-2-ylcarbonyl)pyrazolo[1,5-a]pyrimidin-7-yl]phenyl}amino)ethylcarbamate;
But-3-enyl 2-oxo-2-({3-[3-(thien-2-ylcarbonyl)pyrazolo[1,5-a]pyrimidin-7-yl]phenyl}amino)ethylcarbamate;
4-Chlorobutyl 2-oxo-2-({3-[3-(thien-2-ylcarbonyl)pyrazolo[1,5-a]pyrimidin-7-yl]phenyl}amino)ethylcarbamate;
2-Chloroethyl 2-oxo-2-({3-[3-(thien-2-ylcarbonyl)pyrazolo[1,5-a]pyrimidin-7-yl]phenyl}amino)ethylcarbamate;
Neopentyl 2-oxo-2-({3-[3-(thien-2-ylcarbonyl)pyrazolo[1,5-a]pyrimidin-7-yl]phenyl}amino)ethylcarbamate;
Hexyl 2-oxo-2-({3-[3-(thien-2-ylcarbonyl)pyrazolo[1,5-a]pyrimidin-7-yl]phenyl}amino)ethylcarbamate;
Prop-2-ynyl 2-oxo-2-({3-[3-(thien-2-ylcarbonyl)pyrazolo[1,5-a]pyrimidin-7-yl]phenyl}amino)ethylcarbamate;
Allyl 2-oxo-2-({3-[3-(thien-2-ylcarbonyl)pyrazolo[1,5-a]pyrimidin-7-yl]phenyl}amino)ethylcarbamate;
Benzyl 2-oxo-2-({3-[3-(thien-2-ylcarbonyl)pyrazolo[1,5-a]pyrimidin-7-yl]phenyl}amino)ethylcarbamate;
N-[2-Oxo-2-({3-[3-(thien-2-ylcarbonyl)pyrazolo[1,5-a]pyrimidin-7-yl]phenyl}amino)ethyl]cyclopropanecarboxamide;
N-[2-Oxo-2-({3-[3-(thien-2-ylcarbonyl)pyrazolo[1,5-a]pyrimidin-7-yl]phenyl}amino)ethyl]cyclobutanecarboxamide;
N-[2-Oxo-2-({3-[3-(thien-2-ylcarbonyl)pyrazolo[1,5-a]pyrimidin-7-yl]phenyl}amino)ethyl]cyclopentanecarboxamide;
N-[2-Oxo-2-({3-[3-(thien-2-ylcarbonyl)pyrazolo[1,5-a]pyrimidin-7-yl]phenyl}amino)ethyl]cyclohexanecarboxamide;
2-Methyl-N-[2-oxo-2-({3-[3-(thien-2-ylcarbonyl)pyrazolo[1,5-a]pyrimidin-7-yl]phenyl}amino)ethyl]butanamide;
2-Ethyl-N-[2-oxo-2-({3-[3-(thien-2-ylcarbonyl)pyrazolo[1,5-a]pyrimidin-7-yl]phenyl}amino)ethyl]butanamide;
2,2-Dimethyl-N-[2-oxo-2-({3-[3-(thien-2-ylcarbonyl)pyrazolo[1,5-a]pyrimidin-7-yl]phenyl}amino)ethyl]propanamide;
3-Methyl-N-[2-oxo-2-({3-[3-(thien-2-ylcarbonyl)pyrazolo[1,5-a]pyrimidin-7-yl]phenyl}amino) ethyl]butanamide;
N-[2-Oxo-2-({3-[3-(thien-2-ylcarbonyl)pyrazolo[1,5-a]pyrimidin-7-yl]phenyl}amino)ethyl]adamantane-1-carboxamide;
N-[2-Oxo-2-({3-[3-(thien-2-ylcarbonyl)pyrazolo[1,5-a]pyrimidin-7-yl]phenyl}amino)ethyl]benzamide;
N-2-[(Pentylamino)carbonyl]-N-1-{3-[3-(thien-2-ylcarbonyl)pyrazolo[1,5-A]pyrimidin-7-yl]phenyl}glycinamide;
N-2-[(Ethylamino)carbonyl]-N-1-{3-[3-(thien-2-ylcarbonyl)pyrazolo[1,5-a]pyrimidin-7-yl]phenyl}glycinamide;
N-2-[(Cyclohexylamino)carbonyl]-N-1-{3-[3-(thien-2-ylcarbonyl)pyrazolo[1,5-a]pyrimidin-7-yl]phenyl}glycinamide;
3-Chloro-N-{3-[3-(thien-2-ylcarbonyl)pyrazolo[1,5-a]pyrimidin-7-yl]phenyl}propanamide;
3,5,5-Trimethyl-N-{3-[3-(thien-2-ylcarbonyl)pyrazolo[1,5-a]pyrimidin-7-yl]phenyl}hexanamide;
3-Chloro-2-(chloromethyl)-2-methyl-N-(3-[3-(thien-2-ylcarbonyl)pyrazolo[1,5-a]pyrimidin-7-yl]phenyl}propanamide;
N-{3-[3-(Thien-2-ylcarbonyl)pyrazolo[1,5-a]pyrimidin-7-yl]phenyl}undecanamide;
1-Methyl-N-{3-[3-(thien-2-ylcarbonyl)pyrazolo[1,5-a]pyrimidin-7-yl]phenyl}cyclohexanecarboxamide;
2,3,3-Trichloro-N-{3-[3-(thien-2-ylcarbonyl)pyrazolo[1,5-a]pyrimidin-7-yl]phenyl}acrylamide;
3-Cyclopentyl-N-{3-[3-(thien-2-ylcarbonyl)pyrazolo[1,5-a]pyrimidin-7-yl]phenyl}propanamide;
2-Ethyl-N-{3-[3-(thien-2-ylcarbonyl)pyrazolo[1,5-a]pyrimidin-7-yl]phenyl}hexanamide;
(2E)-N-{3-[3-(Thien-2-ylcarbonyl)pyrazolo[1,5-a]pyrimidin-7-yl]phenyl}but-2-enamide;
N-{3-[3-(Thien-2-ylcarbonyl)pyrazolo[1,5-a]pyrimidin-7-yl]phenyl}piperidine-1-carboxamide;
N-{3-[3-(Thien-2-ylcarbonyl)pyrazolo[1,5-a]pyrimidin-7-yl]phenyl}morpholine-4-carboxamide;
N-(sec-Butyl)-N′-{3-[3-(thien-2-ylcarbonyl)pyrazolo[1,5-a]pyrimidin-7-yl]phenyl}urea;
N-(Cyclohexylmethyl)-N′-{3-[3-(thien-2-ylcarbonyl)pyrazolo[1,5-a]pyrimidin-7-yl]phenyl}urea;
N-[2-(Dimethylamino)ethyl]-N′-{3-[3-(thien-2-ylcarbonyl)pyrazolo[1,5-a]pyrimidin-7-yl]phenyl}urea;
N-Cyclopentyl-N′-{3-[3-(thien-2-ylcarbonyl)pyrazolo[1,5-a]pyrimidin-7-yl]phenyl}urea;
N-(Tetrahydrofuran-2-ylmethyl)-N′-{3-[3-(thien-2-ylcarbonyl)pyrazolo[1,5-a]pyrimidin-7-yl]phenyl}urea;
N-Neopentyl-N′-{3-[3-(thien-2-ylcarbonyl)pyrazolo[1,5-a]pyrimidin-7-yl]phenyl}urea;
N-Pentyl-N′-{3-[3-(thien-2-ylcarbonyl)pyrazolo[1,5-a]pyrimidin-7-yl]phenyl}urea;
4-Methyl-N-{3-[3-(thien-2-ylcarbonyl)pyrazolo[1,5-a]pyrimidin-7-yl]phenyl}piperazine-1-carboxamide;
4-Methyl-N-{3-[3-(thien-2-ylcarbonyl)pyrazolo[1,5-a]pyrimidin-7-yl]phenyl}-1,4-diazepane-1-carboxamide;
N,N-Bis(2-methoxyethyl)-N′-{3-[3-(thien-2-ylcarbonyl)pyrazolo[1,5-a]pyrimidin-7-yl]phenyl}urea;
N-Cyclopropyl-N′-{3-[3-(thien-2-ylcarbonyl)pyrazolo[1,5-a]pyrimidin-7-yl]phenyl}urea;
N-{3-[3-(Thien-2-ylcarbonyl)pyrazolo[1,5-a]pyrimidin-7-yl]phenyl}pyrrolidine-1-carboxamide;
Cyclopentyl 3-[3-(thien-2-ylcarbonyl)pyrazolo[1,5-a]pyrimidin-7-yl]phenylcarbamate;
Cyclohexyl 3-[3-(thien-2-ylcarbonyl)pyrazolo[1,5-a]pyrimidin-7-yl]phenylcarbamate;
Cyclohex-2-en-1-yl 3-[3-(thien-2-ylcarbonyl)pyrazolo[1,5-a]pyrimidin-7-yl]phenylcarbamate;
Cyclobutylmethyl 3-[3-(thien-2-ylcarbonyl)pyrazolo[1,5-a]pyrimidin-7-yl]phenylcarbamate;
Cyclohexylmethyl 3-[3-(thien-2-ylcarbonyl)pyrazolo[1,5-a]pyrimidin 7-yl]phenylcarbamate;
Tetrahydro-2H-pyran-4-yl 3-[3-(thien-2-ylcarbonyl)pyrazolo[1,5-a]pyrimidin-7-yl]phenylcarbamate;
3-Methylbut-2-enyl 3-[3-(thien-2-ylcarbonyl)pyrazolo[1,5-a]pyrimidin-7-yl]phenylcarbamate;
2-(Methylthio)ethyl 3-[3-(thien-2-ylcarbonyl)pyrazolo[1,5-a]pyrimidin-7-yl]phenylcarbamate;
2-Thien-3-ylethyl 3-[3-(thien-2-ylcarbonyl)pyrazolo[1,5-a]pyrimidin-7-yl]phenylcarbamate;
2-Piperidin-1-ylethyl 3-[3-(thien-2-ylcarbonyl)pyrazolo[1,5-a]pyrimidin-7-yl]phenylcarbamate;
2-Morpholin-4-ylethyl 3-[3-(thien-2-ylcarbonyl)pyrazolo[1,5-a]pyrimidin-7-yl]phenylcarbamate;
1-Methylprop-2-enyl 3-[3-(thien-2-ylcarbonyl)pyrazolo[1,5-a]pyrimidin-7-yl]phenylcarbamate;
1-Methylbut-3-ynyl 3-[3-(thien-2-ylcarbonyl)pyrazolo[1,5-a]pyrimidin-7-yl]phenylcarbamate;
(2-Methylcyclopropyl)methyl 3-[3-(thien-2-ylcarbonyl)pyrazolo[1,5-a]pyrimidin-7-yl]phenylcarbamate;
3-Methylcyclopentyl 3-[3-(thien-2-ylcarbonyl)pyrazolo[1,5-a]pyrimidin-7-yl]phenylcarbamate;
1-Methylbut-3-enyl 3-[3-(thien-2-ylcarbonyl)pyrazolo[1,5-a]pyrimidin-7-yl]phenylcarbamate;
1-Cyclopropylethyl 3-[3-(thien-2-ylcarbonyl)pyrazolo[1,5-a]pyrimidin-7-yl]phenylcarbamate;
But-3-ynyl 3-[3-(thien-2-ylcarbonyl)pyrazolo[1,5-a]pyrimidin-7-yl]phenylcarbamate;
Allyl 3-[5-methyl-3-(thien-2-ylcarbonyl)pyrazolo[1,5-a]pyrimidin-7-yl]phenylcarbamate;
2-Chloroethyl 3-[5-methyl-3-(thien-2-ylcarbonyl)pyrazolo[1,5-a]pyrimidin-7-yl]phenylcarbamate;
3-Methyl-N-{3-[5-methyl-3-(thien-2-ylcarbonyl)pyrazolo[1,5-a]pyrimidin-7-yl]phenyl}butanamide;
N-{3-[5-Methyl-3-(thien-2-ylcarbonyl)pyrazolo[1,5-a]pyrimidin-7-yl]phenyl}benzamide;
N-{3-[5-Methyl-3-(thien-2-ylcarbonyl)pyrazolo[1,5-a]pyrimidin-7-yl]phenyl}-2-thien-2-ylacetamide;
N-Butyl-N′-{3-[5-methyl-3-(thien-2-ylcarbonyl)pyrazolo[1,5-a]pyrimidin-7-yl]phenyl}urea;
Allyl 3-[6-methyl-3-(thien-2-ylcarbonyl)pyrazolo[1,5-a]pyrimidin-7-yl]phenylcarbamate;
2-Chloroethyl 3-[6-methyl-3-(thien-2-ylcarbonyl)pyrazolo[1,5-a]pyrimidin-7-yl]phenylcarbamate;
But-3-enyl 3-[6-methyl-3-(thien-2-ylcarbonyl)pyrazolo[1,5-a]pyrimidin-7-yl]phenylcarbamate;
N-{3-[6-Methyl-3-(thien-2-ylcarbonyl)pyrazolo[1,5-a]pyrimidin-7-yl]phenyl}benzamide;
N-{3-[6-Methyl-3-(thien-2-ylcarbonyl)pyrazolo[1,5-a]pyrimidin-7-yl]phenyl}-2-thien-2-ylacetamide;
N-{3-[6-Methyl-3-(thien-2-ylcarbonyl)pyrazolo[1,5-a]pyrimidin-7-yl]phenyl}nicotinamide;
N-Mutyl-N′-{3-[6-methyl-3-(thien-2-ylcarbonyl)pyrazolo[1,5-a]pyrimidin-7-yl]phenyl}urea;
1-[3-[3-(2-Furanylcarbonyl)pyrazolo-[1,5-a]pyrimidin-7-yl]phenyl]-2-piperidinone;
1-[3-(3-Benzoylpyrazolo[1,5-a]-pyrimidin-7-yl)phenyl]-2-piperidinone;
1-[3-[3-(2-Furanylcarbonyl)pyrazolo-[1,5-a]pyrimidin-7-yl]phenyl]-2-pyrrolidinone;
1-[3-(3-Benzoylpyrazolo[1,5-a]-pyrimidin-7-yl)phenyl]-2-pyrrolidinone;
N-[3-(3-Cyanopyrazolo[1,5-a]-pyrimidin-7-yl)phenyl]-N-methyl-cyclobutanecarboxamide;
[7-(2,4-Dimethoxyphenyl)pyrazolo[1,5-a]-pyrimidin-3-yl]-2-thienyl-methanone;
N,N-Diethyl-3-[3-(2-thienylcarbonyl)-pyrazolo[1,5-a]pyrimidin-7-yl]-benzamide;
N-[3-(3-Chloropyrazolo[1,5-a]pyrimidin-7-yl)phenyl]-N-ethyl-cyclopropanecarboxamide;
N-[3-(3-Cyanopyrazolo [1,5-a]pyrimidin-7-yl)phenyl]-N-ethyl-cyclopropanecarboxamide;
[7-[3-(2-Methoxyethoxy)phenyl]pyrazolo[1,5-a]pyrimidin-3-yl]-2-thienyl-methanone;
[7-[3-(Cyclopentyloxy)phenyl]pyrazolo[1,5-a]pyrimidin-3-yl]-2-thienyl-methanone;
2-Furanyl[7-[3-(1H-pyrrol-1-yl)phenyl]pyrazolo[1,5-a]pyrimidin-3-yl]-methanone;
[7-[3-(1H-Pyrrol-1-yl)phenyl]pyrazolo[1,5-a]pyrimidin-3-yl]-2-thienyl-methanone;
N-{3-[3-(2-Thienylcarbonyl)pyrazolo[1,5-a]pyrimidin-7-yl]phenyl}acrylamide;
N-{3-[3-(2-Thienylcarbonyl)pyrazolo[1,5-a]pyrimidin-7-yl]phenyl}-2-butynamide;
N-{3-[3-(2-Thienylcarbonyl)pyrazolo[1,5-a]pyrimidin-7-yl]phenyl}-2-oxiranecarboxamide;
N-{3-[3-(2-thienylcarbonyl)pyrazolo[1,5-a]pyrimidin-7-yl]phenyl}-2-oxiranecarboxamide;
N-{3-[5-Methyl-3-(2-thienylcarbonyl)pyrazolo[1,5-a]pyrimidin-7-yl]phenyl}-2-oxiranecarboxamide;
N-{3-[3-(2-Thienylcarbonyl)pyrazolo[1,5-a]pyrimidin-7-yl]phenyl}-2,5-bis(trifluoromethyl)benzamide;
N-{3-[3-(2-Thienylcarbonyl)pyrazolo[1,5-a]pyrimidin-7-yl]phenyl}-3-(trifluoromethyl)benzamide;
N-{3-[3-(2-Thienylcarbonyl)pyrazolo[1,5-a]pyrimidin-7-yl]phenyl}benzamide;
N-{3-[3-(2-Thienylcarbonyl)pyrazolo[1,5-a]pyrimidin-7-yl]phenyl}-2-furamide;
3-Bromo-N-{3-[3-(2-thienylcarbonyl)pyrazolo[1,5-a]pyrimidin-7-yl]phenyl}benzamide;
4-(tert-Butyl)-N-{3-[3-(2-thienylcarbonyl)pyrazolo[1,5-a]pyrimidin-7-yl]phenyl}benzamide;
3,5-Dinitro-N-{3-[3-(2-thienylcarbonyl)pyrazolo[1,5-a]pyrimidin-7-yl]phenyl}benzamide;
2,4-Dichloro-N-{3-[3-(2-thienylcarbonyl)pyrazolo[1,5-a]pyrimidin-7-yl]phenyl}benzamide;
N-{3-[3-(2-Thienylcarbonyl)pyrazolo[1,5-a]pyrimidin-7-yl]phenyl}nicotinamide;
N-{3-[3-(2-Thienylcarbonyl)pyrazolo[1,5-a]pyrimidin-7-yl]phenyl}isonicotinamide;
N,N′-Dibutyl-N-{3-[3-(2-thienylcarbonyl)pyrazolo[1,5-a]pyrimidin-7-yl]phenyl}dicarbonimidic diamide;
Isopropyl 3-[3-(cyclopentylcarbonyl)pyrazolo[1,5-a]pyrimidin-7-yl]phenylcarbamate;
N-{3-[3-(Cyclopentylcarbonyl)pyrazolo[1,5-a]pyrimidin-7-yl]phenyl}-3-methylbutanamide;
N-{3-[3-(2-Furoyl)pyrazolo[1,5-a]pyrimidin-7-yl]phenyl}benzamide;
Methyl 3-[3-(2-thienylcarbonyl)pyrazolo[1,5-a]pyrimidin-7-yl]benzoate;
N-{2-Methyl-5-[3-(2-thienylcarbonyl)pyrazolo[1,5-a]pyrimidin-7-yl]phenyl}benzamide;
3-Methyl-N-{2-methyl-5-[3-(2-thienylcarbonyl)pyrazolo[1,5-a]pyrimidin-7-yl]phenyl}butanamide
N-Isopropyl-N′-{2-methyl-5-[3-(2-thienylcatbonyl)pyrazolo [1,5-a]pyrimidin-7-yl]phenyl}urea;
N-{2-Chloro-5-[3-(2-thienylcarbonyl)pyrazolo[1,5-a]pyrimidin-7-yl]phenyl}benzamide;
N-{2-Chloro-5-[3-(2-thienylcarbonyl)pyrazolo[1,5-a]pyrimidin-7-yl]phenyl}-3-methylbutanamide;
Isopropyl 2-methyl-5-[3-(2-thienylcarbonyl)pyrazolo[1,5-a]pyrimidin-7-yl]phenylcarbamate;
N-{2-Methoxy-5-[3-(2-thienylcarbonyl)pyrazolo [1,5-a]pyrimidin-7-yl]phenyl}benzamide;
Isopropyl 2-methoxy-5-[3-(2-thienylcarbonyl)pyrazolo[1,5-a]pyrimidin-7-yl]phenylcarbamate;
N-{2-Methoxy-5-[3-(2-thienylcarbonyl)pyrazolo[1,5-a]pyrimidin-7-yl]phenyl}-3-methylbutanamide;
N-Isobutyl-3-[3-(2-thienylcarbonyl)pyrazolo[1,5-a]pyrimidin-7-yl]benzamide;
N-Butyl-3-[3-(2-thienylcarbonyl)pyrazolo[1,5-a]pyrimidin-7-yl]benzamide;
N-Cyclopentyl-3-[3-(2-thienylcarbonyl)pyrazolo[1,5-a]pyrimidin-7-yl]benzamide;
N-Phenyl-3-[3-(2-thienylcarbonyl)pyrazolo[1,5-a]pyrimidin-7-yl]benzamide;
N-Isopropyl-3-[3-(2-thienylcarbonyl)pyrazolo[1,5-a]pyrimidin-7-yl]benzamide;
N-Isopropyl-N′-{2-methoxy-5-[3-(2-thienylcarbonyl)pyrazolo[1,5-a]pyrimidin-7-yl]phenyl}urea;
3-Methyl-N-{5-[3-(2-thienylcarbonyl)pyrazolo[1,5-a]pyrimidin-7-yl]-3-pyridinyl}butanamide;
7-(3-Nitrophenyl)-3-(3-thienyl)pyrazolo[1,5-a]pyrimidine;
3-(3-Bromopyrazolo[1,5-a]pyrimidin-7-yl)aniline;
Isobutyl 3-(3-bromopyrazolo[1,5-a]pyrimidin-7-yl)phenylcarbamate;
[7-(5-Nitro-3-thienyl)pyrazolo[1,5-a]pyrimidin-3-yl](2-thienyl)methanone;
N-Isopropyl-N′-{5-[3-(thien-2-ylcarbonyl)pyrazolo[1,5-a]pyrimidin-7-yl]thien-2-yl}urea;
N-[3-(3-Bromopyrazolo[1,5-a]pyrimidin-7-yl)phenyl]-3-methylbutanamide;
[7-(5-Aminothien-3-yl)pyrazolo[1,5-a]pyrimidin-3-yl](thien-2-yl)methanone;
O-Ethyl 3-[3-(thien-2-ylcarbonyl)pyrazolo[1,5-a]pyrimidin-7-yl]phenylthiocarbamate;
3-Methyl-N-[3-(3-thien-2-ylpyrazolo[1,5-a]pyrimidin-7-yl)phenyl]butanamide;
N-Phenyl-N′-{3-[3-(thien-2-ylcarbonyl)pyrazolo[1,5-a]pyrimidin-7-yl]phenyl}thiourea;
Isopropyl 4-chloro-3-[3-(thien-2-ylcarbonyl)pyrazolo[1,5-a]pyrimidin-7-yl]phenylcarbamate;
[7-(2-Chloro-5-nitrophenyl)pyrazoio[1,5-a]pyrimidin-3-yl](thien-2-yl)methanone;
N-{3-[3-(Pyridin-2-ylcarbonyl)pyrazolo[1,5-a]pyrimidin-7-yl]phenyl}cyclopropanecarboxamide;
N-{3-[3-(Pyridin-2-ylcarbonyl)pyrazolo[1,5-a]pyrimidin-7-yl]phenyl}cyclopropanecarboxamide;
N-{4-Chloro-3-[3-(thien-2-ylcarbonyl)pyrazolo[1,5-a]pyrimidin-7-yl]phenyl}-3-methylbutanamide;
N-{4-Chloro-3-[3-(thien-2-ylcarbonyl)pyrazolo[1,5-a]pyrimidin-7-yl]phenyl}-N′-isopropylthiourea;
N-{4-Chloro-3-[3-(thien-2-ylcarbonyl)pyrazolo[1,5-a]pyrimidin-7-yl]phenyl}benzamide;
N-{2-Fluoro-5-[3-(thien-2-ylcarbonyl)pyrazolo[1,5-a]pyrimidin-7-yl]phenyl}-3-methylbutanamide;
3-Methyl-N-{5-[3-(thien-2-ylcarbonyl)pyrazolo[1,5-a]]pyrimidin-7-yl]thien-2-yl}butanamide;
Isopropyl 5-[3-(thien-2-ylcarbonyl)pyrazolo[1,5-a]pyrimidin-7-yl]thien-2-ylcarbamate;
[7-(5-Aminothien-2-yl)pyrazolo[1,5-a]pyrimidin-3-yl](thien-2-yl)methanone;
4-Chloro-N-{3-[3-(thien-2-ylcarbonyl)pyrazolo[1,5-a]pyrimidin-7-yl]phenyl}butanamide;
Isopropyl 2-fluoro-5-[3-(thien-2-ylcarbonyl)pyrazolo[1,5-a]pyrimidin-7-yl]phenylcarbamate;
Isopropyl 2-(dimethylamino)-5-[3-(thien-2-ylcarbonyl)pyrazolo[1,5-a]pyrimidin-7-yl]phenylcarbamate;
{7-[3-(Benzylamino)phenyl]pyrazolo[1,5-a]pyrimidin-3-yl}(thien-2-yl)methanone;
3-Methyl-N-{4-[3-(thien-2-ylcarbonyl)pyrazolo[1,5-a]pyrimidin-7-yl]thien-2-yl}butanamide;
[7-(3-Amino-4-fluorophenyl)pyrazolo[1,5-a]pyrimidin-3-yl](thien-2-yl)methanone;
N-Cyclohexyl-N′-{3-[3-(thien-2-ylcarbonyl)pyrazolo[1,5-a]pyrimidin-7-yl]phenyl}urea;
4,4,4-Trichloro-N-{3-[3-(thien-2-ylcarbonyl)pyrazolo[1,5-a]pyrimidin-7-yl]phenyl}butanamide;
N-{2-Fluoro-5-[3-(thien-2-ylcarbonyl)pyrazolo[1,5S-a]pyrimidin-7-yl]phenyl}-3,3-dimethylbutanamide;
4,4,4-Trifluoro-N-{2-fluoro-5-[3-(thien-2-ylcarbonyl)pyrazolo[1,5-a]pyrimidin-7-yl]phenyl}-3-methylbutanamide;
Bis(2,2,2-trichloroethyl) 2-fluoro-5-[3-(thien-2-ylcarbonyl)pyrazolo[1,5-a]pyrimidin-7-yl]phenylimidodicarbonate;
2-Fluoro-N-{3-[3-(thien-2-ylcarbonyl)pyrazolo[1,5-a]pyrimidin-7-yl]phenyl}benzamide;
2,6-Difluoro-N-{3-[3-(thien-2-ylcarbonyl)pyrazolo[1,5-a]pyrimidin-7-yl]phenyl}benzamide;
N-{3-[3-(Thien-2-ylcarbonyl)pyrazolo[1,5-a]pyrimidin-7-yl]phenyl}pyridine-2-carboxamide;
N-{5-[3-(Thien-2-ylcarbonyl)pyrazolo[1,5-a]pyrimidin-7-yl]thien-2-yl}benzamide;
(7-{3-[(2-Phenylethyl)amino]phenyl}pyrazolo[1,5-a]pyrimidin-3-yl)(thien-2-yl)methanone;
(7-{3-[(2-Methoxybenzyl)amino]phenyl}pyrazolo[1,5-a]pyrimidin-3-yl)(thien-2-yl)methanone;
N-Butyl-N′-{3-[3-(thien-2-ylcarbonyl)pyrazolo[1,5-a]pyrimidin-7-yl]phenyl}thiourea;
(7-{3-[(3-Chlorobenzyl)amino]phenyl}pyrazolo[1,5-a]pyrimidin-3-yl)(thien-2-yl)methanone;
(7-{3-[(4-Chlorobenzyl)amino]phenyl}pyrazolo[1,5-a]pyrimidin-3-yl)(thien-2-yl)methanone;
(7-{3-[(2-Furylmethyl)amino]phenyl}pyrazolo[1,5-a]pyrimidin-3-yl)(thien-2-yl)methanone;
(7-{3-[(2-Chlorobenzyl)amino]phenyl}pyrazolo[1,5-a]pyrimidin-3-yl)(thien-2-yl)methanone;
(7-{3-[(3-Furylmethyl)amino]phenyl}pyrazolo[1,5-a]pyrimidin-3-yl)(thien-2-yl)methanone;
(7-{3-[(2E)-But-2-enylamino]phenyl}pyrazolo[1,5-a]pyrimidin-3-yl)(thien-2-yl)methanone;
(7-{3-[(3-Methylbut-2-enyl)amino]phenyl) pyrazolo[1,5-a]pyrimidin-3-yl)(thien-2-yl)methanone;
[7-(6-Chloropyridin-3-yl)pyrazolo[1,5-a]pyrimidin-3-yl](thien-2-yl)methanone;
2,6-Difluoro-N-{2-fluoro-5-[3-(thien-2-ylcarbonyl)pyrazolo[1,5-a]pyrimidin-7-yl]phenyl}benzamide;
2-Fluoro-N-{2-fluoro-5-[3-(thien-2-ylcarbonyl)pyrazolo[1,5-a]pyrimidin-7-yl]phenyl}benzamide;
2-Chloro-N-{2-fluoro-5-[3-(thien-2-ylcarbonyl)pyrazolo[1,5-a]pyrimidin-7-yl]phenyl}benzamide;
(7-{3-[(2-Methylbenzyl)amino]phenyl}pyrazolo[1,5-a]pyrimidin-3-yl)(thien-2-yl)methanone;
(7-{3-[(2-Phenylpropyl)amino]phenyl}pyrazolo[1,5-a]pyrimidin-3-yl)(thien-2-yl)methanone;
(7-{3-[(Cyclohexylmethyl)amino]phenyl}pyrazolo[1,5-a]pyrimidin-3-yl)(thien-2-yl)methanone;
2-Chloroethyl 2-fluoro-5-[3-(thien-2-ylcarbonyl)pyrazolo[1,5-a]pyrimidin-7-yl]phenylcarbamate;
N-{2-Fluoro-5-[3-(thien-2-ylcarbonyl)pyrazolo[1,5-a]pyrimidin-7-yl]phenyl}-N′-isopropylurea;
4-(1H-Pyrrol-1-yl)-N-{3-[3-(thien-2-ylcarbonyl)pyrazolo[1,5-a]pyrimidin-7-yl]phenyl}benzamide;
2,6-Dichloro-N-{3-[3-(thien-2-ylcarbonyl)pyrazolo[1,5-a]pyrimidin-7-yl]phenyl}benzamide;
N-{3-[3-(Thien-2-ylcarbonyl)pyrazolo[1,5-a]pyrimidin-7-yl]phenyl}pyrazine-2-carboxamide;
Phenyl N′-cyano-N-{3-[3-(thien-2-ylcarbonyl)pyrazolo[1,5-a]pyrimidin-7-yl]phenyl}imidocarbamate;
1-Methyl-N-{3-[3-(thien-2-ylcarbonyl)pyrazolo[1,5-a]pyrimidin-7-yl]phenyl)-1H-Pyrrole-2-carboxamide;
N-{2-Fluoro-5-[3-(thien-2-ylcarbonyl)pyrazolo[1,5-a]pyrimidin-7-yl]phenyl}isonicotinamide;
N-Isopropyl-N′-{3-[3-(thien-2-ylcarbonyl)pyrazolo[1,5-a]pyrimidin-7-yl]phenyl)thiourea;
(7-Chloropyrazolo[1,5-a]pyrimidin-3-yl)(thien-2-yl)methanone;
2,6-Dichloro-N-(2,6-dichlorobenzoyl)-N-{3-[3-(thien-2-ylcarbonyl)pyrazolo[1,5-a]pyrimidin-7-yl]phenyl}benzamide;
tert-Butyl 4-[2-oxo-2-({3-[3-(thien-2-ylcarbonyl)pyrazolo[1,5-a]pyrimidin-7-yl]phenyl}amino)ethyl]piperidine-1-carboxylate;
2-Piperidin-4-yl-N-{3-[3-(thien-2-ylcarbonyl)pyrazolo[1,5-a]pyrimidin-7-yl]phenyl}acetamide;
[7-(3-{[(2E)-3-(2-Methoxyphenyl)prop-2-enyl]amino}phenyl)pyrazolo[1,5-a]pyrimidin-3-yl](thien-2-yl)methanone;
[7-(3-{[(2E)-3-(4-Methoxyphenyl)prop-2-enyl]amino}phenyl)pyrazolo[1,5-a]pyrimidin-3-yl](thien-2-yl)methanone;
[7-(3-{[(2E)-2-Methyl-3-phenylprop-2-enyl]amino}phenyl)pyrazolo[1,5-a]pyrimidin-3-yl](thien-2-yl)methanone;
[7-(3-{[(2E)-3-(4-Nitrophenyl)prop-2-enyl]amino}phenyl)pyrazolo[1,5-a]pyrimidin-3-yl](thien-2-yl)methanone;
(7-{3-[(3,3-Diphenylprop-2-enyl)amino]phenyl}pyrazolo[1,5-a]pyrimidin-3-yl)(thien-2-yl)methanone;
(7-{3-[(3-Phenylbutyl)amino]phenyl}pyrazolo[1,5-a]pyrimidin-3-yl)(thien-2-yl)methanone;
[7-(3-{[(2E)-3-(4-Hydroxy-3-methoxyphenyl)prop-2-enyl]amino}phenyl)pyrazolo[1,5-a]pyrimidin-3-yl](thien-2-yl)methanone;
tert-Butyl 4-[({3-[3-(thien-2-ylcarbonyl)pyrazolo[1,5-a]pyrimidin-7-yl]phenyl}amino)carbonyl]piperidine-1-carboxylate;
N,N′-Bis{3-[3-(thien-2-ylcarbonyl)pyrazolo[1,5-a]pyrimidin-7-yl]phenyl}thiourea;
Isobutyl 3-(3-thien-2-ylpyrazolo[1,5-a]pyrimidin-7-yl)phenylcarbamate;
N-{3-[3-(Thien-2-ylcarbonyl)pyrazolo[1,5-a]pyrimidin-7-yl]phenyl}piperidine-4-carboxamide;
[7-(3-{[(2E)-2-Hexyl-3-phenylprop-2-enyl]amino}phenyl)pyrazolo[1,5-a]pyrimidin-3-yl](thien-2-yl)methanone;
[7-(3-{[(2E)-2-Pentyl-3-phenylprop-2-enyl]amino}phenyl)pyrazolo[1,5-a]pyrimidin-3-yl](thien-2-yl)methanone;
N-Isopropyl-N′-{3-[3-(thien-2-ylcarbonyl)pyrazolo[1,5-a]pyrimidin-7-yl]phenyl}guanidine;
[7-(3-{[(2E)-3-Phenylprop-2-enyl]amino}phenyl)pyrazolo[1,5-a]pyrimidin-3-yl](thien-2-yl)methanone;
[7-(3-{[(2E)-3-(4-tert-Butylphenyl)-2-methylprop-2-enyl]amino}phenyl)pyrazolo[1,5-a]pyrimidin-3-yl](thien-2-yl)methanone;
N′-Cyano-N-isopropyl-N′-{3-[3-(thien-2-ylcarbonyl)pyrazolo[1,5-a]pyrimidin-7-yl]phenyl}guanidine;
N-{3-[3-(Thien-2-ylcarbonyl)pyrazolo[1,5-a]pyrimidin-7-yl]phenyl}-1H-imidazole-5-carboxamide;
Pyridin-4-ylmethyl 3-[3-(thien-2-ylcarbonyl)pyrazolo[1,5-a]pyrimidin-7-yl]phenylcarbamate;
1-Methyl-N-{3-[3-(thien-2-ylcarbonyl)pyrazolo[1,5-a]pyrimidin-7-yl]phenyl}piperidine-4-carboxamide;
(7-{3-[(2-Ethylhexyl)amino]phenyl}pyrazolo[1,5-a]pyrimidin-3-yl)(thien-2-yl)methanone;
3-(1,1-Dioxidothiomorpholin-4-yl)propyl 3-[3-(thien-2-ylcarbonyl)pyrazolo[1,5-a]pyrimidin-7-yl]phenylcarbamate;
2-Chloro-N-{2-(dimethylamino)-5-[3-(thien-2-ylcarbonyl)pyrazolo[1,5-a]pyrimidin-7-yl]phenyl}benzamide;
4-Morpholin-4-yl-N-{3-[3-(thien-2-ylcarbonyl)pyrazolo[1,5-a]pyrimidin-7-yl]phenyl}butanamide;
Pyridin-3-ylmethyl 3-[3-(thien-2-ylcarbonyl)pyrazolo[1,5-a]pyrimidin-7-yl]phenylcarbamate;
3-Morpholin-4-ylpropyl 3-[3-(thien-2-ylcarbonyl)pyrazolo[1,5-a]pyrimidin-7-yl]phenylcarbamate;
[7-(3-{[(2E)-2-Methylpent-2-enyl]amino}phenyl)pyrazolo[1,5-a]pyrimidin-3-yl](thien-2-yl)methanone;
2-Pyridin-4-yl-N-{3-[3-(thien-2-ylcarbonyl)pyrazolo[1,5-a]pyrimidin-7-yl]phenyl}acetamide;
N-{2-(Dimethylamino)-5-[3-(thien-2-ylcarbonyl)pyrazolo[1,5-a]pyrimidin-7-yl]phenyl}isonicotinamide;
Pyridin-2-ylmethyl 3-[3-(thien-2-ylcarbonyl)pyrazolo[1,5-a]pyrimidin-7-yl]phenylcarbamate;
Thien-2-yl(7-{3-[(3,5,5-trimethylhexyl]amino]phenyl}pyrazolo[1,5-a]pyrimidin-3-yl](thien-2-yl)methanone;
(7-{3-[(Cyclohex-3-en-1-ylmethyl)emino]phenyl}pyrazolo[1,5-a]pyrimidin-3-yl)-(thien-2-yl)-methanone;
1-{3-[3-(Thien-2-ylcarbonyl)pyrazolo[1,5-a]pyrimidin-7-yl]phenyl}pyrrolidin-2-one;
2-Chloro-N-(2-chlorobenzoyl)-N-{2-fluoro-5-[3-(thien-2-ylcarbonyl)pyrazolo[1,5-a]pyrimidin-7-yl]phenyl}benzamide;
2-Pyridin-3-yl-N-{3-[3-(thien-2-ylcarbonyl)pyrazolo[1,5-a]pyrimidin-7-yl]phenyl}acetamide;
3-Pyridin-4-yl-N-{3-[3-(thien-2-ylcarbonyl)pyrazolo[1,5-a]pyrimidin-7-yl]phenyl}propanamide;
N-{3-[3-(Thien-2-ylcarbonyl)pyrazolo[1,5-a]pyrimidin-7-yl]phenyl}-1H-pyrazole-4-arboxamide;
4-oxo-4-{3-[3-(Thien-2-ylcarbonyl)pyrazolo[1,5-a]pyrimidin-7-yl]phenyl}amino)butanoic acid;
2-[({3-[3-(Thien-2-ylcarbonyl)pyrazolo[1,5-a]pyrimidin-7-yl]phenyl}amino)carbonyl]benzoic acid;
5-Oxo-5-({3-[3-(thien-2-ylcarbonyl)pyrazolo[1,5-a]pyrimidin-7-yl]phenyl}amino)pentanoic acid;
3-Methyl-5-oxo-5-({3-[3-(thien-2-ylcarbonyl)pyrazolo[1,5-a]pyramidin-7-yl]phenyl}amino)pentanoic acid;
4,5-Dichloro-2-[({3-[3-(thien-2-ylcarbonyl)pyrazolo[1,5-a]pyrimidin-7-yl]phenyl}amino)carbonyl]benzoic acid;
(7-{3-[(2-Methylundecyl)amino]phenyl}pyrazolo[1,5-a]pyrimidin-3-yl)(thien-2-yl)methanone;
3-Pyridin-3-yl-N-(3-[3-(thien-2-ylcarbonyl)pyrazolo[1,5-a]pyrimidin-7-yl]phenyl}propanamide;
2-(Pyridin-4-ylthio)-N-{3-[3-(thien-2-ylcarbonyl)pyrazolo[1,5-a]pyrimidin-7-yl]phenyl}acetamide;
{7-[4′-(Dimethylamino)-1,1′-biphenyl-3-yl]pyrazolo[1,5-a]pyrimidin-3-yl}(thien-2-yl)methanone;
[7-(3′-Amino-1,1′-biphenyl-3-yl)pyrazolo[1,5-a]pyrimidin-3-yl](thien-2-yl)methanone;
{7-[4′-(Hydroxymethyl)-1,1′-biphenyl-3-yl]pyrazolo[1,5-a]pyrimidin-3-yl}(thien-2-yl)methanone;
{7-[3′-(Dimethylamino)-1,1′-biphenyl-3-yl]pyrazolo[1,5-a]pyrimidin-3-yl}(thien-2-yl)methanone;
{7-[4′-(Dimethylamino)-6-fluoro-1,1′-biphenyl-3-yl]pyrazolo[1,5-a]pyrimidin-3-yl}(thien-2-yl)methanone;
{7-[6-Fluoro-4′-(hydroxymethyl)-1,1′-biphenyl-3-yl]pyrazolo[1,5-a]pyrimidin-3-yl}(thien-2-yl)methanone;
[7-(4′-Ethyl-1,1′-biphenyl-3-yl)pyrazolo[1,5-a]pyrimidin-3-yl](thien-2-yl)methanone;
3-{3′-[3-(Thien-2-ylcarbonyl)pyrazolo[1,5-a]pyrimidin-7-yl]-1,1′-biphenyl-4-yl}propanoic acid;
3′-[3-(Thien-2-ylcarbonyl)pyrazolo[1,5-a]pyrimidin-7-yl]-1,1′-biphenyl-4-carbaldehyde;
{7-[4′-(Morpholin-4-ylmethyl)-1,1′-biphenyl-3-yl]pyrazolo[1,5-a]pyrimidin-3-yl}(thien-2-yl)methanone;
(7-{4′-[(4-Methylpiperazin-1-yl)methyl]-1,1′-biphenyl-3-yl}pyrazolo[1,5-a]pyrimidin-3-yl)(thien-2-yl)methanone;
[7-(4′-{[(2-Morpholin-4-ylethyl)amino]methyl}-1,1′-biphenyl-3-yl)pyrazolo[1,5-a]pyrimidin-3-yl](thien-2-yl)methanone;
(7-{4′-[(Diethylamino)methyl]-1,1′-biphenyl-3-yl}pyrazolo[1,5-a]pyrimidin-3-yl)(thien-2-yl)methanone;
(7-{4′-[(Dimethylamino)methyl]-1,1′-biphenyl-3-yl}pyrazolo[1,5-a]pyrimidin-3-yl)(thien-2-yl)methanone;
N-{3-[3-(3-Aminophenyl)pyrazolo[1,5-a]pyrimidin-7-yl]phenyl}-3-methylbutanamide;
N-(3-{3-[3-(Dimethylamino)phenyl]pyrazolo[1,5-a]pyrimidin-7-yl}phenyl)-3-methylbutanamide;
N-{3-[3-(1H-Indol-5-yl)pyrazolo[1,5-a]pyrimidin-7-yl]phenyl}-3-methylbutanamide;
3-Methyl-N-{3-[3-(3,4,5-trimethoxyphenyl)pyrazolo[1,5-a]pyrimidin-7-yl]phenyl}butanamide;
3-Methyl-N-[3-(3-quinolin-8-ylpyrazolo[1,5-a]pyrimidin-7-yl)phenyl]butanamide;
N-(3-{3-[4-(Hydroxymethyl)phenyl]pyrazolo[1,5-a]pyrimidin-7-yl}phenyl)-3-methylbutanamide;
N-{3-[3-(4-Methoxyphenyl)pyrazolo[1,5-a]pyrimidin-7-yl]phenyl}-3-methylbutanamide;
N-(3-[3-(3-Chlorophenyl)pyrazolo[1,5-a]pyrimidin-7-yl]phenyl}-3-methylbutanamide;
N-{3-[3-(4-Chlorophenyl)pyrazolo[1,5-a]pyrimidin-7-yl]phenyl}-3-methylbutanamide;
N-{3-[3-(2,3-Dichlorophenyl)pyrazolo[1,5-a]pyrimidin-7-yl]phenyl}-3-methylbutanamide;
N-{3-[3-(3,4-Dichlorophenyl)pyrazolo[1,5-a]pyrimidin-7-yl]phenyl}-3-methylbutanamide;
N-{3-[3-(3,5-Dichlorophenyl)pyrazolo[1,5-a]pyrimidin-7-yl]phenyl}-3-methylbutanamide;
N-(3-[3-(3,5-Dimethylphenyl)pyrazolo[1,5-a]pyrimidin-7-yl]phenyl) -3-methylbutanamide;
3-Methyl-N-(3-{3-[3-(trifluoromethyl)phenyl]pyrazolo[1,5-a]pyrimidin-7-yl}phenyl)butanamide;
N-(3-[3-(3-Methoxyphenyl)pyrazolo[1,5-a]pyrimidin-7-yl]phenyl}-3-methylbutanamide;
N-{3-[3-(3-Ethoxyphenyl)pyrazolo[1,5-a]pyrimidin-7-yl]phenyl}-3-methylbutanamide;
3-Methyl-N-{3-[3-(4-phenoxyphenyl)pyrazolo[1,5-a]pyrimidin-7-yl]phenyl}butanamide;
N-{3-[3-(1,3-Benzodioxol-5-yl)pyrazolo[1,5-a]pyrimidin-7-yl]phenyl}-3-methylbutanamide;
N-{3-[3-(3-Cyanophenyl)pyrazolo[1,5-a]pyrimidin-7-yl]phenyl}-3-methylbutanamide;
N-{3-[3-(3-Acetylphenyl)pyrazolo[1,5-a]pyrimidin-7-yl]phenyl}-3-methylbutanamide;
N-{3-[3-(3-Formyl-4-methoxyphenyl)pyrazolo[1,5-a]pyrimidin-7-yl]phenyl}-3-methylbutanamide;
N-{3-[3-(1,1′-Biphenyl-3-yl)pyrazolo[1,5-a]pyrimidin-7-yl]phenyl}-3-methylbutanamide;
N-{3-[3-(1,1′-Biphenyl-4-yl)pyrazolo[1,5-a]pyrimidin-7-yl]phenyl}-3-methylbutanamide;
N-{3-[3-(3-Formylphenyl)pyrazolo[1,5-a]pyrimidin-7-yl]phenyl}-3-methylbutanamide;
3-Methyl-N-(3-{3-[3-(morpholin-4-ylmethyl)phenyl]pyrazolo[1,5-a]pyrimidin-7-yl}phenyl)butanamide;
3-Methyl-N-[3-(3-(3-[(4-methylpiperazin-1-yl)methyl]phenyl}pyrazolo[1,5-a]pyrimidin-7-yl)phenyl]butanamide;
3-[3-(3,5-Dimethylphenyl)pyrazolo[1,5-a]pyrimidin-7-yl]benzonitrile;
3-(3,5-Dimethylphenyl)-7-[3-(1H-tetraazol-5-yl)phenyl]pyrazolo[1,5-a]pyrimidine;
7-{3-[2-(Cyclobutylmethyl)-2H-tetraazol-5-yl]phenyl}-3-(3,5-dimethylphenyl)pyrazolo[1,5-a]pyrimidine;
3-(3,5-Dimethylphenyl)-7-{3-[2-(2-morpholin-4-ylethyl)-2H-tetraazol-5-yl]phenyl}pyrazolo[1,5-a]pyrimidine;
3-(3,5-Dimethylphenyl)-7-{3-[2-(pyridin-3-ylmethyl)-2H-tetrazol-5-yl]phenyl}pyrazolo[1,5-a]pyrimidine;
3-(3,5-Dimethylphenyl)-7-{3-[2-(pyridin-2-ylmethyl)-2H-tetrazol-5-yl]phenyl}pyrazolo[1,5-a]pyrimidine;
3-(3,5-Dimethylphenyl)-7-[3-(2-ethyl-2H-tetrazol-5-yl)phenyl]pyrazolo[1,5-a]pyrimidine;
N-[3-(3-{3-[(Dimethylamino)methyl]phenyl}pyrazolo[1,5-a]pyrimidin-7-yl)phenyl]-3-methylbutanamide;
3-(3,5-Dimethylphenyl)-7-(3-[2-(2-pyrrolidin-1-ylethyl)-2H-tetrazol-5-yl]phenyl}pyrazolo[1,5-a]pyrimidine;
3-(3,5-Dimethylphenyl)-7-{3-[2-(2-piperidin-1-ylethyl)-2H-tetrazol-5-yl]phenyl}pyrazolo[1,5-a]pyrimidine;
3-(3,5-Dimethylphenyl)-7-(3-[2-(pyridin-4-ylmethyl)-2H-tetrazol-5-yl]phenyl}pyrazolo[1,5-a]pyrimidine;
{7-[3-(1H-Tetraazol-5-yl)phenyl]pyrazolo[1,5-a]pyrimidin-3-yl}(thien-2-yl)methanone;
{7-[3-(2-Ethyl-2H-tetraazol-5-yl)phenyl]pyrazolo[1,5-a]pyrimidin-3-yl}(thien-2-yl)methanone;
(7-{3-[2-(Pyridin-3-ylmethyl)-2H-tetraazol-5-yl]phenyl}pyrazolo[1,5-a]pyrimidin-3-yl)(thien-2-yl)methanone;
{7-[3-(2-Isobutyl-2H-tetraazol-5-yl)phenyl]pyrazolo[1,5-a]pyrimidin-3-yl}(thien-2-yl)methanone;
(7-{3-[2-(Cyclobutylmethyl)-2H-tetraazol-5-yl]phenyl}pyrazolo[1,5-a]pyrimidin-3-yl)(thien-2-yl)methanone;
{7-[3-(2-Butyl-2H-tetraazol-5-yl)phenyl]pyrazolo[1,5-a]pyrimidin-3-yl}(thien-2-yl)methanone;
(7-{3-[2-(2-Morpholin-4-ylethyl)-2H-tetraazol-5-yl]phenyl}pyrazolo[1,5-a]pyrimidin-3-yl)(thien-2-yl)methanone;
(7-{3-[1-(Cyclobutylmethyl)-1H-tetraazol-5-yl]phenyl}pyrazolo[1,5-a]pyrimidin-3-yl)(thien-2-yl)methanone;
(7-{3-[2-(Pyridin-2-ylmethyl)-2H-tetraazol-5-yl]phenyl}pyrazolo[1,5-a]pyrimidin-3-yl)(thien-2-yl)methanone;
(7-{3-[2-(Pyridin-4-ylmethyl)-2H-tetraazol-5-yl]phenyl}pyrazolo[1,5-a]pyrimidin-3-yl)(thien-2-yl)methanone;
(7-(3-[2-(2-Pyrrolidin-1-ylethyl)-2H-tetraazol-5-yl]phenyl}pyrazolo[1,5-a]pyrimidin-3-yl)(thien-2-yl)methanone;
(7-{3-[2-(2-Piperidin-1-ylethyl)-2H-tetraazol-5-yl]phenyl}pyrazolo[1,5-a]pyrimidin-3-yl)(thien-2-yl)methanone;
(7-(3-[2-(2-Hydroxyethyl)-2H-tetraazol-5-yl]phenyl}pyrazolo[1,5-a]pyrimidin-3-yl)(thien-2-yl)methanone;
{7-[3-(1H-Pyrazol-3-yl)phenyl]pyrazolo[1,5-a]pyrimidin-3-yl}(thien-2-yl)methanone;
(7-{3-[1-(Cyclobutylmethyl)-1H-pyrazol-3-yl]phenyl}pyrazolo[1,5-a]pyrimidin-3-yl)(thien-2-yl)methanone;
(7-{3-[1-(2-Morpholin-4-ylethyl)-1H-pyrazol-3-yl]phenyl}pyrazolo[1,5-a]pyrimidin-3-yl)(thien-2-yl)methanone;
(7-{3-[1-(Pyridin-2-ylmethyl)-1H-pyrazol-3-yl]phenyl}pyrazolo[1,5-a]pyrimidin-3-yl)(thien-2-yl)methanone;
{7-[3-(1-Ethyl-1H-pyrazol-3-yl)phenyl]pyrazolo[1,5-a]pyrimidin-3-yl}(thien-2-yl)methanone;
(7-{3-[1-(2-Piperidin-1-ylethyl)-1H-pyrazol-3-yl]phenyl}pyrazolo[1,5-a]pyrimidin-3-yl)(thien-2-yl)methanone;
(7-{3-[1-(2-Pyrrolidin-1-ylethyl)-1H-pyrazol-3-yl]phenyl}pyrazolo[1,5-a]pyrimidin-3-yl)(thien-2-yl)methanone.

The compounds of the present invention may be prepared as set forth in the following reaction scheme:

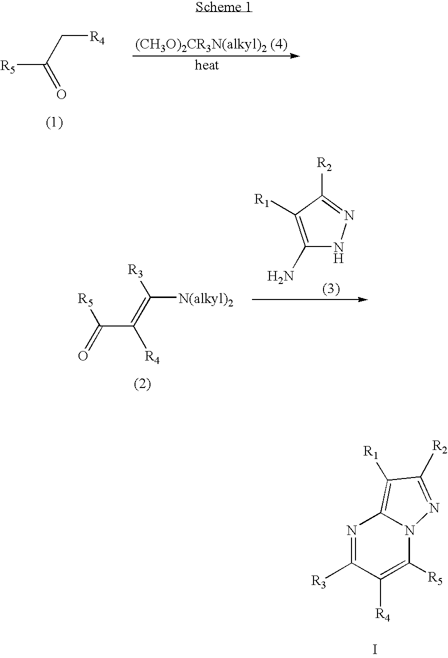

Referring to Scheme 1, the reaction of ketone (1) with acetals of N,N-dialkylformamides or acetals of N,N-dialkylacetamide can be carried out in an inert solvent or without a solvent. The reaction of the substituted 3-aminopyrazole (3) where R₁ and R₂ are herein before defined, and an appropriately substituted 3-dialkylamino-1-(aryl or heteroaryl)-2-propen-1-one (2) where R₃, R₄, and R₅ are herein before defined in weak acid such as glacial acetic acid or in an inert solvent such as toluene, acetonitrile or dimethoxyethane, at reflux temperature for several hours, produces the desired products I where R₁, R₂, R₃, R₄, and R₅ are herein before defined.

Substituted 3-dimethylamino-1-(3-heteroaryl)-2-propen-1-ones are disclosed in U.S. Pat. Nos. 4,281,000 and 4,521,422 and 3-dialkylamino-1-phenyl-2-propen-1-ones are disclosed in U.S. Pat. Nos. 4,178,449 and 4,236,005.

The 3-amino-4-pyrazoles (3) where R₂ is H are disclosed in U.S. Pat. Nos. 4,236,005; 4,281,000; 4,521,422; 4,626,538; 4,654347; and 4,900,836.

Pyrazolo[1,5-a]pyrimidines are prepared by condensation of 3-aminopyrazoles and substituted 3-aminopyrazoles with 1,3-dicarbonyl compounds as described in J. Med. Chem., 18, 645 (1974); J. Med. Chem. 18, 460 (1975); J. Med. Chem., 20, 386 (1977); Synthesis, 673 (1982) and references contained therein.

The preparation of the compounds of this invention encompassed by Formula (7) is described below in Scheme 2 where R₁, R₂, R₃, R₄, R₇, R₈, and R₉ are as defined herein before. The reaction of (5) with substituted 3-aminopyrazole (3) in acetic acid at reflux for several hours gives compounds represented by Formula (6). The reduction of nitro compound (6) with reducing agents such as Fe, SnCl₂-xH₂O, catalytic hydrogenation and the like, gives compounds represented by Formula (7).

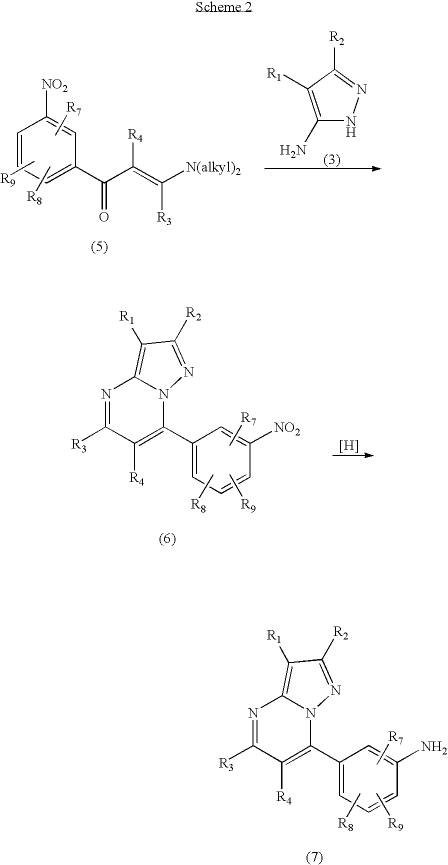

The preparation of the compounds of this invention encompassed by Formula (9) is described below in Scheme 3 where R₁, R₂, R₃, R₄, R₇, R₈, R₉, R₁₁, and R₁₃ are as defined herein before. The reaction of aniline (7) with the acylating agents such as acyl chloride (8) or carboxylic acid anhydride, in the presence of a base such as pyridine, triethylamine, N-methylmorpholine, 4-dimethylaminopyridine and the like, gives compounds represented by Formula (9). Alternatively, (9) can be prepared from the reaction of aniline (7) with carbamate intermediate (12) generated in situ by treating carboxylic acid (10) with alkyl chloroformate (11) in the presence of base as defined above.

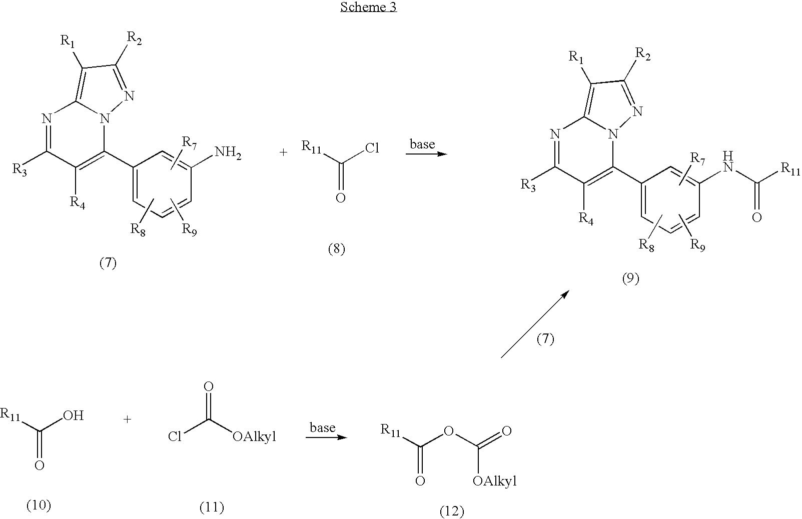

The preparation of the compounds of this invention encompassed by Formula (14) is described below in Scheme 4 where R₁, R₂, R₃, R₄, R₇, R₈, R₉, R₁₁, R₁₂, and R₁₃ are as defined herein before. The reaction of aniline (7) with alkyl isocyanate or aryl isocyanate (13) in the presence of base, such as pyridine, triethylamine, N-methylmorpholine, 4-dimethylaminopyridine and the like, gives compounds represented by Formula (14). Alternatively, (14) can be prepared by treating aniline (7) with phenyl chloroformate or substituted phenyl chloroformate (15) to form carbamate intermediate (16) in the presence of base as defined above, followed by reaction with amine (17).

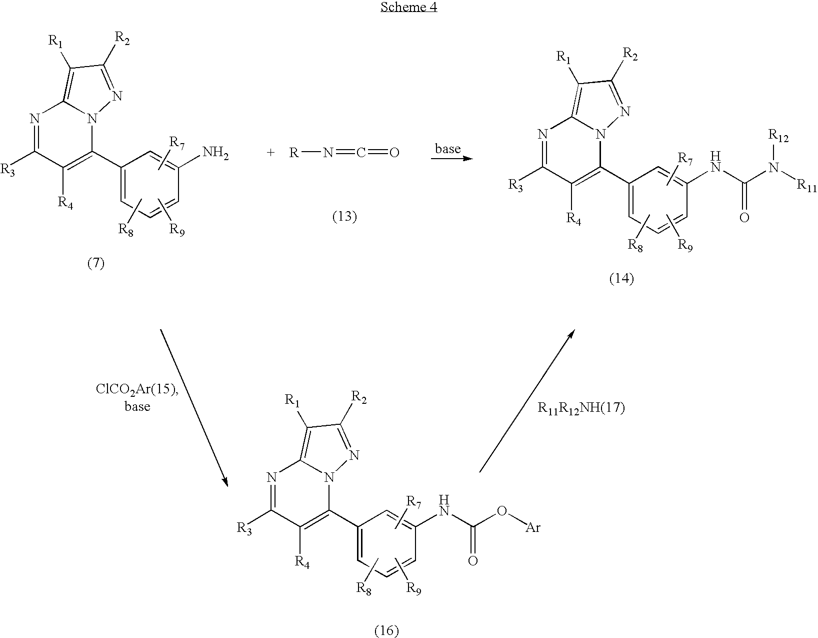

The preparation of the compounds of this invention encompassed by Formula (19) is described below in Scheme 5 where R₁, R₂, R₃, R₄, R₇, R₈, R₉, R₁₁, and R₁₃ are as defined herein before. The reaction of aniline (7) with alkyl chloroformate or aryl chloroformate (18) in the presence of base, such as pyridine, triethylamine, N-methylmorpholine, 4-dimethylaminopyridine and the like, gives compounds represented by Formula (19). Alternatively, (19) can be prepared from the reaction of aniline (7) with carbamate intermediate (22) generated in situ by treating alcohol (20) with aryl chloroformate (21), such as 4-nitrophenyl chloroformate in the presence of base as defined above.

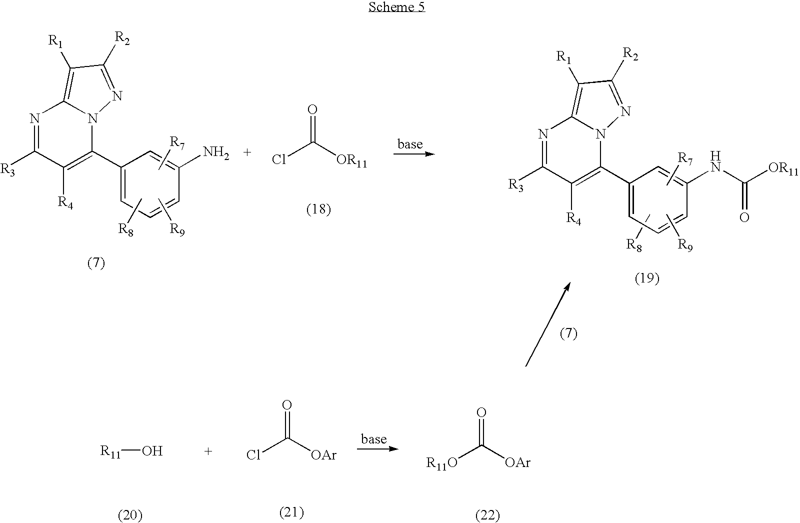

The preparation of the compounds of this invention encompassed by Formula (24) and (25) is described below in Scheme 6 where R₁, R₂, R₃, R₄, R₇, R₈, R₉, R₁₁, and R₁₂ are as defined herein before, with the proviso that R₁, and R₁₂ are not an aryl or heteroaryl group. The reaction of aniline (7) with aldehydes or ketones using a reducing agent (such as sodium cyanoborohydride, sodium triacetoxylborohydride, and the like) and an inert solvent such as tetrahydrofuran, methylene chloride, N,N-dimethylformamide (“DMF”) and the like, gives compounds represented by Formula (24). Compound (25) can be prepared from (24) with the same or different aldehyde or ketone using the same procedure described above.

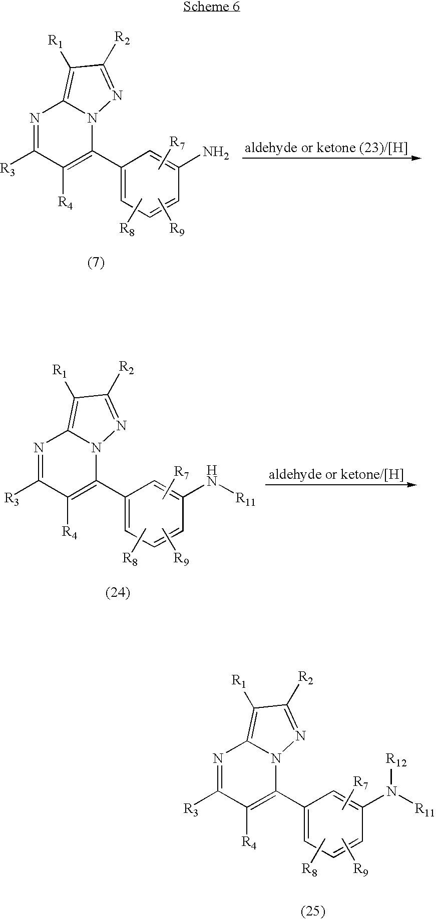

The preparation of the compounds of this invention encompassed by Formula (27) is described below in Scheme 7 where R₁, R₂, R₃, R₄, R₇, R₈, R₉, R₁₁, and R₁₃ are as defined herein before. The reaction of aniline (7) with alkyl sulfonyl chloride or aryl sulfonyl chloride (26) in the presence of base such as pyridine, triethylamine, N-methylmorpholine, 4-dimethylaminopyridine (“DMAP”) and the like, gives compounds represented by Formula (27).

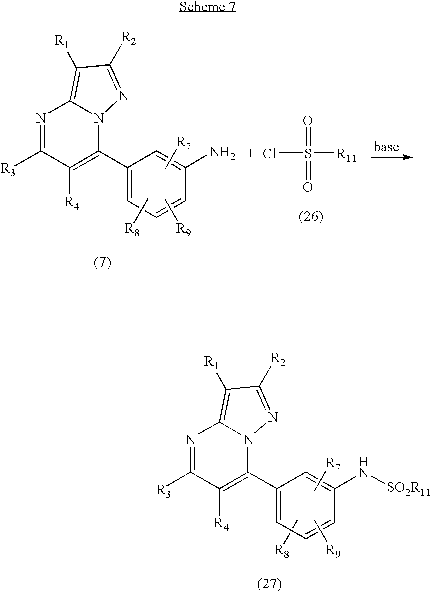

The preparation of the compounds of this invention encompassed by Formula (29) is described below in Scheme 8 where R₁, R₂, R₃, R₄, R₇, R₈, R₉, R₁₁, and R₁₃ are as defined herein before. The reaction of aniline (7) with alkyl isothiocyanate or aryl isothiocyanate (28) in the presence of base such as pyridine, triethylamine, N-methylmorpholine, DMAP and the like, gives compounds represented by Formula (29).

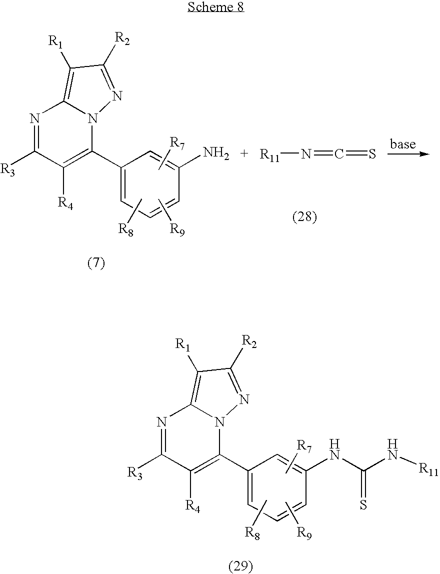

The preparation of the compounds of this invention encompassed by Formula (32) is described below in Scheme 9 where R₁, R₂, R₃, R₄, R₇, R₈, R₉, R₁₁, R₁₂, and R₁₃ are as defined herein before. The reaction of aniline (7) with diarylcyanocarbonimidate, such as diphenylcyanocarbonimidate (30), in an inert solvent such as acetonitrile, DMF, methylene chloride, tetrahydrofuran and the like, gives compounds represented by Formula (31). Compound (31) was subsequently reacted with amine (17) in an inert solvent such as methanol, ethanol, isopropanol, acetonitrile, DMF, methylene chloride, tetrahydrofuran and the like to produce compounds represented by Formula (32).

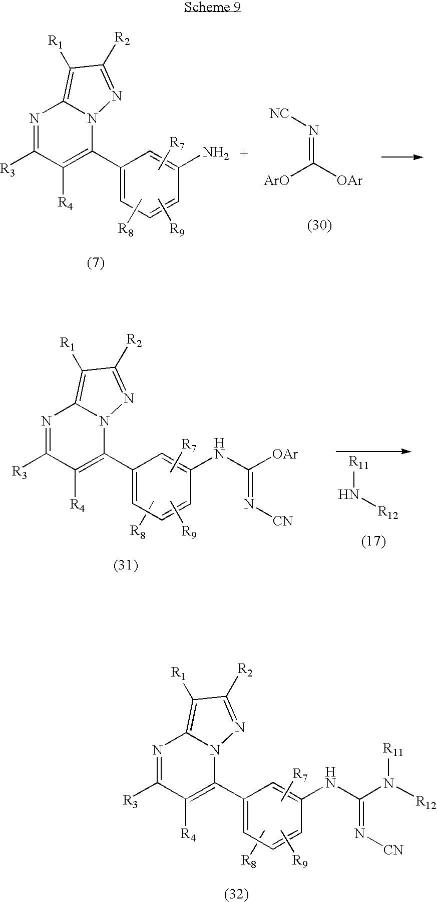

The preparation of the compounds of this invention encompassed by Formula (37) is described below in Scheme 10 where R₁, R₂, R₃, R₄, R₇, R₈, R₉, R₁₁, and R₁₂ are as defined herein before. The reaction of ketone (33) with acetals of N,N-dialkylformamides or acetals of N,N-dialkylacetamide can be carried out in an inert solvent such as DMF, acetonitrile, toluene and the like, or without a solvent. The ketoester (33) where R₄, R₇, R₈, and R₉ are H is disclosed in J. Med. Chem., 13, 674 (1970).

The reaction of (34) with substituted 3-aminopyrazole (3) in acetic acid at reflux for several hours gives compounds represented by Formula (35). Compound (35) may be hydrolyzed in strong aqueous base, such as potassium hydroxide, sodium hydroxide and the like, at temperatures from 20° C.-100° C., to give compounds represented by Formula (36), which may be treated with amine (17) in an inert solvent such as methylene chloride, tetrahydrofuran, DMF, acetonitrile and the like, in the presence of the organic base such as triethylamine, diisopropylethylamine, pyridine and the like, and a catalyst such as benzotriazol-1-yloxytripyrrolidinophosphonium hexafluorophosphate (Py-BOP) and the like, to produce the compounds represented by Formula (37).

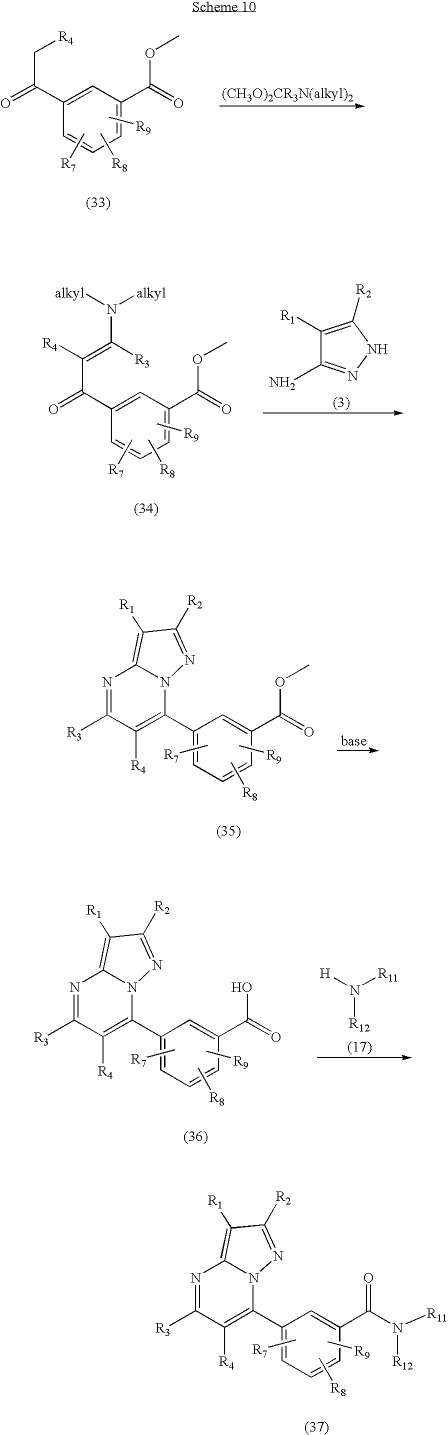

The preparation of the compounds of this invention encompassed by Formulas (42)-(49) is described below in Scheme 11 where R₁, R₂, R₃, R₄, R₇, R₈, R₉, R₁₁, R₁₂, and R₁₃ are as defined herein before. The reaction of thiophene ketone (38) with acetals of N,N-dialkylformamides or acetals of N,N-dialkylacetamide can be carried out in an inert solvent such as DMF, acetonitrile, toluene and the like, or without a solvent. The reaction of (39) with substituted 3-aminopyrazole (3) in acetic acid at reflux for several hours gives compounds represented by Formula (40). The reduction of nitro compound (40) with reducing agents such as Fe, SnCl₂-xH₂O, catalytic hydrogenation and the like, gives compounds represented by Formula (41). Compound (41) may be converted to the corresponding amides, ureas, carbamates, substituted amines, sulphonamides, thioureas, thiocarbamates, cyanoimidates and the like as described in Schemes 3-9.

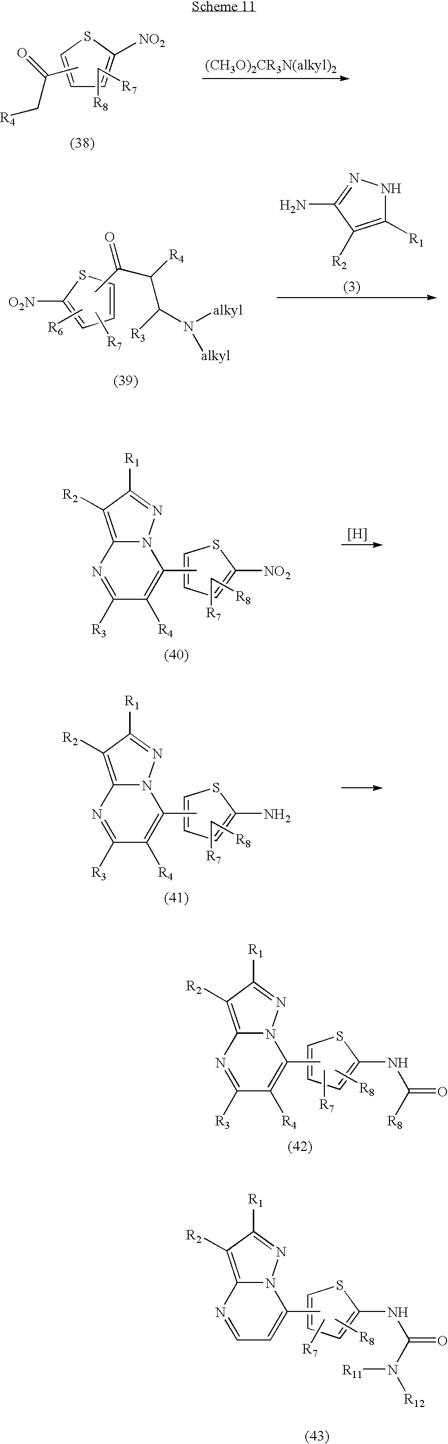

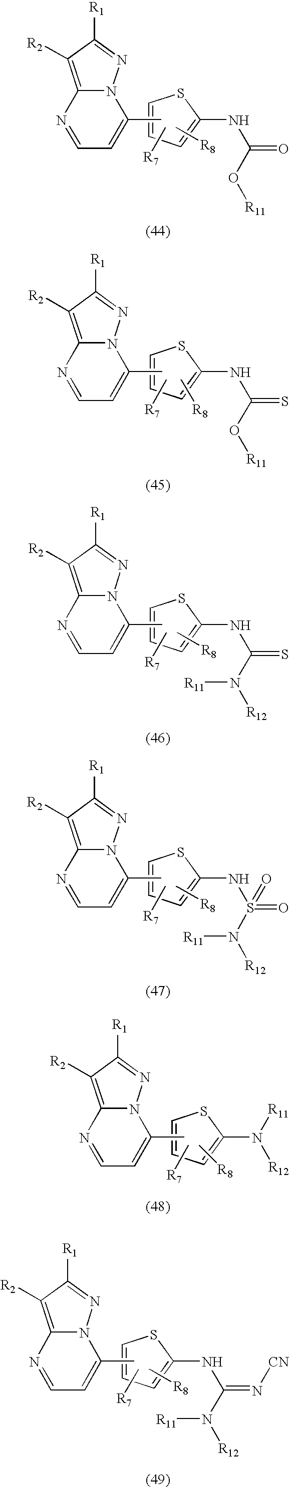

The preparation of the compounds of this invention encompassed by Formulas (57)-(64) is described below in Scheme 12 where R₁, R₂, R₃, R₄, R₇, R₈, R₉, R₁₁, R₁₂, and R₁₃ are as defined herein before. The reaction of pyrimidin-4-one (50) with ketone (51) may be carried out in alcoholic solvent such as methanol, ethanol and the like, in the presence of NH₃ at elevated temperature such as 100° C. in a sealed tube to give compounds represented by Formula (52). The pyrimidin-4-one (50) where R₇, and R₈, are H may be prepared by the procedures disclosed in J. Amer. Chem. Soc. 82, 486 (1960) and Bull. Chem. Soc. Jpn. 69, 1997 (1996). The 3-nitropyridine (52) where R₄, R₇, R₈, and R₉ are H is disclosed in Synthesis, 1277 (1997).

Oxidation of compounds (52) with oxidation agents such as CrO₃, KMnO₄ and the like in an inert solvent such as methylene chloride, chloroform and the like generate 3-acetylpyridine (53). The reaction of ketones (53) with acetals of N,N-dialkylformamides or acetals of N,N-dialkylacetamide can be carried out in an inert solvents such as DMF, acetonitrile, toluene and the like, or without a solvent. The reaction of (54) with substituted 3-aminopyrazole (3) in acetic acid at reflux for several hours gives compounds represented by Formula (55). The reduction of nitro compounds (55) with reducing agents such as Fe, SnCl₂-xH₂O, catalytic hydrogenation and the like, gives compounds represented by Formula (56). Compound (56) may be converted to the corresponding amides, ureas, carbamates, substituted amines, sulphonamides, thioureas, thiocarbamates, cyanoimidates and the like as described in Schemes 3-9.

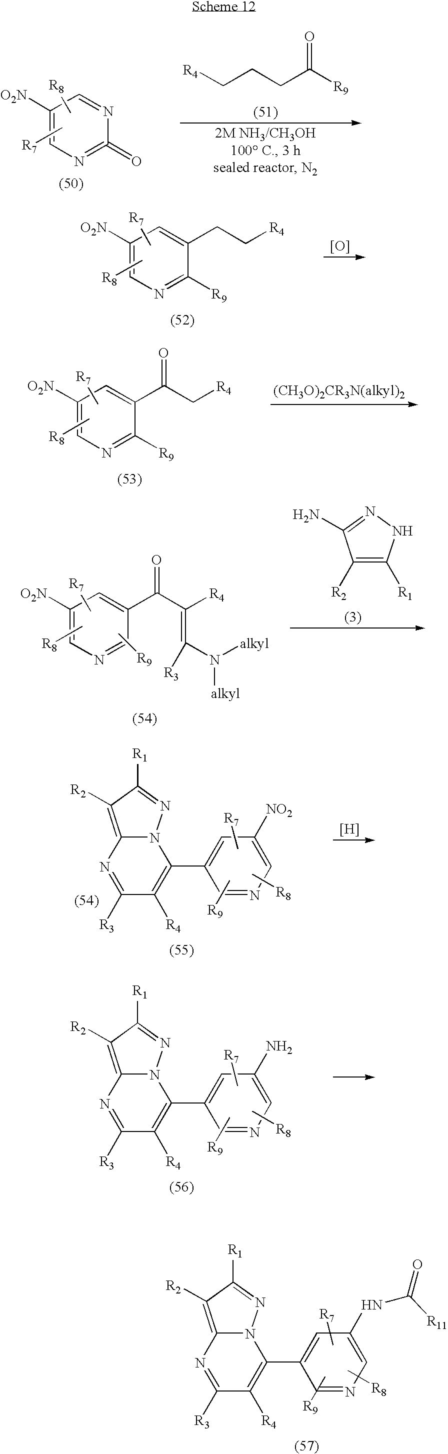

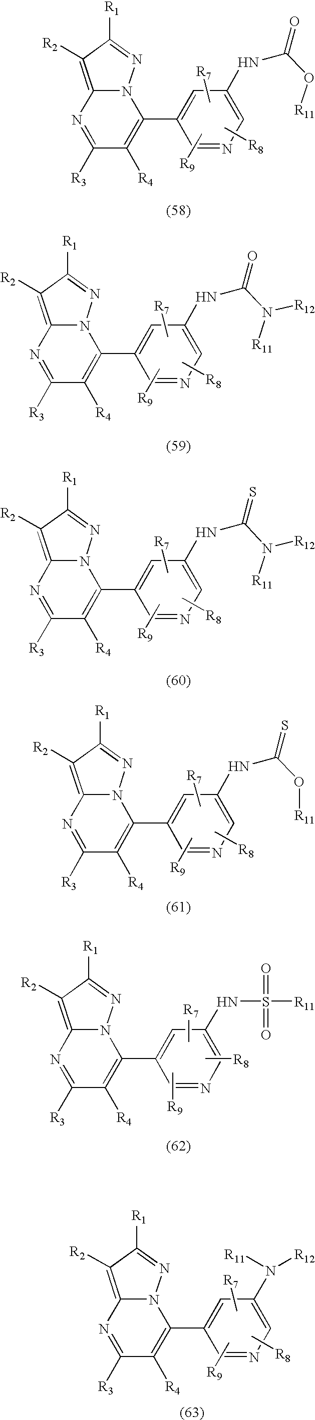

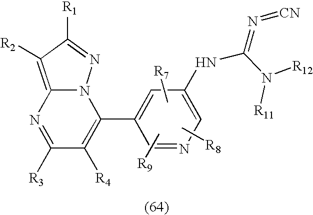

The compounds of this invention may be formulated neat or may be combined with one or more pharmaceutically acceptable carriers for administration. For example, solvents, diluents and the like and may be administered orally in such forms as tablets, capsules, dispersible powders, granules, or suspensions containing, for example, from about 0.05 to 5% of suspending agent, syrups containing, for example, from about 10 to 50% of sugar, and elixirs containing, for example, from about 20 to 50% ethanol, and the like, or parentally in the form of sterile injectable solution or suspension containing from about 0.05 to 5% suspending agent in an isotonic medium. Such pharmaceutical preparations may contain, for example, from about 0.05 up to about 90% of the active ingredient in combination with the carrier, more usually between about 5% and 60% by weight.

The effective dosage of active ingredient employed may vary depending on the particular compound employed, the mode of administration and the severity of the condition being treated. However, in general, satisfactory results are obtained when the compounds of the invention are administered at a daily dosage of from about 0.5 to 1000 mg/kg of animal body weight, optionally given in divided doses two to four times a day, or in sustained release form. For most large mammals the total daily dosage is from about 1 to 1000 mg, preferably from about 2 to 500 mg. Dosage forms suitable for internal use comprise from about 0.5 to 1000 mg of the active compound in intimate admixture with a solid or liquid pharmaceutically acceptable carrier. This dosage regimen may be adjusted to provide the optimal therapeutic response. For example, several divided doses may be administered daily or the dose may be proportionally reduced as indicated by the exigencies of the therapeutic situation.

The compounds of this invention may be administered orally as well as by intravenous, intramuscular, or subcutaneous routes. Solid carriers include starch, lactose, dicalcium phosphate, microcrystalline cellulose, sucrose and kaolin, while liquid carriers include sterile water, polyethylene glycols, non-ionic surfactants and edible oils such as corn, peanut and sesame oils, as are appropriate to the nature of the active ingredient and the particular form of administration desired. Adjuvants customarily employed in the preparation of pharmaceutical compositions may be advantageously included, such as flavoring agents, preserving agents and antioxidants, for example, vitamin E, ascorbic acid, BHT and BHA.

In some cases, it may be desirable to administer the compounds directly to the airways in the form of an aerosol.

The compounds of this invention may also be administered parenterally or intraperitoneally. Solutions or suspensions of these active compounds as a free base or pharmacologically acceptable salt can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemperaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixture thereof, and vegetable oils.

For the treatment of cancer, the compounds of this invention can be administered in combination with other antitumor substances or with radiation therapy. These other substances or radiation can be given at the same or at different times as the compounds of this invention. These combined therapies may effect synergy and result in improved efficacy. For example, the compounds of this invention can be used in combination with mitotic inhibitors such as taxol or vinblastine, alkylating agents such as cisplatin or cyclophosamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, signal transduction inhibitors such as EGFR (epidermal growth factor receptor) antibodies and EGFR inhibitors, and antiestrogens such as tamoxifen.

The compounds of this invention can also be used with other agents useful in treating abnormal cell growth or cancer, including agents capable of enhancing antitumor immune responsed, such as CTLA4 (cytotoxic lymphocyte antigen 4) antibodies, and other agents capable of blocking CTLA4; and anti-proliferative agents such as farnesyl protein transferase inhibitors, and the like.

Representative compounds of this invention were evaluated in several standard pharmaceutical test procedures that showed that the compounds of this invention possess significant activity as inhibitors of the growth of various cancer cells. Based on the activity shown in the standard pharmacological test procedures, the compounds of this invention are therefore useful as antineoplastic agents. In particular, these compounds are useful in treating, inhibiting the growth of, or eradicating neoplasms such as those of the breast, kidney, bladder, mouth, larynx, esophagus, stomach, colon, ovary, lung, pancreas, liver, prostate and skin.

The test procedures and results obtained are shown below.

Cytotoxicity Assay Using Isogenic Cell Lines:

1. 80S14 (p21-deficient) cells were cultured in RPMI 1640 medium (Gibco/Invitrogen Life technologies) supplemented with 10% fetal bovine serum (FBS, Gibco) and 10 μg/ml gentamycin (Gibco). Cells were maintained at 37° C. under 7% CO₂.
2. Cells were plated at 6×10⁴/ml (0.2 ml/well; 12,000 cells/well) in 96-well microtiter plates (Falcon or Corning/Costar) and incubated at 37° C. overnight (18-24 hours).
3. 2-5 μl of the test agent-[200 μg/ml stock in 20% DMSO/20 mM HEPES, pH 7.5 (Gibco)] was added to each well to achieve a final concentration of 2-5 μg/ml. Cells were incubated with the test compounds for 5 days at 37° C.
4. Surviving cells were fixed with 50 μl 50% trichloroacetic acid (Sigma; 10% final concentration) for 1 hour at 4° C.
5. Plates were rinsed twice with water (0.3 ml per wash using MRD8 robot), and allowed to dry.
6. Sulforhodamine B (SRB) (Sigma; 0.4% in 1% acetic acid, 50 μl per well; MRD8 robot) was added and plates kept at room temperature for 10 minutes.
7. Excess dye was removed by washing with 1% acetic acid (3 times, 0.3 ml per wash; MRD8 robot) and allowed to dry.
8. The dye was solubilized in Tris base (Sigma; 10 mM, 150 μl per well). Plates were kept at room temperature for 30 minutes to allow the dye to diffuse.
9. Absorbance was measured at 540 nm.
10. The percentage of cells surviving drug treatment relative to control wells (no drug) was determined.
11. The difference in % cell survival between the two cell lines was calculated.
12. Compounds producing a difference score of >50 were considered to be selective for the p21-deficient cells and were evaluated further.
   Determination of Selectivity of Compounds for p21−/− Cells:
1. 80S14 cells were cultured as described above.
2. Cells were seeded in 96-well microtiter plates at 12,000 cells/well (either 0.15 ml/well of 8×10⁴ cells/ml suspension, or 0.2 ml/well of 6×10⁴ cells/ml suspension). Plates were incubated at 37° C. overnight (18-24 hours).
3. Serial dilutions of the test agent were added to each well. Dilutions were prepared either in 40% DMSO/20% methanol/20 mM HEPES, pH 7.5 (Gibco) or directly in growth medium. One of two dose ranges were used: 5 pM to 50 μM or 170 pg/ml to 10 μg/ml. Cells were incubated with the test compounds for 5 days at 37° C.
4. Surviving cells were fixed with 50 μl 50% trichloroacetic acid (Sigma; 10% final concentration) for 1 hour at 4° C.
5. Plates were rinsed extensively in water, and allowed to dry.
6. Sulforhodamine B (Sigma; 0.4% in 1% acetic acid, 70 μl per well) was added and plates kept at room temperature for 10 minutes.
7. Excess dye was removed by washing 5 times with 1% acetic acid and plates were allowed to dry.
8. The dye was solubilized in Tris base (Sigma; 10 mM, 150 μl per well). Plates were placed on a titer plate shaker (Lab Line Instruments) for 5 minutes to allow the dye to diffuse.
9. Absorbance was measured at 560 nm.

10. The drug concentration which inhibits cell proliferation by 50% relative to untreated controls (IC₅₀) was determined from cytotoxicity curves.

TABLE 1
Cellular activity in 80s14

| | | IC₅₀ for | | |
|---|---|---|---|---|
| | | 80S14 | Mol | Method of |
| Example | Chemical Name | (μM) | Ion | Preparation |
| 1 | N-phenyl-N′-{3-[3-(thien-2- | +++ | 449 | 1 |
| | ylcarbonyl)pyrazolo[1,5-a]pyrimidin-7- | | | |
| | yl]phenyl}urea | | | |
| 2 | N-(2-phenylethyl)-N′-{3-[3-(thien-2- | ++ | 468 | 1 |
| | ylcarbonyl)pyrazolo[1,5-a]pyrimidin-7-yl]- | | | |
| | phenyl}urea | | | |
| 3 | N-cyclohexyl-N′-{3-[3-(thien-2- | +++ | 446 | 1 |
| | ylcarbonyl)pyrazolo[1,5-a]pyrimidin-7- | | | |
| | yl]phenyl}urea | | | |
| 4 | methyl N-[({3-[3-(thien-2- | +++ | 478 | 1 |
| | ylcarbonyl)pyrazolo[1,5-a]pyrimidin-7- | | | |
| | yl]phenyl}amino)carbonyl]-L-valinate | | | |
| 5 | N-allyl-N′-{3-[3-(thien-2- | +++ | 404 | 1 |
| | ylcarbonyl)pyrazolo[1,5-a]pyrimidin-7- | | | |
| | yl]phenyl}urea | | | |
| 6 | N-butyl-N′-{3-[3-(thien-2- | +++ | 420 | 1 |
| | ylcarbonyl)pyrazolo [1,5-a]pyrimidin-7- | | | |
| | yl]phenyl}urea | | | |
| 7 | N-{3-[3-(thien-2-ylcarbonyl)pyrazolo[1,5-a] | ++ | 509 | 1 |
| | pyrimidin-7-yl] phenyl}-N′- | | | |
| | (trichloroacetyl)urea | | | |
| 8 | ethyl N-[({3-[3-(thien-2-ylcarbonyl)pyrazolo | + | 450 | 1 |
| | [1,5-a]pyrimidin-7- | | | |
| | yl]phenyl}amino)carbonyl]glycinate | | | |
| 9 | N-[(1S)-1-phenylethyl]-N′-{3-[3-(thien-2- | ++ | 468 | 1 |
| | ylcarbonyl)pyrazolo [1,5-a]pyrimidin-7- | | | |
| | yl]phenyl}urea | | | |
| 10 | N-[(1S,2R)-2-phenylcyclopropyl]-N′-{3-[3- | +++ | 480 | 1 |
| | (thien-2-ylcarbonyl)pyrazolo[1,5- | | | |
| | a]pyrimidin-7-yl]phenyl}urea | | | |
| 11 | allyl 3-[3-(thien-2-ylcarbonyl)pyrazolo[1,5- | +++ | 405 | 3 |
| | a]pyrimidin-7-yl]phenylcarbamate | | | |
| 12 | 2,2,2-trichloroethyl 3-[3-(thien-2- | +++ | 494, | 3 |
| | ylcarbonyl)pyrazolo[1,5-a]pyrimidin-7- | | 496 | |
| | yl]phenylcarbamate | | | |
| 13 | prop-2-ynyl 3-[3-(thien-2- | +++ | 403 | 3 |
| | ylcarbonyl)pyrazolo[1,5-a]pyrimidin-7- | | | |
| | yl]phenylcarbamate | | | |
| 14 | 3-butenyl 3-[3-(thien-2-ylcarbonyl)pyrazolo | +++ | 419 | 3 |
| | [1,5-a]pyrimidin-7-yl]phenylcarbamate | | | |
| 15 | isopropenyl3-[3-(thien-2- | +++ | 405 | 3 |
| | ylcarbonyl)pyrazolo[1,5-a]pyrimidin-7- | | | |
| | yl]phenylcarbamate | | | |
| 16 | 2-chloroethyl 3-[3-(thien-2- | +++ | 427, | 3 |
| | ylcarbonyl)pyrazolo[1,5-a]pyrimidin-7- | | 429 | |
| | yl]phenylcarbamate | | | |
| 17 | 4-methylphenyl 3-[3-(thien-2- | + | 455 | 3 |
| | ylcarbonyl)pyrazolo[1,5-a]pyrimidin-7- | | | |
| | yl]phenylcarbamate | | | |
| 18 | benzyl 3-[3-(thien-2-ylcarbonyl)pyrazolo[1,5- | +++ | 455, | 3 |
| | a]pyrimidin-7-yl]phenylcarbamate | | | |
| 19 | 4-chlorobutyl 3-[3-(thien-2- | +++ | 455, | 3 |
| | ylcarbonyl)pyrazolo[1,5-a]pyrimidin-7- | | 457 | |
| | yl]phenylcarbamate | | | |
| 20 | N-{3-[3-(thien-2-ylcarbonyl)pyrazolo[1,5- | ++ | 399 | 8 |
| | a]pyrimidin-7-yl]phenyl}methanesulfonamide | | | |
| 21 | N-{3-[-(thien-2-ylcarbonyl)pyrazolo[1,5- | +++ | 413 | 8 |
| | a]pyrimidin-7-yl]phenyl}ethanesulfonamide | | | |
| 22 | N-{3-[3-(thien-2-ylcarbonyl)pyrazolo[1,5- | ++ | 441 | 8 |
| | a]pyrimidin-7-yl]phenyl}butane-1- | | | |
| | sulfonamide | | | |
| 23 | N-{3-[3-(thien-2-ylcarbonyl)pyrazolo[1,5- | ++ | 427 | 8 |
| | a]pyrimidin-7-yl]phenyl}propane-2- | | | |
| | sulfonamide | | | |
| 24 | N-{3-[3-(thien-2-ylcarbonyl)pyrazolo[1,5- | ++ | 461 | 8 |
| | a]pyrimidin-7-yl]phenyl}benzenesulfonamide | | | |
| 25 | 4-methyl-N-{3-[3-(thien-2- | +++ | 475 | 8 |
| | ylcarbonyl)pyrazolo[1,5-a]pyrimidin-7- | | | |
| | yl]phenyl}benzenesulfonamide | | | |
| 26 | 2-methyl-N-{3-[3-(thien-2- | +++ | 475 | 8 |
| | ylcarbonyl)pyrazolo[1,5-a]pyrimidin-7- | | | |
| | yl]phenyl}benzenesulfonamide | | | |
| 27 | 4-fluoro-N-{3-[3-(thien-2- | +++ | 479 | 8 |
| | ylcarbonyl)pyrazolo[1,5-a]pyrimidin-7- | | | |
| | yl]phenyl}benzenesulfonamide | | | |
| 28 | 4-bromo-N-{3-[3-(thien-2- | ++ | 539, | 8 |
| | ylcarbonyl)pyrazolo [1,5-a]pyrimidin-7- | | 541 | |
| | yl]phenyl}benzenesulfonamide | | | |
| 29 | 4-methoxy-N-{3-[3-(thien-2- | ++ | 491 | 8 |
| | ylcarbonyl)pyrazolo [1,5-a]pyrimidin-7- | | | |
| | yl]phenyl}benzenesulfonamide | | | |
| 30 | (E)-2-phenyl-N-{3-[3-(thien-2- | + | 487 | 8 |
| | ylcarbonyl)pyrazolo[1,5-a]pyrimidin-7- | | | |
| | yl]phenyl}ethenesulfonamide | | | |
| 31 | N-{3-[3-(thien-2-ylcarbonyl)pyrazolo[1,5- | ++ | 467 | 8 |
| | a]pyrimidin-7-yl]phenyl} thiophene-2- | | | |
| | sulfonamide | | | |
| 32 | 1-methyl-N-{3-[3-(thien-2- | + | 465 | 8 |
| | ylcarbonyl)pyrazolo[1,5-a]pyrimidin-7- | | | |
| | yl]phenyl}-1H-imidazole-4-sulfonamide | | | |
| 33 | N,N-dimethyl-N′-{3-[3-(thien-2- | ++ | 428 | 8 |
| | ylcarbonyl)pyrazolo[1,5-a]pyrimidin-7- | | | |
| | yl]phenyl}sulfamide | | | |
| 34 | ethyl oxo({3-[3-(thien-2- | + | 421 | 5 |
| | ylcarbonyl)pyrazolo[1,5-a]pyrimidin-7- | | | |
| | yl]phenyl}amino)acetate | | | |
| 35 | methyl 4-oxo-4-({3-[3-(thien-2- | + | 435 | 5 |
| | ylcarbonyl)pyrazolo[1,5-a]pyrimidin-7-yl] | | | |
| | phenyl}anmino)butanoate | | | |
| 36 | 2-methyl-N-{3-[3-(thien-2- | +++ | 389 | 5 |
| | ylcarbonyl)pyrazolo[1,5-a]pyrimidin-7- | | | |
| | yl]phenyl} acrylamide | | | |
| 37 | 3-methyl-N-{3-[3-(thien-2- | +++ | 403 | 5 |
| | ylcarbonyl)pyrazolo[1,5-a]pyrimidin-7- | | | |
| | yl]phenyl}but-2-enamide | | | |
| 38 | 2,2,2-trichloro-N-{3-[3-(thien-2- | ++ | 466 | 5 |
| | ylcarbonyl)pyrazolo[1,5-a]pyrimidin-7- | | | |
| | yl]phenyl} acetamide | | | |
| 39 | 3-(methylthio)-N-{3-[3-(thien-2- | +++ | 423 | 5 |
| | ylcarbonyl)pyrazolo[1,5-a]pyrimidin-7- | | | |
| | yl]phenyl}propanamide | | | |
| 40 | N-{3-[3-(thien-2-ylcarbonyl)pyrazolo[1,5- | +++ | 431 | 5 |
| | a]pyrimidin-7-yl]phenyl}thiophene-2- | | | |
| | carboxamide | | | |
| 41 | N-{3-[3-(thien-2-ylcarbonyl)pyrazolo[1,5- | ++ | 514 | 5 |
| | a]pyrimidin-7-yl]phenyl}-1-(trifluoroacetyl)- | | | |
| | L-prolinamide | | | |
| 42 | N-{3-[3-(thien-2-ylcarbonyl)pyrazolo[1,5- | +++ | 483 | 5 |
| | a]pyrimidin-7-yl]phenyl} adamantane-1- | | | |
| | carboxamide | | | |
| 43 | 4,7,7-trimethyl-3-oxo-N-{13-[3-(thien-2- | +++ | 501 | 5 |
| | ylcarbonyl)pyrazolo[1,5-a]pyrimidin-7- | | | |
| | yl]phenyl}-2-oxabicyclo[2.2.1Jheptane-1- | | | |
| | carboxamide | | | |
| 44 | N-{3-[3-(thien-2-ylcarbonyl)pyrazolo[1,5- | +++ | 403 | 7 |
| | a]pyrimidin-7-yl]phenyl}pent-4-enamide | | | |
| 45 | N-{3-[3-(thien-2-ylcarbonyl)pyrazolo[1,5- | +++ | 301 | 7 |
| | a]pyrimidin-7-yl]phenyl}pent-4-ynamide | | | |
| 46 | 4,4,4-trifluoro-3-methyl-N-{3-[3-(thien-2- | +++ | 459 | 7 |
| | ylcarbonyl)pyrazolo [1,5-a]pyrimidin-7- | | | |
| | yl]phenyl}butanamide | | | |
| 47 | 2-cyano-N-{3-[3-(thien-2- | +++ | 388 | 7 |
| | ylcarbonyl)pyrazolo[1,5-a]pyrimidin-7- | | | |
| | yl]phenyl}acetamide | | | |
| 48 | 2-oxo-2-({3-[3-(thien-2- | ++ | 421 | 7 |
| | ylcarbonyl)pyrazolo[1,5-a]pyrimidin-7- | | | |
| | yl]phenyl}amino)ethyl acetate | | | |
| 49 | 2-(2-methoxyethoxy)-N-{3-[3-(thien-2- | ++ | 437 | 7 |
| | ylcarbonyl)pyrazolo[1,5-a]pyrimidin-7- | | | |
| | yl]phenyl} acetamide | | | |
| 50 | 2-thien-2-yl-N-{3-[3-(thien-2- | +++ | 445 | 7 |
| | ylcarbonyl)pyrazolo[1,5-a]pyrimidin-7- | | | |
| | yl]phenyl}acetamide | | | |
| 51 | 2-(2,3-dihydro-1H-inden-2-yl)-N-{3-[3- | +++ | 479 | 7 |
| | (thien-2-ylcarbonyl)pyrazolo[1,5- | | | |
| | a]pyrimidin-7-yl]phenyl}acetamide | | | |
| 52 | 2-(2-methylphenyl)-N-{3-[3-(thien-2- | +++ | 453 | 7 |
| | ylcarbonyl)pyrazolo[1,5-a]pyrimidin-7- | | | |
| | yl]phenyl}acetamide | | | |
| 53 | 2-(1,3-benzodioxol-5-yl)-N-{3-[3-(thien-2- | +++ | 483 | 7 |
| | ylcarbonyl)pyrazolo[1,5-a]pyrimidin-7- | | | |
| | yl]phenyl}acetamide | | | |
| 54 | N-{3-[3-(thien-2-ylcarbonyl)pyrazolo[1,5- | ++ | 476 | 7 |
| | a]pyrimidin-7-yl]phenyl}quinoline-3- | | | |
| | carboxamide | | | |
| 55 | (2E)-3-phenyl-N-{3-[3-(thien-2- | ++ | 451 | 7 |
| | ylcarbonyl)pyrazolo[1,5-a]pyrimidin-7- | | | |
| | yl]phenyl}prop-2-enamide | | | |
| 56 | 2-cyclohexyl-N-{3-[3-(thien-2- | +++ | 445 | 7 |
| | ylcarbonyl)pyrazolo[1,5-a]pyrimidin-7- | | | |
| | yl]phenyl}acetamide | | | |
| 57 | 2-cycloheptyl-N-{3-[3-(thien-2- | +++ | 459 | 7 |
| | ylcarbonyl)pyrazolo[1,5-a]pyrimidin-7- | | | |
| | yl]phenyl}acetamide | | | |
| 58 | 2-bicyclo[2.2.1]hept-2-yl-N-{3-[3-(thien-2- | +++ | 457 | 7 |
| | ylcarbonyl)pyrazolo[1,5-a]pyrimidin-7- | | | |
| | yl]phenyl}acetamide | | | |
| 59 | N-1--{3-[3-(thien-2-ylcarbonyl)pyrazolo[1,5- | + | 425 | 6 |
| | a]pyrimidin-7-yl]phenyl}glycinamide | | | |
| 60 | N-1-{3-[3-(thien-2-ylcarbonyl)pyrazolo[1,5- | ++ | 439 | 6 |
| | a]pyrimidin-7-yl]phenyl}-L-alaninamide | | | |
| 61 | N-1-{3-[3-(thien-2-ylcarbonyl)pyrazolo[1,5- | + | 455 | 6 |
| | a]pyrimidin-7-yl]phenyl}-L-leucinamide | | | |
| 62 | N-1-{3-[3-(thien-2-ylcarbonyl)pyrazolo[1,5- | ++ | 434 | 6 |
| | a]pyrimidin-7-yl]phenyl}-L-isoleucinamide | | | |
| 63 | N-{3-[3-(thien-2-ylcarbonyl)pyrazolo[1,5- | + | 468 | 6 |
| | a]pyrimidin-7-yl]phenyl}-L- | | | |
| | phenylalaninamide | | | |
| 64 | 3-cyclohexyl-N-1-{3-[3-(thien-2- | + | 474 | 6 |
| | ylcarbonyl)pyrazolo[1,5-a]pyrimidin-7- | | | |
| | yl]phenyl}-L-alaninamide | | | |
| 65 | N-1-{3-[3-(thien-2-ylcarbonyl)pyrazolo[1,5- | ++ | 420 | 6 |
| | a]pyrimidin-7-yl]phenyl}-L-valinamide | | | |
| 66 | 4-amino-N-{3-[3-(thien-2- | + | 406 | 6 |
| | ylcarbonyl)pyrazolo[1,5-a]pyrimidin-7- | | | |
| | yl]phenyl}butanamide | | | |
| 67 | N-{3-[3-(thien-2-ylcarbonyl)pyrazolo[1,5- | ++ | 418 | 6 |
| | a]pyrimidin-7-yl]phenyl}-L-prolinamide | | | |
| 68 | (2S)-2-amino-2-cyclohexyl-N-{3-[3-(thien-2- | ++ | 460 | 6 |
| | ylcarbonyl)pyrazolo[1,5-a]pyrimidin-7- | | | |
| | yl]phenyl}ethanamide | | | |
| 69 | 4-methyl-N-{3-[3-(thien-2- | +++ | 439 | 5 |
| | ylcarbonyl)pyrazolo[1,5-a]pyrimidin-7- | | | |
| | yl]phenyl}benzamide | | | |
| 70 | 4-methoxy-N-{3-[3-(thien-2- | +++ | 455 | 5 |
| | ylcarbonyl)pyrazolo[1,5-a]pyrimidin-7- | | | |
| | yl]phenyl}benzamide | | | |
| 71 | 4-chloro-N-{3-[3-(thien-2- | ++ | 459, | 5 |
| | ylcarbonyl)pyrazolo[1,5-a]pyrimidin-7- | | 461 | |
| | yl]phenyl}benzamide | | | |
| 72 | 4-bromo-N-{3-[3-(thien-2- | ++ | 503, | 5 |
| | ylcarbonyl)pyrazolo[1,5-a]pyrimidin-7- | | 505 | |
| | yl]phenyl}benzamide | | | |
| 73 | 4-(heptyloxy)-N-{3-[3-(thien-2- | + | 539 | 5 |
| | ylcarbonyl)pyrazolo[1,5-a]pyrimidin-7- | | | |
| | yl]phenyl}benzamide | | | |
| 74 | 4-pentyl-N-{3-[3-(thien-2- | + | 495 | 5 |
| | ylcarbonyl)pyrazolo[1,5-a]pyrimidin-7- | | | |
| | yl]phenyl}benzamide | | | |
| 75 | 2-methoxy-N-{3-[3-(thien-2- | +++ | 455 | 5 |
| | ylcarbonyl)pyrazolo[1,5-a]pyrimidin-7- | | | |
| | yl]phenyl}benzamide | | | |
| 76 | 2-methyl-N-{3-[3-(thien-2- | +++ | 439 | 5 |
| | ylcarbonyl)pyrazolo[1,5-a]pyrimidin-7- | | | |
| | yl]phenyl}benzamide | | | |
| 77 | 2-chloro-N-{3-[3-(thien-2- | +++ | 459, | 5 |
| | ylcarbonyl)pyrazolo[1,5-a]pyrimidin-7- | | 461 | |
| | yl]phenyl}benzamide | | | |
| 78 | N-{3-[3-(thien-2-ylcarbonyl)pyrazolo[1,5- | +++ | 493 | 5 |
| | a]pyrimidin-7-yl]phenyl}-2- | | | |
| | (trifluoromethyl)benzamide | | | |
| 79 | 3-methoxy-N-{3-[3-(thien-2- | +++ | 455 | 5 |
| | ylcarbonyl)pyrazolo[1,5-a]pyrimidin-7- | | | |
| | yl]phenyl}benzamide | | | |
| 80 | 3-fluoro-N-{3-[3-(thien-2- | +++ | 443 | 5 |
| | ylcarbonyl)pyrazolo[1,5-a]pyrimidin-7- | | | |
| | yl]phenyl}benzamide | | | |
| 81 | 3,4-dimethoxy-N-{3-[3-(thien-2- | ++ | 485 | 5 |
| | ylcarbonyl)pyrazolo[1,5-a]pyrimidin-7- | | | |
| | yl]phenyl}benzamide | | | |
| 82 | 3,4-dichloro-N-{3-[3-(thien-2- | + | 493, | 5 |
| | ylcarbonyl)pyrazolo[1,5-a]pyrimidin-7- | | 495 | |
| | yl]phenyl}benzamide | | | |
| 83 | 2,6-dimethoxy-N-{3-[3-(thien-2- | +++ | 485 | 5 |
| | ylcarbonyl)pyrazolo[1,5-a]pyrimidin-7- | | | |
| | yl]phenyl}benzamide | | | |
| 84 | 3,4-difluoro-N-{3-[3-(thien-2- | ++ | 461 | 5 |
| | ylcarbonyl)pyrazolo[1,5-a]pyrimidin-7- | | | |
| | yl]phenyl}benzamide | | | |
| 85 | N-{3-[3-(thien-2-ylcarbonyl)pyrazolo[1,5- | +++ | 475 | 5 |
| | a]pyrimidin-7-yl]phenyl}-1-naphthamide | | | |
| 86 | N-{3-[3-(thien-2-ylcarbonyl)pyrazolo[1,5- | + | 475 | 5 |
| | a]pyrimidin-7-yl]phenyl}-2-naphthamide | | | |
| 87 | 2-phenyl-N-{3-[3-(thien-2- | +++ | 439 | 5 |
| | ylcarbonyl)pyrazolo[1,5-a]pyrimidin-7- | | | |
| | yl]phenyl}acetamide | | | |
| 88 | 2-(4-methoxyphenyl)-N-{3-[3-(thien-2- | +++ | 469 | 5 |
| | ylcarbonyl)pyrazolo[1,5-a]pyrimidin-7- | | | |
| | yl]phenyl}acetamide | | | |
| 89 | 2-(3-methoxyphenyl)-N-{3-[3-(thien-2- | +++ | 469 | 5 |
| | ylcarbonyl)pyrazolo[1,5-a]pyrimidin-7- | | | |
| | yl]phenyl}acetamide | | | |
| 90 | 2-(4-chlorophenyl)-N-{3-[3-(thien-2- | +++ | 473, | 5 |
| | ylcarbonyl)pyrazolo[1,5-a]pyrimidin-7- | | 475 | |
| | yl]phenyl}acetamide | | | |
| 91 | 2-(4-fluorophenyl)-N-{3-[3-(thien-2- | +++ | 457 | 5 |
| | ylcarbonyl)pyrazolo[1,5-a]pyrimidin-7- | | | |
| | yl]phenyl}acetamide | | | |
| 92 | 2-(2,5-dimethoxyphenyl)-N-{3-[3-(thien-2- | ++ | 499 | 5 |
| | ylcarbonyl)pyrazolo[1,5-a]pyrimidin-7- | | | |
| | yl]phenyl}acetamide | | | |
| 93 | 2-phenoxy-N-{3-[3-(thien-2- | ++ | 455 | 5 |
| | ylcarbonyl)pyrazolo[1,5-a]pyrimidin-7- | | | |
| | yl]pheriyl}acetamide | | | |
| 94 | 4-fluoro-N-{3-[3-(thien-2- | +++ | 443 | 5 |
| | ylcarbonyl)pyrazolo[1,5-a]pyrimidin-7- | | | |
| | yl]phenyl}benzamide | | | |
| 95 | ethyl 2-oxo-2-({3-[3-(thien-2- | ++ | 450 | 5 |
| | ylcarbonyl)pyrazolo[1,5-a]pyrimidin-7- | | | |
| | yl]phenyl}amino)ethylcarbamate | | | |
| 96 | propyl 2-oxo-2-({3-[3-(thien-2- | + | 464 | 5 |
| | ylcarbonyl)pyrazolo[1,5-a]pyrimidin-7- | | | |
| | yl]phenyl}amino)ethylcarbamate | | | |
| 97 | isopropyl 2-oxo-2-({3-[3-(thien-2- | ++ | 464 | 5 |
| | ylcarbonyl)pyrazolo[1,5-a]pyrimidin-7- | | | |
| | yl]phenyl}amino)ethylcarbamate | | | |
| 98 | butyl 2-oxo-2-({3-[3-(thien-2- | +++ | 478 | 5 |
| | ylcarbonyl)pyrazolo[1,5-a]pyrimidin-7- | | | |
| | yl]phenyl}amino)ethylcarbamate | | | |
| 99 | isobutyl 2-oxo-2-({3-[3-(thien-2- | ++ | 478 | 5 |
| | ylcarbonyl)pyrazolo[1,5-a]pyrimidin-7- | | | |
| | yl]phenyl}amino)ethylcarbamate | | | |
| 100 | But-3-enyl 2-oxo-2-({3-[3-(thien-2- | ++ | 476 | 5 |
| | ylcarbonyl)pyrazolo[1,5-a]pyrimidin-7- | | | |
| | yl]phenyl}amino)ethylcarbamate | | | |
| 101 | 4-chlorobutyl2-oxo-2-({3-[3-(thien-2- | + | 512 | 5 |
| | ylcarbonyl)pyrazolo[1,5-a]pyrimidin-7- | | | |
| | yl]phenyl}amino)ethylcarbamate | | | |
| 102 | 2-chloroethyl 2-oxo-2-({3-[3-(thien-2- | ++ | 484 | 5 |
| | ylcarbonyl)pyrazolo[1,5-a]pyrimidin-7- | | | |
| | yl]phenyl}amino)ethylcarbamate | | | |
| 103 | Neopentyl 2-oxo-2-({3-[3-(thien-2- | ++ | 492 | 5 |
| | ylcarbonyl)pyrazolo[1,5-a]pyrimidin-7- | | | |
| | yl]phenyl}amino)ethylcarbamate | | | |
| 104 | hexyl 2-oxo-2-({3-[3-(thien-2- | ++ | 506 | 5 |
| | ylcarbonyl)pyrazolo[1,5-a]pyrimidin-7- | | | |
| | yl]phenyl}amino)ethylcarbamate | | | |
| 105 | Prop-2-ynyl 2-oxo-2-({3-[3-(thien-2- | ++ | 460 | 5 |
| | ylcarbonyl)pyrazolo[1,5-a]pyrimidin-7- | | | |
| | yl]phenyl}amino)ethylcarbamate | | | |
| 106 | allyl 2-oxo-2-({3-[3-(thien-2- | + | 462 | 5 |
| | ylcarbonyl)pyrazolo[1,5-a]pyrimidin-7- | | | |
| | yl]phenyl}amino)ethylcarbamate | | | |
| 107 | benzyl2-oxo-2-({3-[3-(thien-2- | ++ | 512 | 5 |
| | ylcarbonyl)pyrazolo[1,5-a]pyrimidin-7- | | | |
| | yl]phenyl}amino)ethylcarbamate | | | |
| 108 | N-[2-oxo-2-({3-[3-(thien-2- | + | 446 | 5 |
| | ylcarbonyl)pyrazolo[1,5-a]pyrimidin-7- | | | |
| | yl]phenyl}amino)ethyl]cyclopropanecarboxamide | | | |
| 109 | N-[2-oxo-2-({3-[3-(thien-2- | + | 460 | 5 |
| | ylcarbonyl)pyrazolo[1,5-a]pyrimidin-7- | | | |
| | yl]phenyl}amino)ethyl]cyclobutanecarboxamide | | | |
| 110 | N-[2-oxo-2-({3-[3-(thien-2- | ++ | 474 | 5 |
| | ylcarbonyl)pyrazolo[1,5-a]pyrimidin-7- | | | |
| | yl]phenyl}amino)ethyl]cyclopentanecarboxamide | | | |
| 111 | N-[2-oxo-2-({3-[3-(thien-2- | + | 488 | 5 |
| | ylcarbonyl)pyrazolo[1,5-a]pyrimidin-7- | | | |
| | yl]phenyl}amino)ethyl]cyclohexanecarboxamide | | | |
| 112 | 2-methyl-N-[2-oxo-2-({3-[3-(thien-2- | + | 462 | 5 |
| | ylcarbonyl)pyrazolo[1,5-a]pyrimidin-7- | | | |
| | yl]phenyl}amino)ethyl]butanamide | | | |
| 113 | 2-ethyl-N-[2-oxo-2-({3-[3-(thien-2- | + | 476 | 5 |
| | ylcarbonyl)pyrazolo[1,5-a]pyrimidin-7- | | | |
| | yl]phenyl}amino)ethyl]butanamide | | | |
| 114 | 2,2-dimethyl-N-[2-oxo-2-({3-[3-(thien-2- | ++ | 462 | 5 |
| | ylcarbonyl)pyrazolo[1,5-a]pyrimidin-7- | | | |
| | yl]phenyl}amino)ethyl]propanamide | | | |
| 115 | 3-methyl-N-[2-oxo-2-({3-[3-(thien-2- | ++ | 462 | 5 |
| | ylcarbonyl)pyrazolo[1,5-a]pyrimidin-7- | | | |
| | yl]phenyl}amino)ethyl]butanamide | | | |
| 116 | N-[2-oxo-2-({3-[3-(thien-2- | ++ | 540 | 5 |
| | ylcarbonyl)pyrazolo[1,5-a]pyrimidin-7- | | | |
| | yl]phenyl}amino)ethyl] adamantane-1- | | | |
| | carboxamide | | | |
| 117 | N-[2-oxo-2-({3-[3-(thien-2- | | 482 | 5 |
| | ylcarbonyl)pyrazolo[1,5-a]pyrimidin-7- | | | |
| | yl]phenyl}amino)ethyl]benzamide | | | |
| 118 | N-2-[(pentylamino)carbonyl]-N-1-{3-[3- | + | 491 | 5 |
| | (thien-2-ylcarbonyl)pyrazolo[1,5- | | | |
| | A]pyrimidin-7-yl]phenyl}glycinamide | | | |
| 119 | N-2-[(ethylamino)carbonyl]-N-1-{3-[3- | ++ | 449 | 5 |
| | (thien-2-ylcarbonyl)pyrazolo[1,5- | | | |
| | a]pyrimidin-7-1yl] phenyl}glycinamide | | | |
| 120 | N-2-[(cyclohexylamino)carbonyl]-N-1-{3- | ++ | 503 | 5 |
| | [3-(thien-2-ylcarbonyl)pyrazolo[1,5- | | | |
| | a]pyrimidin-7-yl] phenyl}glycinamide | | | |
| 121 | 3-chloro-N-{3-[3-(thien-2- | +++ | 411, | 5 |
| | ylcarbonyl)pyrazolo[1,5-a]pyrimidin-7- | | 413 | |
| | yl]phenyl}hexanamide | | | |
| 122 | 3,5,5-trimethyl-N-(3-[3-(thien-2- | +++ | 461 | 5 |
| | ylcarbonyl)pyrazolo[1,5-a]pyrimidin-7- | | | |
| | yl]phenyl}hexanamide | | | |
| 123 | 3-chloro-2-(chloromethyl)-2-methyl-N-{3-[3- | +++ | 473, | 5 |
| | (thien-2-ylcarbonyl)pyrazolo[1,5- | | 475 | |
| | a]pyrimidin-7-yl]phenyl}propanamide | | | |
| 124 | N-{3-[3-(thien-2-ylcarbonyl.)pyrazolo[1,5- | + | 489 | 5 |
| | a]pyrimidin-7-yl]phenyl}undecanamide | | | |
| 125 | 1-methyl-N-{3-[3-(thien-2- | +++ | 445 | 5 |
| | ylcarbonyl)pyrazolo[1,5-a]pyrimidin-7- | | | |
| | yl]phenyl}cyclohexanecarboxamide | | | |
| 126 | 2,3,3-trichloro-N-{3-[3-(thien-2- | +++ | 479, | 5 |
| | ylcarbonyl)pyrazolo[1,5-a]pyrimidin-7- | | 481 | |
| | yl]phenyl}acrylamide | | | |
| 127 | 3-cyclopentyl-N-{3-[3-(thien-2- | ++ | 445 | 5 |
| | ylcarbonyl)pyrazolo[1,5-a]pyrimidin-7- | | | |
| | yl]phenyl}propanamide | | | |
| 128 | 2-ethyl-N-{3-[3-(thien-2- | ++ | 447 | 5 |
| | ylcarbonyl)pyrazolo[1,5-a]pyrimidin-7- | | | |
| | yl]phenyl}hexanamide | | | |
| 129 | (2E)-N-{3-[3-(thien-2- | ++ | 389 | 5 |
| | ylcarbonyl)pyrazolo[1,5-a]pyrimidin-7- | | | |
| | yl]phenyl}but-2-enamide | | | |
| 130 | N-{3-[3-(thien-2-ylcarbonyl)pyrazolo[1,5- | +++ | 432 | 2 |
| | a]pyrimidin-7-yl]phenyl}piperidine-1- | | | |
| | carboxamide | | | |
| 131 | N-{3-[3-(thien-2-ylcarbonyl)pyrazolo[1,5- | ++ | 434 | 2 |
| | a]pyrimidin-7-yl]phenyl}morpholine-4- | | | |
| | carboxamide | | | |
| 132 | N-(sec-butyl)-N′-{3-[3-(thien-2- | +++ | 420 | 2 |
| | ylcarbonyl)pyrazolo[1,5-a]pyrimidin-7- | | | |
| | yl]phenyl}urea | | | |
| 133 | N-(cyclohexylmethyl)-N′-{3-[3-(thien-2- | ++ | 460 | 2 |
| | ylcarbonyl)pyrazolo[1,5-a]pyrimidin-7- | | | |
| | yl]phenyl}urea | | | |
| 134 | N-[2-(dimethylamino)ethyl]-N′-{3-[3-(thien- | + | 435 | 2 |
| | 2-ylcarbonyl)pyrazolo[1,5-a]pyrimidin-7- | | | |
| | yl]phenyl}urea | | | |
| 135 | N-cyclopentyl-N′-{3-[3-(thien-2- | +++ | 432 | 2 |
| | ylcarbonyl)pyrazolo[1,5-a]pyrimidin-7- | | | |
| | yl]phenyl}urea | | | |
| 136 | N-(tetrahydrofuran-2-ylmethyl)-N′-{3-[3- | +++ | 448 | 2 |
| | (thien-2-ylcarbonyl)pyrazolo[1,5- | | | |
| | a]pyrimidin-7-yl]phenyl}urea | | | |
| 137 | N-neopentyl-N′-{3-[3-(thien-2- | +++ | 434 | 2 |
| | ylcarbonyl)pyrazolo[1,5-a]pyrimidin-7- | | | |
| | yl]phenyl}urea | | | |
| 138 | N-pentyl-N′-{3-[3-(thien-2- | +++ | 434 | 2 |
| | ylcarbonyl)pyrazolo[1,5-a]pyrimidin-7- | | | |
| | yl]phenyl}urea | | | |
| 139 | 4-methyl-N-{3-[3-(thien-2- | ++ | 447 | 2 |
| | ylcarbonyl)pyrazolo[1,5-a]pyrimidin-7- | | | |
| | yl]phenyl}piperazine-1-carboxamide | | | |
| 140 | 4-methyl-N-{3-[3-(thien-2- | ++ | 461 | 2 |
| | ylcarbonyl)pyrazolo[1,5-a]pyrimidin-7- | | | |
| | yl]phenyl}-1,4-diazepane-1-carboxamide | | | |
| 141 | N,N-bis(2-methoxyethyl)-N′-{3-[3-(thien-2- | + | 480 | 2 |
| | ylcarbonyl)pyrazolo[1,5-a]pyrimidin-7- | | | |
| | yl]phenyl}urea | | | |
| 142 | N-cyclopropyl-N′-{3-[3-(thien-2- | ++ | 404 | 2 |
| | ylcarbonyl)pyrazolo[1,5-a]pyrimidin-7- | | | |
| | yl]phenyl}urea | | | |
| 143 | N-{3-[3-(thien-2-ylcarbonyl)pyrazolo[1,5- | ++ | 418 | 2 |
| | a]pyrimidin-7-yl]phenyl}pyrrolidine-1- | | | |
| | carboxamide | | | |
| 144 | cyclopentyl 3-[3-(thien-2- | +++ | 433 | 4 |
| | ylcarbonyl)pyrazolo[1,5-a]pyrimidin-7- | | | |
| | yl]phenylcarbamate | | | |
| 145 | cyclohexyl 3-[3-(thien-2- | +++ | 447 | 4 |
| | ylcarbonyl)pyrazolo[1,5-a]pyrimidin-7- | | | |
| | yl]phenylcarbamate | | | |
| 146 | cyclohex-2-en-1-yl 3-[3-(thien-2- | +++ | 445 | 4 |
| | ylcarbonyl)pyrazolo[1,5-a]pyrimidin-7- | | | |
| | yl]phenylcarbamate | | | |
| 147 | cyclobutylmethyl 3-[3-(thien-2- | +++ | 433 | 4 |
| | ylcarbonyl)pyrazolo[1,5-a]pyrimidin-7- | | | |
| | yl]phenylcarbamate | | | |
| 148 | Cyclohexylmethyl 3-[3-(thien-2- | +++ | 461 | 4 |
| | ylcarbonyl)pyrazolo[1,5-a]pyrimidin-7- | | | |
| | yl]phenylcarbarmate | | | |
| 149 | tetrahydro-2H-pyran-4-yl 3-[3-(thien-2- | +++ | 449 | 4 |
| | ylcarbonyl)pyrazolo[1,5-a]pyrimidin-7- | | | |
| | yl]phenylcarbamate | | | |
| 150 | 3-methylbut-2-enyl 3-[3-(thien-2- | | 433 | 4 |
| | ylcarbonyl)pyrazolo[1,5-a]pyrimidin-7- | | | |
| | yl]phenylcarbamate | | | |
| 151 | 2-(methylthio)ethyl 3-[3-(thien-2- | +++ | 439 | 4 |
| | ylcarbonyl)pyrazolo[1,5-a]pyrimidin-7- | | | |
| | yl]phenylcarbamate | | | |
| 152 | 2-thien-3-ylethyl 3-[3-(thien-2- | ++ | 475 | 4 |
| | ylcarbonyl)pyrazolo[1,5-a]pyrimidin-7- | | | |
| | yl]phenylcarbamate | | | |
| 153 | 2-piperidin-1-ylethyl 3-[3-(thien-2- | ++ | 476 | 4 |
| | ylcarbonyl)pyrazolo[1,5-a]pyrimidin-7- | | | |
| | yl]phenylcarbamate | | | |
| 154 | 2-morpholin-4-ylethyl 3-[3-(thien-2- | +++ | 478 | 4 |
| | ylcarbonyl)pyrazolo[1,5-a]pyrimidin-7- | | | |
| | yl]phenylcarbamate | | | |
| 155 | 1-methylprop-2-enyl 3-[3-(thien-2- | +++ | 419 | 4 |
| | ylcarbonyl)pyrazolo[1,5-a]pyrimidin-7- | | | |
| | yl]phenylcarbamate | | | |
| 156 | 1-methylbut-3-ynyl 3-[3-(thien-2- | +++ | 431 | 4 |
| | ylcarbonyl)pyrazolo[1,5-a]pyrimidin-7- | | | |
| | yl]phenylcarbamate | | | |
| 157 | (2-methylcyclopropyl)methyl 3-[3-(thien-2- | +++ | 433 | 4 |
| | ylcarbonyl)pyrazolo[1,5-a]pyrimidin-7- | | | |
| | yl]phenylcarbamate | | | |
| 158 | 3-methylcyclopentyl 3-[3-(thien-2- | +++ | 447 | 4 |
| | ylcarbonyl)pyrazolo[1,5-a]pyrimidin-7- | | | |
| | yl]phenylcarbamate | | | |
| 159 | 1-methylbut-3-enyl 3-[3-(thien-2- | +++ | 433 | 4 |
| | ylcarbonyl)pyrazolo[1,5-a]pyrimidin-7- | | | |
| | yl]phenylcarbamate | | | |
| 160 | 1-cyclopropylethyl 3-[3-(thien-2- | +++ | 433 | 4 |
| | ylcarbonyl)pyrazolo[1,5-a]pyrimidin-7- | | | |
| | yl]phenylcarbamate | | | |
| 161 | but-3-ynyl 3-[3-(thien-2- | +++ | 417 | 4 |
| | ylcarbonyl)pyrazolo[1,5-a]pyrimidin-7- | | | |
| | yl]phenylcarbamate | | | |
| 162 | allyl 3-[5-methyl-3-(thien-2- | +++ | 419 | 3 |
| | ylcarbonyl)pyrazolo[1,5-a]pyrimidin-7- | | | |
| | yl]phenylcarbamate | | | |
| 163 | 2-chloroethyl 3-[5-methyl-3-(thien-2- | +++ | 441 | 3 |
| | ylcarbonyl)pyrazolo[1,5-a]pyrimidin-7- | | | |
| | yl]phenylcarbamate | | | |
| 164 | 3-methyl-N-{3-[5-methyl-3-(thien-2- | +++ | 419 | 5 |
| | ylcarbonyl)pyrazolo[1,5-a]pyrimidin-7- | | | |
| | yl]phenyl}butanamide | | | |
| 165 | N-{3-[5-methyl-3-(thien-2- | + | 439 | 5 |
| | ylcarbonyl)pyrazolo[1,5-a]pyrimidin-7- | | | |
| | yl]phenyl}benzamide | | | |
| 166 | N-{3-[5-methyl-3-(thien-2- | ++ | 459 | 5 |
| | ylcarbonyl)pyrazolo[1,5-a]pyrimidin-7- | | | |
| | yl]phenyl}-2-thien-2-ylacetamide | | | |
| 167 | N-butyl-N′-{3-[5-methyl-3-(thien-2- | ++ | 434 | 7 |
| | ylcarbonyl)pyrazolo[1,5-a]pyrimidin-7- | | | |
| | yl]phenyl}urea | | | |
| 168 | allyl 3-[6-methyl-3-(thien-2- | + | 419 | 4 |
| | ylcarbonyl)pyrazolo[1,5-a]pyrimidin-7- | | | |
| | yl]phenylcarbamate | | | |
| 169 | 2-chloroethyl 3-[6-methyl-3-(thien-2- | + | 441 | 4 |
| | ylcarbonyl)pyrazolo[1,5-a]pyrimidin-7- | | | |
| | yl]phenylcarbamate | | | |
| 170 | but-3-enyl 3-[6-methyl-3-(thien-2- | + | 433 | 4 |
| | ylcarbonyl)pyrazolo[1,5-a]pyrimidin-7- | | | |
| | yl]phenylcarbamate | | | |
| 171 | N-{3-[6-methyl-3-(thien-2- | + | 439 | 5 |
| | ylcarbonyl)pyrazolo[1,5-a]pyrimidin-7- | | | |
| | yl]phenyl}benzamide | | | |
| 172 | N-{3-[6-methyl-3-(thien-2- | + | 459 | 5 |
| | ylcarbonyl)pyrazolo[1,5-a]pyrimidin-7- | | | |
| | yl]phenyl}-2-thien-2-ylacetamide | | | |
| 173 | N-{3-[6-methyl-3-(thien-2- | + | 440 | 5 |
| | ylcarbonyl)pyrazolo[1,5-a]pyrimidin-7- | | | |
| | yl]phenyl}nicotinamide | | | |
| 174 | N-butyl-N′-{3-[6-methyl-3-(thien-2- | + | 434 | 7 |
| | ylcarbonyl)pyrazolo[1,5-a]pyrimidin-7- | | | |
| | yl]phenyl}urea | | | |

+++, <1 μM;
++, 1-10 μM;
+, 10-50 μM

Cytotoxicity Assay Using LoVo Colon Cell Line:
1. LoVo Colon cell lines was cultured in RPMI 1640 medium (Gibco/Invitrogen Life Sciences) supplemented with 10% fetal bovine serum (FBS, Gibco) and 50 μg/ml gentamicin (Gibco). Cells were maintained at 37° C. under 7% CO₂.
2. Cells were seeded in 96-well microtiter plates (Corning/Costar) in a volume of 0.15 ml of growth medium (4000 cells/well). Plates were incubated at 37° C. overnight (18-24 hours).
3. Serial dilutions of the test agent were added to each well. Dilutions were prepared in growth medium. One of two dose ranges was used: 5 pM to 20 μM or 170 μg/ml to 10 μg/ml. Cells were incubated with the test compounds for 4 days at 37° C.
4. Surviving cells were processed using the SRB asay, as described above.

5. The drug concentration that inhibits cell proliferation by 50% relative to untreated controls (IC₅₀) was determined from cytotoxicity curves using the LSW Toolbox graphing application.

TABLE 2
Cellular activity in LoVo cell

| Example | | Inhibition |
|---|---|---|
| number | Chemical Name | of LoVo |
| 2 | N-(2-phenylethyl)-N′-{3-[3-(thien-2- | ++ |
| | ylcarbonyl)pyrazolo[1,5-a]pyrimidin-7-yl}phenyl}urea | |
| 3 | N-cyclohexyl-N′-{3-[3-(thien-2-ylcarbonyl)pyrazolo[1,5- | +++ |
| | a]pyrimidin-7-yl]phenyl}urea | |
| 6 | N-butyl-N′-{3-[3-(thien-2-ylcarbonyl)pyrazolo[1,5- | +++ |
| | a]pyrimidin-7-yl]phenyl}urea | |
| 11 | allyl 3-[3-(thien-2-ylcarbonyl)pyrazolo[1,5-a]pyrimidin-7- | +++ |
| | yl]phenylcarbamate | |
| 13 | prop-2-ynyl 3-[3-(thien-2-ylcarbonyl)pyrazolo[1,5- | ++ |
| | a]pyrimidin-7-yl]phenylcarbamate | |
| 16 | 2-chloroethyl 3-[3-(thien-2-ylcarbonyl)pyrazolo[1,5- | +++ |
| | a]pyrimidin-7-yl]phenylcarbamate | |
| 19 | 4-chlorobutyl 3-[3-(thien-2-ylcarbonyl)pyrazolo[1,5- | +++ |
| | a]pyrimidin-7-yl]phenylcarbamate | |
| 36 | 2-methyl-N-{3-[3-(thien-2-ylcarbonyl)pyrazolo[1,5- | +++ |
| | a]pyrimidin-7-yl]phenyl) acrylamide | |
| 40 | N-{3-[3-(thien-2-ylcarbonyl)pyrazolo[1,5-a]pyrimidin-7- | +++ |
| | yl]phenyl}thiophene-2-carboxamide | |
| 54 | 4,7,7-trimethyl-3-oxo-N-{3-[3-(thien-2- | +++ |
| | ylcarbonyl)pyrazolo[1,5-a]pyrimidin-7-yl]phenyl}-2- | |
| | oxabicyclo[2.2.1}heptane-1-carboxamide | |
| 56 | 2-cyclohexyl-N-{3-[3-(thien-2-ylcarbonyl)pyrazolo[1,5- | +++ |
| | a]pyrimidin-7-yl]phenyl}acetamide | |
| 57 | 2-cycloheptyl-N-{3-[3-(thien-2-ylcarbonyl)pyrazolo[1,5- | +++ |
| | a]pyrimidin-7-yl]phenyl}acetamide | |
| 58 | 2-bicyclo[2.2.1]hept-2-yl-N-{3-[3-(thien-2- | +++ |
| | ylcarbonyl)pyrazolo[1,5-a]pyrimidin-7- | |
| | yl]phenyl}acetamide | |
| 59 | 2-methoxy-N-{3-[3-(thien-2-ylcarbonyl)pyrazolo[1,5- | +++ |
| | a]pyrimidin-7-yl]phenyl}benzamide | |
| 76 | 2-methyl-N-{3-[3-(thien-2-ylcarbonyl)pyrazolo[1,5- | +++ |
| | a]pyrimidin-7-yl]phenyl}benzamide | |
| 77 | 2-chloro-N-{3-[3-(thien-2-ylcarbonyl)pyrazolo[1,5-a]pyrimidin- | +++ |
| | 7-yl]phenyl}benzamide | |
| 78 | N-{3-[3-(thien-2-ylcarbonyl)pyrazolo[1,5-a]pyrimidin-7- | +++ |
| | yl]phenyl}-2-(trifluoromethyl)benzamide | |
| 130 | N-{3-[3-(thien-2-ylcarbonyl)pyrazolo[1,5-a]pyrimidin-7- | +++ |
| | yl]phenyl}piperidine-1-carboxamide | |
| 131 | N-{3-[3-(thien-2-ylcarbonyl)pyrazolo[1,5-a]pyrimidin-7- | ++ |
| | yl]phenyl}morpholine-4-carboxamide | |
| 132 | N-(sec-butyl)-N′-{3-[3-(thien-2-ylcarbonyl)pyrazolo[1,5- | +++ |
| | a]pyrimidin-7-yl]phenyl}urea | |
| 133 | N-(cyclohexylmethyl)-N′-{3-[3-(thien-2- | ++ |
| | ylcarbonyl)pyrazolo[1,5-a]pyrimidin-7-yl]phenyl}urea | |
| 135 | N-cyclopentyl-N′-{3-[3-(thien-2-ylcarbonyl)pyrazolo[1,5- | +++ |
| | a]pyrimidin-7-yl]phenyl}urea | |
| 136 | N-(tetrahydrofuran-2-ylmethyl)-N′-{3-[3-(thien-2- | ++ |
| | ylcarbonyl)pyrazolo[1,5-a]pyrimidin-7-yl]phenyl}urea | |
| 137 | N-neopentyl-N′-{3-[3-(thien-2-ylcarbonyl)pyrazolo[1,5- | +++ |
| | a]pyrimidin-7-yl]phenyl}urea | |
| 138 | N-pentyl-N′-{3-[3-(thien-2-ylcarbonyl)pyrazolo[1,5- | +++ |
| | a]pyrimidin-7-yl]phenyl}urea | |
| 141 | N,N-bis(2-methoxyethyl)-N′-{3-[3-(thien-2- | + |
| | ylcarbonyl)pyrazolo[1,5-a]pyrimidin-7-yl]phenyl}urea | |
| 142 | N-cyclopropyl-N′-{3-[3-(thien-2- | ++ |
| | ylcarbonyl)pyrazolo[1,5-a]pyrimidin-7-yl]phenyl}urea | |
| 143 | N-{3-[3-(thien-2-ylcarbonyl)pyrazolo[1,5-a]pyrimidin-7- | ++ |
| | yl]phenyl}pyrrolidine-1-carboxamide | |
| 144 | cyclopentyl 3-[3-(thien-2-ylcarbonyl)pyrazolo[1,5- | +++ |
| | a]pyrimidin-7-yl]phenylcarbamate | |
| 145 | cyclohexyl 3-[3-(thien-2-ylcarbonyl)pyrazolo[1,5- | +++ |
| | a]pyrimidin-7-yl]phenylcarbamate | |
| 146 | cyclohex-2-en-1-yl 3-[3-(thien-2-ylcarbonyl)pyrazolo | +++ |
| | [1,5-a]pyrimidin-7-yl]phenylcarbamate | |
| 147 | cyclobutylmethyl 3-[3-(thien-2-ylcarbonyl)pyrazolo[1,5- | +++ |
| | a]pyrimidin-7-yl]phenylcarbamate | |
| 148 | Cyclohexylmethyl 3-[3-(thien-2-ylcarbonyl)pyrazolo[1,5- | +++ |
| | a]pyrimidin-7-yl]phenylcarbamate | |
| 149 | tetrahydro-2H-pyran-4-yl 3-[3-(thien-2- | +++ |
| | ylcarbonyl)pyrazolo [1,5-a]pyrimidin-7- | |
| | yl]phenylcarbamate | |
| 150 | 3-methylbut-2-enyl 3-[3-(thien-2- | +++ |
| | ylcarbonyl)pyrazolo[1,5-a]pyrimidin-7- | |
| | yl]phenylcarbamate | |
| 151 | 2-(methylthio)ethyl 3-[3-(thien-2-ylcarbonyl)pyrazolo | +++ |
| | [1,5-a]pyrimidin-7-yl]phenylcarbamate | |
| 162 | allyl 3-[5-methyl-3-(thien-2-ylcarbonyl)pyrazolo[1,5- | ++ |
| | a]pyrimidin-7-yl]phenylcarbamate | |
| 163 | 2-chloroethyl 3-[5-methyl-3-(thien-2- | +++ |
| | ylcarbonyl)pyrazolo[1,5-a]pyrimidin-7- | |
| | yl]phenylcarbamate | |
| 164 | 3-methyl-N-{3-[5-methyl-3-(thien-2- | +++ |
| | ylcarbonyl)pyrazolo[1,5-a]pyrimidin-7- | |
| | yl]phenyl}butanamide | |
| 165 | N-{3-[5-methyl-3-(thien-2-ylcarbonyl)pyrazolo[1,5- | ++ |
| | a]pyrimidin-7-yl]phenyl}benzamide | |
| 166 | N-{3-[5-methyl-3-(thien-2-ylcarbonyl)pyrazolo[1,5- | ++ |
| | a]pyrimidin-7-yl]phenyl}-2-thien-2-ylacetamide | |
| 167 | N-butyl-N′-{3-[5-methyl-3-(thien-2-ylcarbonyl)pyrazolo | ++ |
| | [1,5-a]pyrimidin-7-yl]phenyl}urea | |

+++, <0.1 μM;
++, 0.1-1.0 μM;
+, 1.0-20 μM

TABLE 3
Cellular activity in 80s14

| | | IC₅₀ for | |
|---|---|---|---|
| Example | | 80S14 | |
| number | Chemical Name | (μM) | MP (° C.) |
| 175 | 1-[3-[3-(2-furanylcarbonyl)pyrazolo-[1,5- | + | 159-160 |
| | a]pyrimidin-7-yl]phenyl]-2-piperidinone | | |
| 176 | 1-[3-(3-benzoylpyrazolo[1,5-a]-pyrimidin-7- | + | 160-162 |
| | yl)phenyl]-2-piperidinone | | |
| 177 | 1-[3-[3-(2-furanylcarbonyl)pyrazolo-(1,5- | + | 210-211 |
| | a]pyrimidin-7-yl]phenyl]-2-pyrrolidinone | | |
| 178 | 1-[3-(3-benzoylpyrazolo[1,5-a]-pyrimidin-7- | + | 173-174 |
| | yl)phenyl]-2-pyrrolidinone | | |
| 179 | N-[3-(3-cyanopyrazolo[1,5-a]-pyrimidin-7- | ++ | 157-158 |
| | yl)phenyl]-N-methyl-cyclobutanecarboxamide | | |
| 180 | [7-(2,4-dimethoxyphenyl)pyrazolo[1,5-a]-pyrimidin- | + | 146-148 |
| | 3-yl]-2-thienyl-methanone | | |
| 181 | N,N-diethyl-3-[3-(2-thienylcarbonyl)-pyrazolo [1,5- | + | 165-167 |
| | a]pyrimidin-7-yl]-benzamide | | |
| 182 | N-[3-(3-chloropyrazolo[1,5-a]pyrimidin-7- | + | 150-151 |
| | yl)phenyl]-N-ethyl-cyclopropanecarboxamide | | |
| 183 | N-[3-(3-cyanopyrazolo[1,5-a]pyrimidin-7-yl)phenyl]- | + | 166-168 |
| | N-ethyl-cyclopropanecarboxamide | | |
| 184 | [7-[3-(2-methoxyethoxy)phenyl]pyrazolo[1,5- | ++ | 112-116 |
| | a]pyrimidin-3-yl]-2-thienyl-methanone | | |
| 185 | [7-[3-(cyclopentyloxy)phenyl]pyrazolo[1,5- | ++ | 135-138 |
| | a]pyrimidin-3-yl]-2-thienyl-methanone | | |
| 186 | 2-furanyl[7-[3-(1H-pyrrol-1-yl)phenyl]pyrazolo[1,5- | ++ | 204-206 |
| | a]pyrimidin-3-yl]methanone | | |
| 187 | [7-[3-(1H-pyrrol-1-yl)phenyl]pyrazolo[1,5- | ++ | 211-213 |
| | a]pyrimidin-3-yl]-2-thienyl-methanone | | |
| 188 | N-{3-[3-(2-thienylcarbonyl)pyrazolo[1,5- | +++ | 192-194 |
| | a]pyrimidin-7-yl]phenyl}acrylamide | | |
| 189 | N-{3-[3-(2-thienylcarbonyl)pyrazolo[1,5- | +++ | 202-203 |
| | a]pyrimidin-7-yl]phenyl}-2-butynamide | | |
| 190 | N-{3-[3-(2-thienylcarbonyl)pyrazolo[1,5- | +++ | 70 (decomposed) |
| | a]pyrimidin-7-yl]phenyl}-2-oxiranecarboxamide | | |
| 191 | N-{3-[5-methyl-3-(2-thienylcarbonyl)pyrazolo[1,5- | +++ | 70 (decomposed) |
| | a]pyrimidin-7-yl]phenyl}-2-oxiranecarboxamide | | |
| 192 | N-{3-[3-(2-thienylcarbonyl)pyrazolo[1,5- | + | 208-210 |
| | a]pyrimidin-7-yl]phenyl}-2,5- | | |
| | bis(trifluoromethyl)benzamide | | |
| 193 | N-{3-[3-(2-thienylcarbonyl)pyrazolo[1,5- | +++ | 135-140 |
| | a]pyrimidin-7-yl]phenyl}-3- | | |
| | (trifluoromethyl)benzamide | | |
| 194 | N-{3-[3-(2-thienylcarbonyl)pyrazolo[1,5- | +++ | 211-213 |
| | a]pyrimidin-7-yl]phenyl}benzamide | | |
| 195 | N-{3-[3-(2-thienylcarbonyl)pyrazolo[1,5- | +++ | >240 |
| | a]pyrimidin-7-yl]phenyl}-2-furamide | | |
| 196 | 3-bromo-N-{3-[3-(2-thienylcarbonyl)pyrazolo[1,5- | +++ | >300 |
| | a]pyrimidin-7-yl]phenyl}benzamide | | |
| 197 | 4-(tert-butyl)-N-{3-[3-(2- | + | oil |
| | thienylcarbonyl)pyrazolo[1,5-a]pyrimidin-7- | | |
| | yl]phenyl}benzamide | | |
| 198 | 3,5-dinitro-N-{3-[3-(2- | + | dec >145 |
| | thienylcarbonyl)pyrazolo[1,5-a]pyrimidin-7- | | |
| | yl]phenyl}benzamide | | |
| 199 | 2,4-dichloro-N-{3-[3-(2- | ++ | 132-134 |
| | thienylcarbonyl)pyrazolo[1,5-a]pyrimidin-7- | | |
| | yl]phenyl}benzamide | | |
| 200 | N-{3-[3-(2-thienylcarbonyl)pyrazolo[1,5- | +++ | 191-194 |
| | a]pyrimidin-7-yl]phenyl}nicotinamide | | |
| 201 | N-{3-[3-(2-thienylcarbonyl)pyrazolo[1,5- | +++ | 220-222 |
| | a]pyrimidin-7-yl]phenyl}isonicotinamide | | |
| 202 | N,N′-dibutyl-N-{3-[3-(2- | 4.5 | 180-181 |
| | thienylcarbonyl)pyrazolo[1,5-a]pyrimidin-7- | | |
| | yl]phenyl}dicarbonimidic diamide | | |
| 203 | isopropyl 3-[3-(cyclopentylcarbonyl)pyrazolo[1,5- | ++ | 57-59 |
| | a]pyrimidin-7-yl]phenylcarbamate | | |
| 204 | N-{3-[3-(cyclopentylcarbonyl)pyrazolo[1,5- | ++ | 69-71 |
| | a]pyrimidin-7-yl]phenyl}-3-methylbutanamide | | |
| 205 | N-{3-[3-(2-furoyl)pyrazolo[1,5-a]pyrimidin-7- | ++ | 220-222 |
| | yl]phenyl}benzamide | | |
| 206 | methyl 3-[3-(2-thienylcarbonyl)pyrazolo[1,5- | + | 180-184 |
| | a]pyrimidin-7-yl]benzoate | | |
| 207 | N-{2-methyl-5-[3-(2-thienylcarbonyl)pyrazolo[1,5- | ++ | 216-218 |
| | a]pyrimidin-7-yl]phenyl}benzamide | | |
| 208 | 3-methyl-N-{2-methyl-5-[3-(2- | ++ | 185-187 |
| | thienylcarbonyl)pyrazolo[1,5-a]pyrimidin-7- | | |
| | yl]phenyl}butanamide | | |
| 209 | N-isopropyl-N′-{2-methyl-5-[3-(2- | +++ | 225-227 |
| | thienylcarbonyl)pyrazolo[1,5-a]pyrimidin-7- | | |
| | yl]phenyl}urea | | |
| 210 | N-{2-chloro-5-[3-(2-thienylcarbonyl)pyrazolo[1,5- | +++ | 217-218 |
| | a]pyrimidin-7-yl]phenyl}benzamide | | |
| 211 | N-{2-chloro-5-[3-(2-thienylcarbonyl)pyrazolo[1,5- | +++ | 172-175 |
| | a]pyrimidin-7-yl]phenyl}-3-methylbutanamide | | |
| 212 | isopropyl 2-methyl-5-[3-(2- | +++ | 198-200 |
| | thienylcarbonyl)pyrazolo[1,5-a]pyrimidin-7- | | |
| | yl]phenylcarbamate | | |
| 213 | N-{2-methoxy-5-[3-(2- | ++ | 201-203 |
| | thienylcarbonyl)pyrazolo[1,5-a]pyrimidin-7- | | |
| | yl]phenyl}benzamide | | |
| 214 | isopropyl 2-methoxy-5-[3-(2- | ++ | 193-195 |
| | thienylcarbonyl)pyrazolo[1,5-a]pyrimidin-7- | | |
| | yl]phenylcarbamate | | |
| 215 | N-{2-methoxy-5-[3-(2- | ++ | 192-194 |
| | thienylcarbonyl)pyrazolo[1,5-a]pyrimidin-7- | | |
| | yl]phenyl}-3-methylbutanamide | | |
| 216 | N-isobutyl-3-[3-(2-thienylcarbonyl)pyrazolo[1,5- | +++ | 174-177 |
| | a]pyrimidin-7-yl]benzamide | | |
| 217 | N-butyl-3-[3-(2-thienylcarbonyl)pyrazolo[1,5- | +++ | 172-174 |
| | a]pyrimidin-7-yl]benzamide | | |
| 218 | N-cyclopentyl-3-[3-(2- | +++ | 207-209 |
| | thienylcarbonyl)pyrazolo[1,5-a]pyrimidin-7- | | |
| | yl]benzamide | | |
| 219 | N-phenyl-3-[3-(2-thienylcarbonyl)pyrazolo[1,5- | +++ | 211-212 |
| | a]pyrimidin-7-yl]benzamide | | |
| 220 | N-isopropyl-3-[3-(2-thienylcarbonyl)pyrazolo[1,5- | +++ | 218-220 |
| | a]pyrimidin-7-yl]benzamide | | |
| 221 | N-isopropyl-N′-{2-methoxy-5-[3-(2- | ++ | 202-204 |
| | thienylcarbonyl)pyrazolo[1,5-a]pyrimidin-7- | | |
| | yl]phenyl}urea | | |
| 222 | 3-methyl-N-{5-[3-(2-thienylcarbonyl)pyrazolo[1,5- | ++ | 217-219 |
| | a]pyrimidin-7-yl]-3-pyridinyl}butanamide | | |
| 223 | 7-(3-nitrophenyl)-3-(3-thienyl)pyrazolo[1,5- | + | 148-150 |
| | a]pyrimidine | | |
| 224 | 3-(3-bromopyrazolo[1,5-a]pyrmidin-7-yl)aniline | + | 120-121 |
| 225 | Isobutyl 3-(3-bromopyrazolo[1,5-a]pyrimidin-7- | + | 90-92 |
| | yl)phenylcarbamate | | |
| 226 | [7-(5-nitro-3-thienyl)pyrazolo[1,5-a]pyrimidin-3- | ++ | 225-226 |
| | yl](2-thienyl)methanone | | |
| 227 | N-isopropyl-N′-{5-[3-(thien-2- | ++ | 162-165 |
| | ylcarbonyl)pyrazolo[1,5-a]pyrimidin-7-yl]thien-2- | | |
| | yl}urea | | |
| 228 | N-[3-(3-bromopyrazolo[1,5-a]pyrimidin-7- | + | 132-134 |
| | yl)phenyl]-3-methylbutanamide | | |
| 229 | [7-(5-aminothien-3-yl)pyrazolo[1,5-a]pyrimidin-3-yl] | ++ | 174-176 |
| | (thien-2-yl)methanone | | |
| 230 | O-ethyl 3-[3-(thien-2-ylcarbonyl)pyrazolo[1,5- | +++ | 209-211 |
| | a]pyrimidin-7-yl]phenylthiocarbamate | | |
| 231 | 3-methyl-N-[3-(3-thien-2-ylpyrazolo[1,5- | + | 148-150 |
| | a]pyrimidin-7-yl)phenyl]butanamide | | |
| 232 | N-phenyl-N′-{3-[3-(thien-2-ylcarbonyl)pyrazolo[1,5- | +++ | 124-126 |
| | a]pyrimidin-7-yl]phenyl}thiourea | | |
| 233 | isopropyl 4-chloro-3-[3-(thien-2- | +++ | 234-236 |
| | ylcarbonyl)pyrazolo[1,5-a]pyrimidin-7- | | |
| | yl]phenylcarbamate | | |
| 234 | [7-(2-chloro-5-nitrophenyl)pyrazolo[1,5-a]pyrimidin- | + | 162-166 |
| | 3-yl](thien-2-yl)methanone | | |
| 235 | N-{3-[3-(pyridin-2-ylcarbonyl)pyrazolo[1,5- | + | 214-215 |
| | a]pyrimidin-7-yl]phenyl}cyclopropanecarboxamide | | |
| 236 | N-{4-chloro-3-[3-(thien-2-ylcarbonyl)pyrazolo[1,5- | ++ | 162-165 |
| | a]pyrimidin-7-yl]phenyl}-3-methylbutanamide | | |
| 237 | N-{4-chloro-3-[3-(thien-2-ylcarbonyl)pyrazolo[1,5- | ++ | 194-195 |
| | a]pyrimidin-7-yl]phenyl}-N′-isopropylthiourea | | |
| 238 | N-{4-chloro-3-[3-(thien-2-ylcarbonyl)pyrazolo[1,5- | ++ | 194-195 |
| | a]pyrimidin-7-yl]phenyl}benzamide | | |
| 239 | N-{2-fluoro-5-[3-(thien-2-ylcarbonyl)pyrazolo[1,5- | +++ | 192-194 |
| | a]pyrimidin-7-yl]phenyl}-3-methylbutanamide | | |
| 240 | 3-methyl-N-{5-[3-(thien-2-ylcarbonyl)pyrazolo[1,5- | ++ | 268-270 |
| | a]pyrimidin-7-yl]thien-2-yl}butanamide | | |
| 241 | isopropyl 5-[3-(thien-2-ylcarbonyl)pyrazolo[1,5- | ++ | 210-215 |
| | a]pyrimidin-7-yl]thien-2-ylcarbamate | | |
| 242 | [7-(5-aminothien-2-yl)pyrazolo[1,5-a]pyrimidin-3- | + | 232-235 |
| | yl](thien-2-yl)methanone | | (dec.) |
| 243 | 4-chloro-N-{3-[3-(thien-2-ylcarbonyl)pyrazolo[1,5- | ++ | 97-100 |
| | a]pyrimidin-7-yl]phenyl}butanamide | | |
| 244 | isopropyl 2-fluoro-5-[3-(thien-2- | +++ | >200 |
| | ylcarbonyl)pyrazolo[1,5-a]pyrimidin-7- | | |
| | yl]phenylcarbamate | | |
| 245 | isopropyl 2-(dimethylamino)-5-[3-(thien-2- | ++ | 164-165 |
| | ylcarbonyl)pyrazolo[1,5-a]pyrimidin-7- | | |
| | yl]phenylcarbamate | | |
| 246 | {7-[3-(benzylamino)phenyl]pyrazolo[1,5- | ++ | 90-92 |
| | a]pyrimidin-3-yl}(thien-2-yl)methanone | | |
| 247 | 3-methyl-N-{4-[3-(thien-2-ylcarbonyl)pyrazolo[1,5- | ++ | 234-235 |
| | a]pyrimidin-7-yl]thien-2-yl}butanamide | | |
| 248 | [7-(3-amino-4-fluorophenyl)pyrazolo[1,5- | ++ | 241-242 |
| | a]pyrimidin-3-yl](thien-2-yl)methanone | | |
| 249 | N-cyclohexyl-N′-{3-[3-(thien-2- | +++ | 233-236 |
| | ylcarbonyl)pyrazolo[1,5-a]pyrimidin-7- | | |
| | yl]phenyl}urea | | |
| 250 | 4,4,4-trichloro-N-{3-[3-(thien-2- | +++ | 189-191 |
| | ylcarbonyl)pyrazolo[1,5-a]pyrimidin-7- | | |
| | yl]phenyl}butanamide | | |
| 251 | N-{2-fluoro-5-[3-(thien-2-ylcarbonyl)pyrazolo[1,5- | +++ | 180-181 |
| | a]pyrimidin-7-yl]phenyl}-3,3-dimethylbutanamide | | |
| 252 | 4,4,4-trifluoro-N-{2-fluoro-5-[3-(thien-2- | +++ | 185-186 |
| | ylcarbonyl)pyrazolo[1,5-a]pyrimidin-7-yl]phenyl}-3- | | |
| | methylbutanamide | | |
| 253 | bis(2,2,2-trichloroethyl) 2-fluoro-5-[3-(thien-2- | +++ | 94-97 |
| | ylcarbonyl)pyrazolo[1,5-a]pyrimidin-7- | | |
| | yl]phenylimidodicarbonate | | |
| 254 | 2-fluoro-N-{3-[3-(thien-2-ylcarbonyl)pyrazolo[1,5- | +++ | 190-191 |
| | a]pyrimidin-7-yl]phenyl}benzamide | | |
| 255 | 2,6-difluoro-N-{3-[3-(thien-2- | +++ | 159-160 |
| | ylcarbonyl)pyrazolo[1,5-a]pyrimidin-7- | | |
| | yl]phenyl}benzamide | | |
| 256 | N-{3-[3-(thien-2-ylcarbonyl)pyrazolo[1,5- | +++ | 198-200 |
| | a]pyrimidin-7-yl]phenyl}pyridine-2-carboxamide | | |
| 257 | N-{5-[3-(thien-2-ylcarbonyl)pyrazolo[1,5- | + | 162-165 |
| | a]pyrimidin-7-yl]thien-2-yl}benzamide | | |
| 258 | (7-{3-[(2-phenylethyl)amino]phenyl}pyrazolo[1,5- | + | 118-120 |
| | a]pyrimidin-3-yl)(thien-2-yl)methanone | | |
| 259 | (7-{3-[(2-methoxybenzyl)amino]phenyl} | + | 138-140 |
| | pyrazolo[1,5-a]pyrimidin-3-yl)(thien-2-yl)methanone | | |
| 260 | N-butyl-N′-{3-[3-(thien-2-ylcarbonyl)pyrazolo[1,5- | +++ | 155-157 |
| | a]pyrimidin-7-yl]phenyl}thiourea | | |
| 261 | (7-{3-[(3-chlorobenzyl)amino]phenyl}pyrazolo[1,5- | + | 133-135 |
| | a]pyrimidin-3-yl)(thien-2-yl)methanone | | |
| 262 | (7-{3-[(4-chlorobenzyl)amino]phenyl}pyrazolo[1,5- | + | 149-152 |
| | a] pyrimidin-3-yl)(thien-2-yl)methanone | | |
| 263 | (7-{3-[(2-furylmethyl)amino]phenyl}pyrazolo[1,5- | ++ | Oil, |
| | a]pyrimidin-3-yl)(thien-2-yl)methanone | | M + H 401 |
| 264 | (7-{3-[(2-chlorobenzyl)amino]phenyl}pyrazolo[1,5- | ++ | Oil, |
| | a]pyrimidin-3-yl)(thien-2-yl)methanone | | M + H 445 |
| 265 | (7-{3-[(3-furylmethyl)amino]phenyl}pyrazolo[1,5- | ++ | 140-144 |
| | a]pyrimidin-3-yl)(thien-2-yl)methanone | | |
| 266 | (7-{3-[(2E)-but-2-enylamino]phenyl}pyrazolo[1,5- | +++ | 110-113 |
| | a]pyrimidin-3-yl)(thien-2-yl)methanone | | |
| 267 | (7-{3-[(3-methylbut-2- | +++ | 116-118 |
| | enyl)amino]phenyl}pyrazolo[1,5-a]pyrimidin-3- | | |
| | yl(thien-2-yl)methanone | | |
| 268 | [7-(6-chloropyridin-3-yl)pyrazolo[1,5-a]pyrimidin- | + | 145-148 |
| | 3-yl] (thien-2-yl)methanone | | |
| 269 | 2,6-difluoro-N-{2-fluoro-5-[3-(thien-2- | +++ | 198-200 |
| | ylcarbonyl)pyrazolo[1,5-a]pyrimidin-7- | | |
| | yl]phenyl}benzamide | | |
| 270 | 2-fluoro-N-{2-fluoro-5-[3-(thien-2- | +++ | 214-216 |
| | ylcarbonyl)pyrazolo[1,5-a]pyrimidin-7- | | |
| | yl]phenyl}benzamide | | |
| 271 | 2-chloro-N-{2-fluoro-5-[3-(thien-2- | +++ | 166-168 |
| | ylcarbonyl)pyrazolo[1,5-a]pyrimidin-7- | | |
| | yl]phenyl}benzamide | | |
| 272 | (7-{3-[(2- | ++ | 177-179 |
| | methylbenzyl)amino]phenyl}pyrazolo[1,5- | | |
| | a]pyrimidin-3-yl)(thien-2-yl)methanone | | |
| 273 | (7 {3-[(2- | ++ | 108-110 |
| | phenylpropyl)amino]phenyl}pyrazolo[1,5- | | |
| | a]pyrimidin-3-yl)(thien-2-yl)methanone | | |
| 274 | (7-{3- | +++ | 101-104 |
| | [(cyclohexylmethyl)amino]phenyl}pyrazolo[1,5- | | |
| | a]pyrimidin-3-yl)(thien-2-yl)methanone | | |
| 275 | 2-chloroethyl 2-fluoro-5-[3-(thien-2- | +++ | 194-196 |
| | ylcarbonyl)pyrazolo[1,5-a]pyrimidin-7- | | |
| | yl]phenylcarbamate | | |
| 276 | N-{2-fluoro-5-[3-(thien-2-ylcarbonyl)pyrazolo[1,5- | +++ | >200 |
| | a]pyrimidin-7-yl]phenyl}-N′-isopropylurea | | |
| 277 | 4-(1H-pyrrol-1-yl)-N-{3-[3-(thien-2- | ++ | 224-226 |
| | ylcarbonyl)pyrazolo[1,5-a]pyrimidin-7- | | |
| | yl]phenyl}benzamide | | |
| 278 | 2,6-dichloro-N-{3-[3-(thien-2- | ++ | 245-246 |
| | ylcarbonyl)pyrazolo[1,5-a]pyrimidin-7- | | |
| | yl]phenyl}benzamide | | |
| 279 | N-{3-[3-(thien-2-ylcarbonyl)pyrazolo[1,5- | +++ | 222-224 |
| | a]pyrimidin-7-yl]phenyl}pyrazine-2-carboxamide | | |
| 280 | phenyl N′-cyano-N-{3-[3-(thien-2- | ++ | 220(dec) |
| | ylcarbonyl)pyrazolo[1,5-a]pyrimidin-7- | | |
| | yl]phenyl}imidocarbamate | | |
| 281 | 1-methyl-N-{3-[3-(thien-2-ylcarbonyl)pyrazolo[1,5- | +++ | 120(dec) |
| | a]pyrimidin-7-yl]phenyl}-1H-pyrrole-2-carboxamide | | |
| 282 | N-{2-fluoro-5-[3-(thien-2-ylcarbonyl)pyrazolo[1,5- | +++ | 175-179 |
| | a]pyrimidin-7-yl]phenyl}isonicotinamide | | |
| 283 | N-isopropyl-N′-{3-[3-(thien-2- | +++ | 208-210 |
| | ylcarbonyl)pyrazolo[1,5-a]pyrimidin-7- | | |
| | yl]phenyl}thiourea | | |
| 284 | (7-chloropyrazolo[1,5-a]pyrimidin-3-yl)(thien-2- | ++ | 148-150 |
| | yl)methanone | | |
| 285 | 2,6-dichloro-N-(2,6-dichlorobenzoyl)-N-{3-[3-(thien- | + | 238-240 |
| | 2-ylcarbonyl)pyrazolo[1,5-a]pyrimidin-7- | | |
| | yl]phenyl}benzamide | | |
| 286 | tert-butyl 4-[2-oxo-2-({3-[3-(thien-2- | +++ | 173-175 |
| | ylcarbonyl)pyrazolo[1,5-a]pyrimidin-7- | | |
| | yl]phenyl}amino)ethyl]piperidine-1-carboxylate | | |
| 287 | 2-piperidin-4-yl-N-3-[3-(thien-2- | ++ | 216-220 |
| | ylcarbonyl)pyrazolo[1,5-a]pyrimidin-7- | | |
| | yl]phenyl}acetamide | | |
| 288 | [7-(3-{[(2E)-3-(2-methoxyphenyl)prop-2- | + | 128-131 |
| | enyl]amino}phenyl)pyrazolo[1,5- | | |
| | a]pyrimidin-3-yl](thien-2-yl)methanone | | |
| 289 | [7-(3-{[(2E)-3-(4-methoxyphenyl)prop-2- | + | 176-179 |
| | enyl]amino}phenyl)pyrazolo[1,5- | | |
| | a]pyrimidin-3-yl](thien-2-yl)methanone | | |
| 290 | [7-(3-{[(2E)-2-methyl-3-phenylprop-2- | + | 74-76 |
| | enyl]amino}phenyl)pyrazolo[1,5-a]pyrimidin-3- | | |
| | yl](thien-2-yl)methanone | | |
| 291 | [7-(3-{[(2E)-3-(4-nitrophenyl)prop-2- | + | 163-166 |
| | enyl] amino}phenyl)pyrazolo[1,5-a]pyrimidin- | | |
| | 3-yl](thien-2-yl)methanone | | |
| 292 | (7-{3-[(3,3-diphenylprop-2- | + | 156-158 |
| | enyl)amino]phenyl}pyrazolo[1,5-a]pyrimidin-3- | | |
| | yl)(thien-2-yl)methanone | | |
| 293 | (7-{3-[(3-phenylbutyl)amino]phenyl}pyrazolo[1,5- | ++ | 64-68 |
| | a]pyrimidin-3-yl)(thien-2-yl)methanone | | |
| 294 | [7-(3-{[(2E)-3-(4-hydroxy-3-methoxyphenyl)prop- | + | 180-184 |
| | 2-enyl]amino}phenyl)pyrazolo[1,5-a]pyrimidin-3- | | |
| | yl](thien-2-yl)methanone | | |
| 295 | tert-butyl 4-[({3-[3-(thien-2- | ++ | 148-150 |
| | ylcarbonyl)pyrazolo[1,5-a]pyrimidin-7- | | |
| | yl]phenyl}amino)carbonyl]piperidine-1-carboxylate | | |
| 296 | N,N′-bis{3-[3-(thien-2-ylcarbonyl)pyrazolo[1,5- | + | 151-153 |
| | a]pyrimidin-7-yl]phenyl}thiourea | | |
| 297 | isobutyl3-(3-thien-2-ylpyrazolo[1,5-a]pyrimidin-7- | ++ | 140-141 |
| | yl)phenylcarbamate | | |
| 298 | N-{3-[3-(thien-2-ylcarbonyl)pyrazolo[1,5- | ++ | 159-161 |
| | a]pyrimidin-7-yl]phenyl} piperidine-4-carboxamide | | |
| 299 | [7-(3-{[(2E)-2-hexyl-3-phenylprop-2- | + | 110-115 |
| | enyl]amino}phenyl)pyrazolo[1,5-a]pyrimidin-3- | | |
| | yl](thien-2-yl)methanone | | |
| 300 | [7-(3-{[(2E)-2-pentyl-3-phenylprop-2- | + | 110-112 |
| | enyl]amino}phenyl)pyrazolo[1,5-a]pynmidin-3-yl] | | |
| | (thien-2-yl)methanone | | |
| 301 | N-isopropyl-N′-{3-[3-(thien-2- | + | >200 |
| | ylcarbonyl)pyrazolo[1,5-a]pyrimidin-7-yl]phenyl} | | |
| | guanidine | | |
| 302 | [7-(3-{[(2E)-3-phenylprop-2- | + | 123-125 |
| | enyl]amino}phenyl)pyrazolo[1,5-a]pyrimidin-3-yl] | | |
| | (thien-2-yl)methanone | | |
| 303 | [7-(3-{[(2E)-3-(4-tert-butylphenyl)-2-methylprop- | + | 135-137 |
| | 2-enyl]amino}phenyl)pyrazolo[1,5-a]pyrimidin-3- | | |
| | yl](thien-2-yl)methanone | | |
| 304 | N″-cyano-N-isopropyl-N′-{3-[3-(thien-2- | +++ | 240-241 |
| | ylcarbonyl)pyrazolo[1,5-a]pyrimidin-7- | | |
| | yl]phenyl}guanidine | | |
| 305 | N-{3-[3-(thien-2-yicarbonyl)pyrazolo[1,5- | +++ | 280-282 |
| | a]pyrimidin-7-yl]phenyl}-1H-imidazole-5- | | |
| | carboxamide | | |
| 306 | pyridin-4-ylmethyl 3-[3-(thien-2- | +++ | 185(dec) |
| | ylcarbonyl)pyrazolo[1,5-a]pyrimidin-7- | | |
| | yl]phenylcarbamate | | |
| 307 | 1-methyl-N-{3-[3-(thien-2- | + | 236 |
| | ylcarbonyl)pyrazolo[1,5-a]pyrimidin-7- | | |
| | yl]phenyl}piperidine-4-carboxamide | | |
| 308 | (7-{3-[(2-ethylhexyl)amino]phenyl}pyrazolo[1,5- | ++ | 134-138 |
| | a]pyrimidin-3-yl)(thien-2-yl)methanone | | |
| 309 | 3-(1,1-dioxidothiomorpholin-4-yl)propyl 3-[3- | +++ | 148-150 |
| | (thien-2-ylcarbonyl)pyrazolo[1,5-a]pyrimidin-7- | | |
| | yl]phenylcarbamate | | |
| 310 | 2-chloro-N-{2-(dimethylamino)-5-[3-(thien-2- | +++ | 220-222 |
| | ylcarbonyl)pyrazolo[1,5-a]pyrimidin- | | |
| | 7-yl]phenyl}benzamide | | |
| 311 | 4-morpholin-4-yl-N-{3-[3-(thien-2- | ++ | 127-133 |
| | ylcarbonyl)pyrazolo[1,5-a]pyrimidin-7- | | |
| | yl]phenyl}butanamide | | |
| 312 | pyridin-3-ylmethyl 3-[3-(thien-2- | +++ | >200 |
| | ylcarbonyl)pyrazolo[1,5-a]pyrimidin-7- | | |
| | yl]phenylcarbamate | | |
| 313 | 3-morpholin-4-ylpropyl 3-[3-(thien-2- | ++ | 198-199 |
| | ylcarbonyl)pyrazolo[1,5-a]pyrimidin-7- | | |
| | yl]phenylcarbamate | | |
| 314 | [7-(3-{[(2E)-2-methylpent-2- | ++ | 132-133 |
| | enyl]amino}phenyl)pyrazolo[1,5-a]pyrimidin-3- | | |
| | yl](thien-2-yl)methanone | | |
| 315 | 2-pyridin-4-yl-N-{3-[3-(thien-2- | +++ | 130-131 |
| | ylcarbonyl)pyrazolo[1,5-a]pyrimidin-7- | | |
| | yl]phenyl}acetamide | | |
| 316 | N-{2-(dimethylamino)-5-[3-(thien-2- | ++ | 151-153 |
| | ylcarbonyl)pyrazolo[1,5-a]pyrimidin-7- | | |
| | yl]phenyl}isonicotinamide | | |
| 317 | pyridin-2-ylmethyl 3-[3-(thien-2- | ++ | 121-125 |
| | ylcarbonyl)pyrazolo[1,5-a]pyrimidin-7- | | |
| | yl]phenylcarbamate | | |
| 318 | (7-{3-[(2,3- | ++ | 84-86 |
| | dimethylpentyl)amino]phenyl}pyrazolo[1,5- | | |
| | a]pyrimidin-3-yl)(thien-2-yl)methanone | | |
| 319 | [7-(3-{[(2E)-2-methylbut-2- | ++ | 118-120 |
| | enyl]amino}phenyl)pyrazolo[1,5-a]pyrimidin-3- | | |
| | yl](thien-2-yl)methanone | | |
| 320 | (7-{3-[(2E)-pent-2- | +++ | 72-75 |
| | enylamino]phenyl}pyrazolo[1,5-a]pyrimidin-3- | | |
| | yl)(thien-2-yl)methanone | | |
| 321 | thien-2-yl(7-{3-[(3,5,5- | ++ | 81-85 |
| | trimethylhexyl)amino]phenyl}pyrazolo[1,5- | | |
| | a]pyrimidin-3-yl)methanone | | |
| 322 | (7-{3-[(2-methylprop-2- | ++ | 104-106 |
| | enyl)amino]phenyl}pyrazolo[1,5-a]pyrimidin-3- | | |
| | yl)(thien-2-yl)methanone | | |
| 323 | (7-{3-[(2-ethylprop-2- | ++ | 79-82 |
| | enyl)amino]phenyl}pyrazolo[1,5-a]pyrimidin-3- | | |
| | yl)(thien-2-yl)methanone | | |
| 324 | (7-{3-[(cyclohex-3-en-1- | +++ | 78-81 |
| | ylmethyl)emino]phenyl}pyrazolo[1,5-a]pyrimidin- | | |
| | 3-yl)-(thien-2-yl)-methanone | | |
| 325 | 1-{3-[3-(thien-2-ylcarbonyl)pyrazolo[1,5- | +++ | >200 |
| | a]pyrimidin-7-yl]phenyl}pyrrolidin-2-one | | |
| 326 | 2-chloro-N-(2-chlorobenzoyl)-N-{2-fluoro-5-[3- | +++ | 150-151 |
| | (thien-2-ylcarbonyl)pyrazolo[1,5-a]pyrimidin-7- | | |
| | yl]phenyl}benzamide | | |
| 327 | 2-pyridin-3-yl-N-{3-[3-(thien-2- | ++ | >200 |
| | ylcarbonyl)pyrazolo[1,5-a]pyrimidin-7- | | |
| | yl]phenyl}acetamide | | |
| 328 | 3-pyridin-4-yl-N-{3-[3-(thien-2- | ++ | 176-181 |
| | ylcarbonyl)pyrazolo[1,5-a]pyrimidin-7- | | |
| | yl]phenyl}propanamide | | |
| 329 | N-{3-[3-(thien-2-ylcarbonyl)pyrazolo[1,5- | + | 165-170 |
| | a]pyrimidin-7-yl]phenyl}-1H-pyrazole-4- | | |
| | carboxamide | | |
| 330 | 4-oxo-4-({3-[3-(thien-2-ylcarbonyl)pyrazolo[1,5- | + | 240-241 |
| | a]pyrimidin-7-yl]phenyl}amino)butanoic acid | | |
| 331 | 2-[({3-[3-(thien-2-ylcarbonyl)pyrazolo[1,5- | + | 215-216 |
| | a]pyrimidin-7-yl]phenyl}amino)carbonyl]benzoic | | |
| | acid | | |
| 332 | 5-oxo-5-({3-[3-(thien-2-ylcarbonyl)pyrazolo[1,5- | + | 189-190 |
| | a]pyrimidin-7-yl]phenyl}amino)pentanoic acid | | |
| 333 | 3-methyl-5-oxo-5-({3-[3-(thien-2- | + | 135-136 |
| | ylcarbonyl)pyrazolo[1,5-a]pyrimidin-7- | | |
| | yl]phenyl}amino)pentanoic acid | | |
| 334 | 4,5-dichloro-2-[({3-[3-(thien-2- | + | >270 |
| | ylcarbonyl)pyrazolo[1,5-a]pyrimidin-7- | | |
| | yl]phenyl}amino)carbonyl]benzoic acid | | |
| 335 | (7-{3-[(2- | + | 108-110 |
| | methylundecyl)amino]phenyl}pyrazolo[1,5- | | |
| | a]pyrimidin-3-yl)(thien-2-yl)methanone | | |
| 336 | 3-pyridin-3-yl-N-{3-[3-(thien-2- | ++ | >200 |
| | ylcarbonyl)pyrazolo[1,5-a]pyrimidin-7- | | |
| | yl]phenyl}propanamide | | |
| 337 | 2-(pyridin-4-ylthio)-N-{3-[3-(thien-2- | + | 138-144 |
| | ylcarbonyl)pyrazolo[1,5-a]pyrimidin-7- | | |
| | yl]phenyl} acetamide | | |
| 338 | {7-[4′-(dimethylamino)-1,1′-biphenyl-3- | +++ | 210-202 |
| | yl]pyrazolo[1,5-a]pyrimidin-3-yl}(thien-2- | | |
| | yl)methanone | | |
| 339 | [7-(3′-amino-1,1′-biphenyl-3-yl)pyrazolo[1,5- | +++ | 117-121 |
| | a]pyrimidin-3-yl](thien-2-yl)methanone | | |
| 340 | {7-[4′-(hydroxymethyl)-1,1′-biphenyl-3- | +++ | 106-110 |
| | yl]pyrazolo[1,5-a]pyrimidin-3-yl}(thien-2- | | |
| | yl)methanone | | |
| 341 | {7-[3′-(dimethylamino)-1,1′-biphenyl-3- | ++ | 167-169 |
| | yl]pyrazolo[1,5-a]pyrimidin-3-yl}(thien-2- | | |
| | yl)methanone | | |
| 342 | {7-[4′-(dimethylamino)-6-fluoro-1,1′-biphenyl-3- | ++ | 219-221 |
| | yl]pyrazolo[1,5-a]pyrimidin-3-yl}(thien-2- | | |
| | yl)methanone | | |
| 343 | {7-[6-fluoro-4′-(hydroxymethyl)-1,1′-biphenyl-3- | ++ | 140-145 |
| | yl]pyrazolo[1,5-a]pyrimidin-3-yl}(thien-2- | | |
| | yl)methanone | | |
| 344 | [7-(4′-ethyl-1,1′-biphenyl-3-yl)pyrazolo[1,5- | +++ | 117-119 |
| | a]pyrimidin-3-yl](thien-2-yl)methanone | | |
| 345 | 3-{3′-[3-(thien-2-ylcarbonyl)pyrazolo[1,5- | + | 117-119 |
| | a]pyrimidin-7-yl]-1,1′-biphenyl-4-yl}propanoic acid | | |
| 346 | 3′-[3-(thien-2-ylcarbonyl)pyrazolo[1,5-a]pyrimidin-7- | ++ | 156-158 |
| | yl]-1,1′-biphenyl-4-carbaldehyde | | |
| 347 | {7-[4′-(morpholin-4-ylmethyl)-1,1′-biphenyl-3- | +++ | 175-178 |
| | yl]pyrazolo[1,5-a]pyrimidin-3-yl}(thien-2- | | |
| | yl)methanone | | |
| 348 | (7-{4′-[(4-methylpiperazin-1-yl)methyl]-1,1′- | +++ | 199-202 |
| | biphenyl-3-yl}pyrazolo[1,5-a]pyrimidin-3-yl)(thien- | | |
| | 2-yl)methanone | | |
| 349 | [7-(4′-{[(2-morpholin-4-ylethyl)amino]methyl}-1,1′- | ++ | 100-106 |
| | biphenyl-3-yl)pyrazolo[1,5-a]pyrimidin-3-yl](thien- | | |
| | 2-yl)methanone | | |
| 350 | (7-{4′-[(diethylamino)methyl]-1,1′-biphenyl-3- | ++ | 144-146 |
| | yl}pyrazolo[1,5-a]pyrimidin-3-yl)(thien-2- | | |
| | yl)methanone | | |
| 351 | (7-{4′-[(dimethylamino)methyl]-1,1′-biphenyl-3- | +++ | 192-195 |
| | yl}pyrazolo[1,5-a]pyrimidin-3-yl)(thien-2- | | |
| | yl)methanone | | |
| 352 | N-{3-[3-(3-aminophenyl)pyrazolo[1,5-a]pyrimidin-7- | + | 173-175 |
| | yl]phenyl}-3-methylbutanamide | | |
| 353 | N-(3-{3-[3-(dimethylamino)phenyl]pyrazolo[1,5- | +++ | 182-184 |
| | a]pyrimidin-7-yl}phenyl)-3-methylbutanamide | | |
| 354 | N-{3-[3-(1H-indol-5-yl)pyrazolo[1,5-a]pyrimidin-7- | ++ | 167-169 |
| | yl]phenyl}-3-methylbutanamide | | |
| 355 | 3-methyl-N-{3-[3-(3,4,5- | +++ | 86-90 |
| | trimethoxyphenyl)pyrazolo[1,5-a]pyrimidin-7- | | |
| | yl]phenyl}butanamide | | |
| 356 | 3-methyl-N-[3-(3-quinolin-8-ylpyrazolo[1,5- | ++ | 96-99 |
| | a]pyrimidin-7-yl)phenyl]butanamide | | |
| 357 | N-(3-{3-[4-(hydroxymethyl)phenyl]pyrazolo[1,5- | + | 170-172 |
| | a]pyrimidin-7-yl}phenyl)-3-methylbutanamide | | |
| 358 | N-{3-[3-(4-methoxyphenyl)pyrazolo[1,5- | + | 225-227 |
| | a]pyrimidin-7-yl]phenyl}-3-methylbutanamide | | |
| 359 | N-{3-[3-(3-chlorophenyl)pyrazolo[1,5-a]pyrimidin-7- | + | 169-171 |
| | yl]phenyl}-3-methylbutanamide | | |
| 360 | N-{3-[3-(4-chlorophenyl)pyrazolo[1,5-a]pyrimidin-7- | + | 215-217 |
| | yl]phenyl}-3-methylbutanamide | | |
| 361 | N-{3-[3-(2,3-dichlorophenyl)pyrazolo[1,5- | ++ | 154-156 |
| | a]pyrimidin-7-yl]phenyl}-3-methylbutanamide | | |
| 362 | N-{3-[3-(3,4-dichlorophenyl)pyrazolo[1,5- | + | 215-217 |
| | a]pyrimidin-7-yl]phenyl}-3-methylbutanamide | | |
| 363 | N-{3-[3-(3,5-dichlorophenyl)pyrazolo[1,5- | ++ | 223-225 |
| | a]pyrimidin-7-yl]phenyl}-3-methylbutanamide | | |
| 364 | N-{3-[3-(3,5-dimethylphenyl)pyrazolo[1,5- | +++ | 178-180 |
| | a]pyrimidin-7-yl]phenyl}-3-methylbutanamide | | |
| 365 | 3-methyl-N-(3-{3-[3- | + | 170-173 |
| | (trifluoromethyl)phenyl]pyrazolo[1,5-a]pyrimidin-7- | | |
| | yl}phenyl)butanamide | | |
| 366 | N-{3-[3-(3-methoxyphenyl)pyrazolo[1,5- | + | 153-154 |
| | a]pyrimidin-7-yl]phenyl}-3-methylbutanamide | | |
| 367 | N-{3-[3-(3-ethoxyphenyl)pyrazolo[1,5-a]pyrimidin- | + | 156-158 |
| | 7-yl]phenyl}-3-methylbutanamide | | |
| 368 | 3-methyl-N-{3-[3-(4-phenoxyphenyl)pyrazolo[1,5- | + | 175-178 |
| | a]pyrimidin-7-yl]phenyl}butanamide | | |
| 369 | N-{3-[3-(1,3-benzodioxol-5-yl)pyrazolo[1,5- | ++ | 172-173 |
| | a]pyrimidin-7-yl]phenyl}-3-methylbutanamide | | |
| 370 | N-{3-[3-(3-cyanophenyl)pyrazolo[1,5-a]pyrimidin-7- | + | 166-167 |
| | yl]phenyl}-3-methylbutanamide | | |
| 371 | N-{3-[3-(3-acetylphenyl)pyrazolo[1,5-a]pyrimidin-7- | + | 190-192 |
| | yl]phenyl}-3-methylbutanamide | | |
| 372 | N-{3-[3-(3-formyl-4-methoxyphenyl)pyrazolo[1,5- | ++ | 187-189 |
| | a]pyrimidin-7-yl]phenyl}-3-methylbutanamide | | |
| 373 | N-{3-[3-(1,1′-biphenyl-3-yl)pyrazolo[1,5- | + | 182-185 |
| | a]pyrimidin-7-yl]phenyl}-3-methylbutanamide | | |
| 374 | N-{3-[3-(1,1′-biphenyl-4-yl)pyrazolo[1,5- | + | 157-160 |
| | a]pyrimidin-7-yl]phenyl}-3-methylbutanamide | | |
| 375 | N-{3-[3-(3-formylphenyl)pyrazolo[1,5-a]pyrimidin- | + | 163-165 |
| | 7-yl]phenyl}-3-methylbutanamide | | |
| 376 | 3-methyl-N-(3-{3-[3-(morpholin-4- | + | 170-173 |
| | ylmethyl)phenyl]pyrazolo[1,5-a]pyrimidin-7- | | |
| | yl}phenyl)butanamide | | |
| 377 | 3-methyl-N-[3-(3-{3-[(4-methylpiperazin-1- | ++ | 135-138 |
| | yl)methyl]phenyl}pyrazolo[1,5-a]pyrimidin-7- | | |
| | yl)phenyl]butanamide | | |
| 378 | 3-[3-(3,5-dimethylphenyl)pyrazolo[1,5-a]pyrimidin- | + | 147 |
| | 7-yl]benzonitrile | | |
| 379 | 3-(3,5-dimethylphenyl)-7-[3-(1H-tetraazol-5- | ++ | 250(dec) |
| | yl)phenyl]pyrazolo[1,5-a]pyrimidine | | |
| 380 | 7-{3-[2-(cyclobutylmethyl)-2H-tetraazol-5- | ++ | 64 |
| | yl]phenyl}-3-(3,5-dimethylphenyl)pyrazolo[1,5- | | |
| | a]pyrimidine | | |
| 381 | 3-(3,5-dimethylphenyl)-7-{3-[2-(2-morpholin-4- | ++ | 68 |
| | ylethyl)-2H-tetraazol-5-yl]phenyl}pyrazolo[1,5- | | |
| | a]pyrimidine | | |
| 382 | 3-(3,5-dimethylphenyl)-7-{3-[2-(pyridin-3-ylmethyl)- | ++ | 73 |
| | 2H-tetrazol-5-yl]phenyl}pyrazolo[1,5-a]pyrimidine | | |
| 383 | 3-(3,5-dimethylphenyl)-7-{3-[2-(pyridin-2-ylmethyl)- | + | 77 |
| | 2H-tetrazol-5-yl]phenyl}pyrazolo[1,5-a]pyrimidine | | |
| 384 | 3-(3,5-dimethylphenyl)-7-[3-(2-ethyl-2H-tetrazol-5- | ++ | 65 |
| | yl)phenyl]pyrazolo[1,5-a]pyrimidine | | |
| 385 | N-[3-(3-{3- | + | 179-182 |
| | [(dimethylamino)methyl]phenyl}pyrazolo[1,5- | | |
| | a]pyrimidin-7-yl)phenyl]-3-methylbutanamide | | |
| 386 | 3-(3,5-dimethylphenyl)-7-{3-[2-(2-pyrrolidin-1- | ++ | 60 |
| | ylethyl)-2H-tetrazol-5-yl]phenyl}pyrazolo[1,5- | | |
| | a]pyrimidine | | |
| 387 | 3-(3,5-dimethylphenyl)-7-{3-[2-(2-piperidin-1- | ++ | 66 |
| | ylethyl)-2H-tetrazol-5-yl]phenyl}pyrazolo[1,5- | | |
| | a]pyrimidine | | |
| 388 | 3-(3,5-dimethylphenyl)-7-{3-[2-(pyridin-4-ylmethyl)- | ++ | 172 |
| | 2H-tetrazol-5-yl]phenyl}pyrazolo[1,5-a]pyrimidine | | |
| 389 | {7-[3-(1H-tetraazol-5-yl)phenyl]pyrazolo[1,5- | + | 290 |
| | a]pyrimidin-3-yl}(thien-2-yl)methanone | | |
| 390 | {7-[3-(2-ethyl-2H-tetraazol-5- | ++ | 174 |
| | yl)phenyl]pyrazolo[1,5-a]pyrimidin-3-yl}(thien-2- | | |
| | yl)methanone | | |
| 391 | (7-{3-[2-(pyridin-3-ylmethyl)-2H-tetraazol-5- | +++ | 95 |
| | yl]phenyl}pyrazolo[1,5-a]pyrimidin-3-yl)(thien-2- | | |
| | yl)methanone | | |
| 392 | {7-[3-(2-isobutyl-2H-tetraazol-5- | +++ | 141 |
| | yl)phenyl]pyrazolo[1,5-a]pyrimidin-3-yl}(thien-2- | | |
| | yl)methanone | | |
| 393 | (7-{3-[2-(cyclobutylmethyl)-2H-tetraazol-5- | +++ | 170 |
| | yl]phenyl}pyrazolo[1,5-a]pyrimidin-3-yl)(thien-2- | | |
| | yl)methanone | | |
| 394 | {7-[3-(2-butyl-2H-tetraazol-5- | +++ | 90 |
| | yl)phenyl]pyrazolo[1,5-a]pyrimidin-3-yl}(thien-2- | | |
| | yl)methanone | | |
| 395 | (7-{3-[2-(2-morpholin-4-ylethyl)-2H-tetraazol-5- | ++ | 103 |
| | yl]phenyl}pyrazolo[1,5-a]pyrimidin-3-yl)(thien-2- | | |
| | yl)methanone | | |
| 396 | (7-{3-[1-(cyclobutylmethyl)-1H-tetraazol-5- | + | 204 |
| | yl]phenyl}pyrazolo[1,5-a]pyrimidin-3-yl)(thien-2- | | |
| | yl)methanone | | |
| 397 | (7-{3-[2-(pyridin-2-ylmethyl)-2H-tetraazol-5- | +++ | 184 |
| | yl]phenyl}pyrazolo[1,5-a]pyrimidin-3-yl)(thien-2- | | |
| | yl)methanone | | |
| 398 | (7-{3-[2-(pyridin-4-ylmethyl)-2H-tetraazol-5- | +++ | 170 |
| | yl]phenyl}pyrazolo[1,5-a]pyrimidin-3-yl)(thien-2- | | |
| | yl)methanone | | |
| 399 | (7-{3-[2-(2-pyrrolidin-1-ylethyl)-2H-tetraazol-5- | +++ | 98 |
| | yl]phenyl}pyrazolo[1,5-a]pyrimidin-3-yl)(thien-2- | | |
| | yl)methanone | | |
| 400 | (7-{3-[2-(2-piperidin-1-ylethyl)-2H-tetraazol-5- | ++ | 117 |
| | yl]phenyl}pyrazolo[1,5-a]pyrimidin-3-yl)(thien-2- | | |
| | yl)methanone | | |
| 401 | (7-{3-[2-(2-hydroxyethyl)-2H-tetraazol-5- | ++ | 134 |
| | yl]phenyl}pyrazolo[1,5-a]pyrimidin-3-yl)(thien-2- | | |
| | yl)methanone | | |
| 402 | {7-[3-(1H-pyrazol-3-yl)phenyl]pyrazolo[1,5- | +++ | 129 |
| | a]pyrimidin-3-yl}(thien-2-yl)methanone | | |
| 403 | (7-{3-[1-(cyclobutylmethyl)-1H-pyrazol-3- | +++ | 60 |
| | yl]phenyl}pyrazolo[1,5-a]pyrimidin-3-yl)(thien-2- | | |
| | yl)methanone | | |
| 404 | (7-{3-[1-(2-morpholin-4-ylethyl)-1H-pyrazol-3- | +++ | 73 |
| | yl]phenyl}pyrazolo[1,5-a]pyrimidin-3-yl)(thien-2- | | |
| | yl)methanone | | |
| 405 | (7-{3-[1-(pyridin-2-ylmethyl)-1H-pyrazol-3- | +++ | 66 |
| | yl]phenyl}pyrazolo[1,5-a]pyrimidin-3-yl)(thien-2- | | |
| | yl)methanone | | |
| 406 | {7-[3-(1-ethyl-1H-pyrazol-3-yl)phenyl]pyrazolo[1,5- | +++ | 61 |
| | a]pyrimidin-3-yl}(thien-2-yl)methanone | | |
| 407 | (7-{3-[1-(2-piperidin-1-ylethyl)-1H-pyrazol-3- | ++ | 62 |
| | yl]phenyl}pyrazolo[1,5-a]pyrimidin-3-yl)(thien-2- | | |
| | yl)methanone | | |
| 408 | (7-{3-[1-(2-pyrrolidin-1-ylethyl)-1H-pyrazol-3- | ++ | 63 |
| | yl]phenyl}pyrazolo[1,5-a]pyrimidin-3-yl)(thien-2- | | |
| | yl)methanone | | |

+++, <1 μM;
++, 1-10 μM;
+, 10-50 μM

TABLE 4
Cellular activity in LoVo cell

| Example | | Inhibition |
|---|---|---|
| Number | Chemical Name | of LoVo |
| 189 | N-{3-[3-(2-thienylcarbonyl)pyrazolo[1,5-a]pyrimidin-7- | +++ |
| | yl]phenyl}-2-butynamide | |
| 194 | N-{3-[3-(2-thienylcarbonyl)pyrazolo[1,5-a]pyrimidin-7- | +++ |
| | yl]phenyl} benzamide | |
| 195 | N-{3-[3-(2-thienylcarbonyl)pyrazolo[1,5-a]pyrimidin-7- | +++ |
| | yl]phenyl}-2-furamide | |
| 200 | N-{3-[3-(2-thienylcarbonyl)pyrazolo[1,5-a]pyrimidin-7- | ++ |
| | yl]phenyl}nicotinamide | |
| 201 | N-{3-[3-(2-thienylcarbonyl)pyrazolo[1,5-a]pyrimidin-7- | +++ |
| | yl]phenyl}isonicotinamide | |
| 209 | N-isopropyl-N′-{2-methyl-5-[3-(2- | +++ |
| | thienylcarbonyl)pyrazolo[1,5-a]pyrimidin-7- | |
| | yl]phenyl}urea | |
| 210 | N-{2-chloro-5-[3-(2-thienylcarbonyl)pyrazolo[1,5- | +++ |
| | a]pyrimidin-7-yl]phenyl}benzamide | |
| 211 | N-{2-chloro-5-[3-(2-thienylcarbonyl)pyrazolo[1,5- | +++ |
| | a]pyrimidin-7-yl]phenyl}-3-methylbutanamide | |
| 212 | isopropyl 2-methyl-5-[3-(2-thienylcarbonyl)pyrazolo[1,5- | +++ |
| | a]pyrimidin-7-yl]phenylcarbamate | |
| 222 | 3-methyl-N-{5-[3-(2-thienylcarbonyl)pyrazolo[1,5- | ++ |
| | a]pyrimidin-7-yl]-3-pyridinyl}butanamide | |
| 226 | [7-(5-nitro-3-thienyl)pyrazolo[1,5-a]pyrimidin-3-yl](2- | ++ |
| | thienyl)methanone | |
| 227 | N-isopropyl-N′-{5-[3-(thien-2-ylcarbonyl)pyrazolo[1,5- | ++ |
| | a]pyrimidin-7-yl]thien-2-yl}urea | |
| 229 | [7-(5-aminothien-3-yl)pyrazolo[1,5-a]pyrimidin-3-yl] | + |
| | (thien-2-yl)methanone | |
| 230 | O-ethyl 3-[3-(thien-2-ylcarbonyl)pyrazolo[1,5- | +++ |
| | a]pyrimidin-7-yl]phenylthiocarbamate | |
| 232 | N-phenyl-N′-{3-[3-(thien-2-ylcarbonyl)pyrazolo[1,5- | ++ |
| | a]pyrimidin-7-yl]phenyl}thiourea | |
| 233 | isopropyl 4-chloro-3-[3-(thien-2- | ++ |
| | ylcarbonyl)pyrazolo[1,5-a]pyrimidin-7- | |
| | yl]phenylcarbamate | |
| 236 | N-{4-chloro-3-[3-(thien-2-ylcarbonyl)pyrazolo[1,5-a]pyrimidin- | ++ |
| | 7-yl]phenyl}-3-methylbutanamide | |
| 237 | N-{4-chloro-3-[3-(thien-2-ylcarbonyl)pyrazolo[1,5- | ++ |
| | a]pyrimidin-7-yl]phenyl}-N′-isopropylthiourea | |
| 238 | N-{4-chloro-3-[3-(thien-2-ylcarbonyl)pyrazolo[1,5- | ++ |
| | a]pyrimidin-7-yl]phenyl}benzamide | |
| 239 | N-{2-fluoro-5-[3-(thien-2-ylcarbonyl)pyrazolo[1,5- | +++ |
| | a]pyrimidin-7-yl]phenyl}-3-methylbutanamide | |
| 240 | 3-methyl-N-{5-[3-(thien-2-ylcarbonyl)pyrazolo[1,5- | + |
| | a]pyrimidin-7-yl]thien-2-yl}butanamide | |
| 241 | isopropyl 5-[3-(thien-2-ylcarbonyl)pyrazolo[1,5- | + |
| | a]pyrimidin-7-yl]thien-2-ylcarbamate | |
| 242 | [7-(5-aminothien-2-yl)pyrazolo[1,5-a]pyrimidin-3- | + |
| | yl] (thien-2-yl)methanone | |
| 243 | 4-chloro-N-{3-[3-(thien-2-ylcarbonyl)pyrazolo[1,5- | ++ |
| | a]pyrimidin-7-yl]phenyl} butanamide | |
| 244 | isopropyl 2-fluoro-5-[3-(thien-2-ylcarbonyl)pyrazolo[1,5- | +++ |
| | a]pyrimidin-7-yl]phenylcarbamate | |
| 245 | isopropyl 2-(dimethylamino)-5-[3-(thien-2- | ++ |
| | ylcarbonyl)pyrazolo[1,5-a]pyrimidin-7- | |
| | yl]phenylcarbamate | |
| 246 | {7-[3-(benzylamino)phenyl]pyrazolo[1,5-a]pyrimidin-3- | ++ |
| | yl}(thien-2-yl)methanone | |
| 247 | 3-methyl-N-{4-[3-(thien-2-ylcarbonyl)pyrazolo[1,5- | + |
| | a]pyrimidin-7-yl]thien-2-yl}butanamide | |
| 248 | [7-(3-amino-4-fluorophenyl)pyrazolo[1,5-a]pyrimidin-3- | + |
| | yl] (thien-2-yl)methanone | |
| 249 | N-cyclohexyl-N′-{3-[3-(thien-2-ylcarbonyl)pyrazolo[1,5- | +++ |
| | a]pyrimidin-7-yl]phenyl} urea | |
| 250 | 4,4,4-trichloro-N-{3-[3-(thien-2-ylcarbonyl)pyrazolo[1,5- | +++ |
| | a]pyrimidin-7-yl]phenyl}butanamide | |
| 251 | N-{2-fluoro-5-[3-(thien-2-ylcarbonyl)pyrazolo[1,5- | +++ |
| | a]pyrimidin-7-yl]phenyl}-3,3-dimethylbutanamide | |
| 252 | 4,4,4-trifluoro-N-{2-fluoro-5-[3-(thien-2- | +++ |
| | ylcarbonyl)pyrazolo[1,5-a]pyrimidin-7-yl]phenyl}-3- | |
| | methylbutanamide | |
| 253 | bis(2,2,2-trichloroethyl) 2-fluoro-5-[3-(thien-2- | +++ |
| | ylcarbonyl)pyrazolo [1,5-a]pyrimidin-7- | |
| | yl]phenylimidodicarbonate | |
| 254 | 2-fluoro-N-{3-[3-(thien-2-ylcarbonyl)pyrazolo[1,5- | +++ |
| | a]pyrimidin-7-yl]phenyl}benzamide | |
| 255 | 2,6-difluoro-N-{3-[3-(thien-2-ylcarbonyl)pyrazolo[1,5- | +++ |
| | a]pyrimidin-7-yl]phenyl} benzamide | |
| 256 | N-{3-[3-(thien-2-ylcarbonyl)pyrazolo[1,5-a]pyrimidin-7- | +++ |
| | yl]phenyl} pyridine-2-carboxamide | |
| 257 | N-{5-[3-(thien-2-ylcarbonyl)pyrazolo[1,5-a]pyrimidin-7- | + |
| | yl]thien-2-yl}benzamide | |
| 258 | (7-{3-[(2-phenylethyl)amino]phenyl}pyrazolo[1,5- | + |
| | a]pyrimidin-3-yl)(thien-2-yl)methanone | |
| 259 | (7-{3-[(2-methoxybenzyl)amino]phenyl}pyrazolo[1,5- | ++ |
| | a]pyrimidin-3-yl)(thien-2-yl)methanone | |
| 260 | N-butyl-N′-{3-[3-(thien-2-ylcarbonyl)pyrazolo[1,5- | +++ |
| | a]pyrimidin-7-yl]phenyl}thiourea | |
| 261 | (7-{3-[(3-chlorobenzyl)amino]phenyl}pyrazolo[1,5- | + |
| | a]pyrimidin-3-yl)(thien-2-yl)methanone | |
| 262 | (7-{3-[(4-chlorobenzyl)amino]phenyl}pyrazolo[1,5- | + |
| | a]pyrimidin-3-yl)(thien-2-yl)methanone | |
| 263 | (7-{3-[(2-furylmethyl)amino]phenyl}pyrazolo[1,5- | ++ |
| | a]pyrimidin-3-yl)(thien-2-yl)methanone | |
| 264 | (7-{3-[(2-chlorobenzyl)amino]phenyl}pyrazolo[1,5- | ++ |
| | a]pyrimidin-3-yl)(thien-2-yl)methanone | |
| 265 | (7-{3-[(3-furylmethyl)amino]phenyl}pyrazolo[1,5- | ++ |
| | a]pyrimidin-3-yl)(thien-2-yl)methanone | |
| 266 | (7-{3-[(2E)-but-2-enylamino]phenyl}pyrazolo[1,5- | +++ |
| | a]pyrimidin-3-yl)(thien-2-yl)methanone | |
| 267 | (7-{3-[(3-methylbut-2-enyl)amino]phenyl}pyrazolo[1,5- | +++ |
| | a]pyrimidin-3-yl)(thien-2-yl)methanone | |
| 268 | [7-(6-chloropyridin-3-yl)pyrazolo[1,5-a]pyrimidin-3- | + |
| | yl] (thien-2-yl)methanone | |
| 269 | 2,6-difluoro-N-{2-fluoro-5-[3-(thien-2- | +++ |
| | ylcarbonyl)pyrazolo[1,5-a]pyrimidin-7- | |
| | yl]phenyl}benzamide | |
| 270 | 2-fluoro-N-{2-fluoro-5-[3-(thien-2- | +++ |
| | ylcarbonyl)pyrazolo[1,5-a]pyrimidin-7- | |
| | yl]phenyl}benzamide | |
| 271 | 2-chloro-N-{2-fluoro-5-[3-(thien-2- | +++ |
| | ylcarbonyl)pyrazolo[1,5-a]pyrimidin-7- | |
| | yl]phenyl}benzamide | |
| 272 | (7-{3-[(2-methylbenzyl)amino]phenyl}pyrazolo[1,5- | ++ |
| | a]pyrimidin-3-yl)(thien-2-yl)methanone | |
| 273 | (7-{3-[(2-phenylpropyl)amino]phenyl}pyrazolo[1,5- | + |
| | a]pyrimidin-3-yl)(thien-2-yl)methanone | |
| 274 | (7-{3-[(cyclohexylmethyl)amino]phenyl}pyrazolo[1,5- | +++ |
| | a]pyrimidin-3-yl)(thien-2-yl)methanone | |
| 275 | 2-chloroethyl 2-fluoro-5-[3-(thien-2- | +++ |
| | ylcarbonyl)pyrazolo[1,5-a]pyrimidin-7- | |
| | yl]phenylcarbamate | |
| 276 | N-{2-fluoro-5-[3-(thien-2-ylcarbonyl)pyrazolo[1,5- | +++ |
| | a]pyrimidin-7-yl]phenyl}-N′-isopropylurea | |
| 277 | 4-(1H-pyrrol-1-yl)-N-{3-[3-(thien-2- | ++ |
| | ylcarbonyl)pyrazolo[1,5-a]pyrimidin-7- | |
| | yl]phenyl}benzamide | |
| 278 | 2,6-dichloro-N-{3-[3-(thien-2-ylcarbonyl)pyrazolo[1,5- | ++ |
| | a]pyrimidin-7-yl]phenyl}benzamide | |
| 279 | N-{3-[3-(thien-2-ylcarbonyl)pyrazolo[1,5-a]pyrimidin-7- | +++ |
| | yl]phenyl} pyrazine-2-carboxamide | |
| 280 | phenyl N′-cyano-N-{3-[3-(thien-2- | + |
| | ylcarbonyl)pyrazolo[1,5-a]pyrimidin-7- | |
| | yl]phenyl}imidocarbamate | |
| 281 | 1-methyl-N-(3-[3-(thien-2-ylcarbonyl)pyrazolo[1,5- | +++ |
| | a]pyrimidin-7-yl]phenyl}-1H-pyrrole-2-carboxamide | |
| 282 | N-{2-fluoro-5-[3-(thien-2-ylcarbonyl)pyrazolo[1,5- | +++ |
| | a]pyrimidin-7-yl]phenyl}isonicotinamide | |
| 283 | N-isopropyl-N′-{3-[3-(thien-2-ylcarbonyl)pyrazolo[1,5- | +++ |
| | a]pyrimidin-7-yl]phenyl}thiourea | |
| 284 | (7-chloropyrazolo[1,5-a]pyrimidin-3-yl)(thien-2- | + |
| | yl)methanone | |
| 285 | 2,6-dichloro-N-(2,6-dichlorobenzoyl)-N-{3-[3-(thien-2- | + |
| | ylcarbonyl)pyrazolo[1,5-a]pyrimidin-7- | |
| | yl]phenyl}benzamide | |
| 286 | tert-butyl 4-[2-oxo-2-({3-[3-(thien-2- | +++ |
| | ylcarbonyl)pyrazolo[1,5-a]pyrimidin-7- | |
| | yl]phenyl}amino)ethyl]piperidine-1-carboxylate | |
| 287 | 2-piperidin-4-yl-N-{3-[3-(thien-2- | ++ |
| | ylcarbonyl)pyrazolo[1,5-a]pyrimidin-7- | |
| | yl]phenyl}acetamide | |
| 288 | [7-(3-{[(2E)-3-(2-methoxyphenyl)prop-2- | + |
| | enyl]amino}phenyl)pyrazolo[1,5-a]pyrimidin-3-yl](thien- | |
| | 2-yl)methanone | |
| 289 | [7-(3-{[(2E)-3-(4-methoxyphenyl)prop-2- | + |
| | enyl]amino}phenyl)pyrazolo[1,5-a]pyrimidin-3-yl](thien- | |
| | 2-yl)methanone | |
| 291 | [7-(3-{[(2E)-3-(4-nitrophenyl)prop-2- | + |
| | enyl]amino}phenyl)pyrazolo[1,5-a]pyrimidin-3-yl](thien- | |
| | 2-yl)methanone | |
| 292 | (7-{3-[(3,3-diphenylprop-2- | + |
| | enyl)amino]phenyL}pyrazolo[1,5-a]pyrimidin-3-yl)(thien- | |
| | 2-yl)methanone | |
| 308 | (7-{3-[(2-ethylhexyl)amino]phenyl}pyrazolo[1,5- | + |
| | a]pyrimidin-3-yl)(thien-2-yl)methanone | |
| 309 | 3-(1,1-dioxidothiomorpholin-4-yl)propyl 3-[3-(thien-2- | ++ |
| | ylcarbonyl)pyrazolo[1,5-a]pyrimidin-7- | |
| | yl]phenylcarbamate | |
| 310 | 2-chloro-N-{2-(dimethylamino)-5-[3-(thien-2- | ++ |
| | ylcarbonyl)pyrazolo[1,5-a]pyrimidin-7- | |
| | yl]phenyl}benzamide | |
| 311 | 4-morpholin-4-yl-N-{3-[3-(thien-2- | ++ |
| | ylcarbonyl)pyrazolo[1,5-a]pyrimidin-7- | |
| | yl]phenyl}butanamide | |
| 312 | pyridin-3-ylmethyl 3-[3-(thien-2- | + |
| | ylcarbonyl)pyrazolo[1,5-a]pyrimidin-7- | |
| | yl]phenylcarbamate | |
| 313 | 3-morpholin-4-ylpropyl 3-[3-(thien-2- | ++ |
| | ylcarbonyl)pyrazolo[1,5-a]pyrimidin-7- | |
| | yl]phenylcarbamate | |
| 314 | [7-(3-{[(2E)-2-methylpent-2- | ++ |
| | enyl]amino}phenyl)pyrazolo[1,5-a]pyrimidin-3-yl] (thien- | |
| | 2-yl)methanone | |
| 315 | 2-pyridin-4-yl-N-{3-[3-(thien-2-ylcarbonyl)pyrazolo[1,5- | + |
| | a]pyrimidin-7-yl]phenyl}acetamide | |
| 317 | pyridin-2-ylmethyl 3-[3-(thien-2- | ++ |
| | ylcarbonyl)pyrazolo[1,5-a]pyrimidin-7- | |
| | yl]phenylcarbamate | |
| 318 | (7-{3-[(2,3-dimethylpentyl)amino]phenyl}pyrazolo[1,5- | ++ |
| | a]pyrimidin-3-yl)(thien-2-yl)methanone | |
| 319 | [7-(3-{[(2E)-2-methylbut-2- | ++ |
| | enyl]amino}phenyl)pyrazolo[1,5-a]pyrimidin-3-yl] (thien- | |
| | 2-yl)methanone | |
| 320 | (7-{3-[(2E)-pent-2-enylamino]phenyl}pyrazolo[1,5- | +++ |
| | a]pyrimidin-3-yl)(thien-2-yl)methanone | |
| 321 | thien-2-yl(7-{3-[(3,5,5- | ++ |
| | trimethylhexyl)amino]phenyl}pyrazolo[1,5-a]pyrimidin- | |
| | 3-yl)methanone | |
| 322 | (7-{3-[(2-methylprop-2- | ++ |
| | enyl)amino]phenyl}pyrazolo[1,5-a]pyrimidin-3-yl)(thien- | |
| | 2-yl)methanone | |
| 323 | (7-{3-[(2-ethylprop-2-enyl)amino]phenyl}pyrazolo[1,5- | ++ |
| | a]pyrimidin-3-yl)(thien-2-yl)methanone | |
| 324 | (7-{3-[(cyclohex-3-en-1- | +++ |
| | ylmethyl)emino]phenyl} pyrazolo[1,5-a]pyrimidin-3-yl)- | |
| | (thien-2-yl)-methanone | |
| 325 | 1-{3-[3-(thien-2-ylcarbonyl)pyrazolo[1,5-a]pyrimidin-7- | +++ |
| | yl]phenyl} pyrrolidin-2-one | |
| 326 | 2-chloro-N-(2-chlorobenzoyl)-N-{2-fluoro-5-[3-(thien-2- | +++ |
| | ylcarbonyl)pyrazolo[1,5-a]pyrimidin-7- | |
| | yl]phenyl}benzamide | |
| 327 | 2-pyridin-3-yl-N-{3-[3-(thien-2-ylcarbonyl)pyrazolo[1,5- | +++ |
| | a]pyrimidin-7-yl]phenyl} acetamide | |
| 328 | 3-pyridin-4-yl-N-{3-[3-(thien-2-ylcarbonyl)pyrazolo[1,5- | ++ |
| | a]pyrimidin-7-yl]phenyl}propanamide | |
| 329 | N-{3-[3-(thien-2-ylcarbonyl)pyrazolo[1,5-a]pyrimidin-7- | + |
| | yl]phenyl}-1H-pyrazole-4-carboxamide | |
| 330 | 4-oxo-4-({3-[3-(thien-2-ylcarbonyl)pyrazolo[1,5- | + |
| | a]pyrimidin-7-yl]phenyl}amino)butanoic acid | |
| 331 | 2-[({3-[3-(thien-2-ylcarbonyl)pyrazolo[1,5-a]pyrimidin- | + |
| | 7-yl]phenyl}amino)carbonyl]benzoic acid | |
| 332 | 5-oxo-5-({3-[3-(thien-2-ylcarbonyl)pyrazolo[1,5- | + |
| | a]pyrimidin-7-yl]phenyl}amino)pentanoic acid | |
| 333 | 3-methyl-5-oxo-5-({3-[3-(thien-2- | + |
| | ylcarbonyl)pyrazolo[1,5-a]pyrimidin-7- | |
| | yl]phenyl}amino)pentanoic acid | |
| 334 | 4,5-dichloro-2-[({3-[3-(thien-2-ylcarbonyl)pyrazolo[1,5- | + |
| | a]pyrimidin-7-yl]phenyl}amino)carbonyl]benzoic acid | |
| 335 | (7-{3-[(2-methylundecyl)amino]phenyl}pyrazolo[1,5- | + |
| | a]pyrimidin-3-yl)(thien-2-yl)methanone | |
| 336 | 3-pyridin-3-yl-N-{3-[3-(thien-2-ylcarbonyl)pyrazolo[1,5- | ++ |
| | a]pyrimidin-7-yl]phenyl}propanamide | |
| 337 | 2-(pyridin-4-ylthio)-N-{3-[3-(thien-2 | ++ |
| | ylcarbonyl)pyrazolo[1,5-a]pyrimidin-7- | |
| | yl]phenyl} acetamide | |
| 338 | {7-[4′-(dimethylamino)-1,1′-biphenyl-3-yl]pyrazolo[1,5- | +++ |
| | a]pyrimidin-3-yl}(thien-2-yl)methanone | |
| 339 | [7-(3′-amino-1,1′-biphenyl-3-yl)pyrazolo[1,5-a]pyrimidin- | ++ |
| | 3-yl](thien-2-yl)methanone | |
| 340 | {7-[4′-(hydroxymethyl)-1,1′-biphenyl-3-yl]pyrazolo[1,5- | +++ |
| | a]pyrimidin-3-yl}(thien-2-yl)methanone | |
| 341 | {7-[3′-(dimethylamino)-1,1′-biphenyl-3-yl]pyrazolo[1,5- | ++ |
| | a]pyrimidin-3-yl}(thien-2-yl)methanone | |
| 342 | {7-[4′-(dimethylamino)-6-fluoro-1,1′-biphenyl-3- | ++ |
| | yl]pyrazolo[1,5-a]pyrimidin-3-yl}(thien-2-yl)methanone | |
| 343 | {7-[6-fluoro-4′-(hydroxymethyl)-1,1′-biphenyl-3- | ++ |
| | yl]pyrazolo[1,5-a]pyrimidin-3-yl}(thien-2-yl)methanone | |
| 344 | [7-(4′-ethyl-1,1′-biphenyl-3-yl)pyrazolo[1,5-a]pyrimidin-3- | +++ |
| | yl](thien-2-yl)methanone | |
| 345 | 3-{3′-[3-(thien-2-ylcarbonyl)pyrazolo[1,5-a]pyrimidin-7- | ++ |
| | yl]-1,1′-biphenyl-4-yl}propanoic acid | |
| 346 | 3′-[3-(thien-2-ylcarbonyl)pyrazolo[1,5-a]pyrimidin-7-yl]- | ++ |
| | 1,1′-biphenyl-4-carbaldehyde | |
| 347 | {7-[4′-(morpholin-4-ylmethyl)-1,1′-biphenyl-3- | NT |
| | yl]pyrazolo[1,5-a]pyrimidin-3-yl}(thien-2-yl)methanone | |
| 348 | (7-{4′-[(4-methylpiperazin-1-yl)methyl]-1,1′-biphenyl-3- | NT |
| | yl}pyrazolo[1,5-a]pyrimidin-3-yl)(thien-2-yl)methanone | |
| 349 | [7-(4′-{[(2-morpholin-4-ylethyl)amino]methyl}-1,1′- | ++ |
| | biphenyl-3-yl)pyrazolo[1,5-a]pyrimidin-3-yl](thien-2- | |
| | yl)methanone | |
| 350 | (7-{4′-[(diethylamino)methyl]-1,1′-biphenyl-3- | ++ |
| | yl}pyrazolo[1,5-a]pyrimidin-3-yl)(thien-2-yl)methanone | |
| 351 | (7-{4′-[(dimethylamino)methyl]-1,1′-biphenyl-3- | +++ |
| | yl}pyrazolo[1,5-a]pyrimidin-3-yl)(thien-2-yl)methanone | |
| 352 | N-{3-[3-(3-aminophenyl)pyrazolo[1,5-a]pyrimidin-7- | + |
| | yl]phenyl}-3-methylbutanamide | |
| 353 | N-(3-{3-[3-(dimethylamino)phenyl]pyrazolo[1,5- | +++ |
| | a]pyrimidin-7-yl}phenyl)-3-methylbutanamide | |
| 354 | N-{3-[3-(1H-indol-5-yl)pyrazolo[1,5-a]pyrimidin-7- | +++ |
| | yl]phenyl}-3-methylbutanamide | |
| 355 | 3-methyl-N-{3-[3-(3,4,5-trimethoxyphenyl)pyrazolo[1,5- | +++ |
| | a]pyrimidin-7-yl]phenyl}butanamide | |
| 356 | 3-methyl-N-[3-(3-quinolin-8-ylpyrazolo[1,5-a]pyrimidin- | ++ |
| | 7-yl)phenyl]butanamide | |
| 357 | N-(3-{3-[4-(hydroxymethyl)phenyl]pyrazolo[1,5- | + |
| | a]pyrimidin-7-yl}phenyl)-3-methylbutanamide | |
| 358 | N-{3-[3-(4-methoxyphenyl)pyrazolo[1,5-a]pyrimidin-7- | + |
| | yl]phenyl}-3-methylbutanamide | |
| 359 | N-{3-[3-(3-chlorophenyl)pyrazolo[1,5-a]pyrimidin-7- | + |
| | yl]phenyl}-3-methylbutanamide | |
| 360 | N-{3-[3-(4-chlorophenyl)pyrazolo[1,5-a]pyrimidin-7- | + |
| | yl]phenyl}-3-methylbutanamide | |
| 361 | N-{3-[3-(2,3-dichlorophenyl)pyrazolo[1,5-a]pyrimidin-7- | ++ |
| | yl]phenyl}-3-methylbutanamide | |
| 362 | N-{3-[3-(3,4-dichlorophenyl)pyrazolo[1,5-a]pyrimidin-7- | + |
| | yl]phenyl}-3-methylbutanamide | |
| 363 | N-{3-[3-(3,5-dichlorophenyl)pyrazolo[1,5-a]pyrimidin-7- | + |
| | yl]phenyl}-3-methylbutanamide | |
| 364 | N-{3-[3-(3,5-dimethylphenyl)pyrazolo[1,5-a]pyrimidin-7- | +++ |
| | yl]phenyl}-3-methylbutanamide | |
| 365 | 3-methyl-N-(3-{3-[3- | + |
| | (trifluoromethyl)phenyl]pyrazolo[1,5-a]pyrimidin-7- | |
| | yl}phenyl)butanamide | |
| 366 | N-{3-[3-(3-methoxyphenyl)pyrazolo[1,5-a]pyrimidin-7- | + |
| | yl]phenyl}-3-methylbutanamide | |
| 367 | N-{3-[3-(3-ethoxyphenyl)pyrazolo[1,5-a]pyrimidin-7- | + |
| | yl]phenyl}-3-methylbutanamide | |
| 368 | 3-methyl-N-{3-[3-(4-phenoxyphenyl)pyrazolo[1,5- | + |
| | a]pyrimidin-7-yl]phenyl}butanamide | |
| 369 | N-{3-[3-(1,3-benzodioxol-5-yl)pyrazolo[1,5-a]pyrimidin- | ++ |
| | 7-yl]phenyl}-3-methylbutanamide | |
| 370 | N-{3-[3-(3-cyanophenyl)pyrazolo[1,5-a]pyrimidin-7- | + |
| | yl]phenyl}-3-methylbutanamide | |
| 371 | N-{3-[3-(3-acetylphenyl)pyrazolo[1,5-a]pyrimidin-7- | + |
| | yl]phenyl}-3-methylbutanamide | |
| 372 | N-{3-[3-(3-formyl-4-methoxyphenyl)pyrazolo[1,5- | + |
| | a]pyrimidin-7-yl]phenyl}-3-methylbutanamide | |
| 373 | N-{3-[3-(1,1′-biphenyl-3-yl)pyrazolo[1,5-a]pyrimidin-7- | + |
| | yl]phenyl}-3-methylbutanamide | |
| 374 | N-{3-[3-(1,1′-biphenyl-4-yl)pyrazolo[1,5-a]pyrimidin-7- | + |
| | yl]phenyl}-3-methylbutanamide | |
| 375 | N-{3-[3-(3-formylphenyl)pyrazolo[1,5-a]pyrimidin-7- | + |
| | yl]phenyl}-3-methylbutanamide | |
| 376 | 3-methyl-N-(3-{3-[3-(morpholin-4- | + |
| | ylmethyl)phenyl]pyrazolo[1,5-a]pyrimidin-7- | |
| | yl}phenyl)butanamide | |
| 377 | 3-methyl-N-[3-(3-{3-[(4-methylpiperazin-1- | + |
| | yl)methyl]phenyl}pyrazolo[1,5-a]pyrimidin-7- | |
| | yl)phenyl]butanamide | |
| 378 | 3-[3-(3,5-dimethylphenyl)pyrazolo[1,5-a]pyrimidin-7- | + |
| | yl]benzonitrile | |
| 379 | 3-(3,5-dimethylphenyl)-7-[3-(1H-tetraazol-5- | + |
| | yl)phenyl]pyrazolo[1,5-a]pyrimidine | |
| 380 | 7-{3-[2-(cyclobutylmethyl)-2H-tetraazol-5-yl]phenyl}-3- | ++ |
| | (3,5-dimethylphenyl)pyrazolo[1,5-a]pyrimidine | |
| 381 | 3-(3,5-dimethylphenyl)-7-{3-[2-(2-morpholin-4-ylethyl)- | ++ |
| | 2H-tetraazol-5-yl]phenyl}pyrazolo[1,5-a]pyrimidine | |
| 382 | 3-(3,5-dimethylphenyl)-7-{3-[2-(pyridin-3-ylmethyl)-2H- | ++ |
| | tetrazol-5-yl]phenyl}pyrazolo[1,5-a]pyrimidine | |
| 383 | 3-(3,5-dimethylphenyl)-7-{3-[2-(pyridin-2-ylmethyl)-2H- | ++ |
| | tetrazol-5-yl]phenyl}pyrazolo[1,5-a]pyrimidine | |
| 384 | 3-(3,5-dimethylphenyl)-7-[3-(2-ethyl-2H-tetrazol-5- | ++ |
| | yl)phenyl]pyrazolo[1,5-a]pyrimidine | |
| 385 | N-[3-(3-{3-[(dimethylamino)methyl]phenyl}pyrazolo[1,5- | NT |
| | a]pyrimidin-7-yl)phenyl]-3-methylbutanamide | |
| 386 | 3-(3,5-dimethylphenyl)-7-{3-[2-(2-pyrrolidin-1-ylethyl)- | NT |
| | 2H-tetrazol-5-yl]phenyl}pyrazolo[1,5-a]pyrimidine | |
| 387 | 3-(3,5-dimethylphenyl)-7-{3-[2-(2-piperidin-1-ylethyl)- | NT |
| | 2H-tetrazol-5-yl]phenyl}pyrazolo[1,5-a]pyrimidine | |
| 388 | 3-(3,5-dimethylphenyl)-7-{3-[2-(pyridin-4-ylmethyl)-2H- | NT |
| | tetrazol-5-yl]phenyl}pyrazolo[1,5-a]pyrimidine | |
| 389 | {7-[3-(1H-tetraazol-5-yl)phenyl]pyrazolo[1,5-a]pyrimidin- | + |
| | 3-yl}(thien-2-yl)methanone | |
| 390 | {7-[3-(2-ethyl-2H-tetraazol-5-yl)phenyl]pyrazolo[1,5- | ++ |
| | a]pyrimidin-3-yl}(thien-2-yl)methanone | |
| 391 | (7-{3-[2-(pyridin-3-ylmethyl)-2H-tetraazol-5- | ++ |
| | yl]phenyl}pyrazolo[1,5-a]pyrimidin-3-yl)(thien-2- | |
| | yl)methanone | |
| 392 | {7-[3-(2-isobutyl-2H-tetraazol-5-yl)phenyl]pyrazolo[1,5- | ++ |
| | a]pyrimidin-3-yl}(thien-2-yl)methanone | |
| 393 | (7-{3-[2-(cyclobutylmethyl)-2H-tetraazol-5- | +++ |
| | yl]phenyl}pyrazolo[1,5-a]pyrimidin-3-yl)(thien-2- | |
| | yl)methanone | |
| 394 | {7-[3-(2-butyl-2H-tetraazol-5-yl)phenyl]pyrazolo[1,5- | +++ |
| | a]pyrimidin-3-yl}(thien-2-yl)methanone | |
| 395 | (7-{3-[2-(2-morpholin-4-ylethyl)-2H-tetraazol-5- | ++ |
| | yl]phenyl}pyrazolo[1,5-a]pyrimidin-3-yl)(thien-2- | |
| | yl)methanone | |
| 396 | (7-{3-[1-(cyclobutylmethyl)-1H-tetraazol-5- | + |
| | yl]phenyl}pyrazolo[1,5-a]pyrimidin-3-yl)(thien-2- | |
| | yl)methanone | |
| 397 | (7-{3-[2-(pyridin-2-ylmethyl)-2H-tetraazol-5- | ++ |
| | yl]phenyl}pyrazolo[1,5-a]pyrimidin-3-yl)(thien-2- | |
| | yl)methanone | |
| 398 | (7-{3-[2-(pyridin-4-ylmethyl)-2H-tetraazol-5- | +++ |
| | yl]phenyl}pyrazolo[1,5-a]pyrimidin-3-yl)(thien-2- | |
| | yl)methanone | |
| 399 | (7-{3-[2-(2-pyrrolidin-1-ylethyl)-2H-tetraazol-5- | ++ |
| | yl]phenyl}pyrazolo[1,5-a]pyrimidin-3-yl)(thien-2- | |
| | yl)methanone | |
| 400 | (7-{3-[2-(2-piperidin-1-ylethyl)-2H-tetraazol-5- | ++ |
| | yl]phenyl}pyrazolo[1,5-a]pyrimidin-3-yl)(thien-2- | |
| | yl)methanone | |
| 401 | (7-{3-[2-(2-hydroxyethyl)-2H-tetraazol-5- | ++ |
| | yl]phenyl}pyrazolo[1,5-a]pyrimidin-3-yl)(thien-2- | |
| | yl)methanone | |
| 402 | {7-[3-(1H-pyrazol-3-yl)phenyl]pyrazolo[1,5-a]pyrimidin- | ++ |
| | 3-yl}(thien-2-yl)methanone | |
| 403 | (7-{3-[1-(cyclobutylmethyl)-1H-pyrazol-3- | +++ |
| | yl]phenyl}pyrazolo[1,5-a]pyrimidin-3-yl)(thien-2- | |
| | yl)methanone | |
| 404 | (7-{3-[1-(2-morpholin-4-ylethyl)-1H-pyrazol-3- | +++ |
| | yl]phenyl}pyrazolo[1,5-a]pyrimidin-3-yl)(thien-2- | |
| | yl)methanone | |
| 405 | (7-{3-[1-(pyridin-2-ylmethyl)-1H-pyrazol-3- | ++ |
| | yl]phenyl}pyrazolo[1,5-a]pyrimidin-3-yl)(thien-2- | |
| | yl)methanone | |
| 406 | {7-[3-(1-ethyl-1H-pyrazol-3-yl)phenyl]pyrazolo[1,5- | +++ |
| | a]pyrimidin-3-yl}(thien-2-yl)methanone | |
| 407 | (7-{3-[1-(2-piperidin-1-ylethyl)-1H-pyrazol-3- | ++ |
| | yl]phenyl}pyrazolo[1,5-a]pyrimidin-3-yl)(thien-2- | |
| | yl)methanone | |
| 408 | (7-{3-[1-(2-pyrrolidin-1-ylethyl)-1H-pyrazol-3- | ++ |
| | yl]phenyl}pyrazolo[1,5-a]pyrimidin-3-yl)(thien-2- | |
| | yl)methanone | |

+++, <0.1 μM;
++, 0.1-1.0 μM;
+, 1.0-20 μM

Experimental
General Procedures:

General Procedures 1-8 were used for compounds 1-174. The specifically named product for compounds prepared via these procedures can be found in Table 1. The procedures below are illustrative and not intended to limit the scope of the invention in any way.

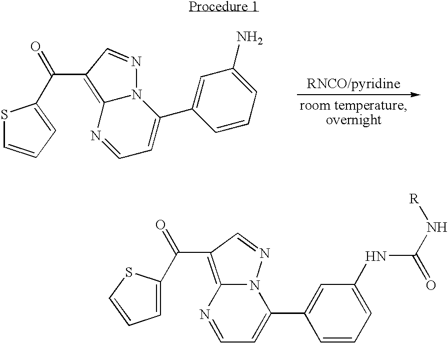

Examples 1-10 were synthesized in parallel as described here: [7-(3-aminophenyl)pyrazolo[1,5-a]pyrimidin-3-yl]-2-thienyl-methanone (0.1 mmole, 32 mg) was dissolved in 2 mL of pyridine and treated with appropriate isocyanate (0.12 mmole) at room temperature. The reaction was stirred at room temperature overnight and the volatiles were removed under reduced pressure. The resulting residue was dissolved in a 1:1 mixture of dimethyl sulfoxide/acetonitrile and purified by preparative HPLC. The fractions were analyzed by LC/MS to identify the product. The mass spectrum data of the isolated products are provided in Table 1.

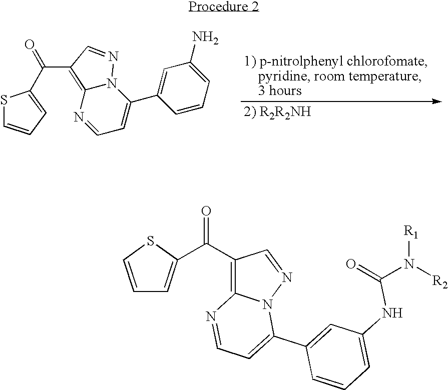

Examples 130-143 were prepared in parallel as described below: [7-(3-aminophenyl)pyrazolo[1,5-a]pyrimidin-3-yl]-2-thienyl-methanone (0.1 mmole, 32 mg) was dissolved in 2 mL of pyridine and treated with p-nitrophenyl chloroformate (0.15 mmole). The mixture was stirred at room temperature for 3 h and treated with appropriate amine (0.2 mmole) and stirring was continued at room temperature overnight. The volatiles were removed under reduced pressure. The resulting residue was dissolved in a 1:1 mixture of dimethyl sulfoxide/acetonitrile and purified by preparative HPLC. The fractions were analyzed by LC/MS to identify the product. The mass spectrum data of the isolated products are provided in Table 1.

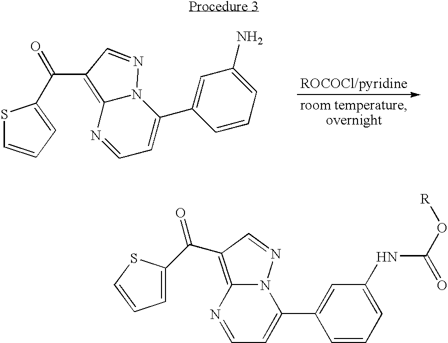

Examples 11-19 and 162-163 were prepared in parallel as described below: [7-(3-aminophenyl)pyrazolo[1,5-a]pyrimidin-3-yl]-2-thienyl-methanone (0.1 mmole, 32 mg) was dissolved in 2 mL of pyridine and was treated with appropriate chloroformates (0.12 mmole) at room temperature. The reaction was stirred at room temperature overnight and the volatiles were removed under reduced pressure. The resulting residue was dissolved in a 1:1 mixture of dimethylsulfoxide/acetonitrile and purified by preparative HPLC. The fractions were analyzed by LC/MS to identify the product. The mass spectra of the isolated products are provided in Table 1.

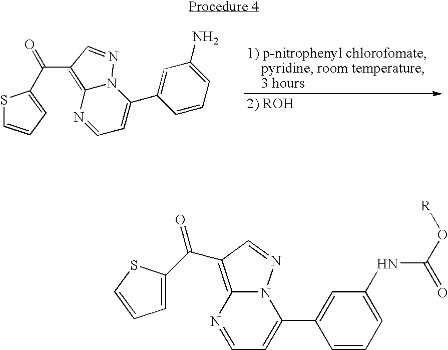

Examples 144-161 and 168-170 were prepared in parallel using the following procedure: [7-(3-aminophenyl)pyrazolo[1,5-a]pyrimidin-3-yl]-2-thienyl-methanone (0.1 mmole, 32 mg) was dissolved in 2 mL of pyridine and treated with p-nitrophenyl chloroformate (0.15 mmole). The mixture was stirred at room temperature for 3 h and treated with appropriate alcohol (0.2 mmole) and stirring was continued overnight. Volatiles were removed under reduced pressure. The resulting residue was dissolved in a 1:1 mixture of dimethyl sulfoxide/acetonitrile and purified by preparative HPLC. The fractions were analyzed by LC/MS to identify the product. The mass spectrum data of the isolated products are provided in Table 1.

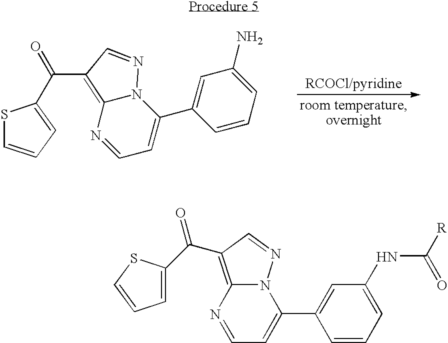

Examples 34-43, 69-129, 164-66, and 171-173 were synthesized in parallel by the procedure described below: [7-(3-aminophenyl)pyrazolo[1,5-a]pyrimidin-3-yl]-2-thienyl-methanone (0.1 mmole, 32 mg) was dissolved in 2 mL of pyridine and was treated with appropriate carboxylic acid chlorides (0.12 mmole) at room temperature. The reaction mixture was stirred at room temperature overnight and the volatiles were removed under reduced pressure. The resulting residue was dissolved in a 1:1 mixture of dimethylsulfoxide/acetonitrile and purified by preparative HPLC. The fractions were analyzed by LC/MS to identify the product. The mass spectra of isolated products are provided in Table 1.

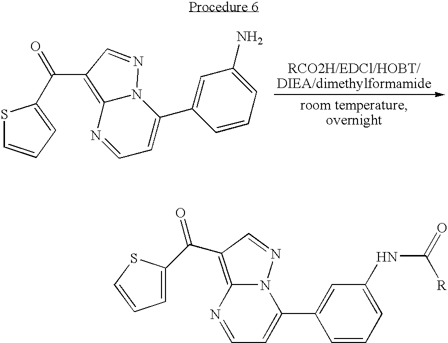

Examples 59-68 were also prepared in parallel by following procedure: [7-(3-aminophenyl)pyrazolo[1,5-a]pyrimidin-3-yl]-2-thienyl-methanone (0.1 mmole, 32 mg) was dissolved in 2 mL of DMF and was treated with carboxylic acids (0.15 mmole) in the presence of EDCI (0.15 mmol), HOBT (0.15 mmol) and DIEA (0.3 mmol). The reaction mixture was stirred at room temperature overnight and the volatiles were removed under reduced pressure. The resulting residue was dissolved in a 1:1 mixture of dimethylsulfoxide and acetonitrile, and was purified by preparative HPLC. The fractions were analyzed by LC/MS to identify the product. The mass spectrum data of the isolated products are provided in Table 1.

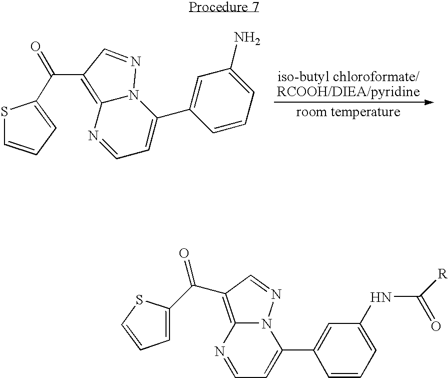

Alternatively Examples 44-58 and 174 were prepared in parallel by the following procedure: [7-(3-aminophenyl)pyrazolo[1,5-a]pyrimidin-3-yl]-2-thienyl-methanone (0.1 mmole) was dissolved in 2 mL of pyridine and was treated with anhydrides formed from carboxylic acids (0.15 mmole), isobutyryl chloroformate (0.15) and DIEA (0.3 mmol) using tetrahydrofuran as the solvent. The reaction mixture was stirred at room temperature overnight and the volatiles were removed under reduced pressure. The resulting residue was dissolved in a 1:1 mixture of dimethylsulfoxide and acetonitrile, and was purified by preparative HPLC. The fractions were analyzed by LC/MS to identify the product. The mass spectrum data of the isolated products are provided in Table 1.

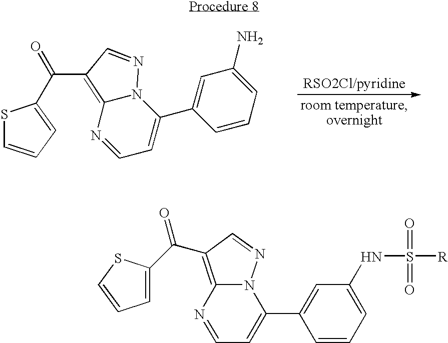

Examples 20-33 were synthesized in parallel by the following procedure: [7-(3-aminophenyl)pyrazolo[1,5-a]pyrimidin-3-yl]-2-thienyl-methanone (0.1 mmole) was dissolved in 2 mL of pyridine and was treated with appropriate sulfonyl chlorides (0.12 mmole) at room temperature. The reaction was stirred at room temperature overnight and the volatiles were removed under reduced pressure. The resulting residue was dissolved in a 1:1 mixture of dimethyl sulfoxide and acetonitrile and purified by preparative HPLC. The fractions were analyzed by LC/MS to identify the product. The mass spectrum data of the isolated products are provided in Table 1.

Detailed Procedures:

Specific compounds are identified by both an example number and their IUPAC name. Exemplary reference methods are labeled A-P. The structures are set forth in the table following the examples. In the event the IUPAC name is unclear or inconsistent, the structure prevails over name.

Reference Method A

### [7-(3-Nitrophenyl)pyrazolo[1,5-a]pyrimidin-3-yl]-2-thienyl-methanone

A mixture of (3-amino-1H-pyrazol-4-yl)-2-thienyl-methanone (2.0 g, 10.4 mmol) and 3-(dimethylamino)-1-(3-nitrophenyl)-2-propen-1-one (2.24 g, 10.4 mmol) in acetic acid (10 mL) was heated at reflux for 3 hours. The reaction mixture was cooled to room temperature and slurried with water. The thick suspension was filtered, washed thoroughly with water and dried to give 3.38 g (93%) of [7-(3-nitrophenyl)pyrazolo[1,5-a]pyrimidin-3-yl]-2-thienyl-methanone as a white solid, mp 199-201° C.

The preparation of (3-amino-1H-pyrazol-4-yl)-2-thienyl-methanone is described in DE patent 3422876.

Reference Method B

### 3-(Dimethylamino)-1-(3-nitrophenyl)-2-propen-1-one

3-Nitroacetophenone (5.0g, 30.3 mmol) in dimethylformamide-dimethylacetal (10 mL) was heated at reflux overnight. The reaction mixture was cooled to room temperature and evaporated to remove the volatiles. The residue was slurried in ethyl ether and the suspension was filtered and washed with ether to give 10.5 g (79%) of 3-(dimethylamino)-1-(3-nitrophenyl)-2-propen-1-one, 104-105° C.

Reference Method C

### [7-(3-Aminophenyl)pyrazolo[1,5-a]pyrimidin-3-yl]-2-thienyl-methanone

A 2.0 L three neck flask equipped with mechanical stirrer was charged [7-(3-nitrophenyl)pyrazolo[1,5-a]pyrimidin-3-yl]-2-thienyl-methanone (30 g, 86 mmol), and ammonium chloride (23 g, 428 mmol) in methanol (200 mL) and water (200 mL). The mixture was stirred for 5 minutes. Iron powder (19.1 g, 343 mmol) was added slowly with stirring followed by an additional 200 mL of methanol and 200 mL of water. The reaction mixture was heated gradually to reflux and maintained at reflux overnight, cooled to room temperature and filtered. The red solid cake was washed thoroughly with hot methanol and hot ethyl acetate. The combined filtrates were evaporated to give 20.1 g (74%) of [7-(3-aminophenyl)pyrazolo[1,5-a]pyrimidin-3-yl]-2-thienyl-methanone as a light brown solid, mp 183-184° C. The crude product was used directly for the next step without further purification.

Reference Method D

### 2-Fluoro-N-{3-[3-(thien-2-ylcarbonyl)pyrazolo[1,5-a]pyrimidin-7-yl]phenyl}benzamide

To a mixture of [7-(3-aminophenyl)pyrazolo[1,5-a]pyrimidin-3-yl]-2-thienyl-methanone (100 mg, 0.31 mmol), 4-dimethylaminopyridine (4 mg, 0.03 mmol) and triethylamine (47 mg, 0.47 mmol) in methylene chloride (2 mL) was added o-fluorobenzoyl chloride (60 mg, 0.38 mmol) in methylene chloride (1 mL) via syringe. The resulting mixture was stirred at room temperature for 2 hours, diluted with methylene chloride (100 mL) and washed with saturated sodium bicarbonate and water. After drying over sodium sulfate, the methylene chloride solution was concentrated and the residue was column chromatographed using ethyl acetate/hexanes (1:2) as the eluting solvent to give 93 mg (67%) of 2-Fluoro-N-{3-[3-(thien-2-ylcarbonyl)pyrazolo[1,5-a]pyrimidin-7-yl]phenyl}benzamide as an off-white solid, mp, 190-191 ° C.

Reference Method E

### N-{3-[3-(2-Thienylcarbonyl)pyrazolo[1,5-a]pyrimidin-7yl]phenyl}isonicotinamide

The mixture of isonicotinic acid (115 mg, 0.94 mmol) and N-methylmorpholine (108 mg, 1.1 mmol) in methylene chloride (2 mL) was cooled with an ice-water bath. A solution of isobutyl chloroformate (128 mg, 0.94 mmol) in methylene chloride (1 mL) was added via syringe. The resulting mixture was stirred at 0-5° C. for 2 hours before adding [7-(3-aminophenyl)pyrazolo[1,5-a]pyrimidin-3-yl]-2-thienyl-methanone (100 mg, 0.31 mmol), triethylamine (47 mg, 0.47 mmol) and 4-dimethylaminopyridine (catalytic amount). The resulting mixture was stirred at room temperature overnight, diluted with methylene chloride (100 mL) and washed with saturated sodium bicarbonate and water. After drying over sodium sulfate, the methylene chloride solution was concentrated and the residue was column chromatographed eluting with a mixture of methanol and methylene chloride to give 85 mg (64%) of N-{3-[3-(2-thienylcarbonyl)pyrazolo[1,5-a]pyrimidin-7-yl]phenyl}isonicotinamide as an off-white solid, mp, 212-215° C.

Reference Method F

### {7-[3-(Benzylamino)phenyl]pyrazolo[1,5-a]pyrimidin-3-yl}(thien-2-yl)methanone

[7-(3-Aminophenyl)pyrazolo[1,5-a]pyrimidin-3-yl]-2-thienyl-methanone (160 mg, 0.5 mmol) and benzaldehyde (56 mg, 0.53 mmol) were mixed in methylene chloride (5.0 mL) at room temperature. Sodium triacetoxyborohydride (530 mg, 2.5 mmol) was added in portions over 30 minutes. The resulting mixture was stirred at room temperature overnight and partitioned between water and ethyl acetate. The combined organics were dried over sodium sulfate and concentrated to give a syrupy residue which was column chromatographed eluting with 1:1 ethyl acetate/hexanes to give 117 mg (73%) of the title compound as a light yellow solid, mp 90-92° C.

Reference Method G

### N-Butyl-N′-{3-[3-(thien-2-ylcarbonyl)pyrazolo[1,5-a]pyrimidin-7-yl)phenyl}urea

[7-(3-Aminophenyl)pyrazolo[1,5-a]pyrimidin-3-yl]-²-thienyl-methanone (1.2 g, 3.75 mmol) was suspended in tetrahydrofuran (100 mL). Butyl isocyanate (1.5 g, 15.0 mmol) was added. Triethylamine (0.76 g, 7.5 mmol) was added with stirring. The resulting mixture was heated to reflux overnight and evaporated. The residue was dissolved in methylene chloride and washed with saturated aqueous sodium bicarbonate. The organic layer was separated, dried over magnesium sulfate, concentrated and column chromatographed eluting with a gradient mixture of methanol and methylene chloride to give 1.11 g (69%) of the title compound as an off-white solid, mp 158-160° C.

Reference Method H

### 2,2,2-Trichloroethyl 3-[3-(thien-2-ylcarbonyl)pyrazolo[1,5-a]pyrimidin-7-yl]phenylcarbamate

[7-(3-Aminophenyl)pyrazolo[1,5-a]pyrimidin-3-yl]-2-thienyl-methanone (410 mg, 1.28 mmol) was suspended in dry tetrahydrofuran (10 mL) followed by addition of triethylamine (142 mg, 1.40 mmol). The mixture was stirred at room temperature for 30 minutes. 2,2,2-Trichloroethyl chloroformate (271 mg; 1.28 mmol) was added slowly and the reaction mixture was stirred overnight at room temperature. The volatiles were removed by evaporation under reduced pressure and the residue was partitioned between water and ether/ethyl acetate. The combined organics were dried over magnesium sulfate and concentrated to give a yellowish semi-solid. The crude solid was recrystallized from ethyl acetate to give 478 mg (75%) of the title compound as an off-white solid, mp 197-200° C.

Reference Method I

### 3-(1,1-Dioxidothiomorpholin-4-yl)propyl 3-[3-(thien-2-ylcarbonyl)pyrazolo[1,5-a]pyrimidin-7-yl]phenylcarbamate

p-Nitrophenyl chloroformate (314 mg, 1.57 mmol) was dissolved in methylene chloride (2.5 mL) and cooled to 0-5° C. 3-(1, 1-Dioxido-4-thiomorpholinyl)-1-propanol (GB patent 0000373) (302 mg, 1.57 mmol) and 4-methylmorpholine (237 mg, 2.35 mmol) were added and the reaction mixture was stirred with cooling for 2 hours. [7-(3-Aminophenyl)pyrazolo[1,5-a]pyrimidin-3-yl]-2-thienyl-methanone (100 mg, 0.31 mmol) was added, followed by addition of pyridine (2.5 mL) and 4-dimethylaminopyridine (catalytic amount). The resultant mixture was heated at 100-105° C. for 2 hours and cooled to room temperature, and diluted with methylene chloride. The organic solution was washed with saturated aqueous sodium carbonate and water, dried over sodium sulfate and concentrated on silica gel. Flash column chromatography afforded 99 mg (59%) of the title compound as an off-white solid, mp 148-159° C.

Reference Method J

### N-Isobutyl-3-[3-(2-thienylcarbonyl)pyrazolo[1,5-a]pyrimidin-7-yl]benzamide

A mixture of 3-[3-(2-thienylcarbonyl)pyrazolo[1,5-a]pyrimidin-7-yl]benzoic acid (150 mg, 0.43 mmol), diisopropylethylamine (167 mg, 1.3 mmol), benzotriazole-1-yloxy-tripyrrolidinophosphonium hexafluorophosphate (290 mg, 0.56 mmol) and isobutylamine (31 mg, 0.43 mmol) in methylene chloride (4 mL) was stirred at room temperature for 5 hours. The reaction mixture was partitioned between saturated aqueous sodium bicarbonate and methylene chloride. The combined organics were dried over sodium sulfate, concentrated and purified by flash column chromatography eluting with a gradient mixture of methanol and methylene chloride to give 170 mg (98%) of the title compound as a white solid.

Preparation of 3-[3-(2-thienylcarbonyl)pyrazolo[1,5-a]pyrimidin-7-yl]benzoic acid: a mixture of methyl 3-[3-(2-thienylcarbonyl)pyrazolo[1,5-a]pyrimidin-7-yl]benzoate (177 mg, 0.49 mmol), potassium hydroxide (1.0 M, 5.0 mL) and methanol (0.3 mL) was stirred at room temperature overnight and then heated at 50° C. for 15 minutes. The reaction mixture was cooled to room temperature and solid precipitated. The resulting suspension was diluted with water until a solution and hydrochloric acid (conc.) was added until acidic pH. The precipitate was collected, washed with water and dried to give 154 mg (90%) of the title compound as a light yellow solid, mp 174-177° C.

Reference Method K

### 3-amino-1H-pyrazol-4-yl)-2-thienyl-methanone

A mixture of 2-acetylthiophene (30.2 g, 239 mmol) and dimethyl formamide-dimethyl acetal 990 mL) was heated at reflux for 15 hours. The reaction mixture was cooled and the volatiles were removed under reduced pressure. The residue was slurried in ether, filtered and washed with ether to give 39.35 g of 3-(dimethylamino)-1-(2-thienyl)-2-propen-1-one as a light orange solid, which was then treated with hydroxylamine hydrochloride (18.1 g, 260 mmol) in methanol (200 mL). The above mixture was heated at reflux for 3 hours and at room temperature overnight to result in a clear yellow solution. Dimethyl formamide-dimethyl acetal (90 mL) was added and the resultant mixture was stirred at reflux for 3 hours. After cooling to room temperature, the precipitate was collected by filtration and washed with ether to give 36.4 g (74%) of α-[(dimethylamino)methylene]-β-oxo-2-thiophenyl-propanenitrile as an orange crystal.

A mixture of α-[(dimethylamino)methylene]-β-oxo-2-thiophenyl-propanenitrile (30.9 g, 150 mmol), aminoguanidine nitrate (24.7 g, 180 mmol) and 10 N sodium hydroxide (18 mL) in ethanol (450 mL) was heated at reflux for 3.5 hours. The reaction mixture was cooled to room temperature and the volatiles were evaporated under reduced pressure. The residue was diluted with water and cooled with an ice-water bath. The precipitate was collected by filtration and washed with ethanol. More solids were obtained from the filtrate after reducing the volume and diluting with water to give a total of 22.2 g (76%) of the title compound as an off-white solid, mp 114-115° C.

Reference Method L

### α-[(Dimethylamino)methylene]-β-oxo-2-furane-propanenitrile

A 50 mL portion of dimethylformamide-dimethylacetal was added to β-oxo-2-furanepropanenitrile (25 g, 198 mmol) slowly. The reaction mixture was stirred at room temperature for 2 hours and the volatiles were removed under reduced pressure. The residue was dissolved in methylene chloride and the solution was passed through a short pad of hydrous magnesium silicate. The eluate was refluxed with the gradual addition of hexanes to the point of turbidity. Cooling and filtration gave 35.2 g of the title compound, mp 117-125° C.

Reference Method M

### {7-[4′-(Dimethylamino)-1,1′-biphenyl-3-yl]pyrazolo[1,5-a]pyrimidin-3-yl}(thien-2-yl)methanone

A mixture of 4-N,N-dimethyaminophenyl boronic acid (0.22 g, 1.3 mmol), [7-(3-bromophenyl)pyrazolo[1,5-a]pyrimidin-3-yl]-2-thienyl-methanone (0.25 g, 0.65 mmol), Pd(PPh₃)₄ (40 mg, 0.03 mmol) and Na₂CO₃ (aq) (2.0 M, 0.33 mL) in EtOH (0.5 mL)/H20 (0.7 mL)/DME (2 mL) was irradiated in a microwave at 100° C. for 4 minutes. After cooling to room temperature, the reaction mixture was diluted with CHCl₃ ( 200 mL), washed with water and saturated aqueous NaHCO_{3.}, dried over Na₂SO₄ and concentrated. The residue was purifed by silica gel flash column chromatography to give 598 mg (72%) of the desired product as a yellow solid, mp 200-202° C.

[7-(3-Bromophenyl)pyrazolo[1,5-a]pyrimidin-3-yl]-2-thienyl-methanone is prepared using a procedure similar to Method A from (5-amino-1H-pyrazol-4-yl)(thien-2-yl)methanone and 1-(3-bromo-phenyl)-3-dimethylamino-propenone, mp 193-195° C.

Reference Method N

### {7-[4′-(Morpholin-4-ylmethyl)-1,1′-biphenyl-3-yl]pyrazolo[1,5-a]pyrimidin-3-yl}(thien-2-yl)methanone

A mixture of 3′-[3-(thien-2-ylcarbonyl)pyrazolo[1,5-a]pyrimidin-7-yl]-1,1′-biphenyl-4-carbaldehyde (100 mg, 0.24 mmol) and morpholine (106 mg, 1.22 mmol) was dissolved in CH₂Cl₂ (15 mL)/DMF (0.2 mL) and stirred for 10 minutes, with the cooling of ice water bath and. Sodium triacetoxyborohydride (311 mg, 1.5 mmol) was added and the mixture was stirred from 30 minutes. Acetic acid (0.2 mL) was added, followed by another 2.5 hours of stirring. The resulting mixture was quenched with water and the organic phase was washed with saturated aqueous NaHCO₃ and water and evaporated. The residue was purifed with silica gel flash column chromatography to give 88 mg (75%) of the desired product as a gray crystal, mp 175-178° C.

Reference Method O

### 3-(3,5-Dimethylphenyl)-7-[3-(1H-tetraazol-5-yl)phenyl]pyrazolo[1,5-a]pyrimidine (WAY-189354)

A mixture of 3-[3-(3,5-dimethylphenyl)pyrazolo[1,5-a]pyrimidin-7-yl]benzonitrile (2.51 g, 7.75 mmol), sodium azide (2.76 g, 42.5 mmol) and ammonium chloride (2.3 g, 5.55 mmol) in DMF (17 mL) was heated at 100° C. under nitrogen overnight. The reaction mixture was cooled to room temperature and was diluted with water. The precipitation was collected by filtration, washed with water and ether to give 2.4 g (84%) of desired product as a red solid, mp 250° C. (dec.).

Reference Method P

### 7-{3-[2-(Cyclobutylmethyl)-2H-tetraazol-5-yl]phenyl}-3-(3,5-dimethylphenyl)pyrazolo[1,5-a]pyrimidine

3-(3,5-Dimethylphenyl)-7-[3-(1H-tetraazol-5-yl)phenyl]pyrazolo[1,5-a]pyrimidine (150 mg, 0.41 mmol) was dissolved in acetonitrile (6 mL) an DMF (6 mL). Cs₂CO₃ (0.2 g, 0.61 mmol) was added followed by cyclobutylmethyl bromide (0.064 mL, 0.57 mmol). The resulting mixture was heated at reflux for 48 hours and partitioned between water and ethyl acetate. The combined organics were dried over Na₂SO₄ and concentrated. The residue was purified by silica gel flash column chromatograph to give 120 mg (67%) of the desired product as a yellow foam, mp 64° C.

### EXAMPLE 175

### 1-[3-[3-(2-Furanylcarbonyl)pyrazolo-[1,5-a]pyrimidin-7-yl]phenyl]-2-piperidinone

The title compound was prepared using a procedure similar to Method A from 1-[3-[3-(dimethylamino)-1-oxo-2-propenyl]phenyl-2-piperidinone and (3-amino-1H-pyrazol-4-yl)-2-furanyl-methanone, mp 159-160° C.

1-[3-[3-(Dimethylamino)-1-oxo-2-propenyl]phenyl-2-piperidino was prepared using a procedure similar to Method B by treating 1-(3-acetylphenyl)tetrahydro-2(1H)-pyridinone with dimethyl formamide-dimethyl acetal.

### EXAMPLE 176

### 1-[3-(3-Benzoylpyrazolo[1,5-a]-pyrimidin-7-yl)phenyl]-2-piperidinone

The title compound was prepared using a procedure similar to Method A from 1-[3-[3-(dimethylamino)-1-oxo-2-propenyl]phenyl-2-piperidinone and (3-amino-1H-pyrazol-4-yl)phenyl-methanone, mp 160-162° C.

### EXAMPLE 177

1-[3-[3-(2-Furanylcarbonyl)pyrazolo-[1,5-a]pyrimidin-7-yl]phenyl]-2-pyrrolidinone

The title compound was prepared using a procedure similar to Method A from 1-[3-[3-(dimethylamino)-1-oxo-2-propenyl]phenyl -2-pyrrolidinone and (3-amino-1H-pyrazol-4-yl)-2-furanyl-methanone, mp 210-211° C.

1-[3-[3-(Dimethylamino)-1-oxo-2-propenyl]phenyl-2 pyrrolidinone was prepared using a procedure similar to Method B by treating 1-(3-acetylphenyl)tetrahydro-2(1H)-pyrrolidinone with dimethyl formamide-dimethyl acetal.

### EXAMPLE 178

### 1-[3-(3-Benzoylpyrazolo[1,5-a]-pyrimidin-7-yl)phenyl]-2-pyrrolidinone

The title compound was prepared using a procedure similar to Method A from 1-[3-[3-(dimethyl amino)-1-oxo-2-propenyl] phenyl-2-pyrrolidinone and (3-amino-1H-pyrazol-4-yl)phenyl-methanone, mp 173-174° C.

### EXAMPLE 179

### N-[3-(3-Cyanopyrazolo[1,5-a]-pyrimidin-7-yl)phenyl]-N-methyl-cyclobutanecarboxamide

The title compound was prepared using a procedure similar to Method A from N-[3-(3-dimethylamino-1-oxo-2-propenyl)phenyl]-N-methyl-cyclobutanecarboxamide and 3-amino-4-cyano-1H-pyrazol mp 157-158° C.

### EXAMPLE 180

### [7-(2,4-Dimethoxyphenyl)pyrazolo[1,5-a]-pyrimidin-3-yl]-2-thienyl-methanone

The title compound was prepared using a procedure analogous to Method A from (3-amino-1H-pyrazol-4-yl)-2-thienyl-methanone (2.0 g, 10.4 mmol) and 3-(dimethylamino)-1-(2,4-dimethoxyphenyl)-2-propen-1-one, mp 146-148° C.

3-(Dimethylamino)-1-(2,4-dimethoxyphenyl)-2-propen-1-one was prepared from 2,4-dimethoxyacetophenone and dimethyl formamide-dimethyl acetal using a procedure analogous to Method B.

### EXAMPLE 181

### N,N′-Diethyl-3-[3-(2-thienylcarbonyl)-pyrazolo[1,5-a]pyrimidin-7-yl]-benzamide

The title compound was prepared using a procedure similar to Method A from 3-[3-(dimethylamino)-1-oxo-2-propenyl]-N,N-diethyl-benzamide and (3-amino-1H-pyrazol-4-yl)-2-thienyl-methanone, mp 165-167° C.

3-[3-(Dimethylamino)-1-oxo-2-propenyl]-N,N-diethyl-benzamide was prepared using an analogous procedure to Method B from methyl (3-acetylphenyl)-N,N-diethyl-benzamide and dimethyl formamide-dimethyl acetal.

### EXAMPLE 182

### N-[3-(3-Chloropyrazolo[1,5-a]pyrimidin-7-yl)phenyl]-N-ethyl-cyclopropanecarboxamide

The title compound was prepared using a procedure similar to Method A from N-[3-(3-dimethylamino-1-oxo-2-propenyl)phenyl]-N-ethyl-cyclopropanecarboxamide and 3-amino-4-chloro-1H-pyrazol, mp 150-151° C.

### EXAMPLE 183

### N-[3-(3-Cyanopyrazolo[1,5-a]pyrimidin-7-yl)phenyl]-N-ethyl-cyclopropanecarboxamide

The title compound was prepared using a procedure similar to Method A from N-[3-(3-dimethylamino-1-oxo-2-propenyl)phenyl]-N-ethyl-cyclopropanecarboxamide and 3-amino-4-cyano-1H-pyrazol, mp 166-168° C.

### EXAMPLE 184

### [7-[3-(2-Methoxyethoxy)phenyl]pyrazolo[1,5-a]pyrimidin-3-yl]-2-thienyl-methanone

The title compound was prepared using a procedure analogous to Method A from (3-amino-1H-pyrazol-4-yl)-2-thienyl-methanone and 3-(dimethylamino)-1-[3-(2-methoxy)ethoxyphenyl]-2-propen-1-one, mp 112-116° C.

3-(Dimethylamino)-1-[3-(2-methoxy)ethoxyphenyl ]-2-propen-1-one was prepared from 3-ethoxyacetophenone and dimethyl formamide-dimethyl acetal using an analogous procedure to Method B.

### EXAMPLE 185

### [7-[3-(Cyclopentyloxy)phenyl]pyrazolo[1,5-a]pyrimidin-3-yl]-2-thienyl-methanone

The title compound was prepared using a procedure analogous to Method A from (3-amino-1H-pyrazol-4-yl)-2-thienyl-methanone and 3-(dimethylamino)-1-[3-(cyclopentyloxy)phenyl]-2-propen-1-one, mp 135-138° C.

3-(Dimethylamino)-1-[3-(cyclopentyloxy)phenyl]-2-propen-1-one was prepared from 3-cyclopentyloxyacetophenone and dimethyl formamide-dimethyl acetal using an analogous procedure to Method B.

### EXAMPLE 186

### 2-Furanyl[7-[3-(1H-pyrrol-1-yl)phenyl]pyrazolo[1,5-a]pyrimidin-3-yl]-methanone

The title compound was prepared using a procedure analogous to Method A from (3-amino-1H-pyrazol-4-yl)-2-furanyl-methanone and 3-(dimethylamino)-1-[3-(1H-pyrrol-1-yl)phenyl]-2-propen-1-one, mp 204-106° C.

3-(Dimethylamino)-1-[3-(1H-pyrrol-1-yl)phenyl]-2-propen-1-one was prepared from 3-(1H-pyrrol-1-yl)acetophenone and dimethyl formamide-dimethyl acetal using an analogous procedure to Method B.

### EXAMPLE 187

### [7-[3-( 1H-Pyrrol-1-yl)phenyl]pyrazolo[1,5-a]pyrimidin-3-yl]-2-thienyl-methanone

The title compound was prepared using a procedure analogous to Method A from (3-amino-1H-pyrazol-4-yl)-2-thienyl-methanone and 3-(dimethylamino)-1-[3-(1H-pyrrol-1-yl)phenyl]-2-propen-1-one, mp 211-213° C.

### EXAMPLE 188

### N-{3-[3-(2-Thienylcarbonyl)pyrazolo[1,5-a]pyrimidin-7-yl]phenyl}acrylamide

The title compound was prepared using a procedure analogous to Method D from [7-(3-aminophenyl)pyrazolo[1,5-a]pyrimidin-3-yl]-2-thienyl-methanone and acryloyl chloride, mp 192-194° C.

### EXAMPLE 189

### N-{3-[3-(2-Thienylcarbonyl)pyrazolo[1,5-a]pyrimidin-7-yl]phenyl}-2-butynamide

The title compound was prepared using an analogous procedure to Method E from [7-(3-aminophenyl)-5-methyl-pyrazolo[1,5-a]pyrimidin-3-yl]-2-thienyl-methanone and 2-butynoic acid, mp 202-203° C.

### EXAMPLE 190

### N-{3-[3-(2-Thienylcarbonyl)pyrazolo[1,5-a]pyrimidin-7-yl]phenyl}-2-oxiranecarboxamide

The title compound was prepared using an analogous procedure to Method E from [7-(3-aminophenyl)-5-methyl-pyrazolo[1,5-a]pyrimidin-3-yl]-2-thienyl-methanone and glycidic acid, mp 70° C. (decomposition).

### EXAMPLE 191

### {3-[5-Methyl-3-(2-thienylcarbonyl)pyrazolo[1,5-a]pyrimidin-7-yl]phenyl}-2-oxiranecarboxamide

The title compound was prepared using an analogous procedure to Method D from [7-(3-aminophenyl)-5-methyl -pyrazolo[1,5-a]pyrimidin-3-yl]-2-thienyl-methanone and cyclopropanecarbonyl chloride, mp 70° C. (decomposition).

### EXAMPLE 192

### N-{3-[3-(2-Thienylcarbonyl)pyrazolo[1,5-a]pyrimidin-7-yl]phenyl}-2,5-bis(trifluoromethyl)benzamide

The title compound was prepared using a procedure analogous to Method D from [7-(3-aminophenyl)pyrazolo[1,5-a]pyrimidin-3-yl]-2-thienyl-methanone and 2,5-bis(trifluoromethyl)benzoyl chloride, mp 208-210° C.

### EXAMPLE 193

### N-{3-[3-(2-Thienylcarbonyl)pyrazolo[1,5-a]pyrimidin-7-yl]phenyl}-3-trifluoromethyl)benzamide

The title compound was prepared using a procedure analogous to Method D from [7-(3-aminophenyl)pyrazolo[1,5-a]pyrimidin-3-yl]-2-thienyl-methanone and 3-(trifluoromethyl)benzoyl chloride, mp 135-140° C.

### EXAMPLE 194

### N-{3-[3-(2-Thienylcarbonyl)pyrazolo[1,5-a]pyrimidin-7-yl]phenyl}benzamide

The title compound was prepared using a procedure analogous to Method D from [7-(3-aminophenyl)pyrazolo[1,5-a]pyrimidin-3-yl]-2-thienyl-methanone and benzoyl chloride, mp 211-213° C.

### EXAMPLE 195

### N-{3-[3-(2-Thienylcarbonyl)pyrazolo[1,5-a]pyrimidin-7-yl]phenyl}-2-furamide

The title compound was prepared using a procedure analogous to Method D from [7-(3-aminophenyl)pyrazolo[1,5-a]pyrimidin-3-yl]-2-thienyl-methanone and furoyl chloride, mp >240° C.

### EXAMPLE 196

### 3-Bromo-N-{3-[3-(2-thienylcarbonyl)pyrazolo[1,5-a]pyrimidin-7-yl]phenyl}benzamide

The title compound was prepared using a procedure analogous to Method D from [7-(3-aminophenyl)pyrazolo[1,5-a]pyrimidin-3-yl]-2-thienyl-methanone and 3-bromobenzoyl chloride, mp >300° C.

### EXAMPLE 197

### 4-(tert-Butyl)-N-{3-[3-(2-thienylcarbonyl)pyrazolo[1,5-a]pyrimidin-7-yl]phenyl}benzamide

The title compound was prepared using a procedure analogous to Method D from [7-(3-aminophenyl)pyrazolo[1,5-a]pyrimidin-3-yl]-2-thienyl-methanone and 4-tert-butylbenzoyl chloride as a yellow foam, M+H 491.

### EXAMPLE 198

### 3.5-Dinitro-N-{3-[3-(2-thienylcarbonyl)pyrazolo[1,5-a]pyrimidin-7-yl]phenyl}benzamide

The title compound was prepared using a procedure analogous to Method D from [7-(3-aminophenyl)pyrazolo[1,5-a]pyrimidin-3-yl]-2-thienyl-methanone and 3,5-dinitrobenzoyl chloride, mp 145° C. (dec.).

### EXAMPLE 199

### 2,4-Dichloro-N-{3-[3-(2-thienylcarbonyl)pyrazolo[1,5-a]pyrimidin-7-yl]phenyl}benzamide

The title compound was prepared using a procedure analogous to Method D from [7-(3-aminophenyl)pyrazolo[1,5-a]pyrimidin-3-yl]-2-thienyl-methanone and 2,4-dichlorobenzoyl chloride, mp 132-134° C.

### EXAMPLE 200

### N-{3-[3-(2-Thienylcarbonyl)pyrazolo[1,5-a]pyrimidin-7-yl]phenyl}nicotinamide

The title compound was prepared using a procedure analogous to Method D from [7-(3-aminophenyl)pyrazolo[1,5-a]pyrimidin-3-yl]-2-thienyl-methanone and nicotinoyl chloride hydrochloride, mp 191-194° C.

### EXAMPLE 201

### N-{3-[3-(2-Thienylcarbonyl)pyrazolo[1,5-a]pyrimidin-7-yl]phenyl}isonicotinamide

The title compound was prepared using a procedure analogous to Method D from [7-(3-aminophenyl)pyrazolo[1,5-a]pyrimidin-3-yl]-2-thienyl-methanone and isonicotinoyl chloride hydrochloride or prepared from [7-(3-aminophenyl-5-methyl)pyrazolo[1,5-a]pyrimidin-3-yl]-2-thienyl-methanone and isonicotinic acid as described in Method E, mp 220-222° C.

### EXAMPLE 202

### N,N′-Dibutyl-N-{3-[3-(2-thienylcarbonyl)pyrazolo[1,5-a]pyrimidin-7-yl]phenyl}dicarbonimidic diamide

The title compound was prepared using a procedure analogous to Method G from [7-(3-aminophenyl)pyrazolo[1,5-a]pyrimidin-3-yl]-2-thienyl-methanone and excess of butyl isocyanate, mp 180-181° C.

### EXAMPLE 203

### Isopropyl 3-[3-(cyclopentylcarbonyl)pyrazolo[1,5-a]pyrimidin-7-yl]phenylcarbamate

The title compound was prepared using a procedure analogous to Method H from [7-(3-aminophenyl)pyrazolo[1,5-a]pyrimidin-3-yl]cyclopentyl-methanone and isopropyl chloroformate, mp 57-59° C.

[7-(3-Aminophenyl)pyrazolo[1,5-a]pyrimidin-3-yl]cyclopentyl methanone was prepared from [7-(3-nitrophenyl)pyrazolo[1,5-a]pyrimidin-3-yl]cyclopentylumethanone by an analogous procedure to Method C.

[7-(3-Nitrophenyl)pyrazolo[1,5-a]pyrimidin-3-yl]cyclopentylmethanone was prepared from 3-(dimethylamino)-1-(3-nitrophenyl)-2-propen-1-one and (5-amino-1H-pyrazol-4-yl)(cyclopentyl)methanone by an analogous procedure to Method A.

(5-Amino-1H-pyrazol-4-yl)(cyclopentyl)methanone was prepared using an analogous procedure described in Method K from (2E)-2-(cyclopentylcarbonyl)-3-dimethylamino-2-propenenitrile.

(2E)-2-(Cyclopentylcarbonyl)-3-dimethylamino-2-propenenitrile was prepared from (β-oxo-cyclopentanepropanenitrile (EP patent 157260) by an analogous procedure to Method L.

### EXAMPLE 204

### N-{3-[3-(Cyclopentylcarbonyl)pyrazolo[1,5-a]pyrimidin-7-yl]phenyl}-3-methylbutanamide

The title compound was prepared using a procedure analogous to Method H from [7-(3-aminophenyl)pyrazolo[1,5-a]pyrimidin-3-yl]cyclopentyl-methanone (Example 295) and isobutyl chloroformate, mp 69-71° C.

### EXAMPLE 205

### N-{3-[3-(2-Furoyl)pyrazolo[1,5-a]pyrimidin-7-yl]phenyl}benzamide

The title compound was prepared using a procedure analogous to Method D from [7-(3-aminophenyl)pyrazolo[1,5-a]pyrimidin-3-yl]-2-furanyl-methanone and benzoyl chloride, mp 220-222° C.

### EXAMPLE 206

### Methyl 3-[3-(2-thienylcarbonyl)pyrazolo[1,5-a]pyrimidin-7-yl]benzoate

The title compound was prepared using a procedure analogous to Method A from (3-amino-1H-pyrazol-4-yl)-2-thienyl-methanone and methyl 3-[3-(dimethylamino)-1-oxo-2-propenyl]benzoate, mp 180-184° C.

Methyl 3-[3-(imethylamino)-1-oxo-2-propenyl]benzoate was prepared from methyl 3-acetylbenzoate and dimethyl formamide-dimethyl acetal using an analogous procedure to Method B.

### EXAMPLE 207

### N-{2-Methyl-5-[3-(2-thienylcarbonyl)pyrazolo [1,5-a]pyrimidin-7-yl]phenyl}benzamide

The title compound was prepared using a procedure analogous to Method D from {[7-(3-amino-4-methyl)phenyll]pyrazolo[1,5-a]pyrimidin-3-yl}-2-thienyl-methanone and benzoyl chloride, mp 216-218° C.

### EXAMPLE 208

### 3-Methyl-N-{2-methyl-5-[3-(2-thienylcarbonyl)pyrazolo[1,5-a]pyrimidin-7-yl]phenyl}butanamide

The title compound was prepared using a procedure analogous to Method D from {[7-(3-amino-4-methyl)phenyl]pyrazolo[1,5-a]pyrimidin-3-yl}-2-thienyl-methanone and isovaleryl chloride, mp 185-187° C.

### EXAMPLE 209

### N-Isopropyl-N′-{2-methyl-5-[3-(2-thienylcarbonyl)pyrazolo[1,5-a]pyrimidin-7-yl]phenyl}urea

The title compound was prepared using a procedure analogous to Method G from {[7-(3-amino-4-methyl)phenyl]pyrazolo[1,5-a]pyrimidin-3-yl}-2-thienyl-methanone and isopropyl isocyanate, mp 225-227° C.

### EXAMPLE 210

### N-{2-Chloro-5-[3-(2-thienylcarbonyl)pyrazolo[1,5-a]pyrimidin-7-yl]phenyl}benzamide

The title compound was prepared using a procedure analogous to Method D from {[7-(3-amino-4-chloro)phenyl]pyrazolo[1,5-a]pyrimidin-3-yl}-2-thienyl-methanone and benzoyl chloride, mp 217-218° C.

### EXAMPLE 211

### N-{2-Chloro-5-[3-(2-thienylcarbonyl)pyrazolo[1,5-a]pyrimidin-7-yl]phenyl}-3-Methylbutanamide

The title compound was prepared using a procedure analogous to Method D from {[7-(3-amino-4-chloro)phenyl]pyrazolo[1,5-a]pyrimidin-3-yl}-2-thienyl-methanone and isovaleryl chloride, mp 172-175° C.

### EXAMPLE 212

### Isopropyl 2-methyl-5-[3-(2-thienylcarbonyl)pyrazolo[1,5-a]pyrimidin-7-yl]phenylcarbamate

The title compound was prepared using a procedure analogous to Method H from {[7-(3-amino-4-methyl)phenyl]pyrazolo [1,5-a]pyrimidin-3-yl }-2-thienyl-methanone and isopropyl chloroformate, mp 198-200° C.

### EXAMPLE 213

### N-{2-Methoxy-5-[3-(2-thienylcarbonyl)pyrazolo[1,5-a]pyrimidin-7-yl]phenyl}benzamide

The title compound was prepared using a procedure analogous to Method D from {[7-(3-amino-4-methoxy)phenyl]pyrazolo[1,5-a]pyrimidin-3-yl}-2-thienyl-methanone and benzoyl chloride, mp 201-203° C.

### EXAMPLE 214

### Isopropyl 2-methoxy-5-[3-(2-thienylcarbonyl)pyrazolo[1,5-a]pyrimidin-7-yl]phenylcarbamate

The title compound was prepared using a procedure analogous to Method H from {[7-(3-amino-4-methoxy)phenyl]pyrazolo[1,5-a]pyrimidin-3-yl}-2-thienyl-methanone and isopropyl chloroformate, mp 193-195° C.

### EXAMPLE 215

### N-{2-Methoxy-5-[3-(2-thienylcarbonyl)pyrazolo[1,5-a]pyrimidin-7-yl]phenyl}-3-methylbutanamide

The title compound was prepared using a procedure analogous to Method D from ([7-(3-amino-4-methoxy)phenyl]pyrazolo[1,5-a]pyrimidin-3-yl }-2-thienyl-methanone and isovaleryl chloride, mp 192-194° C.

### EXAMPLE 216

### N-Isobutyl-3-[3-(2-thienylcarbonyl)pyrazolo[1,5-a]pyrimidin-7-yl]benzamide

The title compound was prepared as described in Method J from 3-[3-(2-thienylcarbonyl)pyrazolo[1,5-a]pyrimidin-7-yl]benzoic acid and isobutyl amine, mp 174-177° C.

### EXAMPLE 217

### N-Butyl-3-[3-(2-thienylcarbonyl)pyrazolo[1,5-a]pyrimidin-7-yl]benzamide

The title compound was prepared using an analogous procedure to Method J from 3-[3-(2-thienylcarbonyl)pyrazolo[1,5-a]pyrimidin-7-yl]benzoic acid and n-butyl amine, mp 172-174° C.

### EXAMPLE 218

### N-Cyclopentyl-3-[3-(2-thienylcarbonyl)pyrazolo[1,5-a]pyrimidin-7-yl]benzamide

The title compound was prepared using an analogous procedure to Method J from 3-[3-(2-thienylcarbonyl)pyrazolo[1,5-a]pyrimidin-7-yl]benzoic acid and cyclopentylamine, mp 207-209° C.

### EXAMPLE 219

### N-Phenyl-3-[3-(2-thienylcarbonyl)pyrazolo[1,5-a]pyrimidin-7-yl]benzamide

The title compound was prepared using an analogous procedure to Method J from 3-[3-(2-thienylcarbonyl)pyrazolo[1,5-a]pyrimidin-7-yl]benzoic acid and aniline, mp 211-212° C.

### EXAMPLE 220

### N-Isopropyl-3-[3-(2-thienylcarbonyl)pyrazolo[1,5-a]pyrimidin-7-yl]benzamide

The title compound was prepared using an analogous procedure to Method J from 3-[3-(2-thienylcarbonyl)pyrazolo[1,5-a]pyrimidin-7-yl]benzoic acid and isopropylamine, mp 218-220° C.

### EXAMPLE 221

### N-Isopropyl-N′-{2-methoxy-5-[3-(2-thienylcarbonyl)pyrazolo[1,5-a]pyrimidin-7-yl]phenyl}urea

The title compound was prepared using a procedure analogous to Method G from {[7-(3-amino-4-methoxy)phenyl]pyrazolo[1,5-a]pyrimidin-3-yl}-2-thienyl-methanone and isopropyl isocyanate, mp 202-204° C.

### EXAMPLE 222

### 3-Methyl-N-{5-[3-(2-thienylcarbonyl)pyrazolo[1,5-a]pyrimidin-7-yl]-3-pyridinyl}butanamide

The title compound was prepared using a procedure analogous to Method D from [7-(5-amino-3-pyridinyl)pyrazolo[1,5-1]pyrimidin-3-yl](2-thienyl)methanone and isovaleryl isocyanate, mp 217-219° C.

### EXAMPLE 223

### 7-(3-Nitrophenyl)-3-(3-thienyl)pyrazolo[1,5-a]pyrimidine

A mixture of 3-bromo-7-(3-nitrophenylpyrazolo[1,5-a]pyrimidine (240 mg, 0.75 mmol), 3-thiopheneboronic acid (192 mg, 1.5 mmol) and palladium tetrakis(triphenylphosphine) (58 mg, 0.05 mmol) in dimethoxy ethane (6 mL) and saturated aqueous sodium bicarbonate (3 mL) was heated at 100-105° C. under nitrogen for 2 0 hours. The reaction mixture was partitioned between water and ethyl acetate. The combined organics were dried, concentrated and purified by flash column chromatography eluting with a gradient mixture of ethyl acetate/hexanes to give 246 mg (76%) of the title compound as an orange solid, mp 148-150° C.

### EXAMPLE 224

### 3-(3-Bromopyrazolo [1,5-a]pyrimidin-7-yl)aniline

The title compound was prepared using an analogous procedure to Method C from 3-bromo-7-(3-nitrophenylpyrazolo[1,5-a]pyrimidine, mp 120-121° C.

### EXAMPLE 225

### Isobutyl 3-(3-bromopyrazolo[1,5-a]pyrimidin-7-vl)phenvlcarbamate

The title compound was prepared using an analogous procedure to Method H from 3-(3-bromopyrazolo[1,5-a]pyrimidin-7-yl)aniline and isobutyl chlorofomate, mp 90-92° C.

### EXAMPLE 226

### [7-(5-Nitro-3-thienyl)pyrazolo[1,5-a]pyrimidin-3-yl](2-thienyl)methanone

The title compound was prepared using a procedure analogous to Method A from (3-amino-1H-pyrazol-4-yl)-2-thienyl-methanone and 3-(dimethylamino)-1-[5-nitro-3-thienyl]-2-propen-1-one, mp 225-226° C.

3-(Dimethylamino)-1-[5-nitril-3-thienyl]-2-propen-1-one was prepared using an analogous procedure to Method B from 2-acetyl-4-nitrothiophene and dimethyl formamide-dimethyl acetal.

### EXAMPLE 227

### N-Isopropyl-N′-{5-[3-(thien-2-ylcarbonyl)pyrazolo[1,5-a]pyrimidin-7-yl]thien-2-yl}urea

The title compound was prepared using a procedure analogous to Method G from and [7-(5-amino-2-thienyl)pyrazolo[1,5-a]pyrimidin-3-yl](thien-2-yl)methanone and isopropyl isocyanate, mp 162-165° C.

### EXAMPLE 228

### N-[3-(3-Bromopyrazolo[1,5-a]pyrimidin-7-yl)phenyl]-3-methylbutanamide

The title compound was prepared using an analogous procedure to Method D from 3-(3-bromopyrazolo[1,5-a]pyrimidin-7-yl)aniline and isovaleryl chloride, mp 132-134° C.

### EXAMPLE 229

### [7-(5-Aminothien-3-yl)pyrazolo[1,5-a]pyrimidin-3-yl](thien-2-yl)methanone

The title compound was prepared using an analogous procedure to Method C from [7-(5-nitro-3-thienyl)pyrazolo[1,5-a]pyrimidin-3-yl](2-thienyl)methanone, mp 174-176° C.

### EXAMPLE 230

### Ethyl 3-[3-(thien-2-ylcarbonyl)pyrazolo[1,5-a]pyrimidin-7-yl]phenylthiocarbamate

A mixture of [7-(3-aminophenyl)pyrazolo[1,5-a]pyrimidin-3-yl]-2-thienyl-methanone (320 mg, 1.0 mmol) and phenyl thioisocyanate (135 mg, 1.0 mmol) in ethanol (5 mL)and tetrahydrofuran (10 mL) was heated at reflux overnight. Additional phenyl thioisocyanate (135 mG, 1.0 mmol) was added and the reflux was continued for another 24 hours. The reaction mixture was diluted with methylene chloride, concentrated on silica gel and purified by flash column chromatography eluting with 1:6 ethyl acetate/hexanes to give 48 mg (12%) of the title compound as an off-white solid, mp 209-211° C.

### EXAMPLE 231

### 3-Methyl-N-[3-(3-thien-2-ylpyrazolo[1,5-a]pyrimidin-7-yl)phenyl]butanamide

The title compound was prepared using a procedure analogous to Example 325 from N-[3-(3-bromopyrazolo[1,5-a]pyrimidin-7-yl)phenyl]-3-methylbutanamide and 2-thiopheneboronic acid, mp 148-150° C.

### EXAMPLE 232

### N-Phenyl-N′-{3-[3-(thien-2-ylcarbonyl)pyrazolo[1,5-a]pyrimidin-7-yl]phenyl}thiourea

A mixture of [7-(3-aminophenyl)pyrazolo[1,5-a]pyrimidin-3-yl]-2-thienyl-methanone (320 mg, 1.0 mmol) and phenyl thioisocyanate (135 mg, 1.0 mmol) in tetrahydrofuran (5 mL) and pyridine (1 mL) was heated for 2 hours. The reaction mixture was diluted with saturated aqueous sodium bicarbonate. The precipitated solid was collected by filtration and washed with water. The crude solid was purified by purified by flash column chromatography eluting with 1:1 ethyl acetate/hexanes to give 181 mg (40%) of the title compound as an off-white solid, mp 124-126° C.

### EXAMPLE 233

### Isopropyl 4-chloro-3-[3-(thien-2-ylcarbonyl)pyrazolo[1,5-a]pyrimidin-7-yl]phenylcarbamate

The title compound was prepared using an analogous procedure to Method H from {[7-(5-amino-2-chloro)phenyl]pyrazolo[1,5-a]pyrimidin-3-yl}-2-thienyl-methanone and isobutyl chlorofomate, mp 234-236° C.

### EXAMPLE 234

### [7-(2-Chloro-5-nitrophenyl)pyrazolo[1,5-a]pyrimidin-3-yl](thien-2-yl)methanone

The title compound was prepared using an analogous procedure to Method A from (3-amino-1H-pyrazol-4-yl)-2-thienyl-methanone and 3-(dimethylamino)-1-(2-chloro-5-nitrophenyl)-2-propen-1-one, mp 162-166° C.

3-(Dimethylamino)-1-(2-chloro-5-nitrophenyl)-2-propen-1-one was prepared using an analogous procedure to Method B from 2-chloro-5-nitroacetophenone and dimethyl formamide-dimethyl acetal.

### EXAMPLE 235

### N-{3-[3-(Pyridin-2-ylcarbonyl)pyrazolo[1,5-a]pyrimidin-7-yl]phenyl}cyclopropanecarboxamide

The title compound was prepared using an analogous procedure to Method D from {[7-(3-aminophenyl)-2-methyl]pyrazolo[1,5-a]pyrimidin-3-yl}-2-pyridinyl-methanone and cyclopropanecarbonyl chloride, mp 214-215° C.

{[7-(3-Aminophenyl)-2-methyl]pyrazolo[1,5-a]pyrimidin-3-yl}-2-pyridinyl-methanone was prepared from (3-amino-1H-pyrazol-4-yl)-2-pyridinyl-methanone and 3-(dimethylamino)-1-(3-nitrophenyl)-2-propen-1-one using procedures analogous to Method A and C.

### EXAMPLE 236

### N-{4-Chloro-3-[3-(thien-2-ylcarbonyl)pyrazolo[1,5-a]pyrimidin-7-yl]phenyl}-3-methylbutanamide

The title compound was prepared using an analogous procedure to Method D from {[7-(5-amino-2-chloro)phenyl]pyrazolo[1,5-a]pyrimidin-3-yl}-2-thienyl-methanone and isovaleryl chloride, mp 162-165° C.

### EXAMPLE 237

### N-{4-Chloro-3-[3-(thien-2-ylcarbonyl)pyrazolo[1,5-a]pyrimidin-7-yl]phenyl}-N′-isopropylthiourea

The title compound was prepared using an analogous procedure to Example 334 from {[7-(5-amino-2-chloro)phenyl]pyrazolo[1,5-a]pyrimidin-3-yl}-2-thienyl-methanone and isopropyl thioisocyanate, mp 194-195° C.

### EXAMPLE 238

### N-{4-Chloro-3-[3-(thien-2-ylcarbonyl)pyrazolo[1,5-a]pyrimidin-7-yl]phenyl}benzamide

The title compound was prepared using an analoous procedure to Method D from {[7-(5-amino-2-chloro)phenyl]pyrazolo[1,5-a]pyrimidin-3-yl}-2-thienyl-methanone and benzoyl chloride, mp 194-195° C.

### EXAMPLE 239

### N-{2-Fluoro-5-[3-(thien-2-ylcarbonyl)pyrazolo[1,5-a]pyrimidin-7-yl]phenyl}-3-methylbutanamide

The title compound was prepared using a procedure analogous to Method D from {[7-(3-amino-4-fluoro)phenyl]pyrazolo[1,5-a]pyrimidin-3-yl}-2-thienyl-methanone and isovaleryl chloride, mp 192-194° C.

### EXAMPLE 240

### 3-Methyl-N-{⁵-[3-(thien-2-ylcarbonyl)pyrazolo[1,5-a]pyrimidin-7-yl]thien-2-yl}butanamide

The title compound was prepared using a procedure analogous to Method D from and [7-(5-amino-2-thienyl)pyrazolo[1,5-a]pyrimidin-3-yl](thien-2-yl)methanone and isovaleryl chloride, mp 268-270° C.

### EXAMPLE 241

### Isopropyl 5-[3-(thien-2-ylcarbonyl)pyrazolo[1,5-a]pyrimidin-7-yl]thien-2-ylcarbamate

The title compound was prepared using a procedure analogous to Method H from and [7-(5-amino-2-thienyl)pyrazolo[1,5-a]pyrimidin-3-yl](thien-2-yl)methanone and isopropyl chloroformate, mp 210-215° C.

### EXAMPLE 242

### [7-(5-Aminothien-2-yl)pyrazolo [1,5-a]pyrimidin-3-yl] (thien-2-yl)methanone

The title compound was prepared using an analogous procedure to Method C from [7-(5-nitro-2-thienyl)pyrazolo[1,5-a]pyrimidin-3-yl](2-thienyl)methanone, mp 232-235° C. (dec.).

### EXAMPLE 243

### 4-Chloro-N-(3-[3-(thien-2-ylcarbonyl)pyrazolo[1,5-a]pyrimidin-7-yl]phenyl}butanamide

The title compound was prepared using a procedure analogous to Method D from [7-(3-aminophenyl)pyrazolo[1,5-a]pyrimidin-3-yl]-2-thienyl-methanone and 4-chlorobutyryl chloride, mp 97-100° C.

### EXAMPLE 244

### Isopropyl 2-fluoro-5-[3-(thien-2-ylcarbonyl)pyrazolo[1,5-a]pyrimidin-7-yl]phenylcarbamate

The title compound was prepared using a procedure analogous to Method H from {[7-(3-amino-4-fluoro)phenyl]pyrazolo[1,5-a]pyrimidin-3-yl}-2-thienyl-methanone and isopropyl chloroformate, mp >200° C. (dec).

### EXAMPLE 245

### Isopropyl 2-(dimethylamino)-5-[3-(thien-2-ylcarbonyl)pyrazolo[1,5-a]pyrimidin-7-yl]phenylcarbamate

The title compound was prepared using a procedure analogous to Method H from ([7-(3-amino-4-dimethylamino)phenyl]pyrazolo[1,5-a]pyrimidin-3-yl}-2-thienyl-methanone and isopropyl chloroformate, mp 164-165° C.

{[7-(3-Amino-4-dimethylamino)phenyl]pyrazolo[1,5-a]pyrimidin-3-yl}-2-thienyl-methanone was prepared using an analogous procedure to Method C from {[7-(4-dimethylamino-3-nitro)phenyl]pyrazolo[1,5-a]pyrimidin-3-yl}-2-thienyl-methanone.

{[7-(4-Dimethylamino-3-nitro)phenyl]pyrazolo[1,5-a]pyrimidin-3-yl}-2-thienyl-methanone was obtained as an additional product in the reaction of (3-amino-1H-pyrazol-4-yl)-2-thienyl-methanone and 3-(dimethylamino)-1-(4-fluoro-3-nitrophenyl)-2-propen-1-one in acetic acid.

### EXAMPLE 246

### 184858

### {7-[3-(Benzylamino)phenyl]pyrazolo[1,5-a]pyrimidin-3-yl}(thien-2-yl)methanone

The title compound was prepared as described in Method F, mp 90-92° C.

### EXAMPLE 247

### 3-Methyl-N-{4-[3-(thien-2-ylcarbonyl)pyrazolo[1,5-a]pyrimidin-7-yl]thien-2-yl}butanamide

The title compound was prepared using a procedure analogous to Method D from and [7-(5-amino-3-thienyl)pyrazolo[1,5-a]pyrimidin-3-yl](thien-2-yl)methanone and isovaleryl chloride, mp 234-235° C.

### EXAMPLE 248

### [7-(3-Amino-4-fluorophenyl)pyrazolo[1,5-a]pyrimidin-3-yl](thien-2-yl)methanone

The title compound was prepared using an analogous procedure to Method C from {[7-(4-fluoro-3-nitro)phenyl]pyrazolo[1,5-a]pyrimidin-3-yl}-2-thienyl-methanone, mp 241-242° C.

### EXAMPLE 249

### N-Cyclohexyl-N′-{3-[3-(thien-2-ylcarbonyl)pyrazolo[1,5-a]pyrimidin-7-yl]phenyl}urea

The title compound was prepared using a procedure analogous to Method D from {[7-(3-amino-4-fluoro)phenyl]pyrazolo[1,5-a]pyrimidin-3-yl}-2-thienyl-methanone and tert-butylacetyl chloride, mp 233-236° C.

### EXAMPLE 250

### 4,4,4-Trichloro-N-{3-[3-(thien-2-ylcarbonyl)pyrazolo[1,5-a]pyrimidin-7-yl]phenyl}butanamide

The title compound was prepared using a procedure analogous to Method D from [7-(3-aminophenyl)pyrazolo[1,5-a]pyrimidin-3-yl]-2-thienyl-methanone and 4,4,4-trichlorobutyryl chloride, mp 189-191° C.

### EXAMPLE 251

### N-{2-Fluoro-5-[3-(thien-2-ylcarbonyl)pyrazolo[1,5-a]pyrimidin-7-yl]phenyl}-3,3-dimethylbutanamide

The title compound was prepared using a procedure analogous to Method D from {[7-(3-amino-4-fluoro)phenyl]pyrazolo[1,5-a]pyrimidin-3-yl}-2-thienyl-methanone and tert-butylacetyl chloride, mp 180-181° C.

### EXAMPLE 252

### 4,4,4-Trifluoro-N-{2-fluoro-5-[3-(thien-2-ylcarbonyl)pyrazolo[1,5-a]pyrimidin-7-yl]phenyl}-3-methylbutanamide

The title compound was prepared using a procedure analoous to Method E from {[7-(3-amino-4-fluoro)phenyl]pyrazolo[1,5-a]pyrimidin-3-yl}-2-thienyl-methanone and 3-methyl-4,4,4-trifluorobutyric acid, mp 185-186° C.

### EXAMPLE 253

### bis(2,2,2-Trichloroethyl) 2-fluoro-5-[3-(thien-2-ylcarbonyl)pyrazolo[1,5-a]pyrimidin-7-yl]phenylimidodicarbonate

The title compound was prepared using a procedure analogous to Method H from {[7-(3-amino-4-fluoro)phenyl]pyrazolo[1,5-a]pyrimidin-3-yl}-2-thienyl-methanone and excess of 2,2,2-trichloroethyl chloroformate, mp 94-97° C.

### EXAMPLE 254

### 2-Fluoro-N-{3-[3-(thien-2-ylcarbonyl)pyrazolo[1,5-a]pyrimidin-7-yl]phenyl}benzamide

The title compound was prepared as described in Method D, mp 190-191° C.

### EXAMPLE 255

### 2,6-Difluoro-N-{3-[3-(thien-2-ylcarbonyl)pyrazolo[1,5-a]pyrimidin-7-yl]phenyl}benzamide

The title compound was prepared using a procedure analogous to Method D from {[7-(3-amino-4-fluoro)phenyl]pyrazolo[1,5-a]pyrimidin-3-yl}-2-thienyl-methanone and 2,6-difluorobenzoyl chloride, mp 159-160° C.

### EXAMPLE 256

### N-{3-[3-(Thien-2-ylcarbonyl)pyrazolo[1,5-a]pyrimidin-7-yl]phenyl}pyridine-2-carboxamide

The title compound was prepared using a procedure analogous to Method E from {[7-(3-amino-4-fluoro)phenyl]pyrazolo[1,5-a]pyrimidin-3-yl}-2-thienyl-methanone and picolinic acid, mp 198-200° C.

### EXAMPLE 257

### N-{5-[3-(Thien-2-ylcarbonyl)pyrazolo[1,5-a]pyrimidin-7-yl]thien-2-yl}benzamide

The title compound was prepared using a procedure analogous to Method D from and [7-(5-amino-2-thienyl)pyrazolo[1,5-a]pyrimidin-3-yl](thien-2-yl)methanone and benzoyl chloride, mp 162-165° C.

### EXAMPLE 258

### (7-{3-[(2-Phenylethyl)amino]phenyl}pyrazolo[1,5-a]pyrimidin-3-yl)(thien-2-yl)methanone

The title compound was prepared using an analogous procedure to Method F from [7-(3-aminophenyl)pyrazolo[1,5-a]pyrimidin-3-yl]-2-thienyl-methanone and phenyl acetaldehyde, mp 118-120° C.

### EXAMPLE 259

### (7-{3-[(2-Methoxybenzyl)amino]phenyl}pyrazolo[1,5-a]pyrimidin-3-yl)(thien-2-yl)methanone

The title compound was prepared using an analogous procedure to Method F from [7-(3-aminophenyl)pyrazolo[1,5-a]pyrimidin-3-yl]-2-thienyl-methanone and 2-methoxybenzaldehyde, mp 138-140° C.

### EXAMPLE 260

### N-Butyl-N′-{3-[3-(thien-2-ylcarbonyl)pyrazolo[1,5-a]pyrimidin-7-yl]phenyl}thiourea

The title compound was prepared using an analogous procedure to Example 334 from [7-(3-aminophenyl)pyrazolo[1,5-a]pyrimidin-3-yl]-2-thienyl-methanone and butyl thioisocyanate, mp 155-157° C.

### EXAMPLE 261

### (7-{3-[(3-Chlorobenzyl)amino]phenyl}pyrazolo[1,5-a]pyrimidin-3-yl)(thien-2-yl)methanone

The title compound was prepared using an analogous procedure to Method F from [7-(3-aminophenyl)pyrazolo[1,5-a]pyrimidin-3-yl]-2-thienyl-methanone and 3-chlorobenzaldehyde, mp 133-135° C.

### EXAMPLE 262

### (7-{3-[(4-Chlorobenzyl)amino]phenyl}pyrazolo[1,5-a]pyrimidin-3-yl)(thien-2-yl)methanone

The title compound was prepared using an analogous procedure to Method F from [7-(3-aminophenyl)pyrazolo[1,5-a]pyrimidin-3-yl]-2-thienyl-methanone and 4-chlorobenzaldehyde, mp 149-152° C.

### EXAMPLE 263

### (7-{3-[(2-Furylmethyl)amino]phenyl}pyrazolo[1,5-a]pyrimidin-3-yl)(thien-2-yl)methanone

The title compound was prepared using an analogous procedure to Method F from [7-(3-aminophenyl)pyrazolo[1,5-a]pyrimidin-3-yll-2-thienyl-methanone and 2-furylaldehyde, as an off-white semi-solid, mp 124-128° C. and MS (M+1)=401.1.

### EXAMPLE 264

### (7-{3-[(2-Chlorobenzyl)amino]phenyl}pyrazolo[1,5-a]pyrimidin-3-yl)(thien-2-yl)methanone

The title compound was prepared using an analogous procedure to Method F from [7-(3-aminophenyl)pyrazolo[1,5-a]pyrimidin-3-yl]-2-thienyl-methanone and 2-chlorobenzaldehyde, as an off-white semi-solid, MS (M+1) of 445.1 (no mp).

### EXAMPLE 265

### (7-{3-[(3-Furylmethyl)amino]phenyl}pyrazolo[1,5-a]pyrimidin-3-yl)(thien-2-yl)methanone

The title compound was prepared using an analogous procedure to Method F from [7-(3-aminophenyl)pyrazolo[1,5-a]pyrimidin-3-yl]-2-thienyl-methanone and 3-furylaldehyde, mp 140-144° C.

### EXAMPLE 266

### (7-{3-[(2E)-But-2-enylamino]phenyl}pyrazolo[1,5-a]pyrimidin-3-yl)(thien-2-yl)methanone

The title compound was prepared using an analogous procedure to Method F from [7-(3-aminophenyl)pyrazolo[1,5-a]pyrimidin-3-yl]-2-thienyl-methanone and crotonaldehyde, mp 110-113° C.

### EXAMPLE 267

### (7-{3-[(3-Methylbut-2-enyl)amino]phenyl}pyrazolo[1,5-a]pyrimidin-3-yl)(thien-2-yl)methanone

The title compound was prepared using an analogous procedure to Method F from [7-(3-aminophenyl)pyrazolo[1,5-a]pyrimidin-3-yl]-2-thienyl-methanone and 3-methyl-3-butenal, mp 116-118° C.

### EXAMPLE 268

### [7-(6-Chloropyridin-3-yl)pyrazolo[1,5-a]pyrimidin-3-yl](thien-2-yl)methanone

The title compound was prepared from (3-amino-1H-pyrazol-4-yl)-2-thienyl-methanone and (2E)-1-(6-chloropyridine-3-yl)-3-(dimethylamino)prop-2-en-1-one by an analogous procedure to Method A, mp 145-148° C.

(2E)-1-(6-Chloropyridine-3-yl)-3-(dimethylamino)prop-2-en-1-one was prepared from 6-chloro-3-acetyl-pyridine and dimethylformamide-dimethylacetal using an analogous to Method B.

### EXAMPLE 269

### 2,6-Difluoro-N-{2-fluoro-5-[3-(thien-2-ylcarbonyl)pyrazolo[1,5-a]pyrimidin-7-yl]phenyl}benzamide

The title compound was prepared using a procedure analogous to Method D from {[7-(3-amino-4-fluoro)phenyl]pyrazolo[1,5-a]pyrimidin-3-yl}-2-thienyl-methanone and 2,6-difluorobenzoyl chloride, mp 198-200° C.

### EXAMPLE 270

### 2-fluoro-N-{2-fluoro-5-[3-(thien-2-ylcarbonyl)pyrazolo[1,5-a]pyrimidin-7-yl]phenyl}benzamide

The title compound was prepared using a procedure analogous to Method D from {[7-(3-amino-4-fluoro)phenyl]pyrazolo[1,5-a]pyrimidin-3-yl}-2-thienyl-methanone and 2-fluorobenzoyl chloride, mp 214-216° C.

### EXAMPLE 271

### 2-Chloro-N-{2-fluoro-5-[3-(thien-2-ylcarbonyl)pyrazolo[1,5-a]pyrimidin-7-yl]phenyl}benzamide

The title compound was prepared using a procedure analogous to Method D from {[7-(3-amino-4-fluoro)phenyl]pyrazolo[1,5-a]pyrimidin-3-yl}-2-thienyl-methanone and 2-chlorobenzoyl chloride, mp 166-168° C.

### EXAMPLE 272

### (7-{3-[(2-Methylbenzyl)amino]phenyl}pyrazolo[1,5-a]pyrimidin-3-yl)(thien-2-yl)methanone

The title compound was prepared using an analogous procedure to Method F from [7-(3-aminophenyl)pyrazolo[1,5-a]pyrimidin-3-yl]-2-thienyl-methanone and 2-methylbenzaldehyde, mp 177-179° C.

### EXAMPLE 273

### (7-{3-[(2-Phenylpropyl)amino]phenyl}pyrazolo[1,5-a]pyrimidin-3-yl)(thien-2-yl)methanone

The title compound was prepared using an analogous procedure to Method F from [7-(3-aminophenyl)pyrazolo[1,5-a]pyrimidin-3-yl]-2-thienyl-methanone and 2-phenylpropinonaldehyde, mp 108-110° C.

### EXAMPLE 274

### (7-{3-[(Cyclohexylmethyl)amino]phenyl}pyrazolo[1,5-a]pyrimidin-3-yl)(thien-2-yl)methanone

The title compound was prepared using an analogous procedure to Method F from [7-(3-aminophenyl)pyrazolo[1,5-a]pyrimidin-3-yl]-2-thienyl-methanone and cyclohexanecarboxaldehyde, mp 101-104° C.

### EXAMPLE 275

### 2-Chloroethyl 2-fluoro-5-[3-(thien-2-ylcarbonyl)pyrazolo [1,5-a]pyrimidin-7-yl]phenylcarbamate

The title compound was prepared using a procedure analogous to Method H from {[7-(3-amino-4-fluoro)phenyl]pyrazolo[1,5-a]pyrimidin-3-yl}-2-thienyl-methanone and 2-chloroethyl chloroformate, mp 194-196° C.

### EXAMPLE 276

### WAY0185720

### N-{2-Fluoro-5-[3-(thien-2-ylcarbonyl)pyrazolo[1,5-a]pyrimidin-7-yl]phenyl}-N′-isopropylurea

The title compound was prepared using a procedure analogous to Method G from ([7-(3-amino-4-fluoro)phenyl]pyrazolo[1,5-a]pyrimidin-3-yl}-2-thienyl-methanone and isopropyl isocyanate, mp >200° C.

### EXAMPLE 277

### 4-(1H-pyrrol-1-yl)-N-{3-[3-(thien-2-ylcarbonyl)pyrazolo[1,5-a]pyrimidin-7-yl]phenyl}benzamide

The title compound was prepared using an analogous procedure to Method E from [7-(3-aminophenyl)pyrazolo[1,5-a]pyrimidin-3-yl]-2-thienyl-methanone and 4-(1H-pyrrol-1-yl)benzoic acid, mp 224-226° C.

### EXAMPLE 278

### 2,6-Dichloro-N-{3-[3-(thien-2-ylcarbonyl)pyrazolo[1,5-a]pyrimidin-7-yl]phenyl}benzamide

The title compound was prepared using an analogous procedure to Method D from [7-(3-aminophenyl)pyrazolo[1,5-a]pyrimidin-3-yl]-2-thienyl-methanone and 2,6-dichlorobenzoyl chloride, mp 245-246° C.

### EXAMPLE 279

### N-{3-[3-(Thien-2-ylcarbonyl)pyrazolo[1,5-a]pyrimidin-7-yl]phenyl}pyrazine-2-carboxamide

The title compound was prepared using an analogous procedure to Method E from [7-(3-aminophenyl)pyrazolo [1,5-a]pyrimidin-3-yl]-2-thienyl-methanone and 2-pyrazinecarboxylic acid, mp 222-224° C.

### EXAMPLE 280

### Phenyl N′-cyano-N-{3-[3-(thien-2-ylcarbonyl)pyrazolo[1,5-a]pyrimidin-7-yl]phenyl}imidocarbamate

To a mixture of [7-(3-aminophenyl)pyrazolo[1,5-a]pyrimidin-3-yl]-2-thienyl-methanone (200 mg, 0.63 mmol) and diphenylcyanocarbonimidate (149 mg, 0.63 mmol) was added acetonitrile (3 mL). The reaction mixture was heated at reflux for 3 hours, then stirred at room temperature overnight. The resultant suspension was slurried with acetonitrile, filtered and washed with acetonitrile. The crude solid was further purified with flash column chromatography eluting with 0.5% methanol/methylene chloride to give 160 mg (55%) of phenyl N′-cyano-N′-{3-[3-(thien-2-ylcarbonyl)pyrazolo[1,5-a]pyrimidin-7-yl]phenyl}imidocarbamate as an off-white solid, mp 220° C. (dec.).

### EXAMPLE 281

### 1-Methyl-N-{3-[3-(thien-2-ylcarbonyl)pyrazolo[1,5-a]pyrimidin-7-yl]phenyl}-1H-pyrrole-2-carboxamide

The title compound was prepared using an analogous procedure to Method E from [7-(3-aminophenyl)pyrazolo[1,5-a]pyrimidin-3-yl]-2-thienyl-methanone and N-methyl-2-pyrrolcarboxylic acid, mp 120° C. (dec.).

### EXAMPLE 282

### N-{2-Fluoro-5-[3-(thien-2-ylcarbonyl)pyrazolo[1,5-a]pyrimidin-7-yl]phenyl}isonicotinamide

The title compound was prepared using an analogous procedure to Method D from {[7-(3-amino-4-fluoro)phenyl]pyrazolo[1,5-a]pyrimidin-3-yl}-2-thienyl-methanone and isonicotinyl chloride, mp 175-179° C.

### EXAMPLE 283

### N-Isopropyl-N′-{3-[3-(thien-2-ylcarbonyl)pyrazolo[1,5-a]pyrimidin-7-yl]phenyl}thiourea

The title compound was prepared using an analogous procedure to Example 334 from [7-(3-aminophenyl)pyrazolo[1,5-a]pyrimidin-3-yl]-2-thienyl-methanone and isopropyl thioisocyanate, mp 208-210° C.

### EXAMPLE 284

### (7-Chloropyrazolo[1,5-a]pyrimidin-3-yl)(thien-2-yl)methanone

A mixture of (4-oxo-pyrazolo[1,5-a]pyrimidin-3-yl)(thien-2-yl)methanone (100 mg, 0.41 mmol), phosphorous oxychloride (313 mg, 2.0 mmol) and N,N-diethylaniline (73 mg, 0.49 mmol) in toluene (4 mL) was heated at reflux for 6 hours followed by stirring at room temperature overnight. The resultant suspension was filtered and the filtrate was partitioned between water and ethyl acetate. The combined organics were dried over sodium sulfate, concentrated and purified by flash column chromatography to give 27 mg (25%) of the title compound as a light yellow solid, mp 148-150° C.

Preparation of (4-oxo-pyrazolo[1,5-a]pyrimidin-3-yl)(thien-2-yl)methanone: Sodium suspension (30% in toluene) (794 mg, 10.4 mmol) was diluted with toluene (50 mL). Ethyl acetate was added via syringe dropwise, followed by the addition of ethyl formate (766 mg, 10.4 mmol) over 5 minutes. The reaction mixture was stirred at room temperature overnight. (3-Amino-1H-pyrazol-4-yl)-2-thienyl-methanone (1.0 g, 5.2 mmol) in ethanol (40 mL) was added via syringe and the reaction mixture was heated at reflux for 24 hours. Volatiles were evaporated under reduced pressure and the residue was slurried in water. The mixture was acidified with acetic acid to pH 4-5. The precipitate was collected by filtration, washed with diluted acetic acid, water and ether and dried to give 464 mg (37%) of (4-oxo-pyrazolo[1,5-a]pyrimidin-3-yl)(thien-2-yl)methanone as a light pink solid.

### EXAMPLE 285

### 2,6-Dichloro-N-(2,6-dichlorobenzoyl)-N-{3-[3-(thien-2-ylcarbonyl)pyrazolo[1,5-a]pyrimidin-7-yl]phenyl}benzamide

The title compound was prepared using an analogous procedure to Method D from [7-(3-aminophenyl)pyrazolo[1,5-a]pyrimidin-3-yl]-2-thienyl-methanone and excess of 2,6-dichlorobenzoyl chloride, mp 238-240° C.

### EXAMPLE 286

### tert-Butyl 4-[2-oxo-2-({3-[3-(thien-2-ylcarbonyl)pyrazolo[1.5-a]pyrimidin-7-yl]phenyl}amino)ethyl]piperidine-1-carboxylate

The title compound was prepared using an analogous procedure to Method E from [7-(3-aminophenyl)pyrazolo[1,5-a]pyrimidin-3-yl]-2-thienyl-methanone and 1-t-Boc-piperidineacetic acid, mp 173-175° C.

### EXAMPLE 287

### 2-Piperidin-4-yl-N-{3-[3-(thien-2-ylcarbonyl)pyrazolo[1,5-a]pyrimidin-7-yl]phenyl}acetamide

A mixture of tert-butyl 4-[2-oxo-2-({3-[3-(thien-2-ylcarbonyl)pyrazolo[1,5-a]pyrimidin-7-yl]phenyl}amino)ethyl]piperidine-1-carboxylate (Example 286) (280 mg, 0.48 mmol) and trifluoroacetic acid (0.37 mL) in methylene chloride (6 ml) was stirred at room temperature overnight. The reaction mixture was evaporated under reduced pressure and the residue was slurried in saturated aqueous sodium bicarbonate. The solid was collected by filtration and recrystalized with ethyl acetate/hexanes to give 210 mg (91%) of the title compound as an off-white solid, mp 216-220° C.

### EXAMPLE 288

### [7-(3-{[(2E)-3-(2-Methoxyphenyl)prop-2-enyl]amino}phenyl)pyrazolo[1,5-a]pyrimidin-3-yl](thien-2-yl)methanone

The title compound was prepared using an analogous procedure to Method F from [7-(3-aminophenyl)pyrazolo[1,5-a]pyrimidin-3-yl]-2-thienyl-methanone and 2-methoxycinnamaldehyde, mp 228-131° C.

### EXAMPLE 289

### [7-(3-{[(2E)-3-(4-Methoxyphenyl)prop-2-enyl]amino}phenyl)pyrazolo[1,5-a]pyrimidin-3-yl](thien-2-yl)methanone

The title compound was prepared using an analogous procedure to Method F from [7-(3-aminophenyl)pyrazolo[1,5-a]pyrimidin-3-yl]-2-thienyl-methanone and 4-methoxycinnamaldehyde, mp 176-179° C.

### EXAMPLE 290

### [7-(3-{[(2E)-2-Methyl-3-phenylprop-2-enyl]amino}phenyl)pyrazolo[1,5-a]pyrimidin-3-yl](thien-2-yl)methanone

The title compound was prepared using an analogous procedure to Method F from [7-(3-aminophenyl)pyrazolo[1,5-a]pyrimidin-3-yl]-2-thienyl-methanone and α-methyl-trans-cinnamaldehyde, mp 74-76° C.

### EXAMPLE 291

### [7-(3-{[(2E)-3-(4-Nitrophenyl)prop-2-enyl]amino}phenyl)pyrazolo[1,5-a]pyrimidin-3-yl](thien-2-yl)methanone

The title compound was prepared using an analogous procedure to Method F from [7-(3-aminophenyl)pyrazolo[1,5-a]pyrimidin-3-yl]-2-thienyl-methanone and 4-nitrocinnamaldehyde, mp 163-166° C.

### EXAMPLE 292

### (7-{3-[(3,3-Diphenylprop-2-enyl)amino]phenyl}pyrazolo[1,5-a]pyrimidin-3-yl)(thien-2-yl)methanone

The title compound was prepared using an analogous procedure to Method F from [7-(3-aminophenyl)pyrazolo[1,5-a]pyrimidin-3-yl]-2-thienyl-methanone and β-phenylcinnamaldehyde, mp 156-158° C.

### EXAMPLE 293

### (7-{3-[(3-Phenylbutyl)amino]phenyl}pyrazolo[1,5-a]pyrimidin-3-yl)(thien-2-yl)methanone

The title compound was prepared using an analogous procedure to Method F from [7-(3-aminophenyl)pyrazolo[1,5-a]pyrimidin-3-yl]-2-thienyl-methanone and 3- phenylbutyraldehyde, mp 64-68° C.

### EXAMPLE 294

### [7-(3-{[(2E)-3-(4-Hydroxy-3-methoxyphenyl)prop-2-enyl]amino}phenyl)pyrazolo[1,5-a]pyrimidin-3-yl](thien-2-yl)methanone

The title compound was prepared using an analogous procedure to Method F from [7-(3-aminophenyl)pyrazolo[1,5-a]pyrimidin-3-yl]-2-thienyl-methanone and 3,4-dimethoxycinnamaldehyde, mp 180-184° C.

### EXAMPLE 295

### tert-Butyl 4-[({3-[3-[3-(thien-2-ylcarbonyl)pyrazolo[1,5-a]pyrimidin-7-yl]phenyl}amino)carbonyl]piperidine-1-carboxylate

The title compound was prepared using an analogous procedure to Method E from [7-(3-aminophenyl)pyrazolo[1,5-a]pyrimidin-3-yl]-2-thienyl-methanone and 1-t-Boc-carboxylic acid, mp 148-150° C.

### EXAMPLE 296

### N,N′-bis{3-[3-(Thien-2-ylcarbonyl)pyrazolo[1,5-a]pyrimidin-7-yl]phenyl}thiourea

The title compound was obtained as an additional product in the preparation of Example 334, mp 151-153° C.

### EXAMPLE 297

### Isobutyl 3-(3-thien-2-ylpyrazolo[1,5-a]pyrimidin-7-yl)phenylcarbamate

The title compound was prepared using a procedure analogous to Example 325 from isobutyl 3-(3-bromopyrazolo[1,5-a]pyrimidin-7-yl)phenylcarbamate and 2-thiopheneboronic acid, mp 140-141° C.

### EXAMPLE 298

### N-{3-[3-(Thien-2-ylcarbonyl)pyrazolo[1,5-a]pyrimidin-7-yl]phenyl}piperidine-4-carboxamide

The title compound was prepared using an analogous procedure to Example 293 from tert-butyl 4-[({3-[3-(thien-2-ylcarbonyl)pyrazolo[1,5-a]pyrimidin-7-yl]phenyl}amino)carbonyl]-piperidine-1-carboxylate, mp 159-161° C.

### EXAMPLE 299

### [7-(3-{[(2E)-2-Hexyl-3-phenylprop-2-enyl]amino}phenyl)pyrazolo[1,5-a]pyrimidin-3-yl](thien-2-yl)methanone

The title compound was prepared using an analogous procedure to Method F from [7-(3-aminophenyl)pyrazolo[1,5-a]pyrimidin-3-yl]-2-thienyl-methanone and 2-hexylcinnamaldehyde, mp 110-115° C.

### EXAMPLE 300

### [7-(3-{[(2E)-2-Pentyl-3-phenylprop-2-enyl]amino}phenyl)pyrazolo[1,5-a]pyrimidin-3-yl](thien-2-yl)methanone

The title compound was prepared using an analogous procedure to Method F from [7-(3-aminophenyl)pyrazolo[1,5-a]pyrimidin-3-yl]-2-thienyl-methanone and 2-pentylcinnamaldehyde, mp 110-112° C.

### EXAMPLE 301

### N-Isopropyl-N′-{3-[3-(thien-2-ylcarbonyl)pyrazolo[1,5-a]pyrimidin-7-yl]phenyl}guanidine

[7-(3-Aminophenyl)pyrazolo[1,5-a]pyrimidin-3-yl]-2-thienyl-methanone (100 mg, 0.31 mmol) was suspended in methanol (10 mL) and cooled to 0° C. with an ice bath. Hydrogen chloride gas was bubbled into the suspension for 30 minutes. The volatiles were evaporated under reduced pressure and the residue was dried to give an orange solid which was used for the next step without further purification, mp >200° C.

[7-(3-Aminophenyl)pyrazolo[1,5-a]pyrimidin-3-yl]-2-thienyl-methanone hydrochloride salt (100 mg, 0.31 mmol) and isopropyl cyanamide (35 mg, 0.41 mmol) in methylene chloride (5.0 mL) were heated at reflux overnight. The reaction mixture was cooled, concentrated on silica gel and purified by flash column chromatography eluting with a gradient mixture of methanol/methylene chloride to give 84 mg (55%) of the title compound as an off-white solid.

Preparation of isopropyl cyanamide: Cyanogen bromide (1.13 g, 10.7 mmol) in ether (5 mL) was added slowly to a solution of isopropylamine (1 g, 16.9 mmol) in ether (10 mL) with cooling of an ice bath. The reaction mixture was stirred overnight at room temperature, filtered and washed with ether. The combined organics were concentrated to give a yellow liquid which was used for the next step directly.

### EXAMPLE 302

### [7-(3-{[(2E)-3-Phenylprop-2-enyl]amino}phenyl)pyrazolo[1,5-a]pyrimidin-3-yl](thien-2-yl)methanone

The title compound was prepared using an analogous procedure to Method F from [7-(3-aminophenyl)pyrazolo[1,5-a]pyrimidin-3-yl]-2-thienyi-methanone and cinnamaldehyde, mp 123-125° C.

### EXAMPLE 303

### [7-(3-{[(2E)-3-(4-tert-Butylphenyl)-2-methylprop-2-enyl]amino}phenyl)pyrazolo[1,5-a]pyrimidin-3-yl](thien-2-yl)methanone

The title compound was prepared using an analogous procedure to Method F from [7-(3-aminophenyl)pyrazolo[1,5-a]pyrimidin-3-yl]-2-thienyi-methanone and α-methyl-4-tert-butylcinnamaldehyde, mp 135-137° C.

### EXAMPLE 304

### N″-Cyano-N-isopropyl-N′-{3-[3-(thien-2-ylcarbonyl)pyrazolo[1,5-a]pyrimidin-7-yl]phenyl}guanidine

A mixture of phenyl N′-cyano-N′-{3-[3-(thien-2-ylcarbonyl)pyrazolo[1,5-a]pyrimidin-7-yl]phenyl}imidocarbamate (Example 386) (120 mg, 0.26 mmol) and isopropylamine (30 mg, 0.52 mmol) in 2-propanol (3 mL) were heated at reflux for 72 hours. The resultant mixture was cooled and filtered. The crude solid was further purified by flash column chromatography eluting with a gradient mixture of methanol and methylene chloride to give 45 mg (41%) of the title compound as a white solid.

### EXAMPLE 305

### N-{3-[3-(Thien-2-ylcarbonyl)pyrazolo[1,5-a]pyrimidin-7-yl]phenyl}-1H-imidazole-5-carboxamide

A mixture of [7-(3-aminophenyl)pyrazolo[1,5-a]pyrimidin-3-yl]-2-thienyl-methanone (100 mg, 0.31 mmol) and 4-imidazolecarbonyl chloride-HCl (157 mg, 0.94 mmol) (J. Org. Chem. 1998:8084) was suspended in acetic acid (30 mL). Sodium acetate (256 mg, 3.13 mmol) in acetic acid (10 mL) was added dropwise via a dropping funnel over 60 minutes. The resultant mixture was stirred at room temperature for 3 days and evaporated under reduced pressure to remove the volatiles. The residue was purified by flash column chromatography eluting with 2% methanol/methylene chloride to give 63 mg (49%) of the title compound as a white solid, mp 280-282° C.

### EXAMPLE 306

### Pyridin-4-ylmethyl 3-[3-(thien-2-ylcarbonyl)pyrazolo[1,5-a]pyrimidin-7-yl]phenylcarbamate

The title compound was prepared using an analogous procedure to Method I from [7-(3-aminophenyl)pyrazolo[1,5-a]pyrimidin-3-yl]-2-thienyl-methanone and 4-pyridylcarbinol, mp 185° C. (dec.).

### EXAMPLE 307

### 1-Methyl-N-{3-[3-(thien-2-ylcarbonyl)pyrazolo[1,5-a]pyrimidin-7-yl]phenyl}piperidine-4-carboxamide Example

The title compound was prepared using an analogous procedure to Method E from [7-(3-aminophenyl)pyrazolo[1,5-a]pyrimidin-3-yl]-2-thienyl-methanone and N-methylpiperazinecarboxylic acid, mp 236° C.

### EXAMPLE 308

### (7-{3-[(2-Ethylhexyl)amino]phenyl}pyrazolo[1,5-a]pyrimidin-3-yl)(thien-2-yl)methanone

The title compound was prepared using an analogous procedure to Method F from [7-(3-aminophenyl)pyrazolo[1,5-a]pyrimidin-3-yl]-2-thienyl-methanone and 2-ethylhexanal, mp 134-138° C.

### EXAMPLE 309

### 3-(1,1-Dioxidothiomorpholin-4-yl)propyl 3-[3-(thien-2-ylcarbonyl)pyrazolo[1,5-a]pyrimidin-7-yl]phenylcarbamate

The title compound was prepared using an analogous procedure to Method I from [7-(3-aminophenyl)pyrazolo[1,5-a]pyrimidin-3-yl]-2-thienyl-methanone and (1,1-dioxidothiomorpholin-4-yl)propanol (International publication WO 98/13354), mp 148-150° C.

### EXAMPLE 310

### 2-Chloro-N-{2-(dimethylamino)-5-[3-(thien-2-ylcarbonyl)pyrazolo[1,5-a]pyrimidin-7-yl]phenyl}benzamide

The title compound was prepared using an analogous procedure to Method D from [7-(3-amino-4-dimethylaminophenyl)pyrazolo[1,5-a]pyrimidin-3-yl]-2-thienyl-methanone and 2-chlorobenzoyl chloride, mp 220-222° C.

### EXAMPLE 311

### 4-Morpholin-4-yl-N-{3-[3-(thien-2-ylcarbonyl)pyrazolo [1,5-a]pyrimidin-7-yl]phenyl}butanamide

The title compound was prepared using an analogous procedure to Method E from [7-(3-aminophenyl)pyrazolo[1,5-a]pyrimidin-3-yl]-2-thienyl-methanone and 3-(1-morpholine)butyric acid, mp 127-133° C.

### EXAMPLE 312

### Pyridin-3-ylmethyl-3-[3-(thien-2-ylcarbonyl)pyrazolo[1,5-a]pyrimidin-7-yl]phenylcarbamate

The title compound was prepared using an analogous procedure to Method I from [7-(3-aminophenyl)pyrazolo[1,5-a]pyrimidin-3-yl]-2-thienyl-methanone and 3-pyridylcarbinol, mp >200° C.

### EXAMPLE 313

### 3-Morpholin-4-ylpropyl 3-[3-(thien-2-ylcarbonyl)pyrazolo[1,5-a]pyrimidin-7-yl]phenylcarbamate

The title compound was prepared using an analogous procedure to Method I from [7-(3-aminophenyl)pyrazolo[1,5-a]pyrimidin-3-yl]-2-thienyl-methanone and 3-morpholinepropanol, mp 198-199° C.

### EXAMPLE 314

### [7-(3-{[(2E)-2-Methylpent-2-enyl]amino}phenyl)pyrazolo[1,5-a]pyrimidin-3-yl](thien-2-yl)methanone

The title compound was prepared using an analogous procedure to Method F from [7-(3-aminophenyl)pyrazolo[1,5-a]pyrimidin-3-yl]-2-thienyl-methanone and 2-methyl-2-pentenal, mp 132-133° C.

### EXAMPLE 315

### 2-Pyridin-4-yl-N-{3-[3-(thien-2-ylcarbonyl)pyrazolo[1,5-a]pyrimidin-7-yl]phenyl}acetamide

The title compound was prepared using an analogous procedure to Method E from [7-(3-aminophenyl)pyrazolo[1,5-a]pyrimidin-3-yl]-2-thienyl-methanone and 4-pyridineacetic acid, mp 130-131° C.

### EXAMPLE 316

### N-{2-(Dimethylamino)-5-[3-(thien-2-ylcarbonyl)pyrazolo[1,5-a]pyrimidin-7-yl]phenyl}isonicotinamide

The title compound was prepared using an analogous procedure to Method D from [7-(3-amino-4-dimethylaminophenyl)pyrazolo[1,5-a]pyrimidin-3-yl]-2-thienyl-methanone and isonicotinyl chloride, mp 151-153° C.

### EXAMPLE 317

### Pyridin-2-ylmethyl 3-[3-(thien-2-ylcarbonyl)pyrazolo[1,5-a]pyrimidin-7-yl]phenylcarbamate

The title compound was prepared using an analogous procedure to Method I from [7-(3-aminophenyl)pyrazolo[1,5-a]pyrimidin-3-yl]-2-thienyl-methanone and 2-pyridylcarbinol, mp 121-125° C.

### EXAMPLE 321

### 7

### Thien-2-yl(7-{3-[(3,5,5-trimethylhexyl]amino]phenyl}pyrazolo[1,5-a]pyrimidin-3-yl](thien-2-yl)methanone

The title compound was prepared using an analogous procedure to Method F from [7-(3-aminophenyl)pyrazolo[1,5-a]pyrimidin-3-yl]-2-thienyl-methanone and 3.5,5-trimethylhexanal, mp 81-85° C.

### EXAMPLE 324

### (7-{3-[(cyclohex-3-en-1-ylmethyl)emnino]phenyl}pyrazolo[1,5-a]pyrimidin-3-yl)-(thien-2-yl)-methanone

The title compound was prepared using an analogous procedure to Method F from [7-(3-aminophenyl)pyrazolo[1,5-a]pyrimidin-3-yl]-2-thienyl-methanone and 3-cyclohexenecarboxyaldehyde, mp 78-81° C.

### EXAMPLE 325

### 1-{3-[3-(Thien-2-ylcarbonyl)pyrazolo[1,5-a]pyrimidin-7-yl]phenyl}pyrrolidin-2-one

The title compound was prepared from [7-(3-aminophenyl)pyrazolo[1,5-a]pyrimidin-3-yl]-2-thienyl-methanone and 4-bromobutyryl chloride by an analogous procedure to Method D, mp>200° C.

### EXAMPLE 326

### 2-Chloro-N-(2-chlorobenzoyl)-N-{2-fluoro-5-[3-(thien-2-ylcarbonyl)pyrazolo[1,5-a]pyrimidin-7-yl]phenyl}benzamide

The title compound was prepared from [7-(3-aminophenyl)pyrazolo[1,5-a]pyrimidin-3-yl]-2-thienyl-methanone and excess amount of 2-chlorobenzoyl chloride by an analogous procedure to Method D, mp 150-151° C.

### EXAMPLE 327

### 2-Pyridin-3-yl-N-{3-[3-(thien-2-ylcarbonyl)pyrazolo[1,5-a]pyrimidin-7-yl]phenyl}acetamide

The title compound was prepared using an analogous procedure to Method E from [7-(3-aminophenyl)pyrazolo[1,5-a]pyrimidin-3-yl]-2-thienyl-methanone and 3-pyridineacetic acid, mp>200° C.

### EXAMPLE 328

### 3-Pyridin-4-yl-N-{3-[3-(thien-2-ylcarbonyl)pyrazolo[1,5-a]pyrimidin-7-yl]phenyl}propanamide

The title compound was prepared using an analogous procedure to Method E from [7-(3-aminophenyl)pyrazolo[1,5-a]pyrimidin-3-yl]-2-thienyl-methanone and 4-pyridinepropionic acid, mp 176-181° C.

### EXAMPLE 329

### N-{3-[3-(Thien-2-ylcarbonyl)pyrazolo[1,5-a]pyrimidin-7-yl]phenyl}-1H-pyrazole-4-carboxamide

The title compound was prepared using an analogous procedure to Method E from [7-(3-aminophenyl)pyrazolo[1,5-a]pyrimidin-3-yl]-2-thienyl-methanone and 3-pyrazolecarboxylic acid, mp 165-170° C.

### EXAMPLE 330

### 4-Oxo-4-({3-[3-(thien-2-ylcarbonyl)pyrazolo[1,5-a]pyrimidin-7-yl]phenyl}amino)butanoic acid

To a suspension of [7-(3-aminophenyl)pyrazolo[1,5-a]pyrimidin-3-yl]-2-thienyl-methanone (100 mg, 0.31 mmol) in dioxane (6 mL) was added succinic anhydride (47 mg, 0.47 mmol). The reaction mixture was stirred at room temperature overnight followed by stirring at 60° C. for 12 hours, cooled and diluted with ether. The precipitate was collected by filtration and washed with ether to afford 106 mg (82%) of the title compound as an off-white solid, mp 240-241° C.

### EXAMPLE 331

### 2-[({3-[3-(Thien-2-ylcarbonyl)pyrazolo[1,5-a]pyrimidin-7-yl]phenyl}amino)carbonyl]benzoic acid

The title compound was prepared from [7-(3-aminophenyl)pyrazolo [1,5-a]pyrimidin-3-yl]-2-thienyl-methanone and phthalic anhydride by an analogous procedure described in

### EXAMPLE 330, mp 215-216° C.

### EXAMPLE 332

### 5-Oxo-5-({3-[3-(thien-2-ylcarbonyl)pyrazolo[1,5-a]pyrimidin-7-yl]phenyl}amino)pentanoic acid

The title compound was prepared from [7-(3-aminophenyl)pyrazolo[1,5-a]pyrimidin-3-yl]-2-thienyl-methanone and glutaric anhydride by an analogous procedure described in Example 330, mp 189-190° C.

### EXAMPLE 333

### 3-Methyl-5-oxo-5-({3-[3-(thien-2-ylcarbonyl)pyrazolo[1,5-a]pyrimidin-7-yl]phenyl}amino)pentanoic acid

The title compound was prepared from [7-(3-aminophenyl)pyrazolo[1,5-a]pyrimidin-3-yl]-2-thienyl-methanone and 3-methylglutaric anhydride by an analogous procedure described in Example 330, mp 135-136° C.

### EXAMPLE 334

### 4,5-Dichloro-2-[({3-[3-(thien-2-ylcarbonyl)pyrazolo[1,5-a]pyrimidin-7-yl]phenyl}amino)carbonyl]benzoic acid

The title compound was prepared from [7-(3-aminophenyl)pyrazolo[1,5-a]pyrimidin-3-yl]-2-thienyl-methanone and 4, 5-dichlorophthalic arlydride by an analogous procedure described in Example 330, mp>270° C.

### EXAMPLE 335

### (7-{3-[(2-Methylundecyl)amino]phenyl}pyrazolo[1,5-a]pyrimidin-3-yl)(thien-2-yl)methanone

The title compound was prepared from [7-(3-aminophenyl)pyrazolo[1,5-a]pyrimidin-3-yl]-2-thienyl-methanone and 2-methylundecanal by an analogous procedure described in Method F, mp 108-110° C.

### EXAMPLE 336

### 3-Pyridin-3-yl-N-{3-[3-(thien-2-ylcarbonyl)pyrazolo [1,5-a]pyrimidin-7-yl]phenyl}propanamide

The title compound was prepared using an analogous procedure to Method E from [7-(3-aminophenyl)pyrazolo[1,5-a]pyrimidin-3-yl]-2-thienyl-methanone and 3-pyridinepropionic acid, mp>200° C.

### EXAMPLE 337

### 2-(Pyridin-4-ylthio)-N-{3-[3-(thien-2-ylcarbonyl)pyrazolo[1,5-a]pyrimidin-7-yl]phenyl}acetamide

The title compound was prepared using an analogous procedure to Method E from [7-(3-aminophenyl)pyrazolo[1,5-a]pyrimidin-3-yl]-2-thienyl-methanone and (4-pyridylthio)acetic acid, mp 138-144° C.

### EXAMPLE 338

### {7-[4′-(Dimethylamino)-1,1′-biphenyl-3-yl]pyrazolo[1,5-a]pyrimidin-3-yl}(thien-2-yl)methanone

The title compound was prepared as described Method M from 4-N,N-dimethyaminophenyl boronic acid and [7-(3-bromophenyl)pyrazolo[1,5-a]pyrimidin-3-yl]-2-thienyl- methanone, mp 200-202° C.

### EXAMPLE 339

### [7-(3′-Amino-1,1′-biphenyl-3-yl)pyrazolo[1,5-a]pyrimidin-3-yl](thien-2-yl)methanone

The title compound was prepared using an analogous procedure to Method M from 3-aminophenyl boronic acid and [7-(3-bromophenyl)pyrazolo[1,5-a]pyrimidin-3-yl]-2-thienyl- methanone, mp 117-121° C.

### EXAMPLE 340

### {7-[4′-(Hydroxymethyl)-1,1′-biphenyl-3-yl]pyrazolo[1,5-a]pyrimidin-3-yl}(thien-2-yl)methanone

The title compound was prepared using an analogous procedure to Method M from 4-(hydroxymethyl)phenyl boronic acid and [7-(3-bromophenyl)pyrazolo[1,5-a]pyrimidin-3-yl]-2-thienyl- methanone, mp 106-110° C.

### EXAMPLE 341

### {7-[3′-(Dimethylamino)-1,1′-biphenyl-3-yl]pyrazolo[1,5-a]pyrimidin-3-yl}(thien-2-yl)methanone

The title compound was prepared using an analogous procedure to Method M from 4-(N,N-dimethylamino)phenyl boronic acid and [7-(3-bromophenyl)pyrazolo[1,5-a]pyrimidin-3-yl]-2-thienyl- methanone, mp 167-169° C.

### EXAMPLE 342

### {7-[4′-(Dimethylamino)-6-fluoro-1,1′-biphenyl-3-yl]pyrazolo[1,5-a]pyrimidin-3-yl}(thien-2-yl)methanone

The title compound was prepared using an analogous procedure to Method M from 4-(N,N-dimethylamino)phenyl boronic acid and [7-(3-bromo-4-fluorophenyl)pyrazolo[1,5-a]pyrimidin-3-yl]-2-thienyl- methanone, mp 219-221° C.

[7-(3-bromo-4-fluorophenyl)pyrazolo[1,5-a]pyrimidin-3-yl]-2-thienyl-methanone was prepared using an analogous procedure to Method A from (5-amino-1H-pyrazol-4-yl)(thien-2-yl)methanone and 1-(3-bromo-4-fluoro-phenyl)-3-dimethylamino-propenone, mp 74-76° C

### EXAMPLE 343

### {7-[6-Fluoro-4′-(hydroxymethyl)-1,1′-biphenyl-3-yl]pyrazolo[1,5-a]pyrimidin-3-yl}(thien-2-yl)methanone

The title compound was prepared using an analogous procedure to Method M from 4-(hydroxymethyl)phenyl boronic acid and [7-(3-bromo-4-fluorophenyl)pyrazolo[1,5-a]pyrimidin-3-yl]-2-thienyl- methanone, mp 140-145° C.

### EXAMPLE 344

### [7-(4′-Ethyl-1,1′-biphenyl-3-yl)pyrazolo[1,5-a]pyrimidin-3-yl](thien-2-yl)methanone

The title compound was prepared using an analogous procedure to Method M from 4-ethylphenyl boronic acid and [7-(3-bromo-4-fluorophenyl)pyrazolo[1,5-a]pyrimidin-3-yl]-2-thienyl- methanone, mp 117-119° C.

### EXAMPLE 345

### 3-{3′-[3-(Thien-2-ylcarbonyl)pyrazolo[1,5-a]pyrimidin-7-yl]-1,1′-biphenyl-4-yl}propanoic acid

The title compound was prepared using an analogous procedure to Method M from 4-(2-carboxyethyl)phenyl boronic acid and [7-(3-bromo-4-fluorophenyl)pyrazolo[1,5-a]pyrimidin-3-yl]-2-thienyl- methanone, mp 117-119° C.

### EXAMPLE 346

### 3′-[3-(Thien-2-ylcarbonyl)pyrazolo[1,5-a]pyrimidin-7-yl]-1,1′-biphenyl-4-carbaldehyde

The title compound was prepared using an analogous procedure to Method M from 4-formylphenyl boronic acid and [7-(3-bromo-4-fluorophenyl)pyrazolo[1,5-a]pyrimidin-3-yl]-2-thienyl- methanone, mp 156-158° C.

### EXAMPLE 347

### {7-[4′-(Morpholin-4-ylmethyl)-1,1′-biphenyl-3-yl]pyrazolo[1,5-a]pyrimidin-3-yl}(thien-2-yl)methanone

The title compound was prepared as described Method N from 3′-[3-(thien-2-ylcarbonyl)pyrazolo[1,5-a]pyrimidin-7-yl]-1,1′-biphenyl-4-carbaldehyde and morpholine, mp 175-178° C.

### EXAMPLE 348

### (7-{4′-[(4-Methylpiperazin-1-yl)methyl]-1,1′-biphenyl-3-yl}pyrazolo[1,5-a]pyrimidin-3-yl)(thien-2-yl)methanone

The title compound was prepared using an analogous procedure to Method N from 3′-[3-(thien-2-ylcarbonyl)pyrazolo[1,5-a]pyrimidin-7-yl]-1,1′-biphenyl-4-carbaldehyde and N-methylpiperazine, mp 199-202° C.

### EXAMPLE 349

### [7-(4′-{[(2-Morpholin-4-ylethyl)amino]methyl}-1,1′-biphenyl-3-yl)pyrazolo[1,5-a]pyrimidin-3-yl](thien-2-yl)methanone

The title compound was prepared using an analogous procedure to Method N from 3′-[3-(thien-2-ylcarbonyl)pyrazolo[1,5-a]pyrimidin-7-yl]-1,1′-biphenyl-4-carbaldehyde and 4-(2-aminoethyl)morpholine, mp 100-106° C.

### EXAMPLE 350

### (7-{4′-[(Diethylamino)methyl]-1,1′-biphenyl-3-yl}pyrazolo[1,5-a]pyrimidin-3-yl)(thien-2-yl)methanone

The title compound was prepared using an analogous procedure to Method N from 3′-[3-(thien-2-ylcarbonyl)pyrazolo[1,5-a]pyrimidin-7-yl]-1,1′-biphenyl-4-carbaldehyde and diethylamine, mp 144-146° C.

### EXAMPLE 351

### (7-{4′-[(Dimethylamino)methyl]-1,1′-biphenyl-3-yl}pyrazolo[1,5-a]pyrimidin-3-yl)(thien-2-yl)methanone

The title compound was prepared using an analogous procedure to Method N from 3′-[3-(thien-2-ylcarbonyl)pyrazolo[1,5-a]pyrimidin-7-yl]-1,1′-biphenyl-4-carbaldehyde and dimethylamine, mp 192-195° C.

### EXAMPLE 352

### N-{3-[3-(3-Aminophenyl)pyrazolo[1,5-a]pyrimidin-7-yl]phenyl}-3-methylbutanamide

The title compound was prepared as described Method M from N-[3-(3-bromopyrazolo[1,5-a]pyrimidin-7-yl)phenyl]-3-methylbutanamide and 3-aminophenyl boronic acid, mp 173-175° C.

N-[3-(3-Bromopyrazolo[1,5-a]pyrimidin-7-yl)phenyl]-3-methylbutanamide was prepared using an analogous procedure to Method E from 3-(3-bromopyrazolo[1,5-a]pyrimidin-7-yl)aniline and isobutyl chloroformate, mp 132-134° C.

3-(3-Bromopyrazolo[1,5-a]pyrimidin-7-yl)aniline was prepared using an analogous procedure to Method C from 3-bromo-7-(3-nitrophenyl)pyrazolo[1,5-a]pyrimidine, mp 120-121° C.

3-Bromo-7-(3-nitrophenyl)pyrazolo[1,5-a]pyrimidine was prepared as follows: To a solution of 7-(3-nitrophenyl)pyrazolo[1,5-a]pyrimidine (2.0 g, 8.3 mmol) in chloroform (20 mL) was added N-bromosuccinimide (1.56 g, 8.75 mmol) in portions and thre reaction mixture was heated at reflux for 20 minutes to resulted in a thick suspension. The solid was collected via filtration and was further purifed by silica gel flash column chromatography to give 1.98 g (75%) of the desired product as a yellow solid, mp 237-238° C.

7-(3-Nitrophenyl)pyrazolo[1,5-a]pyrimidine was prepared using an analogous procedure to Method A from 1-(3-nitro-phenyl)-3-dimethylamino-propenone and 3-aminopyrazole, mp 204-206° C.

### EXAMPLE 353

### N-(3-{3-[3-(Dimethylamino)phenyl]pyrazolo[1,5-a]pyrimidin-7-yl}phenyl)-3-methylbutanamide

The title compound was prepared using an analogous procedure to Method M from N-[3-(3-bromopyrazolo[1,5-a]pyrimidin-7-yl)phenyl]-3-methylbutanamide and 3-N,N-dimethylaminophenyl boronic acid, mp 182-184° C.

### EXAMPLE 354

### N-{3-[3-(1H-Indol-5-yl)pyrazolo[1,5-a]pyrimidin-7-yl]phenyl}-3-methylbutanamide

The title compound was prepared using an analogous procedure to Method M from N-[3-(3-bromopyrazolo[1,5-a]pyrimidin-7-yl)phenyl]-3-methylbutanamide and 5-Indolylboronic acid, mp 167-169° C.

### EXAMPLE 355

### 3-Methyl-N-{3-[3-(3,4,5-trimethoxyphenyl)pyrazolo[1,5-a]pyrimidin-7-yl]phenyl}butanamide

The title compound was prepared using an analogous procedure to Method M from N-[3-(3-bromopyrazolo[1,5-a]pyrimidin-7-yl)phenyl]-3-methylbutanamide and 3,4,5-trimethoxyphenyl boronic acid, mp 86-90° C.

### EXAMPLE 356

### 3-Methyl-N-[3-(3-quinolin-8-ylpyrazolo[1,5-a]pyrimidin-7-yl)phenyl]butanamide

The title compound was prepared using an analogous procedure to Method M from N-[3-(3-bromopyrazolo[1,5-a]pyrimidin-7-yl)phenyl]-3-methylbutanamide and 8-quinolineboronic acid, mp 96-99° C.

### EXAMPLE 357

### N-(3-{3-[4-(Hydroxymethyl)phenyl]pyrazolo[1,5-a]pyrimidin-7-yl}phenyl)-3-methylbutanamide

The title compound was prepared using an analogous procedure to Method M from N-[3-(3-bromopyrazolo[1,5-a]pyrimidin-7-yl)phenyl]-3-methylbutanamide and 4-hydroxymethylboronic acid, mp 170-172° C.

### EXAMPLE 358

### N-{3-[3-(4-Methoxyphenyl)pyrazolo[1,5-a]pyrimidin-7-yl]phenyl}-3-methylbutanamide

The title compound was prepared using an analogous procedure to Method M from N-[3-(3-bromopyrazolo[1,5-a]pyrimidin-7-yl)phenyl]-3-methylbutanamide and 4-methoxyphenyl boronic acid, mp 225-227° C.

### EXAMPLE 359

### N-{3-[3-(3-Chlorophenyl)pyrazolo[1,5-a]pyrimidin-7-yl]phenyl}-3-methylbutanamide

The title compound was prepared using an analogous procedure to Method M from N-[3-(3-bromopyrazolo[1,5-a]pyrimidin-7-yl)phenyl]-3-methylbutanamide and 3-chlorophenyl boronic acid, mp 169-171° C.

### EXAMPLE 360

### N-{3-[3-(4-Chlorophenyl)pyrazolo[1,5-a]pyrimidin-7-yl]phenyl}-3-methylbutanamide

The title compound was prepared using an analogous procedure to Method M from N-[3-(3-bromopyrazolo[1,5-a]pyrimidin-7-yl)phenyl]-3-methylbutanamide and 4-chlorophenyl boronic acid, mp 215-217° C.

### EXAMPLE 361

### N-{3-[3-(2,3-Dichlorophenyl)pyrazolo[1,5-a]pyrimidin-7-yl]phenyl}-3-methylbutanamide

The title compound was prepared using an analogous procedure to Method M from N-[3-(3-bromopyrazolo[1,5-a]pyrimidin-7-yl)phenyl]-3-methylbutanamide and 2, 3-dichlorophenyl boronic acid, mp 154-156° C.

### EXAMPLE 362

### N-{3-[3-(3,4-Dichlorophenyl)pyrazolo[1,5-a]pyrimidin-7-yl]phenyl}-3-methylbutanamide

The title compound was prepared using an analogous procedure to Method M from N-[3-(3-bromopyrazolo[1,5-a]pyrimidin-7-yl)phenyl]-3-methylbutanamide and 3, 4-dichlorophenyl boronic acid, mp 215-217° C.

### EXAMPLE 363

### N-{3-[3-(3,5-Dichlorophenyl)pyrazolo[1,5-a]pyrimidin-7-yl]phenyl}-3-methylbutanamide

The title compound was prepared using an analogous procedure to Method M from N-[3-(3-bromopyrazolo[1,5-a]pyrimidin-7-yl)phenyl]-3-methylbutanamide and 3, 5-dichlorophenyl boronic acid, mp 223-225° C.

### EXAMPLE 364

### N-{3-[3-(3,5-Dimethylphenyl)pyrazolo[1,5-a]pyrimidin-7-yl]phenyl}-3-methylbutanamide

The title compound was prepared using an analogous procedure to Method M from N-[3-(3-bromopyrazolo[1,5-a]pyrimidin-7-yl)phenyl]-3-methylbutanamide and 3, 5-dimethylphenyl boronic acid, mp 178-180° C.

### EXAMPLE 365

### 3-Methyl-N-(3-{3-[3-(trifluoromethyl)phenyl]pyrazolo[1,5-a]pyrimidin-7-yl}phenyl)butanamide

The title compound was prepared using an analogous procedure to Method M from N-[3-(3-bromopyrazolo[1,5-a]pyrimidin-7-yl)phenyl]-3-methylbutanamide and 3-trifluoromethylphenyl boronic acid, mp 170-173° C.

### EXAMPLE 366

### N-{3-[3-(3-Methoxyphenyl)pyrazolo[1,5-a]pyrimidin-7-yl]phenyl}-3-methylbutanamide

The title compound was prepared using an analogous procedure to Method M from N-[3-(3-bromopyrazolo[1,5-a]pyrimidin-7-yl)phenyl]-3-methylbutanamide and 3-methoxyphenyl boronic acid, mp 153-154° C.

### EXAMPLE 367

### N-{3-[3-(3-Ethoxyphenyl)pyrazolo[1,5-a]pyrimidin-7-yl]phenyl}-3-methylbutanamide

The title compound was prepared using an analogous procedure to Method M from N-[3-(3-bromopyrazolo[1,5-a]pyrimidin-7-yl)phenyl]-3-methylbutanamide and 3-ethoxyphenyl boronic acid, mp 156-158° C.

### EXAMPLE 368

### 3-Methyl-N-{3-[3-(4-phenoxyphenyl)pyrazolo[1,5-a]pyrimidin-7-yl]phenyl}butanamide

The title compound was prepared using an analogous procedure to Method M from N-[3-(3-bromopyrazolo[1,5-a]pyrimidin-7-yl)phenyl]-3-methylbutanamide and 4-phenoxyphenyl boronic acid, mp 175-178° C.

### EXAMPLE 369

### N-{3-[3-(1,3-Benzodioxol-5-yl)pyrazolo[1,5-a]pyrimidin-7-yl]phenyl}-3-methylbutanamide

The title compound was prepared using an analogous procedure to Method M from N-[3-(3-bromopyrazolo[1,5-a]pyrimidin-7-yl)phenyl]-3-methylbutanamide and 3,4-methylenedioxyphenyl boronic acid, mp 172-173° C.

### EXAMPLE 370

### N-{3-[3-(3-Cyanophenyl)pyrazolo[1,5-a]pyrimidin-7-yl]phenyl}-3-methylbutanamide

The title compound was prepared using an analogous procedure to Method M from N-[3-(3-bromopyrazolo[1,5-a]pyrimidin-7-yl)phenyl]-3-methylbutanamide and 3-cyanophenyl boronic acid, mp 166-167° C.

### EXAMPLE 371

### N-{3-[3-(3-Acetylphenyl)pyrazolo[1,5-a]pyrimidin-7-yl]phenyl}-3-methylbutanamide

The title compound was prepared using an analogous procedure to Method M from N-[3-(3-bromopyrazolo[1,5-a]pyrimidin-7-yl)phenyl]-3-methylbutanamide and 3-acetylphenyl boronic acid, mp 190-192° C.

### EXAMPLE 372

### N-{3-[3-(3-Formyl-4-methoxyphenyl)pyrazolo[1,5-a]pyrimidin-7-yl]phenyl}-3-methylbutanamide

The title compound was prepared using an analogous procedure to Method M from N-[3-(3-bromopyrazolo[1,5-a]pyrimidin-7-yl)phenyl]-3-methylbutanamide and 3-formyl-4-methoxyphenyl boronic acid, mp 187-189° C.

### EXAMPLE 373

### N-{3-[3-(1,1′-Biphenyl-3-yl)pyrazolo[1,5-a]pyrimidin-7-yl]phenyl}-3-methylbutanamide

The title compound was prepared using an analogous procedure to Method M from N-[3-(3-bromopyrazolo[1,5-a]pyrimidin-7-yl)phenyl]-3-methylbutanamide and 3-biphenyl boronic acid, mp 182-185° C.

### EXAMPLE 374

### N-{3-[3-(1,1′-Biphenyl-4-yl)pyrazolo[1,5-a]pyrimidin-7-yl]phenyl}-3-methylbutanamide

The title compound was prepared using an analogous procedure to Method M from N-[3-(3-bromopyrazolo[1,5-a]pyrimidin-7-yl)phenyl]-3-methylbutanamide and 4-biphenyl boronic acid, mp 157-160° C.

### EXAMPLE 375

### N-{3-[3-(3-Formylphenyl)pyrazolo[1,5-a]pyrimidin-7-yl]phenyl}-3-methylbutanamide

The title compound was prepared using an analogous procedure to Method M from N-[3-(3-bromopyrazolo[1,5-a]pyrimidin-7-yl)phenyl]-3-methylbutanamide and 3-formylphenyl boronic acid, mp 163-165° C.

### EXAMPLE 376

### 3-Methyl-N-(3-{3-[3-(morpholin-4-ylmethyl)phenyl]pyrazolo[1,5-a]pyrimidin-7-yl}phenyl)butanamide

The title compound was prepared using an analogous procedure to Method N from N-(3-[3-(3-formylphenyl)pyrazolo[1,5-a]pyrimidin-7-yl]phenyl}-3-methylbutanamide and morpholine, mp 170-173° C.

### EXAMPLE 377

### 3-Methyl-N-[3-(3-{3-[(4-methylpiperazin-1-yl)methyl]phenyl}pyrazolo[1,5-a]pyrimidin-7-yl)phenyl]butanamide

The title compound was prepared using an analogous procedure to Method N from N-{3-[3-(3-formylphenyl)pyrazolo[1,5-a]pyrimidin-7-yl]phenyl}-3-methylbutanamide and N-methylpipeprazine, mp 135-138° C.

### EXAMPLE 378

### 3-[3-(3,5-Dimethylphenyl)pyrazolo[1,5-a]pyrimidin-7-yl]benzonitrile

The title compound was prepared using an analogous procedure to Method M from 3-(3-bromopyrazolo[1,5-a]pyrimidin-7-yl)benzonitrile and 3,5-dimethylphenylboronic acid, mp 147° C.

### EXAMPLE 379

### 3-(3,5-Dimethylphenyl)-7-[3-(1H-tetraazol-5-yl)phenyl]pyrazolo[1,5-a]pyrimidine

The title compound was prepared as described in Method O from 3-[3-(3,5-dimethylphenyl)pyrazolo[1,5-a]pyrimidin-7-yl]benzonitrile and sodium azide, mp 250° C. (dec.).

### EXAMPLE 380

### 7-{3-[2-(Cyclobutylmethyl)-2H-tetraazol-5-yl]phenyl}-3-(3,5-dimethylphenyl)pyrazolo[1,5-a]pyrimidine

The title compound was prepared as described in Method P from 3-(3,5-dimethylphenyl)-7-[3-(1H-tetraazol-5-yl)phenyl]pyrazolo[1,5-a]pyrimidine and cyclobutylmethyl bromide, mp 64° C.

### EXAMPLE 381

### 7-{3-[2-(Cyclobutylmethyl)-2H-tetraazol-5-yl]phenyl}-3-(3,5-dimethylphenyl)pyrazolo[1,5-a]pyrimidine

The title compound was prepared using an analogous procedure to Method P from 3-(3,5-dimethylphenyl)-7-[3-(1H-tetraazol-5-yl)phenyl]pyrazolo[1,5-a]pyrimidine and 4-(2-chloroethyl)morpholine, mp 68° C.

### EXAMPLE 382

### 3-(3,5-Dimethylphenyl)-7-{3-[2-(pyridin-3-ylmethyl)-2H-tetrazol-5-yl]phenyl}pyrazolo[1,5-a]pyrimidine

The title compound was prepared using an analogous procedure to Method P from 3-(3,5-dimethylphenyl)-7-[3-(1H-tetraazol-5-yl)phenyl]pyrazolo[1,5-a]pyrimidine and 3-bromomethylpyridine, mp 73° C.

### EXAMPLE 383

### 3-(3,5-Dimethylphenyl)-7-{3-[2-(pyridin-2-ylmethyl)-2H-tetrazol-5-yl]phenyl}pyrazolo[1,5-a]pyrimidine

The title compound was prepared using an analogous procedure to Method P from 3-(3,5-dimethylphenyl)-7-[3-(1H-tetraazol-5-yl)phenyl]pyrazolo[1,5-a]pyrimidine and 2-bromomethylpyridine, mp 77° C.

### EXAMPLE 384

### 3-(3,5-Dimethylphenyl)-7-[3-(2-ethyl-2H-tetrazol-5-yl)phenyllpyrazolo[1,5-a]pyrimidine

The title compound was prepared using an analogous procedure to Method P from 3-(3,5-dimethylphenyl)-7-[3-(1H-tetraazol-5-yl)phenyl]pyrazolo[1,5-a]pyrimidine and bromoethane, mp 65° C.

### EXAMPLE 385

### N-[3-(3-{3-[(Dimethylamino)methyl]phenyl}pyrazolo[1,5-a]pyrimidin-7-yl)phenyl]-3-methylbutanamide

The title compound was prepared using an analogous procedure to Method N from N-{3-[3-(3-formylphenyl)pyrazolo[1,5-a]pyrimidin-7-yl]phenyl}-3-methylbutanamide and dimethylamine, mp 179-182° C.

### EXAMPLE 386

### 3-(3,5-Dimethylphenyl)-7-{3-[2-(2-pyrrolidin-1-ylethyl)-2H-tetrazol-5-yl]phenyl}pyrazolo[1,5-a]pyrimidine

The title compound was prepared using an analogous procedure to Method P from 3-(3,5-dimethylphenyl)-7-[3-(1H-tetraazol-5-yl)phenyl]pyrazolo[1,5-a]pyrimidine and 1-(2-chloroethyl)pyrrolidine, mp 60° C.

### EXAMPLE 387

### 3-(3,5-Dimethylphenyl)-7-{3-[2-(2-piperidin-1-ylethyl)-2H-tetrazol-5-yl]phenyl}pyrazolo[1,5-a]pyrimidine

The title compound was prepared using an analogous procedure to Method P from 3-(3,5-dimethylphenyl)-7-[3-(1H-tetraazol-5-yl)phenyl]pyrazolo[1,5-a]pyrimidine and 1-(2-chloroethyl)piperidine, mp 66° C.

### EXAMPLE 388

### 3-(3,5-Dimethylphenyl)-7-{3-[2-(2-piperidin-1-ylethyl)-2H-tetrazol-5-yl]phenyl}pyrazolo[1,5-a]pyrimidine

The title compound was prepared using an analogous procedure to Method P from 3-(3,5-dimethylphenyl)-7-[3-(1H-tetraazol-5-yl)phenyl]pyrazolo[1,5-a]pyrimidine and 4-bromoethylpyridine, mp 172° C.

### EXAMPLE 389

### {7-[3-(1H-Tetraazol-5-yl)phenyl]pyrazolo[1,5-a]pyrimidin-3-yl}(thien-2-yl)methanone

The title compound was prepared using an analogous procedure to Method O from 3-[3-(2-thienylcarbonyl)pyrazolo[1,5- a]-pyrimidin-7-yl]-benzonitrile and sodium azide, mp 290° C.

### EXAMPLE 390

### {7-[3-(2-Ethyl-2H-tetraazol-5-yl)phenyl]pyrazolo[1,5-a]pyrimidin-3-yl}(thien-2-yl)methanone

The title compound was prepared using an analogous procedure to Method P from {7-[3-(1H-tetraazol-5-yl)phenyl]pyrazolo[1,5-a]pyrimidin-3-yl}(thien-2-yl)methanone and ethyl iodide, mp 174° C.

### EXAMPLE 391

### (7-{3-[2-(Pyridin-3-ylmethyl)-2H-tetraazol-5-yl]phenyl}pyrazolo[1,5-a]pyrimidin-3-yl)(thien-2-yl)methanone

The title compound was prepared using an analogous procedure to Method P from {7-[3-(1H-tetraazol-5-yl)phenyl]pyrazolo[1,5-a]pyrimidin-3-yl}(thien-2-yl)methanone and 3-bromomethylpyridine, mp 95° C.

### EXAMPLE 392

### {7-[3-(2-Isobutyl-2H-tetraazol-5-yl)phenyl]pyrazolo[1,5-a]pyrimidin-3-yl}(thien-2-yl)methanone

The title compound was prepared using an analogous procedure to Method P from {7-[3-(1H-tetraazol-5-yl)phenyl]pyrazolo[1,5-a]pyrimidin-3-yl}(thien-2-yl)methanone and isobutyl bromide, mp 141° C.

### EXAMPLE 393

### (7-{3-[2-(Cyclobutylmethyl)-2H-tetraazol-5-yl]phenyl}pyrazolo[1,5-a]pyrimidin-3-yl)(thien-2-yl)methanone

The title compound was prepared using an analogous procedure to Method P from {7-[3-(1H-tetraazol-5-yl)phenyl]pyrazolo[1,5-a]pyrimidin-3-yl}(thien-2-yl)methanone and cyclobutylmethyl bromide, mp 170° C.

### EXAMPLE 394

### {7-[3-(2-Butyl-2H-tetraazol-5-yl)phenyl]pyrazolo[1,5-a]pyrimidin-3-yl }(thien-2-yl)methanone

The title compound was prepared using an analogous procedure to Method P from {7-[3-(1H-tetraazol-5-yl)phenyl]pyrazolo[1,5-a]pyrimidin-3-yl}(thien-2-yl)methanone and butyl iodide, mp 90° C.

### EXAMPLE 395

### (7-{3-[2-(2-Morpholin-4-ylethyl)-2H-tetraazol-5-yl]phenyl}pyrazolo[1,5-a]pyrimidin-3-yl)(thien-2-yl)methanone

The title compound was prepared using an analogous procedure to Method P from {7-[3-(1H-tetraazol-5-yl)phenyl]pyrazolo[1,5-a]pyrimidin-3-yl}(thien-2-yl)methanone and 4-(2-chloroethyl)morpholine, mp 103° C.

### EXAMPLE 396

### (7-{3-[1-(Cyclobutylmethyl)-1H-tetraazol-5-yl]phenyl}pyrazolo[1,5-a]pyrimidin-3-yl)(thien-2-yl)methanone

The title compound was prepared as a minor product using an analogous procedure to Method P from (7-[3-(1H-tetraazol-5-yl)phenyl]pyrazolo[1,5-a]pyrimidin-3-yl}(thien-2-yl)methanone and cyclobutylmethyl bromide, mp 204° C.

### EXAMPLE 397

### (7-{3-[2-(Pyridin-2-ylmethyl)-2H-tetraazol-5-yl]phenyl}pyrazolo[1,5-a]pyriridin-3-yl)(thien-2-yl)methanone

The title compound was prepared using an analogous procedure to Method P from {7-[3-(1H-tetraazol-5-yl)phenyl]pyrazolo[1,5-a]pyrimidin-3-yl}(thien-2-yl)methanone and 2-bromomethylpyridine, mp 184° C.

### EXAMPLE 398

### (7-{3-[2-(Pyridin-4-ylmethyl)-2H-tetraazol-5-yl]phenyl}pyrazolo[1,5-a]pyrimidin-3-yl)(thien-2-yl)methanone

The title compound was prepared using an analogous procedure to Method P from {7-[3-( 1H-tetraazol-5-yl)phenyl]pyrazolo[1,5-a]pyrimidin-3-yl}(thien-2-yl)methanone and 4-bromomethylpyridine, mp 170° C.

### EXAMPLE 399

### (7-{3-[2-(2-Pyrrolidin-1-ylethyl)-2H-tetraazol-5-yl]phenyl}pyrazolo[1,5-a]pyrimidin-3-yl)(thien-2-yl)methanone

The title compound was prepared using an analogous procedure to Method P from {7-[3-(1H-tetraazol-5-yl)phenyl]pyrazolo[1,5-a]pyriridin-3-yl}(thien-2-yl)methanone and 1-(2-chloroethyl)pyrrolidine, mp 98° C.

### EXAMPLE 400

### (7-{3-[2-(2-Piperidin-1-ylethyl)-2H-tetraazol-5-yl]phenyl}pyrazolo[1,5-a]pyrimidin-3-yl)(thien-2-yl)methanone

The title compound was prepared using an analogous procedure to Method P from {7-[3-(1H-tetraazol-5-yl)phenyl]pyrazolo[1,5-a]pyrimidin-3-yl}(thien-2-yl)methanone and 1-(2-chloroethyl)piperidine, mp 117° C.

### EXAMPLE 401

### (7-{3-[2-(2-Hydroxyethyl)-2H-tetraazol-5-yl]phenyl}pyrazolo[1,5-a]pyrimidin-3-yl)(thien-2-yl)methanone

The title compound was prepared using an analogous procedure to Method P from {7-[3-(1H-tetraazol-5-yl)phenyl]pyrazolo[1,5-a]pyrimidin-3-yl}(thien-2-yl)methanone and 2-bromoethanol, mp 134° C.

### EXAMPLE 402

### (7-{3-[2-(2-Hydroxyethyl)-2H-tetraazol-5-yl]phenyl}pyrazolo[1,5-a]pyrimidin-3-yl)(thien-2-yl)methanone

The title compound was prepared using an analogous procedure to Method P from (7-(3-[2-(2-{[tert-butyl(dimethyl)silyl]oxy}ethyl)-2H-tetraazol-5-yl]phenyl}pyrazolo[1,5-a]pyrimidin-3-yl)(thien-2-yl)methanone and 2-bromoethanol, mp 134° C.

### EXAMPLE 403

### {7-[3-(1H-Pyrazol-3-yl)phenyl]pyrazolo[1,5-a]pyrimidin-3-yl}(thien-2-yl)methanone

The title compound was prepared using an analogous procedure to Method A from (2E)-3-(dimethylamino)-1-[3-(1H-pyrazol-3-yl)phenyl]prop-2-en-1-one and (5-amino-1H-pyrazol-4-yl)(thien-2-yl)methanone, mp 129° C.

(2E)-3-(Dimethylamino)-1-[3-( 1H-pyrazol-3-yl)phenyl]prop-2-en-1-one was prepared as described below: 3-dimethylamino-1-[3-(3-dimethylamino-acryloyl)-phenyl]-propenone (prepared from 1,3-diacetylbenzene and DMF-DMA) (2.0 g, 7.35 mmol) was dissolved in ethanol (100 mL) and hydrazine (0.50 mL, 10.28 mmol) wad added. The resulting mixture was heated at 60° C. for 6 hours and evaporated to remove ethanol. The residue was purifed by silica gel flash column chromatograph to give 830 mg (47%) of the desired product as a yellow solid, mp 155° C.

### EXAMPLE 404

### (7-{3-[1-(Cyclobutylmethyl)-1H-pyrazol-3-yl]phenyl}pyrazolo[1,5-a]pyrimidin-3-yl)(thien-2-yl)methanone

The title compound was prepared using an analogous procedure to Method P from {7-[3-(1H-Pyrazol-3-yl)phenyl]pyrazolo[1,5-a]pyrimidin-3-yl}(thien-2-yl)methanone and cyclobutylmethyl bromide, mp 60° C.

### EXAMPLE 405

### (7-{3-[1-(Pyridin-2-ylmethyl)-1H-pyrazol-3-yl]phenyl}pyrazolo[1,5-a]pyrimidin-3-yl)(thien-2-yl)methanone

The title compound was prepared using an analogous procedure to Method P from {7-[3-(1H-Pyrazol-3-yl)phenyl]pyrazolo[1,5-a]pyrimidin-3-yl}(thien-2-yl)methanone and 2-bromomethylpyridine, mp 66° C.

### EXAMPLE 406

### (7-{3-[1-(Pyridin-2-ylmethyl)-1H-pyrazol-3-yl]phenyl}pyrazolo[1,5-a]pyrimidin-3-yl)(thien-2-yl)methanone

The title compound was prepared using an analogous procedure to Method P from {7-[3-(1H-Pyrazol-3-yl)phenyl]pyrazolo[1,5-a]pyrimidin-3-yl}(thien-2-yl)methanone and 2-bromomethylpyridine, mp 66° C.

### EXAMPLE 407

### {7-[3-(1-Ethyl-1H-pyrazol-3-yl)phenyl]pyrazolo[1,5-a]pyrimidin-3-yl}(thien-2-yl)methanone

The title compound was prepared using an analogous procedure to Method P from {7-[3-(1H-Pyrazol-3-yl)phenyl]pyrazolo[1,5-a]pyrimidin-3-yl}(thien-2-yl)methanone and bromoethane, mp 61° C.

### EXAMPLE 408

### (7-{3-[1-(2-Pyrrolidin-1-ylethyl)-1H-pyrazol-3-yl]phenyl}pyrazolo[1,5-a]pyrimidin-3-yl)(thien-2-yl)methanone

The title compound was prepared using an analogous procedure to Method P from {7-[3-(1H-Pyrazol-3-yl)phenyl]pyrazolo[1,5-a]pyrimidin-3-yl}(thien-2-yl)methanone and 1-(2-chloroethyl)pyrrolidine, mp 63° C.

What is claimed is:

1. A compound of Formula I having the structure: 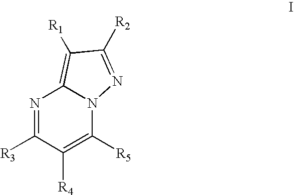
and pharmaceutically acceptable salts and prodrugs thereof,
wherein
R₁ is selected from the groups consisting of hydrogen, cyano, halogen, carbamoyl, formyl, carboxy, C(O)O-alkyl, C(O)O-cycloalkyl, C(O)cycloalkyl, R₆, C(O)R₆, and C(S)R₆;
R₆ is unsubstituted, monosubstituted or disubstituted aryl or heteroaryl wherein the aryl or heteroaryl is phenyl, naphthalenyl, thiazolyl, biphenyl, thienyl, furanyl, and pyridinyl; and the substituents are selected from the groups consisting of hydrogen, halogen, nitro, cyano, CF₃, OCF₃, alkyl, alkoxy, alkanol, trifluoromethyl, alkylamino, alkylthio, dialkylamino, methylenedioxy, alkylsulfonyl and alkanoylamino;
R₂, R₃, and R₄ are independently hydrogen, CF₃, or alkyl; R₅ is unsubstituted aryl or heteroaryl, or aryl or heteroaryl substituted by R₇, R₈, R₉, or R₁₀ with the proviso that R₁₀ must be present if the aryl or heteroaryl is substituted;
R₇, R₈, and R₉ are independently selected from the groups consisting of hydrogen, nitro, cyano, carboxy, carbamoyl, halogen, N(CH₃)₂, CF₃, OCF₃, alkyl, and alkoxy;
R₁₀ is selected from nitro, cyano, carboxy, carbamoyl, halogen, CF₃, OCF₃, alkyl, alkoxy, alkanol, NR₁₁R₁₂, N(R₁₃)COR₁₁, N(R₁₃)CONR₁₁R₁₂, OCONR₁₁R₁₂, N(R₁₃)CO₂R₁₁, N(R₁₃)CSR₁₁, N(R₁₃)CS(NR₁₁R₁₂), N(R₁₃)CS(OR₁₁), N(R₁₃)SO₂R₁₁, N(CONR₁₃R₁₁)₂, N(CO₂R₁₁)₂, N(COR₁₁)₂, N(CONR₁₃R₁₁)₂, CONR₁₁R₁₂, CO₂R₁₁, NHC(═NH)NHR₁₁, NHC(═NH—CN)NR₁₁R₁₂, NHC(═NH—CN)OR₁₁, C(═NH)NHR₁₁, C(═NH)NR₁₁R₁₂, LCONH₂, LCONR₁₁R₁₂, or LCO₂R₁₁ wherein L is alkyl, alkenyl or alkynyl or R₁₀ is selected from unsubstituted, monosubstituted, or disubstituted aryl or heteroaryl which is a 5- or 6-membered aromatic ring moiety containing at least 1-4 heteroatoms selected from O, S, and N;
R₁₁ and R₁₂ are independently selected from the groups consisting of hydrogen, alkyl, alkenyl, alkynyl, cycloalkyl, Q₁, Q₂, -L-Q₁ and -L-Q₂; wherein Q₁ is unsubstituted aryl or heteroaryl, monosubstituted or disubstituted aryl or heteroaryl; wherein Q₁ comprises phenyl, naphthalenyl, biphenyl, thiazolyl, oxazolyl, pyrrolyl, pyrrazolyl, thienyl, furanyl, 1,2,3-triazolyl, 1,2,4-triazolyl, tetrazolyl, pyrrolidinyl, imidazolyl, and pyridinyl; the substituents are selected from halogen, alkyl, CF₃, OCF₃, cyano, nitro, carboxy, hydroxy, alkoxy; wherein Q₂ is alkyl or heterocycle containing at least one and up to 4 heteroatoms selected from O, S, and N, optionally including their common protecting groups; Q₂ further comprises alkyl or cycloalkyl containing or substituted by functional groups selected from halogen, carboxy, carbamoyl, hydroxy, carbonyl, and cycloalkyl with a site of unsaturation;
R₁₁ and R₁₂ together with the N to which they are attached may join to form a 3 to 8 membered ring; and
R₁₃ is hydrogen, alkyl, alkenyl, alkynyl or cycloalkyl; R₁₁ and R₁₃ together with the N to which they are attached may join to form a 3 to 8 membered ring;
with the provisos that:
when R₁ is C(O)R₆ and when R₆ is unsubstituted phenyl; phenyl monosubstituted or di-substituted by halogen, alkoxy or alkyl; phenyl monosubstituted by alkylthio, alkylamino, dialkylamino, methylenedioxy, alkylsulfonyl, alkanoylamino; naphthalenyl; thiazolyl; biphenyl; thienyl; furanyl; pyridinyl; substituted thiazolyl; substituted biphenyl; thienyl or pyridinyl substituted by one or two of the group consisting of halogen, alkoxy and alkyl, then R₅ may not be unsubstituted phenyl, phenyl monosubstituted by halogen, CF₃, alkoxy, amino, alkyl, alkylamino, dialkylamino, alkanoylamino, N-alkylalkanoylamino, cyano or alkylthio; unsubstituted furanyl; unsubstituted thienyl; unsubstituted pyridinyl; or unsubstituted pyridine-1-oxide; or
when R₁ is hydrogen, fluoro, chloro, bromo, formyl, carboxy, cyano, C(O)alkyl, or CO(O)alkyl, then R₅ may not be phenyl monosubstituted by halogen, CF₃, alkoxy; unsubstituted pyridinyl, pyridine N-oxide, thienyl, furanyl; or pyridinyl, pyridine N-oxide, thienyl and furanyl monosubstituted by alkyl; or
when R₁ is hydrogen, halogen, cyano, formyl, C(O)alkyl, CO(O)alkyl and R₅ is phenyl substituted by R₁₀; and when R₁₀ is N(R₁₃)COR₁₁ where R₁₃ is hydrogen, alkyl, alkenyl, propynyl, or cycloalkylmethyl, then R₁₁ may not be alkyl, cycloalkyl, alkoxy, alkylamino, dialkylamino, —(CH₂)ₙO-alkyl, —(CH₂)ₙ—NH-alkyl, —(CH₂)ₙ—N-dialkyl wherein n is an integer 1 to 3 inclusive; or
when R₁ is hydrogen, cyano, chloro, or C(O)R₆ wherein R₆ is phenyl, phenyl monosubstituted by halogen, alkoxy, alkyl, trifluoromethyl, alkylthio, alkylamino, or dialkylamino; thienyl, furanyl, pyridinyl; furanyl, thienyl, pyridinyl monosubstituted by halogen, alkoxy or alkyl, then R₅ may not be meta-substituted phenyl wherein the substituent is an imidazolyl or imidazolyl substituted by 1-3 alkyl groups with the imidazolyl nitrogen directly attached to the phenyl ring; or
when R₁ is C(O)R₆ and R₆ is unsubstituted phenyl; phenyl monosubstituted or di-substituted by halogen, alkoxy or alkyl; phenyl monosubstituted by alkylthio, alkylamino, dialkylamino, methylenedioxy, alkylsulfonyl, alkanoylamino; naphthalenyl; thiazolyl; biphenyl; thienyl; furanyl; pyridinyl; substituted thiazolyl; substituted biphenyl; thienyl or pyridinyl substituted by one or two of the group consisting of halogen, alkoxy and alkyl; and when R₅ is phenyl substituted by R₁₀; wherein R₁₀ is N(R₁₃)COR₁₁, R₁₃ is hydrogen, alkenyl, propynyl, or cycloalkylmethyl, then R₁₁ may not be alkyl, cycloalkyl, alkoxy, alkylamino, dialkylamino, —(CH₂)ₙ—O-alkyl, —(CH₂)ₙ—NH-alkyl, —(CH₂)ₙ—N-dialkyl wherein n is an integer 1 to 3 inclusive.

2. A compound of Formula II having the structure: 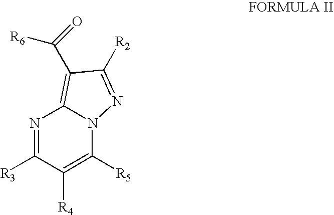
and pharmaceutically acceptable salts and prodrugs thereof,
wherein
R₂, R₃, and R₄ are hydrogen, CF₃, or alkyl;
R₆ is unsubstituted, monosubstituted or disubstituted aryl or heteroaryl wherein the aryl or heteroaryl group is phenyl, naphthalenyl, thiazolyl, biphenyl, thienyl, furanyl, or pyridinyl; and the substituents on the aryl or heteroaryl ring are selected from the groups consisting of halogen, nitro, cyano, CF₃, OCF₃, alkyl, alkoxy, trifluoromethyl, alkanol, alkylamino, alkylthio, dialkylamino, methylenedioxy, alkylsulfonyl and alkanoylamino;
R₅ is unsubstituted aryl or heteroaryl, or aryl or heteroaryl substituted by R₇, R₈, R₉, or R₁₀ with the proviso that R₁₀ must be present if the aryl or heteroaryl is substituted; wherein the aryl or heteroaryl is phenyl, naphthalenyl, thiazolyl, biphenyl, thienyl, furanyl, or pyridinyl;
R₇, R₈, and R₉ are independently selected from the groups consisting of hydrogen, nitro, cyano, carbamoyl, halogen, N(CH₃)₂; CF₃, OCF₃, alkyl, alkoxy, and carboxy;
R₁₀ is selected from nitro, cyano, carboxy, carbamoyl, halogen, CF₃, OCF₃, alkyl, alkoxy, alkanol, NR₁₁R₁₂, N(R₁₃)COR₁₁, N(R₁₃)CONR₁₁R₁₂, OCONR₁₁R₁₂, N(R₁₃)CO₂R₁₁, N(R₁₃)CSR₁₁, N(R₁₃)CS(NR₁₁R₁₂), N(R₁₃)CS(OR₁₁), N(R₁₃)SO₂R₁₁, N(CONR₁₃R₁₁)₂, N(CO₂R₁₁)₂, N(COR₁₁)₂, N(CONR₁₃R₁₁)₂, CONR₁₁R₁₂, CO₂R₁₁, NHC(═NH)NHR₁₁, NHC(═NH—CN)NR₁₁R₁₂, NHC(═NH—CN)OR₁₁, C(═NH)NHR₁₁, C(═NH)NR₁₁R₁₂, LCONH₂, LCONR₁₁R₁₂, or LCO₂R₁₁ wherein L is alkyl, alkenyl or alkynyl or R₁₀ is selected from unsubstituted, monosubstituted or disubstituted aryl or heteroaryl which is a 5- or 6-membered aromatic ring moiety containing at least 1-4 heteroatoms selcted from O, S, and N;
R₁₁ and R₁₂ are independently selected from the groups consisting of hydrogen, alkyl, alkenyl, alkynyl, cycloalkyl, Q₁, Q₂, -L-Q₁ and -L-Q₂; wherein Q₁ is unsubstituted aryl or heteroaryl, monosubstituted or disubstituted aryl or heteroaryl; wherein Q₁ comprises phenyl, naphthalenyl, biphenyl, thiazolyl, oxazolyl, pyrrolyl, pyrrazolyl, thienyl, furanyl, 1,2,3-triazolyl, 1,2,4-triazolyl, tetrazolyl, pyrrolidinyl, imidazolyl, and pyridinyl; the substituents are selected from halogen, alkyl, CF₃, OCF₃, cyano, nitro, carboxy, hydroxy, alkoxy; wherein Q₂ is alkyl or heterocycle containing at least one and up to 4 heteroatoms selected from O, S, and N, optionally including their common protecting groups; Q₂ further comprises alkyl or cycloalkyl containing or substituted by functional groups selected from halogen, carboxy, carbamoyl, hydroxy, carbonyl, and cycloalkyl with a site of unsaturation;
R₁₁ and R₁₂ together with the N to which they are attached may join to form a 3 to 8 membered ring; and
R₁₃ is hydrogen, alkyl, alkenyl, alkynyl or cycloalkyl; R₁₁ and R₁₃ together with the N to which they are attached may join to form a 3 to 8 membered ring;
with the provisos that:
when R₁ is C(O)R₆ and when R₆ is unsubstituted phenyl; phenyl monosubstituted or di-substituted by halogen, alkoxy or alkyl; phenyl monosubstituted by alkylthio, alkylamino, dialkylamino, methylenedioxy, alkylsulfonyl, alkanoylamino; naphthalenyl; thiazolyl; biphenyl; thienyl; furanyl; pyridinyl; substituted thiazolyl; substituted biphenyl; thienyl or pyridinyl substituted by one or two of the group consisting of halogen, alkoxy and alkyl, then R₅ may not be unsubstituted phenyl, phenyl monosubstituted by halogen, CF₃, alkoxy, amino, alkyl, alkylamino, dialkylamino, alkanoylamino, N-alkylalkanoylamino, cyano or alkylthio; unsubstituted furanyl; unsubstituted thienyl; unsubstituted pyridinyl; or unsubstituted pyridine-1-oxide; or
when R₁ is hydrogen, fluoro, chloro, bromo, formyl, carboxy, cyano, C(O)alkyl, or CO(O)alkyl, then R₅ may not be phenyl monosubstituted by halogen, CF₃, alkoxy; unsubstituted pyridinyl, pyridine N-oxide, thienyl, furanyl; or pyridinyl, pyridine N-oxide, thienyl and furanyl monosubstituted by alkyl; or
when R₁ is hydrogen, halogen, cyano, formyl, C(O)alkyl, CO(O)alkyl and R₅ is phenyl substituted by R₁₀; and when R₁₀ is N(R₁₃)COR₁₁ where R₁₃ is hydrogen, alkyl, alkenyl, propynyl, or cycloalkylmethyl, then R₁₁ may not be alkyl, cycloalkyl, alkoxy, alkylamino, dialkylamino, —(CH₂)ₙ—O-alkyl, —(CH₂)ₙ—NH-alkyl, —(CH₂)ₙ—N-dialkyl wherein n is an integer 1 to 3 inclusive; or
when R₁ is hydrogen, cyano, chloro, or C(O)R₆ wherein R₆ is phenyl, phenyl monosubstituted by halogen, alkoxy, alkyl, trifluoromethyl, alkylthio, alkylamino, or dialkylamino; thienyl, furanyl, pyridinyl; furanyl, thienyl, pyridinyl monosubstituted by halogen, alkoxy or alkyl, then R₅ may not be meta-substituted phenyl wherein the substituent is an imidazolyl or imidazolyl substituted by 1-3 alkyl groups with the imidazolyl nitrogen directly attached to the phenyl ring; or
when R₁ is C(O)R₆ and R₆ is unsubstituted phenyl; phenyl monosubstituted or di-substituted by halogen, alkoxy or alkyl; phenyl monosubstituted by alkylthio, alkylamino, dialkylamino, methylenedioxy, alkylsulfonyl, alkanoylamino; naphthalenyl; thiazolyl; biphenyl; thienyl; furanyl; pyridinyl; substituted thiazolyl; substituted biphenyl; thienyl or pyridinyl substituted by one or two of the group consisting of halogen, alkoxy and alkyl; and when R₅ is phenyl substituted by R₁₀; wherein R₁₀ is N(R₁₃)COR₁₁, R₁₃ is hydrogen, alkenyl, propynyl, or cycloalkylmethyl, then R₁₁ may not be alkyl, cycloalkyl, alkoxy, alkylamino, dialkylamino, —(CH₂)ₙ—O-alkyl, —(CH₂)ₙ—NH-alkyl, —(CH₂)ₙ—N-dialkyl wherein n is an integer 1 to 3 inclusive.

3. A compound of Formula III having the structure: 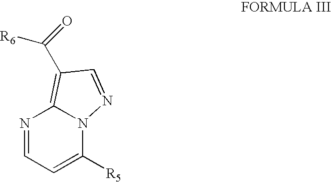
and pharmaceutically acceptable salts and prodrugs thereof,
wherein
R₆ is unsubstituted, monosubstituted or disubstituted aryl or heteroaryl wherein aryl or heteroaryl group is phenyl, naphthalenyl, thiazolyl, biphenyl, thienyl, furanyl, or pyridinyl; and the substituents are selected from the groups consisting of halogen, nitro, cyano, CF₃, OCF₃, alkyl, alkoxy, trifluoromethyl, alkylamino, alkanol, alkylthio, dialkylamino, methylenedioxy, alkylsulfonyl and alkanoylamino;
R₅ is unsubstituted aryl or heteroaryl, or aryl or heteroaryl substituted by R₇, R₈, R₉, or R₁₀ with the proviso that R₁₀ must be present if the aryl or heteroaryl is substituted; wherein the aryl or heteroaryl is phenyl, naphthalenyl, thiazolyl, biphenyl, thienyl, furanyl, or pyridinyl;
R₇, R₈, and R₉ are independently selected from the groups consisting of hydrogen, nitro, cyano, carboxy, carbamoyl, halogen, N(CH₃)₂, CF₃, OCF₃, alkyl, and alkoxy;
R₁₀ is selected from nitro, cyano, carboxy, carbamoyl, halogen, CF₃, OCF₃, alkyl, alkoxy, alkanol, NR₁₁R₁₂, N(R₁₃)COR₁₁, N(R₁₃)CONR₁₁R₁₂, OCONR₁₁R₁₂, N(R₁₃)CO₂R₁₁, N(R₁₃)CSR₁₁, N(R₁₃)CS(NR₁₁R₁₂), N(R₁₃)CS(OR₁₁), N(R₁₃)SO₂R₁₁, N(CONR₁₃R₁₁)₂, N(CO₂R₁₁)₂, N(COR₁₁)₂, N(CONR₁₃R₁₁)₂, CONR₁₁R₁₂, CO₂R₁₁, NHC(═NH)NHR₁₁, NHC(═NH—CN)NR₁₁R₁₂, NHC(═NH—CN)OR₁₁, C(═NH)NHR₁₁, C(═NH)NR₁₁R₁₂, LCONH₂, LCONR₁₁R₁₂, or LCO₂R₁₁ wherein L is alkyl, alkenyl or alkynyl or R₁₀ is selected from unsubstituted, monosubstituted or disubstituted aryl or heteroaryl which is a 5- or 6-membered aromatic ring moiety containing at least 1-4 heteroatoms selected from O, S, and N;
R₁₁ and R₁₂ are independently selected from the groups consisting of hydrogen, alkyl, alkenyl, alkynyl, cycloalkyl, Q₁, Q₂, -L-Q₁ and -L-Q₂; wherein Q₁ is unsubstituted aryl or heteroaryl, monosubstituted or disubstituted aryl or heteroaryl; wherein Q₁ comprises phenyl, naphthalenyl, biphenyl, thiazolyl, oxazolyl, pyrrolyl, pyrrazolyl, thienyl, furanyl, 1,2,3-triazolyl, 1,2,4-triazolyl, tetrazolyl, pyrrolidinyl, imidazolyl, and pyridinyl; the substituents are selected from halogen, alkyl, CF₃, OCF₃, cyano, nitro, carboxy, hydroxy, alkoxy; wherein Q₂ is alkyl or heterocycle containing at least one and up to 4 heteroatoms selected from O, S, and N, optionally including their common protecting groups; Q₂ further comprises alkyl or cycloalkyl containing or substituted by functional groups selected from halogen, carboxy, carbamoyl, hydroxy, carbonyl, and cycloalkyl with a site of unsaturation;
R₁₁ and R₁₂ together with the N to which they are attached may join to form a 3 to 8 membered ring; and
R₁₃ is hydrogen, alkyl, alkenyl, alkynyl or cycloalkyl; R₁₁ and R₁₃ together with the N to which they are attached may join to form a 3 to 8 membered ring;
with the provisos that:
when R₁ is C(O)R₆ and when R₆ is unsubstituted phenyl; phenyl monosubstituted or di-substituted by halogen, alkoxy or alkyl; phenyl monosubstituted by alkylthio, alkylamino, dialkylamino, methylenedioxy, alkylsulfonyl, alkanoylamino; naphthalenyl; thiazolyl; biphenyl; thienyl; furanyl; pyridinyl; substituted thiazolyl; substituted biphenyl; thienyl or pyridinyl substituted by one or two of the group consisting of halogen, alkoxy and alkyl, then R₅ may not be unsubstituted phenyl, phenyl monosubstituted by halogen, CF₃, alkoxy, amino, alkyl, alkylamino, dialkylamino, alkanoylamino, N-alkylalkanoylamino, cyano or alkylthio; unsubstituted furanyl; unsubstituted thienyl; unsubstituted pyridinyl; or unsubstituted pyridine-1-oxide; or
when R₁ is hydrogen, fluoro, chloro, bromo, formyl, carboxy, cyano, C(O)alkyl, or CO(O)alkyl, then R₅ may not be phenyl monosubstituted by halogen, CF₃, alkoxy; unsubstituted pyridinyl, pyridine N-oxide, thienyl, furanyl; or pyridinyl, pyridine N-oxide, thienyl and furanyl monosubstituted by alkyl; or
when R₁ is hydrogen, halogen, cyano, formyl, C(O)alkyl, CO(O)alkyl and R₅ is phenyl substituted by R₁₀; and when R₁₀ is N(R₁₃)COR₁₁ where R₁₃ is hydrogen, alkyl, alkenyl, propynyl, or cycloalkylmethyl, then R₁₁ may not be alkyl, cycloalkyl, alkoxy, alkylamino, dialkylamino, —(CH₂)ₙ—O-alkyl, —(CH₂)ₙ—NH-alkyl, —(CH₂)ₙ—N-dialkyl wherein n is an integer 1 to 3 inclusive; or
when R₁ is hydrogen, cyano, chloro, or C(O)R₆ wherein R₆ is phenyl, phenyl monosubstituted by halogen, alkoxy, alkyl, trifluoromethyl, alkylthio, alkylamino, or dialkylamino; thienyl, furanyl, pyridinyl; furanyl, thienyl, pyridinyl monosubstituted by halogen, alkoxy or alkyl, then R₅ may not be meta-substituted phenyl wherein the substituent is an imidazolyl or imidazolyl substituted by 1-3 alkyl groups with the imidazolyl nitrogen directly attached to the phenyl ring; or
when R₁ is C(O)R₆ and R₆ is unsubstituted phenyl; phenyl monosubstituted or di-substituted by halogen, alkoxy or alkyl; phenyl monosubstituted by alkylthio, alkylamino, dialkylamino, methylenedioxy, alkylsulfonyl, alkanoylamino; naphthalenyl; thiazolyl; biphenyl; thienyl; furanyl; pyridinyl; substituted thiazolyl; substituted biphenyl; thienyl or pyridinyl substituted by one or two of the group consisting of halogen, alkoxy and alkyl; and when R₅ is phenyl substituted by R₁₀; wherein R₁₀ is N(R₁₃)COR₁₁, R₁₃ is hydrogen, alkenyl, propynyl, or cycloalkylmethyl, then R₁₁ may not be alkyl, cycloalkyl, alkoxy, alkylamino, dialkylamino, —(CH₂)ₙ—O-alkyl, —(CH₂)ₙ—NH-alkyl, —(CH₂)ₙ—N-dialkyl wherein n is an integer 1 to 3 inclusive.

4. The compounds of claim 1, wherein the compounds are selected from the group consisting of:
N-Phenyl-N′-{3-[3-(thien-2-ylcarbonyl)pyrazolo[1,5-a]pyrimidin-7-yl]phenyl}urea;
N-(2-Phenylethyl)-N′-{3-[3-(thien-2-ylcarbonyl)pyrazolo[1,5-a]pyrimidin-7-yl]phenyl}urea;
N-Cyclohexyl-N′-{3-[3-(thien-2-ylcarbonyl)pyrazolo[1,5-a]pyrimidin-7-yl]phenyl}urea;
Methyl N-[({3-[3-(thien-2-ylcarbonyl)pyrazolo[1,5-a]pyrimidin-7-yl]phenyl}amino)carbonyl]-L valinate;
N-Allyl-N′-{3-[3-(thien-2-ylcarbonyl)pyrazolo[1,5-a]pyrimidin-7-yl]phenyl}urea;
N-Butyl-N′-{3-[3-(thien-2-ylcarbonyl)pyrazolo[1,5-a]pyrimidin-7-yl]phenyl}urea;
N-{3-[3-(Thien-2-ylcarbonyl)pyrazolo[1,5-a]pyrimidin-7-yl]phenyl}-N′-(trichloroacetyl)urea;
Ethyl N-[({3-[3-(thien-2-ylcarbonyl)pyrazolo[1,5-a]pyrimidin-7-yl]phenyl}amino)carbonyl]glycinate;
N-[(1S)-1-Phenylethyl]-N′-{3-[3-(thien-2-ylcarbonyl)pyrazolo[1,5-a]pyrimidin-7-yl]phenyl}urea;
N-[(1S,2R)-2-Phenylcyclopropyl]-N′-{3-[3-(thien-2-ylcarbonyl)pyrazolo[1,5-a]pyrimidin-7-yl]phenyl}urea;
Allyl 3-[3-(thien-2-ylcarbonyl)pyrazolo[1,5-a]pyrimidin-7-yl]phenylcarbamate;
2,2,2-Trichloroethyl 3-[3-(thien-2-ylcarbonyl)pyrazolo[1,5-a]pyrimidin-7-yl]phenylcarbamate;
Prop-2-ynyl 3-[3-(thien-2-ylcarbonyl)pyrazolo[1,5-a]pyrimidin-7-yl]phenylcarbamate;
3-Butenyl 3-[3-(2-thienylcarbonyl)pyrazolo[1,5-a]pyrimidin-7-yl]phenylcarbamate;
Isopropenyl 3-[3-(thien-2-ylcarbonyl)pyrazolo[1,5-a]pyrimidin-7-yl]phenylcarbamate;
2-Chloroethyl 3-[3-(thien-2-ylcarbonyl)pyrazolo[1,5-a]pyrimidin-7-yl]phenylcarbamate;
4-Methylphenyl 3-[3-(thien-2-ylcarbonyl)pyrazolo[1,5-a]pyrimidin-7-yl]phenylcarbamate;
Benzyl 3-[3-(thien-2-ylcarbonyl)pyrazolo[1,5-a]pyrimidin-7-yl]phenylcarbamate;
4-Chlorobutyl 3-[3-(thien-2-ylcarbonyl)pyrazolo[1,5-a]pyrimidin-7-yl]phenylcarbamate;
N-{3-[3-(Thien-2-ylcarbonyl)pyrazolo[1,5-a]pyrimidin-7-yl]phenyl}methanesulfonamide;
N-{3-[3-(Thien-2-ylcarbonyl)pyrazolo[1,5-a]pyrimidin-7-yl]phenyl}ethanesulfonamide;
N-{3-[3-(Thien-2-ylcarbonyl)pyrazolo[1,5-a]pyrimidin-7-yl]phenyl}butane-1-sulfonamide;
N-{3-[3-(Thien-2-ylcarbonyl)pyrazolo[1,5-a]pyrimidin-7-yl]phenyl}propane-2-sulfonamide;
N-{3-[3-(Thien-2-ylcarbonyl)pyrazolo[1,5-a]pyrimidin-7-yl]phenyl}benzenesulfonamide;
4-Methyl-N-{3-[³-(thien-2-ylcarbonyl)pyrazolo[1,5-a]pyrimidin-7-yl]phenyl}benzenesulfonamide;
2-Methyl-N-{3-[3-(thien-2-ylcarbonyl)pyrazolo[1,5-a]pyrimidin-7-yl]phenyl}benzenesulfonamide;
4-Fluoro-N-{³-[3-(thien-2-ylcarbonyl)pyrazolo[1,5-a]pyrimidin-7-yl]phenyl}benzenesulfonamide;
4-Bromo-N-{3-[3-(thien-2-ylcarbonyl)pyrazolo[1,5-a]pyrimidin-7-yl]phenyl}benzenesulfonamide;
4-Methoxy-N-{3-[3-(thien-2-ylcarbonyl)pyrazolo[1,5-a]pyrimidin-7-yl]phenyl}benzenesulfonamide;
(E)-2-Phenyl-N-{3-[3-(thien-2-ylcarbonyl)pyrazolo[1,5-a]pyrimidin-7-yl]phenyl}ethenesulfonamide;
N-{3-[3-(Thien-2-ylcarbonyl)pyrazolo[1,5-a]pyrimidin-7-yl]phenyl}thiophene-2-sulfonamide;
1-Methyl-N-{3-[3-(thien-2-ylcarbonyl)pyrazolo[1,5-a]pyrimidin-7-yl]phenyl}-1H-imidazole-4-sulfonamide;
N,N-Dimethyl-N′-{3-[3-(thien-2-ylcarbonyl)pyrazolo[1,5-a]pyrimidin-7-yl]phenyl}sulfamide;
Ethyl oxo({3-[3-(thien-2-ylcarbonyl)pyrazolo[1,5-a]pyrimidin-7-yl]phenyl}amino)acetate;
Methyl 4-oxo-4-({3-[3-(thien-2-ylcarbonyl)pyrazolo[1,5-a]pyrimidin-7-yl]phenyl}amino)butanoate;
2-Methyl-N-{3-[3-(thien-2-ylcarbonyl)pyrazolo[1,5-a]pyrimidin-7-yl]phenyl}acrylamide;
3-Methyl-N-{3-[3-(thien-2-ylcarbonyl)pyrazolo[1,5-a]pyrimidin-7-yl]phenyl}but-2-enamide;
2,2,2-Trichloro-N-{3-[3-(thien-2-ylcarbonyl)pyrazolo[1,5-a]pyrimidin-7-yl]phenyl}acetamide;
3-(Methylthio)-N-{3-[3-(thien-2-ylcarbonyl)pyrazolo[1,5-a]pyrimidin-7-yl]phenyl}propanamide;
N-{3-[3-(Thien-2-ylcarbonyl)pyrazolo[1,5-a]pyrimidin-7-yl]phenyl}thiophene-2-carboxamide;
N-{3-[3-(Thien-2-ylcarbonyl)pyrazolo[1,5-a]pyrimidin-7-yl]phenyl}-1-(trifluoroacetyl)-L-prolinamide;
N-{3-[3-(Thien-2-ylcarbonyl)pyrazolo[1,5-a]pyrimidin-7-yl]phenyl}adamantane-1-carboxamide;
4,7,7-Trimethyl-3-oxo-N-{3-[3-(thien-2-ylcarbonyl)pyrazolo[1,5-a]pyrimidin-7-yl]phenyl}-2-oxabicyclo[2.2.1]heptane-1-carboxamide;
N-{3-[3-(Thien-2-ylcarbonyl)pyrazolo[1,5-a]pyrimidin-7-yl]phenyl}pent-4-enamide;
N-{3-[3-(Thien-2-ylcarbonyl)pyrazolo[1,5-a]pyrimidin-7-yl]phenyl}pent-4-ynamide;
4,4,4-Trifluoro-3-methyl-N-{3-[3-(thien-2-ylcarbonyl)pyrazolo[1,5-a]pyrimidin-7-yl]phenyl}butanamide;
2-Cyano-N-{3-[3-(thien-2-ylcarbonyl)pyrazolo[1,5-a]pyrimidin-7-yl]phenyl}acetamide;
2-Oxo-2-({3-[3-(thien-2-ylcarbonyl)pyrazolo[1,5-a]pyrimidin-7-yl]phenyl}amino)ethyl acetate;
2-(2-Methoxyethoxy)-N-{3-[3-(thien-2-ylcarbonyl)pyrazolo[1,5-a]pyrimidin-7-yl]phenyl}acetamide;
2-Thien-2-yl-N-{3-[3-(thien-2-ylcarbonyl)pyrazolo[1,5-a]pyrimidin-7-yl]phenyl}acetamide;
2-(2,3-Dihydro-1H-inden-2-yl)-N-{3-[3-(thien-2-ylcarbonyl)pyrazolo[1,5-a]pyrimidin-7-yl[phenyl}acetamide;
2-(2-Methylphenyl)-N-{3-[3-(thien-2-ylcarbonyl)pyrazolo[1,5-a]pyrimidin-7-yl]phenyl}acetamide;
2-(1,3-Benzodioxol-5-yl)-N-{3-[3-(thien-2-ylcarbonyl)pyrazolo[1,5-a]pyrimidin-7-yl]phenyl}acetamide;
N-{3-[3-(Thien-2-ylcarbonyl)pyrazolo[1,5-a]pyrimidin-7-yl]phenyl}quinoline-3-carboxamide;
(2E)-3-Phenyl-N-{3-[3-(thien-2-ylcarbonyl)pyrazolo[1,5-a]pyrimidin-7-yl]phenyl}prop-2-enamide;
2-Cyclohexyl-N-{3-[3-(thien-2-ylcarbonyl)pyrazolo[1,5-a]pyrimidin-7-yl]phenyl}acetamide;
2-Cycloheptyl-N-{3-[3-(thien-2-ylcarbonyl)pyrazolo[1,5-a]pyrimidin-7-yl]phenyl}acetamide;
2-Bicyclo[2.2.1]hept-2-yl-N-{3-[3-(thien-2-ylcarbonyl)pyrazolo[1,5-a]pyrimidin-7-yl]phenyl}acetamide;
N-1-{3-[3-(Thien-2-ylcarbonyl)pyrazolo[1,5-a]pyrimidin-7-yl]phenyl}glycinamide;
N-1-{3-[3-(Thien-2-ylcarbonyl)pyrazolo[1,5-a]pyrimidin-7-yl]phenyl}-L-alaninamide;
N-1-{3-[3-(Thien-2-ylcarbonyl)pyrazolo[1,5-a]pyrimidin-7-yl]phenyl}-L-leucinamide;
N-1-{3-[3-(Thien-2-ylcarbonyl)pyrazolo[1,5-a]pyrimidin-7-yl]phenyl}-L-isoleucinamide;
N-{3-[3-(Thien-2-ylcarbonyl)pyrazolo[1,5-a]pyrimidin-7-yl]phenyl}-L-phenylalaninamide;
3-Cyclohexyl-N-1-{3-[3-(thien-2-ylcarbonyl)pyrazolo[1,5-a]pylimidin-7-yl]phenyl}-L-alaninamide;
N-1-{3-[3-(Thien-2-ylcarbonyl)pyrazolo[1,5-a]pyrimidin-7-yl]phenyl}-L-valinamide;
4-Amino-N-{3-[3-(thien-2-ylcarbonyl)pyrazolo[1,5-a]pyrimidin-7-yl]phenyl}butanamide;
N-{3-[3-(Thien-2-ylcarbonyl)pyrazolo[1,5-a]pyrimidin-7-yl]phenyl}-L-prolinamide;
(2S)-2-Amino-2-cyclohexyl-N-{3-[3-(thien-2-ylcarbonyl)pyrazolo[1,5-a]pyrimidin-7-yl]phenyl}ethanamide;
4-Methyl-N-{3-[3-(thien-2-ylcarbonyl)pyrazolo[1,5-a]pyrimidin-7-yl]phenyl}benzamide;
4-Methoxy-N-(3-[3-(thien-2-ylcarbonyl)pyrazolo[1,5-a]pyrimidin-7-yl]phenyl}benzamide;
4-Chloro-N-{3-[3-(thien-2-ylcarbonyl)pyrazolo[1,5-a]pyrimidin-7-yl]phenyl}benzamide;
4-Bromo-N-{3-[3-(thien-2-ylcarbonyl)pyrazolo[1,5-a]pyrimidin-7-yl]phenyl}benzamide;
4-(Heptyloxy)-N-{3-[3-(thien-2-ylcarbonyl)pyrazolo[1,5-a]pyrimidin-7-yl]phenyl}benzamide;
4-Pentyl-N-{3-[3-(thien-2-ylcarbonyl)pyrazolo[1,5-a]pyrimidin-7-yl]phenyl}benzamide;
2-Methoxy-N-{3-[3-(thien-2-ylcarbonyl)pyrazolo[1,5-a]pyrimidin-7-yl]phenyl}benzamide;
2-Methyl-N-{3-[3-(thien-2-ylcarbonyl)pyrazolo[1,5-a]pyrimidin-7-yl]phenyl}benzamide;
2-Chloro-N-{3-[3-(thien-2-ylcarbonyl)pyrazolo[1,5-a]pyrimidin-7-yl]phenyl}benzamide;
N-{3-[3-(Thien-2-ylcarbonyl)pyrazolo[1,5-a]pyrimidin-7-yl]phenyl}-2-(trifluoromethyl)benzamide;
3-Methoxy-N-{3-[3-(thien-2-ylcarbonyl)pyrazolo[1,5-a]pyrimidin-7-yl]phenyl}benzamide;
3-Fluoro-N-(3-[3-(thien-2-ylcarbonyl)pyrazolo[1,5-a]pyrimidin-7-yl]phenyl}benzamide;
3,4-Dimethoxy-N-{3-[3-(thien-2-ylcarbonyl)pyrazolo[1,5-a]pyrimidin-7-yl]phenyl}benzamide;
3,4-Dichloro-N-{3-[3-(thien-2-ylcarbonyl)pyrazolo[1,5-a]pyrimidin-7-yl]phenyl}benzamide;
2,6-Dimethoxy-N-{3-[3-(thien-2-ylcarbonyl)pyrazolo[1,5-a]pyrimidin-7-yl]phenyl}benzamide;
3,4-Difluoro-N-{3-[3-(thien-2-ylcarbonyl)pyrazolo[1,5-a]pyrimidin-7-yl]phenyl}benzamide;
N-{3-[3-(Thien-2-ylcarbonyl)pyrazolo[1,5-a]pyrimidin-7-yl]phenyl}-1-naphthamide;
N-{3-[3-(Thien-2-ylcarbonyl)pyrazolo[1,5-a]pyrimidin-7-yl]phenyl}-2-naphthamide;
2-Phenyl-N-{3-[3-(thien-2-ylcarbonyl)pyrazolo[1,5-a]pyrimidin-7-yl]phenyl}acetamide;
2-(4-Methoxyphenyl)-N-{3-[3-(thien-2-ylcarbonyl)pyrazolo[1,5-a]pyrimidin-7-yl]phenyl}acetamide;
2-(3-Methoxyphenyl)-N-{3-[3-(thien-2-ylcarbonyl)pyrazolo[1,5-a]pyrimidin-7-yl]phenyl}acetamide;
2-(4-Chlorophenyl)-N-{3-[3-(thien-2-ylcarbonyl)pyrazolo[1,5-a]pyrimidin-7-yl]phenyl}acetamide;
2-(4-Fluorophenyl)-N-{3-[3-(thien-2-ylcarbonyl)pyrazolo[1,5-a]pyrimidin-7-yl]phenyl}acetamide;
2-(2,5-Dimethoxyphenyl)-N-{3-[3-(thien-2-ylcarbonyl)pyrazolo[1,5-a]pyrimidin-7-yl]phenyl}acetamide;
2-Phenoxy-N-{3-[3-(thien-2-ylcarbonyl)pyrazolo[1,5-a]pyrimidin-7-yl]phenyl}acetamide;
4-Fluoro-N-{3-[3-(thien-2-ylcarbonyl)pyrazolo[1,5-a]pyrimidin-7-yl]phenyl}benzamide;
Ethyl 2-oxo-2-({3-[3-(thien-2-ylcarbonyl)pyrazolo[1,5-a]pyrimidin-7-yl]phenyl}amino)ethylcarbamate;
Propyl 2-oxo-2-({3-[3-(thien-2-ylcarbonyl)pyrazolo[1,5-a]pyrimidin-7-ylphenyl}amino)ethylcarbamate;
Isopropyl 2-oxo-2-({3-[3-(thien-2-ylcarbonyl)pyrazolo[1,5-a]pyrimidin-7-yl]phenyl}amino)ethylcarbamate;
Butyl 2-oxo-2-({3-[3-(thien-2-ylcarbonyl)pyrazolo[1,5-a]pyrimidin-7-yl]phenyl}amino)ethylcarbamate;
Isobutyl 2-oxo-2-({3-[3-(thien-2-ylcarbonyl)pyrazolo[1,5-a]pyrimidin-7-yl]phenyl}amino)ethylcarbamate;
But-3-enyl 2-oxo-2-({3-[3-(thien-2-ylcarbonyl)pyrazolo[1,5-a]pyrimidin-7-yl]phenyl}amino)ethylcarbamate;
4-Chlorobutyl 2-oxo-2-({3-[3-(thien-2-ylcarbonyl)pyrazolo[1,5-a]pyrimidin-7-yl]phenyl}amino)ethylcarbamate;
2-Chloroethyl 2-oxo-2-({3-[3-(thien-2-ylcarbonyl)pyrazolo[1,5-a]pyrimidin-7-yl]phenyl}amino)ethylcarbamate;
Neopentyl 2-oxo-2-({3-[3-(thien-2-ylcarbonyl)pyrazolo[1,5-a]pyrimidin-7-yl]phenyl}amino)ethylcarbamate;
Hexyl 2-oxo-2-({3-[3-(thien-2-ylcarbonyl)pyrazolo[1,5-a]pyrimidin-7-yl]phenyl}amino)ethylcarbamate;
Prop-2-ynyl 2-oxo-2-({3-[3-(thien-2-ylcarbonyl)pyrazolo[1,5-a]pyrimidin-7-yl]phenyl}amino)ethylcarbamate;
Allyl 2-oxo-2-({3-[3-(thien-2-ylcarbonyl)pyrazolo[1,5-a]pyrimidin-7-yl]phenyl}amino)ethylcarbamate;
Benzyl 2-oxo-2-({3-[3-(thien-2-ylcarbonyl)pyrazolo[1,5-a]pyrimidin-7-yl]phenyl}amino)ethylcarbamate;
N-[2-Oxo-2-({3-[3-(thien-2-ylcarbonyl)pyrazolo[1,5-a]pyrimidin-7-yl]phenyl}amino)ethyl]cyclopropanecarboxamide;
N-[2-Oxo-2-({3-[3-(thien-2-ylcarbonyl)pyrazolo[1,5-a]pyrimidin-7-yl]phenyl}amino)ethyl]cyclobutanecarboxamide;
N-[2-Oxo-2-({3-[3-(thien-2-ylcarbonyl)pyrazolo[1,5-a]pyrimidin-7-yl]phenyl}amino)ethyl]cyclopentanecarboxamide;
N-[2-Oxo-2-({3-[3-(thien-2-ylcarbonyl)pyrazolo[1,5-a]pyrimidin-7-yl]phenyl}amino)ethyl]cyclohexanecarboxamide;
2-Methyl-N-[2-oxo-2-({3-[3-(thien-2-ylcarbonyl)pyrazolo[1,5-a]pyrimidin-7-yl]phenyl}amino)ethyl]butanamide;
2-Ethyl-N-[2-oxo-2-({3-[3-(thien-2-ylcarbonyl)pyrazolo[1,5-a]pyrimidin-7-yl]phenyl}amino)ethyl]butanamide;
2,2-Dimethyl-N-[2-oxo-2-({3-[3-(thien-2-ylcarbonyl)pyrazolo[1,5-a]pyrimidin-7-yl]phenyl}amino)ethyl]propanamide;
3-Methyl-N-[2-oxo-2-({3-[3-(thien-2-ylcarbonyl)pyrazolo[1,5-a]pyrimidin-7-yl]phenyl}amino)ethyl]butanamide;
N-[2-Oxo-2-({3-[3-(thien-2-ylcarbonyl)pyrazolo[1,5-a]pyrimidin-7-yl]phenyl}amino)ethyl]adamantane-1-carboxamide;
N-[2-Oxo-2-({3-[3-(thien-2-ylcarbonyl)pyrazolo[1,5-a]pyrimidin-7-yl]phenyl}amino)ethyl]benzamide;
N-2-[(Pentylamino)carbonyl]-N-1-{3-[3-(thien-2-ylcarbonyl)pyrazolo[1,5-A]pyrimidin-7-yl]phenyl}glycinamide;
N-2-[(Ethylamino)carbonyl]-N-1-{3-[3-(thien-2-ylcarbonyl)pyrazolo[1,5-a]pyrimidin-7-yl]phenyl}glycinamide;
N-2-[(Cyclohexylamino)carbonyl]-N-1-{3-[3-(thien-2-ylcarbonyl)pyrazolo[1,5-a]pyrimidin-7-yl]phenyl}glycinamide;
3-Chloro-N-{3-[3-(thien-2-ylcarbonyl)pyrazolo[1,5-a]pyrimidin-7-yl]phenyl}propanamide;
3,5,5-Trimethyl-N-{3-[3-(thien-2-ylcarbonyl)pyrazolo[1,5-a]pyrimidin-7-yl]phenyl}hexanamide;
3-Chloro-2-(chloromethyl)-2-methyl-N-{3-[3-(thien-2-ylcarbonyl)pyrazolo[1,5-a]pyrimidin-7-yl]phenyl}propanamide;
N-{3-[3-(Thien-2-ylcarbonyl)pyrazolo[1,5-a]pyrimidin-7-yl]phenyl}undecanamide;
1-Methyl-N-{3-[3-(thien-2-ylcarbonyl)pyrazolo[1,5-a]pyrimidin-7-yl]phenyl}cyclohexanecarboxamide;
2,3,3-Trichloro-N-{3-[3-(thien-2-ylcarbonyl)pyrazolo[1,5-a]pyrimidin-7-yl]phenyl}acrylamide;
3-Cyclopentyl-N-{3-[3-(thien-2-ylcarbonyl)pyrazolo[1I5-a]pyrimidin-7-yl]phenyl}propanamide;
2-Ethyl-N-{3-[3-(thien-2-ylcarbonyl)pyrazolo[1,5-a]pyrimidin-7-yl]phenyl}hexanamide;
(2E)-N-{3-[3-(Thien-2-ylcarbonyl)pyrazolo[1,5-a]pyrimidin-7-yl]phenyl}but-2-enamide;
N-{3-[3-(Thien-2-ylcarbonyl)pyrazolo[1,5-a]pyrimidin-7-yl]phenyl}piperidine-1-carboxamide;
N-{3-[3-(Thien-2-ylcarbonyl)pyrazolo[1,5-a]pyrimidin-7-yl]phenyl}morpholine-4-carboxamide;
N-(sec-Butyl)-N′-{3-[3-(thien-2-ylcarbonyl)pyrazolo[1,5-a]pyrimidin-7-yl]phenyl}urea;
N-(Cyclohexylmethyl)-N′-{3-[3-(thien-2-ylcarbonyl)pyrazolo[1,5-a]pyrimidin-7-yl]phenyl}urea;
N-[2-(Dimethylamino)ethyl]-N′-{3-[3-(thien-2-ylcarbonyl)pyrazolo[1,5-a]pyrimidin-7-yl]phenyl}urea;
N-Cyclopentyl-N′-{3-[3-(thien-2-ylcarbonyl)pyrazolo[1,5-a]pyrimidin-7-yl]phenyl}urea;
N-(Tetrahydrofuran-2-ylmethyl)-N′-{3-[3-(thien-2-ylcarbonyl)pyrazolo[1,5-a]pyrimidin-7-yl]phenyl}urea;
N-Neopentyl-N′-{3-[3-(thien-2-ylcarbonyl)pyrazolo[1,5-a]pyrimidin-7-yl]phenyl}urea;
N-Pentyl-N′-{3-[3-(thien-2-ylcarbonyl)pyrazolo[1,5-a]pyrimidin-7-yl]phenyl}urea;
4-Methyl-N-{3-[3-(thien-2-ylcarbonyl)pyrazolo[1,5-a]pyrimidin-7-yl]phenyl}piperazine-1-carboxamide;
4-Methyl-N-{3-[3-(thien-2-ylcarbonyl)pyrazolo[1,5-a]pyrithidin-7-yl]phenyl}-1,4-diazepane-1-carboxamide;
N,N-Bis(2-methoxyethyl)-N′-{3-[3-(thien-2-ylcarbonyl)pyrazolo[1,5-a]pyrimidin-7-yl]phenyl}urea;
N-Cyclopropyl-N′-{3-[3-(thien-2-ylcarbonyl)pyrazolo[1,5-a]pyrimidin-7-yl]phenyl}urea;
N-{3-[3-(Thien-2-ylcarbonyl)pyrazolo[1,5-a]pyrimidin-7-yl]phenyl}pyrrolidine-1-carboxamide;
Cyclopentyl 3-[3-(thien-2-ylcarbonyl)pyrazolo[1,5-a]pyrimidin-7-yl]phenylcarbamate;
Cyclohexyl 3-[3-(thien-2-ylcarbonyl)pyrazolo[1,5-a]pyrimidin-7-yl]phenylcarbamate;
Cyclohex-2-en-1-yl 3-[3-(thien-2-ylcarbonyl)pyrazolo[1,5-a]pyrimidin-7-yl]phenylcarbamate;
Cyclobutylmethyl 3-[3-(thien-2-ylcarbonyl)pyrazolo[1,5-a]pyrimidin-7-yl]phenylcarbamate;
Cyclohexylmethyl 3-[3-(thien-2-ylcarbonyl)pyrazolo[1,5-a]pyrimidin 7-yl]phenylcarbamate;
Tetrahydro-2H-pyran-4-yl 3-[3-(thien-2-ylcarbonyl)pyrazolo[1,5-a]pyrimidin-7-yl]phenylcarbamate;
3-Methylbut-2-enyl 3-[3-(thien-2-ylcarbonyl)pyrazolo[1,5-a]pyrimidin-7-yl]phenylcarbamate;
2-(Methylthio)ethyl 3-[3-(thien-2-ylcarbonyl)pyrazolo[1,5-a]pyrimidin-7-yl]phenylcarbamate;
2-Thien-3-ylethyl 3-[3-(thien-2-ylcarbonyl)pyrazolo[1,5-a]pyrimidin-7-yl]phenylcarbamate;
2-Piperidin-1-ylethyl 3-[3-(thien-2-ylcarbonyl)pyrazolo[1,5-a]pyrimidin-7-yl]phenylcarbamate;
2-Morpholin-4-ylethyl 3-[3-(thien-2-ylcarbonyl)pyrazolo[1,5-a]pyrimidin-7-yl]phenylcarbamate;
1-Methylprop-2-enyl 3-[3-(thien-2-ylcarbonyl)pyrazolo[1,5-a]pyrimidin-7-yl]phenylcarbamate;
1-Methylbut-3-ynyl 3-[3-(thien-2-ylcarbonyl)pyrazolo[1,5-a]pyrimidin-7-yl]phenylcarbamate;
(2-Methylcyclopropyl)methyl 3-[3-(thien-2-ylcarbonyl)pyrazolo[1,5-a]pyrimidin-7-yl]phenylcarbamate;
3-Methylcyclopentyl 3-[3-(thien-2-ylcarbonyl)pyrazolo[1,5-a]pyrimidin-7-yl]phenylcarbamate;
1-Methylbut-3-enyl 3-[3-(thien-2-ylcarbonyl)pyrazolo[1,5-a]pyrimidin-7-yl]phenylcarbamate;
1-Cyclopropylethyl 3-[3-(thien-2-ylcarbonyl)pyrazolo[1,5-a]pyrimidin-7-yl]phenylcarbamate;
But-3-ynyl 3-[3-(thien-2-ylcarbonyl)pyrazolo[1,5-a]pyriridin-7-yl]phenylcarbamate;
Allyl 3-[5-methyl-3-(thien-2-ylcarbonyl)pyrazolo[1,5-a]pyrimidin-7-yl]phenylcarbamate;
2-Chloroethyl 3-[5-methyl-3-(thien-2-ylcarbonyl)pyrazolo[1,5-a]pyrimidin-7-yl]phenylcarbarnate;
3-Methyl-N-{3-[5-methyl-3-(thien-2-ylcarbonyl)pyrazolo[1,5-a]pyrimidin-7-yl]phenyl}butanamide;
N-{3-[5-Methyl-3-(thien-2-ylcarbonyl)pyrazolo[1,5-a]pyrimidin-7-yl]phenyl}benzamide;
N-{3-[5-Methyl-3-(thien-2-ylcarbonyl)pyrazolo[1,5-a]pyrimidin-7-yl]phenyl}-2-thien-2-ylacetamide;
N-Butyl-N′-{3-[5-methyl-3-(thien-2-ylcarbonyl)pyrazolo[1,5-a]pyrimidin-7-yl]phenyl}urea;
Allyl 3-[6-methyl-3-(thien-2-ylcarbonyl)pyrazolo[1,5-a]pyrimidin-7-yl]phenylcarbamate;
2-Chloroethyl 3-[6-methyl-3-(thien-2-ylcarbonyl)pyrazolo[1,5-a]pyrimidin-7-yl]phenylcarbamate;
But-3-enyl 3-[6-methyl-3-(thien-2-ylcarbonyl)pyrazolo[1,5-a]pyrimidin-7-yl]phenylcarbamate;
N-{3-[6-Methyl-3-(thien-2-ylcarbonyl)pyrazolo[1,5-a]pyrimidin-7-yl]phenyl}benzamide;
N-{3-[6-Methyl-3-(thien-2-ylcarbonyl)pyrazolo[1,5-a]pyrimidin-7-yl]phenyl}-2-thien-2-ylacetamide;
N-{3-[6-Methyl-3-(thien-2-ylcarbonyl)pyrazolo[1,5-a]pyrimidin-7-yl]phenyl}nicotinamide;
N-Mutyl-N′-{3-[6-methyl-3-(thien-2-ylcarbonyl)pyrazolo[1,5-a]pyrimidin-7-yl]phenyl}urea;
1-[3-[3-(2-Furanylcarbonyl)pyrazolo-[1,5-a]pyrimidin-7-yl]phenyl]-2-piperidinone;
1-[3-(3-Benzoylpyrazolo[1,5-a]-pyrimidin-7-yl)phenyl]-2-piperidinone;
1-[3-[3-(2-Furanylcarbonyl)pyrazolo-[1,5-a]pyrimidin-7-yl]phenyl]-2-pyrrolidinone;
1-[3-(3-Benzoylpyrazolo[1,5-a]-pyrimidin-7-yl)phenyl]-2-pyrrolidinone;
N-[3-(3-Cyanopyrazolo[1,5-a]-pyrimidin-7-yl)phenyl]-N-methyl-cyclobutanecarboxamide;
[7-(2,4-Dimethoxyphenyl)pyrazolo[1,5-a]-pyrimidin-3-yl]-2-thienyl-methanone;
N,N-Diethyl-3-[3-(2-thienylcarbonyl)-pyrazolo[1,5-a]pyrimidin-7-yl]-benzamide;
N-[3-(3-Chloropyrazolo[1,5-a]pyrimidin-7-yl)phenyl]-N-ethyl-cyclopropanecarboxamide;
N-[3-(3-Cyanopyrazolo [1,5-a]pyrimidin-7-yl)phenyl]-N-ethyl-cyclopropanecarboxamide;
[7-[3-(2-Methoxyethoxy)phenyl]pyrazolo[1,5-a]pyrimidin-3-yl]-2-thienyl-methanone;
[7-[3-(Cyclopentyloxy)phenyl]pyrazolo[1,5-a]pyrimidin-3-yl]-2-thienyl-methanone;
2-Furanyl[7-[3-(1H-pyrrol-1-yl)phenyl]pyrazolo[1,5-a]pyrimidin-3-yl]-methanone;
[7-[3-(1H-Pyrrol-1-yl)phenyl]pyrazolo[1,5-a]pyrimidin-3-yl]-2-thienyl-methanone;
N-{3-[3-(2-Thienylcarbonyl)pyrazolo[1,5-a]pyrimidin-7-yl]phenyl}acrylamide;
N-{3-[3-(2-Thienylcarbonyl)pyrazolo[1,5-a]pyrimidin-7-yl]phenyl}-2-butynamide;
N-{3-[3-(2-Thienylcarbonyl)pyrazolo[1,5-a]pyrimidin-7-yl]phenyl}-2-oxiranecarboxamide;
N-{3-[3-(2-thienylcarbonyl)pyrazolo[1,5-a]pyrimidin-7-yl]phenyl}-2-oxiranecarboxamide;
N-{3-[5-Methyl-3-(2-thienylcarbonyl)pyrazolo[1,5-a]pyrimidin-7-yl]phenyl}-2-oxiranecarboxamide;
N-{3-[3-(2-Thienylcarbonyl)pyrazolo[1,5-a]pyrimidin-7-yl]phenyl}-2,5-bis(trifluoromethyl)benzamide;
N-{3-[3-(2-Thienylcarbonyl)pyrazolo[1,5-a]pyrimidin-7-yl]phenyl}-3-(trifluoromethyl)benzamide;
N-{3-[3-(2-Thienylcarbonyl)pyrazolo[1,5-a]pyrimidin-7-yl]phenyl}benzamide;
N-{3-[3-(2-Thienylcarbonyl)pyrazolo[1,5-a]pyrimidin-7-yl]phenyl}-2-furamide;
3-Bromo-N-{3-[3-(2-thienylcarbonyl)pyrazolo[1,5-a]pyrimidin-7-yl]phenyl}benzamide;
4-(tert-Butyl)-N-{3-[3-(2-thienylcarbonyl)pyrazolo[1,5-a]pyrimidin-7-yl]phenyl}benzamide;
3,5-Dinitro-N-{3-[3-(2-thienylcarbonyl)pyrazolo[1,5-a]pyrimidin-7-yl]phenyl}benzamide;
2,4-Dichloro-N-{3-[3-(2-thienylcarbonyl)pyrazolo[1,5-a]pyrimidin-7-yl]phenyl}benzamide;
N-{3-[3-(2-Thienylcarbonyl)pyrazolo[1,5-a]pyrimidin-7-yl]phenyl}nicotinamide;
N-{3-[3-(2-Thienylcarbonyl)pyrazolo[1,5-a]pyrimidin-7-yl]phenyl}isonicotinamide;
N,N′-Dibutyl-N-(3-[3-(2-thienylcarbonyl)pyrazolo[1,5-a]pyrimidin-7-yl]phenyl}dicarbonimidic diamide;
Isopropyl 3-[3-(cyclopentylcarbonyl)pyrazolo[1,5-a]pyrimidin-7-yl]phenylcarbamate;
N-{3-[3-(Cyclopentylcarbonyl)pyrazolo[1,5-a]pyrimidin-7-yl]phenyl}-3-methylbutanamide;
N-{3-[3-(2-Furoyl)pyrazolo[1,5-a]pyrimidin-7-yl]phenyl}benzamide;
Methyl 3-[3-(2-thienylcarbonyl)pyrazolo[1,5-a]pyrimidin-7-yl]benzoate;
N-{2-Methyl-5-[3-(2-thienylcarbonyl)pyrazolo[1,5-a]pyrimidin-7-yl]phenyl}benzamide;
3-Methyl-N-{2-methyl-5-[3-(2-thienylcarbonyl)pyrazolo[1,5-a]pyrimidin-7-yl]phenyl}butanamide
N-Isopropyl-N′-{2-methyl-5-[3-(2-thienylcarbonyl)pyrazolo[1,5-a]pyrimidin-7-yl]phenyl}urea;
N-{2-Chloro-5-[3-(2-thienylcarbonyl)pyrazolo[1,5-a]pyrimidin-7-yl]phenyl}benzamide;
N-{2-Chloro-5-[3-(2-thienylcarbonyl)pyrazolo[1,5-a]pyrimidin-7-yl]phenyl}-3-methylbutanamide;
Isopropyl 2-methyl-5-[3-(2-thienylcarbonyl)pyrazolo[1,5-a]pyrimidin-7-yl]phenylcarbamate;
N-(2-Methoxy-5-[3-(2-thienylcarbonyl)pyrazolo[1,5-a]pyrimidin-7-yl]phenyl}benzamide;
Isopropyl 2-methoxy-5-[3-(2-thienylcarbonyl)pyrazolo[1,5-a]pyrimidin-7-yl]phenylcarbamate;
N-{2-Methoxy-5-[3-(2-thienylcarbonyl)pyrazolo[1,5-a]pyrimidin-7-yl]phenyl}-3-methylbutanamide;
N-Isobutyl-3-[3-(2-thienylcarbonyl)pyrazolo[1,5-a]pyrimidin-7-yl]benzamide;
N-Butyl-3-[3-(2-thienylcarbonyl)pyrazolo[1,5-a]pyrimidin-7-yl]benzamide;
N-Cyclopentyl-3-[3-(2-thienylcarbonyl)pyrazolo[1,5-a]pyrimidin-7-yl]benzamide;
N-Phenyl-3-[3-(2-thienylcarbonyl)pyrazolo[1,5-a]pyrimidin-7-yl]benzamide;
N-Isopropyl-3-[3-(2-thienylcarbonyl)pyrazolo[1,5-a]pyrimidin-7-yl]benzamide;
N-Isopropyl-N′-{2-methoxy-5-[3-(2-thienylcarbonyl)pyrazolo[1,5-a]pyrimidin-7-yl]phenyl}urea;
3-Methyl-N-{5-[3-(2-thienylcarbonyl)pyrazolo[1,5-a]pyrimidin-7-yl]-3-pyridinyl}butanamide;
7-(3-Nitrophenyl)-3-(3-thienyl)pyrazolo[1,5-a]pyrimidine;
3-(3-Bromopyrazolo[1,5-a]pyrimidin-7-yl)aniline;
Isobutyl 3-(3-bromopyrazolo[1,5-a]pyrimidin-7-yl)phenylcarbamate;
[7-(5-Nitro-3-thienyl)pyrazolo[1,5-a]pyrimidin-3-yl](2-thienyl)methanone;
N-Isopropyl-N′-{5-[3-(thien-2-ylcarbonyl)pyrazolo[1,5-a]pyrimidin-7-yl]thien-2-yl}urea;
N-[3-(3-Bromopyrazolo[1,5-a]pyrimidin-7-yl)phenyl]-3-methylbutanamide;
[7-(5-Aminothien-3-yl)pyrazolo[1,5-a]pyrimidin-3-yl](thien-2-yl)methanone;
O-Ethyl 3-[3-(thien-2-ylcarbonyl)pyrazolo[1,5-a]pyrimidin-7-yl]phenylthiocarbamate;
3-Methyl-N-[3-(3-thien-2-ylpyrazolo[1,5-a]pyrimidin-7-yl)phenyl]butanamide;
N-Phenyl-N′-{3-[3-(thien-2-ylcarbonyl)pyrazolo[1,5-a]pyrimidin-7-yl]phenyl}thiourea;
Isopropyl 4-chloro-3-[3-(thien-2-ylcarbonyl)pyrazolo[1,5-a]pyrimidin-7-yl]phenylcarbamate;
[7-(2-Chloro-5-nitrophenyl)pyrazolo[1,5-a]pyrimidin-3-yl](thien-2-yl)methanone;
N-{3-[3-(Pyridin-2-ylcarbonyl)pyrazolo[1,5-a]pyrimidin-7-yl]phenyl}cyclopropanecarboxamide;
N-{3-[3-(Pyridin-2-ylcarbonyl)pyrazolo[1,5-a]pyrimidin-7-yl]phenyl}cyclopropanecarboxamide;
N-{4-Chloro-3-[3-(thien-2-ylcarbonyl)pyrazolo[1,5-a]pyrimidin-7-yl]phenyl}-3-methylbutanamide;
N-{4-Chloro-3-[3-(thien-2-ylcarbonyl)pyrazolo[1,5-a]pyrimidin-7-yl]phenyl}-N′-isopropylthiourea;
N-{4-Chloro-3-[3-(thien-2-ylcarbonyl)pyrazolo [1,5-a]pyrimidin-7-yl]phenyl}benzamide;
N-{2-Fluoro-5-[3-(thien-2-ylcarbonyl)pyrazolo[1,5-a]pyrimidin-7-yl]phenyl}-3-methylbutanamide;
3-Methyl-N-{5-[3-(thien-2-ylcarbonyl)pyrazolo[1,5-a]]pyrimidin-7-yl]thien-2-yl}butanamide;
Isopropyl 5-[3-(thien-2-ylcarbonyl)pyrazolo[1,5-a]pyrimidin-7-yl]thien-2-ylcarbamate;
[7-(5-Aminothien-2-yl)pyrazolo[1,5-a]pyrimidin-3-yl](thien-2-yl)methanone;
4-Chloro-N-{3-[3-(thien-2-ylcarbonyl)pyrazolo[1,5-a]pyrimidin-7-yl]phenyl}butanamide;
Isopropyl 2-fluoro-5-[3-(thien-2-ylcarbonyl)pyrazolo[1,5-a]pyrimidin-7-yl]phenylcarbamate;
Isopropyl 2-(dimethylamino)-5-[3-(thien-2-ylcarbonyl)pyrazolo[1,5-a]pyrimidin-7-yl]phenylcarbamate;
{7-[3-(Benzylamino)phenyl]pyrazolo[1,5-a]pyrimidin-3-yl}(thien-2-yl)methanone;
3-Methyl-N-{4-[3-(thien-2-ylcarbonyl)pyrazolo[1,5-a]pyrimidin-7-yl]thien-2-yl}butanamide;
[7-(3-Amino-4-fluorophenyl)pyrazolo[1,5-a]pyrimidin-3-yl](thien-2-yl)methanone;
N-Cyclohexyl-N′-{3-[3-(thien-2-ylcarbonyl)pyrazolo[1,5-a]pyrimidin-7-yl]phenyl}urea;
4,4,4-Trichloro-N-{3-[3-(thien-2-ylcarbonyl)pyrazolo[1,5-a]pyrimidin-7-yl]phenyl}butanamide;
N-{2-Fluoro-5-[3-(thien-2-ylcarbonyl)pyrazolo[1,5-a]pyrimidin-7-yl]phenyl}-3,3-dimethylbutanamide;
4,4,4-Trifluoro-N-{2-fluoro-5-[3-(thien-2-ylcarbonyl)pyrazolo[1,5-a]pyrimidin-7-yl]phenyl}-3-methylbutanamide;
Bis(2,2,2-trichloroethyl) 2-fluoro-5-[3-(thien-2-ylcarbonyl)pyrazolo[1,5-a]pyrimidin-7-yl]phenylimidodicarbonate;
2-Fluoro-N-{3-[3-(thien-2-ylcarbonyl)pyrazolo[1,5-a]pyrimidin-7-yl]phenyl}benzamide;
2,6-Difluoro-N-{3-[3-(thien-2-ylcarbonyl)pyrazolo[1,5-a]pyrimidin-7-yl]phenyl}benzamide;
N-{3-[3-(Thien-2-ylcarbonyl)pyrazolo[1,5-a]pyrimidin-7-yl]phenyl}pyridine-2-carboxamide;
N-{5-[3-(Thien-2-ylcarbonyl)pyrazolo[1,5-a]pyrimidin-7-yl]thien-2-yl}benzamide;
(7-{3-[(2-Phenylethyl)aminolphenyl}pyrazolo[1,5-a]pyrimidin-3-yl)(thien-2-yl)methanone;
(7-{3-[(2-Methoxybenzyl)amino]phenyl}pyrazolo[1,5-a]pyrimidin-3-yl)(thien-2-yl)methanone;
N-Butyl-N′-{3-[3-(thien-2-ylcarbonyl)pyrazolo[1,5-a]pyrimidin-7-yl]phenyl}thiourea;
(7-{3-[(3-Chlorobenzyl)amino]phenyl}pyrazolo[ I,5-a]pyrimidin-3-yl)(thien-2-yl)methanone;
(7-{3-[(4-Chlorobenzyl)amino]phenyl}pyrazolo[1,5-a]pyrimidin-3-yl)(thien-2-yl)methanone;
(7-{3-[(2-Furylmethyl)amino]phenyl}pyrazolo[1,5-a]pyrimidin-3-yl)(thien-2-yl)methanone;
(7-{3-[(2-Chlorobenzyl)amino]phenyl}pyrazolo[1,5-a]pyrimidin-3-yl)(thien-2-yl)methanone;
(7-{3-[(3-Furylmethyl)amino]phenyl}pyrazolo[1,5-a]pyrimidin-3-yl)(thien-2-yl)methanone;
(7-{3-[(2E)-But-2-enylamino]phenyl}pyrazolo[1,5-a]pyrimidin-3-yl)(thien-2-yl)methanone;
(7-{3-[(3-Methylbut-2-enyl)amino]phenyl}pyrazolo[1,5-a]pyrimidin-3-yl)(thien-2-yl)methanone;
[7-(6-Chloropyridin-3-yl)pyrazolo[1,5-a]pyrimidin-3-yl](thien-2-yl)methanone;
2,6-Difluoro-N-{2-fluoro-5-[3-(thien-2-ylcarbonyl)pyrazolo[1,5-a]pyrimidin-7-yl]phenyl}benzamide;
2-Fluoro-N-{2-fluoro-5-[3-(thien-2-ylcarbonyl)pyrazolo[1,5-a]pyrimidin-7-yl]phenyl}benzamide;
2-Chloro-N-{2-fluoro-5-[3-(thien-2-ylcarbonyl)pyrazolo[1,5-a]pyrimidin-7-yl]phenyl}benzamide;
(7-{3-[(2-Methylbenzyl)amino]phenyl}pyrazolo[1,5-a]pyrimidin-3-yl)(thien-2-yl)methanone;
(7-{3-[(2-Phenylpropyl)amino]phenyl}pyrazolo[1,5-a]pyrimidin-3-yl)(thien-2-yl)methanone;
(7-{3-[(Cyclohexylmethyl)amino]phenyl}pyrazolo[1,5-a]pyrimidin-3-yl)(thien-2-yl)methanone;
2-Chloroethyl 2-fluoro-5-[3-(thien-2-ylcarbonyl)pyrazolo[1,5-a]pyrimidin-7-yl]phenylcarbamate;
N-{2-Fluoro-5-[3-(thien-2-ylcarbonyl)pyrazolo[1,5-a]pyrimidin-7-yl]phenyl}-N′-isopropylurea;
4-(1H-Pyrrol-1-yl)-N-{3-[3-(thien-2-ylcarbonyl)pyrazolo[1,5-a]pyrimidin-7-yl]phenyl}benzamide;
2,6-Dichloro-N-(3-[3-(thien-2-ylcarbonyl)pyrazolo[1,5-a]pyrimidin-7-yl]phenyl}benzamide;
N-(3-[3-(Thien-2-ylcarbonyl)pyrazolo[1,5-a]pyrimidin-7-yl]phenyl}pyrazine-2-carboxamide;
Phenyl N′-cyano-N-{3-[3-(thien-2-ylcarbonyl)pyrazolo[1,5-a]pyrimidin-7-yl]phenyl}imidocarbamate;
1-Methyl-N-{3-[3-(thien-2-ylcarbonyl)pyrazolo[1,5-a]pyrimidin-7-yl]phenyl)-1H-Pyrrole-2-carboxamide;
N-{2-Fluoro-5-[3-(thien-2-ylcarbonyl)pyrazolo[1,5-a]pyrimidin-7-yl]phenyl}isonicotinamide;
N-Isopropyl-N′-(3-[3-(thien-2-ylcarbonyl)pyrazolo[1,5-a]pyrimidin-7-yl]phenyl)thiourea;
(7-Chloropyrazolo[1,5-a]pyrimidin-3-yi)(thien-2-yl)methanone;
2,6-Dichloro-N-(2,6-dichlorobenzoyl)-N-{3-[3-(thien-2-ylcarbonyl)pyrazolo[1,5-a]pyrimidin-7-yl]phenyl}benzamide;
tert-Butyl 4-[2-oxo-2-({3-[3-(thien-2-ylcarbonyl)pyrazolo[1,5-a]pyrimidin-7-yl]phenyl}amino)ethyl]piperidine-1-carboxylate;
2-Piperidin-4-yl-N-(3-[3-(thien-2-ylcarbonyl)pyrazolo[1,5-a]pyrimidin-7-yl]phenyl}acetamide;
[7-(3-{[(2E)-3-(2-Methoxyphenyl)prop-2-enyl]amino}phenyl)pyrazolo[1,5-a]pyrimidin-3-yl](thien-2-yl)methanone;
[7-(3-{[(2E)-3-(4-Methoxyphenyl)prop-2-enyl]amino}phenyl)pyrazolo[1,5-a]pyrimidin-3-yl](thien-2-yl)methanone;
[7-(3-{[(2E)-2-Methyl-3-phenylprop-2-enyl]amino}phenyl)pyrazolo[1,5-a]pyrimidin-3-yl](thien-2-yl)methanone;
[7-(3-{[(2E)-3-(4-Nitrophenyl)prop-2-enyl]amino}phenyl)pyrazolo[1,5-a]pyrimidin-3-yl](thien-2-yl)methanone;
(7-{3-[(3,3-Diphenylprop-2-enyl)amino]phenyl}pyrazolo[1,5-a]pyrimidin-3-yl)(thien-2-yl)methanone;
(7-{3-[(3-Phenylbutyl)amino]phenyl}pyrazolo[1,5-a]pyrimidin-3-yl)(thien-2-yl)methanone;
[7-(3-{[(2E)-3-(4-Hydroxy-3-methoxyphenyl)prop-2-enyl]amino}phenyl)pyrazolo[1,5-a]pyrimidin-3-yl](thien-2-yl)methanone;
tert-Butyl 4-[({3-[3-(thien-2-ylcarbonyl)pyrazolo[1,5-a]pyrimidin-7-yl]phenyl}amino)carbonyl]piperidine-1-carboxylate;
N,N′-Bis {3-[3-(thien-2-ylcarbonyl)pyrazolo[1,5-a]pyrimidin-7-yl]phenyl}thiourea;
Isobutyl 3-(3-thien-2-ylpyrazolo[1,5-a]pyrimidin-7-yl)phenylcarbamate;
N-{3-[3-(Thien-2-ylcarbonyl)pyrazolo[1,5-a]pyrimidin-7-yl]phenyl}piperidine-4-carboxamide;
[7-(3-{[(2E)-2-Hexyl-3-phenylprop-2-enyl]amino}phenyl)pyrazolo[1,5-a]pyrimidin-3-yl](thien-2-yl)methanone;
[7-(3-{[(2E)-2-Pentyl-3-phenylprop-2-enyl]amino}phenyl)pyrazolo[1,5-a]pyrimidin-3-yl](thien-2-yl)methanone;
N-Isopropyl-N′-{3-[3-(thien-2-ylcarbonyl)pyrazolo[1,5-a]pyrimidin-7-yl]phenyl}guanidine;
[7-(3-{[(2E)-3-Phenylprop-2-enyl]amino)phenyl)pyrazolo[1,5-a]pyrimidin-3-yl](thien-2-yl)methanone;
[7-(3-{[(2E)-3-(4-tert-Butylphenyl)-2-methylprop-2-enyl]amino}phenyl)pyrazolo[1,5-a]pyrimidin-3-yl](thien-2-yl)methanone;
N′-Cyano-N-isopropyl-N′-{3-[3-(thien-2-ylcarbonyl)pyrazolo[1,5-a]pyrimidin-7-yl]phenyl}guanidine;
N-{3-[3-(Thien-2-ylcarbonyl)pyrazolo[1,5-a]pyrimidin-7-yl]phenyl}-1H-imidazole-5-carboxamide;
Pyridin-4-ylmethyl 3-[3-(thien-2-ylcarbonyl)pyrazolo[1,5-a]pyrimidin-7-yl]phenylcarbamate;
1-Methyl-N-{3-[3-(thien-2-ylcarbonyl)pyrazolo[1,5-a]pyrimidin-7-yl]phenyl}piperidine-4-carboxamide;
(7-{3-[(2-Ethylhexyl)amino]phenyl}pyrazolo[1,5-a]pyrimidin-3-yl)(thien-2-yl)methanone;
3-(1,1-Dioxidothiomorpholin-4-yl)propyl 3-[3-(thien-2-ylcarbonyl)pyrazolo[1,5-a]pyrimidin-7-yl]phenylcarbamate;
2-Chloro-N-{2-(dimethylamino)-5-[3-(thien-2-ylcarbonyl)pyrazolo[1,5-a]pyrimidin-7-yl]phenyl}benzamide;
4-Morpholin-4-yl-N-{3-[3-(thien-2-ylcarbonyl)pyrazolo[1,5-a]pyrimidin-7-yl]phenyl}butanamide;
Pyridin-3-ylmethyl 3-[3-(thien-2-ylcarbonyl)pyrazolo[1,5-a]pyrimidin-7-yl]phenylcarbamate;
3-Morpholin-4-ylpropyl 3-[3-(thien-2-ylcarbonyl)pyrazolo[1,5-a]pyrimidin-7-yl]phenylcarbamate;
[7-(3-{[(2E)-2-Methylpent-2-enyl]amino}phenyl)pyrazolo[1,5-a]pyrimidin-3-yl](thien-2-yl)methanone;
2-Pyridin-4-yl-N-{3-[3-(thien-2-ylcarbonyl)pyrazolo[1,5-a]pyrimidin-7-yl]phenyl}acetamide;
N-{2-(Dimethylamino)-5-[3-(thien-2-ylcarbonyl)pyrazolo[1,5-a]pyrimidin-7-yl]phenyl}isonicotinamide;
Pyridin-2-ylmethyl 3-[3-(thien-2-ylcarbonyl)pyrazolo[1,5-a]pyrimidin-7-yl]phenylcarbamate;
Thien-2-yl(7-{3-[(3,5,5-trimethylhexyl]amino]phenyl}pyrazolo[1,5-a]pyrimidin-3-yl](thien-2-yl)methanone;
(7-{3-[(Cyclohex-3-en-1-ylmethyl)emino]phenyl}pyrazolo[1,5-a]pyrimidin-3-yl)-(thien-2-yl)-methanone;
1-{3-[3-(Thien-2-ylcarbonyl)pyrazolo[1,5-a]pyrimidin-7-yl]phenyl}pyrrolidin-2-one;
2-Chloro-N-(2-chlorobenzoyl)-N-{2-fluoro-5-[3-(thien-2-ylcarbonyl)pyrazolo[1,5-a]pyrimidin-7-yl]phenyl}benzamide;
2-Pyridin-3-yl-N-{3-[3-(thien-2-ylcarbonyl)pyrazolo[1,5-a]pyrimidin-7-yl]phenyl}acetamide;
3-Pyridin-4-yl-N-{3-[3-(thien-2-ylcarbonyl)pyrazolo[1,5-a]pyrimidin-7-yl]phenyl}propanamide;
N-{3-[3-(Thien-2-ylcarbonyl)pyrazolo[1,5-a]pyrimidin-7-yl]phenyl}-1H-pyrazole-4-arboxamide;
4-oxo-4-{3-[3-(Thien-2-ylcarbonyl)pyrazolo[1,5-a]pyrimidin-7-yl]phenyl}amino)butanoic acid;
2-[({3-[3-(Thien-2-ylcarbonyl)pyrazolo[1,5-a]pyrimidin-7-yl]phenyl}amino)carbonyl]benzoic acid;
5-Oxo-5-({3-[3-(thien-2-ylcarbonyl)pyrazolo[1,5-a]pyrimidin-7-yl]phenyl}amino)pentanoic acid;
3-Methyl-5-oxo-5-({3-[3-(thien-2-ylcarbonyl)pyrazolo[1,5-a]pyramidin-7-yl]phenyl}amino)pentanoic acid;
4,5-Dichloro-2-[({3-[3-(thien-2-ylcarbonyl)pyrazolo[1,5-a]pyrimidin-7-yl]phenyl}amino)carbonyl]benzoic acid;
(7-{3-[(2-Methylundecyl)amino]phenyl}pyrazolo[1,5-a]pyrimidin-3-yl)(thien-2-yl)methanone;
3-Pyridin-3-yl-N-{3-[3-(thien-2-ylcarbonyl)pyrazolo[1,5-a]pyrimidin-7-yl]phenyl}propanamide;
2-(Pyridin-4-ylthio)-N-{3-[3-(thien-2-ylcarbonyl)pyrazolo[1,5-a]pyrimidin-7-yl]phenyl}acetamide;
{7-[4′-(Dimethylamino)-1,1′-biphenyl-3-yl]pyrazolo[1,5-a]pyrimidin-3-yl }(thien-2-yl)methanone;
[7-(3′-Amino-1,1′-biphenyl-3-yl)pyrazolo[1,5-a]pyrimidin-3-yl] (thien-2-yl)methanone;
{7-[4′-(Hydroxymethyl)-1,1′-biphenyl-3-yl]pyrazolo[1,5-a]pyrimidin-3-yl}(thien-2-yl)methanone;
{7-[3′-(Dimethylamino)-1,1′-biphenyl-3-yl]pyrazolo[1,5-a]pyrimidin-3-yl}(thien-2-yl)methanone;
{7-[4′-(Dimethylamino)-6-fluoro-1,1′-biphenyl-3-yl]pyrazolo[1,5-a]pyrimidin-3-yl}(thien-2-yl)methanone;
{7-[6-Fluoro-4′-(hydroxymethyl)-1,1′-biphenyl-3-yl]pyrazolo[1,5-a]pyrimidin-3-yl}(thien-2-yl)methanone;
[7-(4′-Ethyl-1,1′-biphenyl-3-yl)pyrazolo[1,5-a]pyrimidin-3-yl] (thien-2-yl)methanone;
3-{3′-[3-(Thien-2-ylcarbonyl)pyrazolo[1,5-a]pyrimidin-7-yl]-1,1′-biphenyl-4-yl}propanoic acid;
3′-[3-(Thien-2-ylcarbonyl)pyrazolo[1,5-a]pyrimidin-7-yl]-1,1′-biphenyl-4-carbaldehyde;
{7-[4′-(Morpholin-4-ylmethyl)-1,1′-biphenyl-3-yl]pyrazolo[1,5-a]pyrimidin-3-yl}(thien-2-yl)methanone;
(7-{4′-[(4-Methylpiperazin-1-yl)methyl]-1,1′-biphenyl-3-yl}pyrazolo[1,5-a]pyrimidin-3-yl)(thien-2-yl)methanone;
[7-(4′-{[(2-Morpholin-4-ylethyl)amino]methyl}-1,1′-biphenyl-3-yl)pyrazolo[1,5-a]pyrimidin-3-yl](thien-2-yl)methanone;
(7-{4′-[(Diethylamino)methyl]-1,1′-biphenyl-3-yl}pyrazolo[1,5-a]pyrimidin-3-yl)(thien-2-yl)methanone;
(7-(4′-[(Dimethylamino)methyl]-1,1′-biphenyl-3-yl}pyrazolo[1,5-a]pyrimidin-3-yl)(thien-2-yl)methanone;
N-{3-[3-(3-Aminophenyl)pyrazolo[1,5-a]pyrimidin-7-yl]phenyl}-3-methylbutanamide;
N-(3-{3-[3-(Dimethylamino)phenyl]pyrazolo[1,5-a]pyrimidin-7-yl}phenyl)-3-methylbutanamide;
N-{3-[3-( 1H-Indol-5-yl)pyrazolo[1,5-a]pyrimidin-7-yl]phenyl}-3-methylbutanamide;
3-Methyl-N-{3-[3-(3,4,5-trimethoxyphenyl)pyrazolo[1,5-a]pyrimidin-7-yl]phenyl}butanamide;
3-Methyl-N-[3-(3-quinolin-8-ylpyrazolo[1,5-a]pyrimidin-7-yl)phenyl]butanamide;
N-(3-{3-[4-(Hydroxymethyl)phenyl]pyrazolo[1,5-a]pyrimidin-7-yl}phenyl)-3-methylbutanamide;
N-{3-[3-(4-Methoxyphenyl)pyrazolo[1,5-a]pyrimidin-7-yl]phenyl}-3-methylbutanamide;
N-{3-[3-(3-Chlorophenyl)pyrazolo[1,5-a]pyrimidin-7-yl]phenyl}-3-methylbutanamide;
N-{3-[3-(4-Chlorophenyl)pyrazolo[1,5-a]pyrimidin-7-yl]phenyl}-3-methylbutanamide;
N-(3-[3-(2,3-Dichlorophenyl)pyrazolo[1,5-a]pyrimidin-7-yl]phenyl}-3-methylbutanamide;
N-{3-[3-(3,4-Dichlorophenyl)pyrazolo[1,5-a]pyrimidin-7-yl]phenyl}-3-methylbutanamide;
N-(3-[3-(3,5-Dichlorophenyl)pyrazolo[1,5-a]pyrimidin-7-yl]phenyl}-3-methylbutanamide;
N-{3-[3-(3,5-Dimethylphenyl)pyrazolo[1,5-a]pyrimidin-7-yl]phenyl}-3-methylbutanamide;
3-Methyl-N-(3-{3-[3-(trifluoromethyl)phenyl]pyrazolo[1,5-a]pyrimidin-7-yl}phenyl)butanamide;
N-{3-[3-(3-Methoxyphenyl)pyrazolo[1,5-a]pyrimidin-7-yl]phenyl}-3-methylbutanamide;
N-{3-[3-(3-Ethoxyphenyl)pyrazolo[1,5-a]pyrimidin-7-yl]phenyl}-3-methylbutanamide;
3-Methyl-N-{3-[3-(4-phenoxyphenyl)pyrazolo[1,5-a]pyrimidin-7-yl]phenyl}butanamide;
N-{3-[3-(1,3-Benzodioxol-5-yl)pyrazolo[1,5-a]pyrimidin-7-yl]phenyl}-3-methylbutanamide;
N-{3-[3-(3-Cyanophenyl)pyrazolo[1,5-a]pyrimidin-7-yl]phenyl}-3-methylbutanamide;
N-{3-t3-(3-Acetylphenyl)pyrazolo[1,5-a]pyrimidin-7-yl]phenyl}-3-methylbutanamide;
N-{3-[3-(3-Formyl-4-methoxyphenyl)pyrazolo[1,5-a]pyrimidin-7-yl]phenyl}-3-methylbutanamide;
N-{3-[3-(1,1′-Biphenyl-3-yl)pyrazolo[1,5-a]pyrimidin-7-yl]phenyl}-3-methylbutanamide;
N-{3-[3-(1,1′-Biphenyl-4-yl)pyrazolo[1,5-a]pyrimidin-7-yl]phenyl}-3-methylbutanamide;
N-{3-[3-(3-Formylphenyl)pyrazolo[1,5-a]pyrimidin-7-yl]phenyl}-3-methylbutanamide;
3-Methyl-N-(3-{3-[3-(morpholin-4-ylmethyl)phenyl]pyrazolo[1,5-a]pyrimidin-7-yl}phenyl)butanamide;
3-Methyl-N-[3-(3-{3-[(4-methylpiperazin-1-yl)methyl]phenyl}pyrazolo[1,5-a]pyrimidin-7-yl)phenyl]butanamide;
3-[3-(3,5-Dimethylphenyl)pyrazolo[1,5-a]pyrimidin-7-yl]benzonitrile;
3-(3,5-Dimethylphenyl)-7-[3-(1H-tetraazol-5-yl)phenyl]pyrazolo[1,5-a]pyrimidine;
7-{3-[2-(Cyclobutylmethyl)-2H-tetraazol-5-yl]phenyl}-3-(3,5-dimethylphenyl)pyrazolo[1,5-a]pyrimidine;
3-(3,5-Dimethylphenyl)-7-{3-[2-(2-morpholin-4-ylethyl)-2H-tetraazol-5-yl]phenyl)pyrazolo[1,5-a]pyrimidine;
3-(3,5-Dimethylphenyl)-7-{3-[2-(pyridin-3-ylmethyl)-2H-tetrazol-5-yl]phenyl}pyrazolo[1,5-a]pyrimidine;
3-(3,5-Dimethylphenyl)-7-{3-[2-(pyridin-2-ylmethyl)-2H-tetrazol-5-yl]phenyl}pyrazolo[1,5-a]pyrimidine;
3-(3,5-Dimethylphenyl)-7-[3-(2-ethyl-2H-tetrazol-5-yl)phenyl]pyrazolo[1,5-a]pyrimidine;
N-[3-(3-{3-[(Dimethylamino)methyl]phenyl}pyrazolo[1,5-a]pyrimidin-7-yl)phenyl]-3-methylbutanamide;
3-(3,5-Dimethylphenyl)-7-{3-[2-(2-pyrrolidin-1-ylethyl)-2H-tetrazol-5-yl]phenyl}pyrazolo[1,5-a]pyrimidine;
3-(3,5-Dimethylphenyl)-7-{3-[2-(2-piperidin-I -ylethyl)-2H-tetrazol-5-yl]phenyl}pyrazolo[1,5-a]pyrimidine;
3-(3,5-Dimethylphenyl)-7-{3-[2-(pyridin-4-ylmethyl)-2H-tetrazol-5-yl]phenyl}pyrazolo[1,5-a]pyrimidine;
{7-[3-(1H-Tetraazol-5-yl)phenyl]pyrazolo[1,5-a]pyrimidin-3-yl}(thien-2-yl)methanone;
{7-[3-(2-Ethyl-2H-tetraazol-5-yl)phenyl]pyrazolo[1,5-a]pyrimidin-3-yl}(thien-2-yl)methanone;
(7-{3-[2-(Pyridin-3-ylmethyl)-2H-tetraazol-5-yl]phenyl}pyrazolo[1,5-a]pyrimidin-3-yl)(thien-2-yl)methanone;
{7-[3-(2-Isobutyl-2H-tetraazol-5-yl)phenyl]pyrazolo[1,5-a]pyrimidin-3-yl}(thien-2-yl)methanone;
(7-{3-[2-(Cyclobutylmethyl)-2H-tetraazol-5-yl]phenyl}pyrazolo[1,5-a]pyrimidin-3-yl)(thien-2-yl)methanone;
{7-[3-(2-Butyl-2H-tetraazol-5-yl)phenyl]pyrazolo[1,5-a]pyrimidin-3-yl}(thien-2-yl)methanone;
(7-{3-[2-(2-Morpholin-4-ylethyl)-2H-tetraazol-5-yl]phenyl}pyrazolo[1,5-a]pyrimidin-3-yl)(thien-2-yl)methanone;
(7-{3-[1-(Cyclobutylmethyl)-1H-tetraazol-5-yl]phenyl}pyrazolo[1,5-a]pyrimidin-3-yl)(thien-2-yl)methanone;
(7-{3-[2-(Pyridin-2-ylmethyl)-2H-tetraazol-5-yl]phenyl}pyrazolo[1,5-a]pyrimidin-3-yl)(thien-2-yl)methanone;
(7-{3-[2-(Pyridin-4-ylmethyl)-2H-tetraazol-5-yl]phenyl}pyrazolo[1,5-a]pyrimidin-3-yl)(thien-2-yl)methanone;
(7-{3-[2-(2-Pyrrolidin-1-ylethyl)-2H-tetraazol-5-yl]phenyl}pyrazolo[1,5-a]pyrimidin-3-yl)(thien-2-yl)methanone;
(7-{3-[2-(2-Piperidin-1-ylethyl)-2H-tetraazol-5-yl]phenyl}pyrazolo[1,5-a]pyrimidin-3-yl)(thien-2-yl)methanone;
(7-{3-[2-(2-Hydroxyethyl)-2H-tetraazol-5-yl]phenyl}pyrazolo[1,5-a]pyrimidin-3-yl)(thien-2-yl)methanone;
{7-[3-(1H-Pyrazol-3-yl)phenyl]pyrazolo[1,5-a]pyrimidin-3-yl}(thien-2-yl)methanone;
(7-{3-[1-(Cyclobutylmethyl)- 1H-pyrazol-3-yl]phenyl}pyrazolo[1,5-a]pyrimidin-3-yl)(thien-2-yl)methanone;
(7-{3-[1-(2-Morpholin-4-ylethyl)-1H-pyrazol-3-yl]phenyl}pyrazolo[1,5-a]pyrimidin-3-yl)(thien-2-yl)methanone;
(7-{3-[1-(Pyridin-2-ylmethyl)-1H-pyrazol-3-yl]phenyl}pyrazolo[1,5-a]pyrimidin-3-yl)(thien-2-yl)methanone;
{7-[3-(1-Ethyl-1H-pyrazol-3-yl)phenyl]pyrazolo[1,5-a]pyrimidin-3-yl}(thien-2-yl)methanone;
(7-{3-[1-(2-Piperidin-1-ylethyl)- 1H-pyrazol-3-yl]phenyl}pyrazolo[1,5-a]pyrimidin-3-yl)(thien-2-yl)methanone;
(7-{3-[1-(2-Pyrrolidin-1-ylethyl)-1H-pyrazol-3-yl]phenyl}pyrazolo[1,5-a]pyrimidin-3-yl)(thien-2-yl)methanone;
and pharmaceutically acceptable salts and prodrugs thereof.

5. A compound according to claim 1, wherein the unsubstituted or substituted aryl or heteroaryl of R₅ is selected from the group consisting of phenyl, naphthalenyl, thiazolyl, biphenyl, thienyl, furanyl and pyridinyl.

6. A compound according to any one of claims 1, 2 and 3, wherein the unsubstituted, monosubstituted or disubstituted aryl or heteroaryl of R₁₀ is selected from the group consisting of phenyl, naphthalenyl, thiazolyl, biphenyl, thienyl, furanyl and pyridinyl.

7. A compound according to claim 6, wherein the one or two substituents of the aryl or heteroaryl of R₁₀ are independently selected from the group consisting of halogen, nitro, cyano, CF₃, OCF₃, carboxy, carbamoyl, alkyl, alkoxy, alkanol, trifluoromethyl, alkylamino, alkylthio, dialkylamino, methylenedioxy, alkylsulfonyl and alkanoylamino.

8. A method of treating or inhibiting the growth of p21 deficient cells comprising administering at least one compound of claim 1 or a biologically active metabolite thereof.

9. A method of treating or inhibiting the growth of a neoplasm comprising administering to a mammal in need thereof a therapeutically effective amount of at least one compound of claim 1 or a physiologically acceptable salt, prodrug or biologically active metabolites thereof.

10. The method according to claim 9, wherein the neoplasm is selected from the group consisting of colon, breast, kidney, bladder, mouth, larynx,.esophagus, stomach, colon, ovary, and lung.

11. A method of preparing a compound of Formula I having the structure: 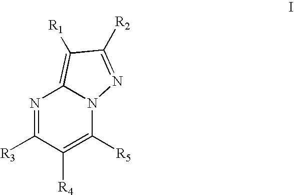
and pharmaceutically acceptable salts and prodrugs thereof,
wherein
R₁ is selected from the groups consisting of hydrogen, cyano, halogen, carbamoyl, formyl, carboxy, C(O)O-alkyl, C(O)O-cycloalkyl, C(O)cycloalkyl , R₆, C(O)R₆, and C(S)R₆;
R₆ is unsubstituted, monosubstituted or disubstituted aryl or heteroaryl wherein aryl or heteroaryl is phenyl, naphthalenyl, thiazolyl, biphenyl, thienyl, furanyl, or pyridinyl; and the substituents are selected from the groups consisting of halogen, nitro, cyano, CF₃, OCF₃, alkyl, alkoxy, trifluoromethyl, alkanol, alkylamino, alkylthio, dialkylamino, methylenedioxy, alkylsulfonyl and alkanoylamino;
R₂, R₃, and R₄ are hydrogen, CF₃, or alkyl;
R₅ is unsubstituted aryl or heteroaryl, or aryl or heteroaryl substituted by R₇, R₈, R₉, or R₁₀ with the proviso that R₁₀ must be present if aryl or heteroaryl is substituted;
R₇, R₈, and R₉ are independently selected from the groups consisting of hydrogen, nitro, cyano, carbamoyl, halogen, N(CH₃)₂, CF₃, OCF₃, alkyl, alkoxy, and carboxy;
R₁₀ is selected from nitro, cyano, carboxy, carbamoyl, halogen, CF₃, OCF₃, alkyl, alkoxy, alkoxyalkoxy, alkanol, NR₁₁R₁₂, N(R₁₃)COR₁₁, N(R₁₃)CONR₁₁R₁₂, OCONR₁₁R₁₂, N(R₁₃)CO₂R₁₁, N(R₁₃)CSR₁₁, N(R₁₃)CS(NR₁₁R₁₂), N(R₁₃)CS(OR₁₁), N(R₁₃)SO₂R₁₁, N(CONR₁₃R₁₁)₂, N(CO₂R₁₁)₂, N(COR₁₁)₂, N(CONR₁₃R₁₁)₂, CONR₁₁R₁₂, CO₂R₁₁, NHC(═NH)NHR₁₁, NHC(═NH—CN)NR₁₁R₁₂, NHC(═NH—CN)OR₁₁, C(═NH)NHR₁₁, C(═NH)NR₁₁R₁₂, LCONH₂, LCONR₁₁R₁₂, or LCO₂R₁₁ wherein L is alkyl, alkenyl or alkynyl or R₁₀ is selected from unsubstituted, monosubstituted, or disubstituted aryl or heteroaryl which is a 5- or 6-membered ring moiety containing at least 1-4 heteroatoms selected from O, S, and N;
R₁₁ and R₁₂ are independently selected from the groups consisting of hydrogen, alkyl, alkenyl, alkynyl, cycloalkyl, Q₁, Q₂, -L-Q₁ and -L-Q₂; wherein Q₁ is unsubstituted aryl or heteroaryl, monosubstituted or disubstituted aryl or heteroaryl; wherein Q₁ comprises phenyl, naphthalenyl, biphenyl, thiazolyl, oxazolyl, pyrrolyl, pyrrazolyl, thienyl, furanyl, 1,2,3-triazolyl, 1,2,4-triazolyl, tetrazolyl, pyrrolidinyl, imidazolyl, and pyridinyl; the substituents are selected from halogen, alkyl, CF₃, OCF₃, cyano, nitro, carboxy, hydroxy, alkoxy; wherein Q₂ is alkyl or heterocycle containing at least one and up to 4 heteroatoms selected from O, S, and N, optionally including their common protecting groups; Q₂ further comprises alkyl or cycloalkyl containing or substituted by functional groups selected from halogen, carboxy, carbamoyl, hydroxy, carbonyl, and cycloalkyl with a site of unsaturation;
R₁₁ and R₁₂ together with the N to which they are attached may join to form a 3 to 8 membered ring; and
R₁₃ is hydrogen, alkyl, alkenyl, alkynyl or cycloalkyl; R₁₁ and R₁₃ together with the N to which they are attached may join to form a 3 to 8 membered ring;
with the provisos that:
when R₁ is C(O)R₆ and when R₆ is unsubstituted phenyl; phenyl monosubstituted or di-substituted by halogen, alkoxy or alkyl; phenyl monosubstituted by alkylthio, alkylamino, dialkylamino, methylenedioxy, alkylsulfonyl, alkanoylamino; naphthalenyl; thiazolyl; biphenyl; thienyl; furanyl; pyridinyl; substituted thiazolyl; substituted biphenyl; thienyl or pyridinyl substituted by one or two of the group consisting of halogen, alkoxy and alkyl, then R₅ may not be unsubstituted phenyl, phenyl monosubstituted by halogen, CF₃, alkoxy, amino, alkyl, alkylamino, dialkylamino, alkanoylamino, N-alkylalkanoylamino, cyano or alkylthio; unsubstituted furanyl; unsubstituted thienyl; unsubstituted pyridinyl; or unsubstituted pyridine-1-oxide; or
when R₁ is hydrogen, fluoro, chloro, bromo, formyl, carboxy, cyano, C(O)alkyl, or CO(O)alkyl, then R₅ may not be phenyl monosubstituted by halogen, CF₃, alkoxy; unsubstituted pyridinyl, pyridine N-oxide, thienyl, furanyl; or pyridinyl, pyridine N-oxide, thienyl and furanyl monosubstituted by alkyl; or
when R₁ is hydrogen, halogen, cyano, formyl, C(O)alkyl, CO(O)alkyl and R₅ is phenyl substituted by R₁₀; and when R₁₀ is N(R₁₃)COR₁₁ where R₁₃ is hydrogen, alkyl, alkenyl, propynyl, or cycloalkylmethyl, then R₁₁ may not be alkyl, cycloalkyl, alkoxy, alkylamino, dialkylamino, —(CH₂)ₙ—O-alkyl, —(CH₂)ₙ—NH-alkyl, —(CH₂)ₙ—N-dialkyl wherein n is an integer 1 to 3 inclusive; or
when R₁ is hydrogen, cyano, chloro, or C(O)R₆ wherein R₆ is phenyl, phenyl monosubstituted by halogen, alkoxy, alkyl, trifluoromethyl, alkylthio, alkylamino, or dialkylamino; thienyl, furanyl, pyridinyl; furanyl, thienyl, pyridinyl monosubstituted by halogen, alkoxy or alkyl, then R₅ may not be meta-substituted phenyl wherein the substituent is an imidazolyl or imidazolyl substituted by 1-3 alkyl groups with the imidazolyl nitrogen directly attached to the phenyl ring; or
when R₁ is C(O)R₆ and R₆ is unsubstituted phenyl; phenyl mono substituted or di-substituted by halogen, alkoxy or alkyl; phenyl monosubstituted by alkylthio, alkylamino, dialkylamino, methylenedioxy, alkylsulfonyl, alkanoylamino; naphthalenyl; thiazolyl; biphenyl; thienyl; furanyl; pyridinyl; substituted thiazolyl; substituted biphenyl; thienyl or pyridinyl substituted by one or two of the group consisting of halogen, alkoxy and alkyl; and when R₅ is phenyl substituted by R₁₀; wherein R₁₀ is N(R₁₃)COR₁₁, R₁₃ is hydrogen, alkenyl, propynyl, or cycloalkylmethyl, then R₁₁ may not be alkyl, cycloalkyl, alkoxy, alkylamino, dialkylamino, —(CH₂)ₙ—O-alkyl, —(CH₂)ₙ—NH-alkyl, —(CH₂)ₙ—N-dialkyl wherein n is an integer 1 to 3 inclusive, said method comprising the steps of reacting the compound of formula (2) 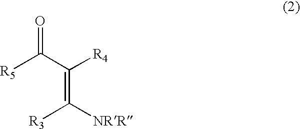
wherein R′ and R″ are independently alkyl in a weak acid or inert solvent;
with the compound of formula (3) 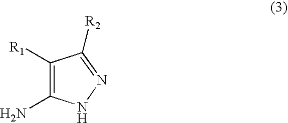
under conditions sufficient to form the compound of Formula I.

12. The method according to claim 1, wherein R₁ is C(O)R₆.

13. The method according to claim 11, wherein R₁ is C(O)R₆, R₂ is H, R₃ is H, and R₄ is H.

14. The method according to claim 11, wherein R₅ of the compound of formula (2) is 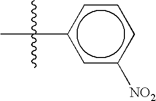
and the method further comprises reducing the nitro group to an amino group after the reaction of the compound of formula (2) with the compound of formula (3) to form the compound of formula (4)

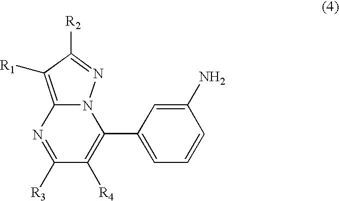

15. The method according to claim 14, further comprising the step of reacting the compound of formula (4) with a compound selected from the group consisting of isocyanate, chloroformate, acid chloride, carboxylic acid, diarylcyanocarbonimidate, and sulfonyl chloride under conditions sufficient to obtain a compound of Formula I wherein R₅ is 3-substituted phenyl.

16. The use of a compound according to claim 1, for regulating expression of p21 in a eukaryotic cell.
